(19) United States
(12) Patent Application Publication
(10) Pub. No.: US 2026/0199273 A1
(43) Pub. Date: Jul. 16, 2026

(54) Compounds for the Treatment of Neuromuscular Disorders

(71) Applicant: NMD PHARMA A/S, Aarhus N (DK)

(72) Inventors: Nicholas Michael KELLY, Aarhus N (DK); Lars J.S. KNUTSEN, Aarhus N (DK); Neerja SARASWAT, Aarhus N (DK); Richard J. HAMLYN, Aarhus N (DK); Farman ULLAH, Aarhus N (DK); Dastagiri DUDEKULA, Aarhus N (DK); Penchal NANDALURU, Aarhus N (DK); Rajesh KUMAR, Aarhus N (DK)

(73) Assignee: NMD PHARMA A/S, Aarhus N (DK)

(21) Appl. No.: 19/135,687

(22) PCT Filed: Dec. 5, 2023
(86) PCT No.: PCT/EP2023/084298

§ 371 Date: Jun. 4, 2025

(30) Foreign Application Priority Data
Dec. 5, 2022 (EP) 22211387.0

(51) Int. Cl. A61K 31/192; A61K 31/335; A61K 31/343; A61K 31/352; A61K 31/382; A61K 31/4035; A61K 31/404; A61K 31/47; A61K 31/4709; A61P 25/14; C07C 63/49; C07C 63/72; C07D 209/08; C07D 209/44; C07D 215/02; C07D 307/79; C07D 311/04; C07D 313/08; C07D 335/06; C07D 409/06

(52) U.S. Cl. CPC A61K 31/192; A61K 31/335; A61K 31/343; A61K 31/352; A61K 31/382; A61K 31/4035; A61K 31/404; A61K 31/47; A61K 31/4709; A61P 25/14; C07C 63/49; C07C 63/72; C07D 209/08; C07D 209/44; C07D 215/02; C07D 307/79; C07D 311/04; C07D 313/08; C07D 335/06; C07D 409/06; C07C 2602/08; C07C 2602/10; C07C 2602/12; C07C 2603/68

(57) ABSTRACT

The present disclosure relates to compounds suitable for treating, ameliorating and/or preventing neuromuscular disorders, including the reversal of drug-induced neuromuscular blockade. The compounds as defined herein can inhibit the CIC-1 ion channel.

### TECHNICAL FIELD

The present disclosure relates to compounds and their use in treating, ameliorating and/or preventing neuromuscular disorders, including the reversal of drug-induced neuromuscular blockade. The compounds as defined herein can inhibit the CIC-1 ion channel. The disclosure further relates to methods of treating, preventing and/or ameliorating neuromuscular disorders, by administering said composition to a person in need thereof.

### BACKGROUND

Walking, breathing, and eye movement are examples of essential everyday physiological activities that are powered by the contractile activity of skeletal muscle.

Skeletal muscles are inherently in a resting state and contractile activity occurs exclusively in response to commands from the central nervous system (CNS). Such neuronal commands take the form of action potentials that travel from the brain to the muscle fibres in several steps. The neuromuscular junction (NMJ) is a highly specialized membrane area on muscle fibres where motor neurons come into close contact with the muscle fibres, and it is at the NMJ where neuronal action potentials are transmitted to muscular action potentials in a one-to-one fashion via synaptic transmission.

Neuromuscular transmission refers to the sequence of cellular events at the NMJ whereby an action potential in the lower motor neuron is transmitted to a corresponding action potential in a muscle fibre (Wood S J, Slater C R. Safety factor at the neuromuscular junction. Prog. Neurobiol. 2001, 64, 393-429). When a neuronal action potential arrives at the pre-synaptic terminal it triggers influx of Ca²⁺ through voltage gated P/Q-type Ca²⁺ channels in the nerve terminal membrane. This influx causes a rise in cytosolic Ca²⁺ in the nerve terminal that triggers exocytosis of acetylcholine (ACh). Released ACh next diffuses across the synaptic cleft to activate nicotinic ACh receptors in the post-synaptic, muscle fibre membrane. Upon activation, ACh receptors convey an excitatory current flow of Na⁺ into the muscle fibre, which results in a local depolarization of the muscle fibre at the NMJ that is known as the endplate potential (EPP). If the EPP is sufficiently large, voltage gated Na⁺ channels in the muscle fibre will activate and an action potential in the muscle fibre will ensue. This action potential then propagates from the NMJ throughout the muscle fibre and triggers release of Ca²⁺ release from the sarcoplasmic reticulum. The released Ca²⁺ activates the contractile proteins within the muscle fibres, thus resulting in contraction of the fibre.

Failure of neuromuscular transmission can arise from both pre-synaptic dysfunction [Lambert Eaton syndrome (Titulaer M J, Lang B, Verschuuren J J. Lambert-Eaton myasthenic syndrome: from clinical characteristics to therapeutic strategies. Lancet Neurol. 2011, 10, 1098-107), amyotrophic lateral sclerosis (Killian J M, Wilfong A A, Burnett L, Appel S H, Boland D. Decremental motor responses to repetitive nerve stimulation in ALS. Muscle Nerve, 1994, 17, 747-754), spinal muscular atrophy (Wadman R I, Vrancken A F, van den Berg L H, van der Pol W L. Dysfunction of the neuromuscular junction in spinal muscular atrophy types 2 and 3. Neurology, 2012, 79, 2050-2055) and as a result of post-synaptic dysfunction as occurs in myasthenia gravis (Le Panse R, Berrih-Aknin S. Autoimmune myasthenia gravis: autoantibody mechanisms and new developments on immune regulation. Curr Opin Neurol., 2013, 26, 569-576)]. Failure to excite and/or propagate action potentials in muscle can also arise from reduced muscle excitability such as in critical illness myopathy (CIM) (Latronico, N., Bolton, C. F. Critical illness polyneuropathy and myopathy: a major cause of muscle weakness and paralysis. Lancet Neurol. 2011, 10, 931-941). In Lambert Eaton syndrome, an autoimmune attack against the pre-synaptic P/Q-type Ca²⁺ channels results in markedly reduced Ca²⁺ influx into the nerve terminal during the pre-synaptic action potential and consequently a reduced release of ACh into the synaptic cleft. In myasthenia gravis, the most common finding is an autoimmune attack on the post-synaptic membrane either against the nicotinic ACh receptors or the musk-receptor in the muscle fibre membrane³. Congenital forms of myasthenia are also known⁵. Common to disorders with neuromuscular transmission failure (Lambert Eaton syndrome, amyotrophic lateral sclerosis, spinal muscular atrophy and myasthenia gravis) is that the current flow generated by ACh receptor activation is markedly reduced, and EPPs therefore become insufficient to trigger muscle fibre action potentials.

Neuromuscular blocking agents also reduce EPP by antagonizing ACh receptors. In CIM with reduced muscle excitability, the EPP may be of normal amplitude but they are still insufficient to trigger muscle fibre action potentials because the membrane potential threshold for action potential excitation has become more depolarized because of loss of function of voltage gated Na⁺ channels in the muscle fibres.

While ACh release (Lambert Eaton, amyotrophic lateral sclerosis, spinal muscular atrophy), ACh receptor function (myasthenia gravis, neuromuscular blockade) and function of voltage gated Na⁺ channels (CIM) are essential components in the synaptic transmission at NMJ, the magnitude of the EPP is also affected by inhibitory currents flowing in the NMJ region of muscle fibres. These currents tend to outbalance excitatory current through ACh receptors and, expectedly, they thereby tend to reduce EPP amplitude. The most important ion channel for carrying such inhibitory membrane currents in muscle fibres is the muscle-specific CIC-1 Cl⁻ ion channel (Kwiecinski H, Lehmann-Horn F, Rudel R. Membrane currents in human intercostal muscle at varied extracellular potassium. Muscle Nerve. 1984, 7, 465-469; Kwiecinski H, Lehmann-Horn F, Rudel R. Drug-induced myotonia in human intercostal muscle. Muscle Nerve. 1988, 11, 576-581; Pedersen, T. H., F. de Paoli, and O. B. Nielsen. Increased excitability of acidified skeletal muscle: role of chloride conductance. J. Gen. Physiol., 2005, 125, 237-246).

ACh esterase (AChE) inhibitors are traditionally used in the treatment of myasthenia gravis. This treatment leads to improvement in most patients but it is associated with side effects, some of which are serious (Mehndiratta M M, Pandey S, Kuntzer T. Acetylcholinesterase inhibitor treatment for myasthenia gravis. Cochrane Database Syst Rev. 2014, Oct. 13; 10). Because ACh is an import neurotransmitter in the autonomic nervous system, delaying its breakdown can lead to gastric discomfort, diarrhea, salivation and muscle cramping. Overdosing is a serious concern as it can lead to muscle paralysis and respiratory failure, a situation commonly referred to as cholinergic crisis. Despite the serious side effects of AChE inhibitors, these drugs are today the treatment of choice for a number of disorders involving neuromuscular impairment. In patients where pyridostigmine (a parasympathomimetic and a reversible ACHE inhibitor) is insufficient, corticosteroid treatment (prednisone) and immunosuppressive treatment (azathioprine) is used. Plasma exchange can be used to obtain a fast but transient improvement.

Unfortunately, all of the currently-employed drug regimens for treatment of myasthenia gravis are associated with deleterious long-term consequences (Howard, J. F. Jr. Adverse drug effects on neuromuscular transmission. Semin Neurol. 1990, 10, 89-102) despite research to identify new treatments (Gilhus, N. E. New England Journal of Medicine, 2016, 375, 2570-2581).

The CIC-1 ion channel (Pedersen, T. H., Riisager, A., Vincenzo de Paoli, F., Chen, T-Y, Nielsen, O. B. Role of physiological CIC-1 Cl⁻ ion channel regulation for the excitability and function of working skeletal muscle. J. Gen. Physiol. 2016, 147, 291-308) is emerging as a target for potential drugs, although its potential has been largely unrealized.

### SUMMARY

The present disclosure comprises a series of compounds that can alleviate disorders of the neuromuscular junction through inhibition of CIC-1 channels.

It has been found that compounds that inhibit CIC-1 ion channels are capable of restoring neuromuscular transmission, as evidenced by the data generated by investigation of the compound set in biological models described herein. These compounds thus constitute a group of potential drugs that can be used to treat and/or ameliorate muscle weakness and/or muscle fatigue in neuromuscular junction disorders caused by disease or by neuromuscular blocking agents.

The present disclosure is directed to CIC-1 ion channel inhibitors with application in the treatment of a range of conditions, such as reversal of neuromuscular block, ALS, spinal muscular atrophy, Charcot-Marie Tooth disease and myasthenic conditions, in which muscle activation by the nervous system is compromised and symptoms of weakness and fatigue are prominent.

In one aspect, the disclosure concerns a compound of Formula (I):

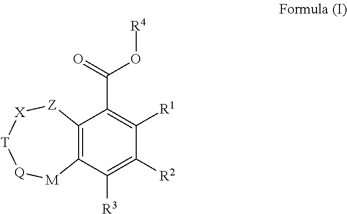

wherein:
   M is CR⁵R⁶, NR⁹, O or S;
   Q is CR⁷R⁶, NR¹⁰, O or S;
   T is absent, CR⁷R⁶, NR¹⁰, O or S;
   X is absent, CR⁷R⁶, NR¹⁰, O or S;
   Z is CR⁸R⁶, NR¹¹, O or S;
wherein no more than one of M, Q, T, X or Z is selected from the group consisting of NR⁹, NR¹⁰, NR¹¹, O or S;
wherein R⁵ and R⁸, or R⁵ and R¹¹, or R⁹ and R⁸ are optionally joined together to form a ring by —CH₂—, —CH₂CH₂- or —CH₂CH₂CH₂—;
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R⁵ is selected from the group consisting of H, deuterium, F, and C₁₋₃ alkyl optionally substituted with one or more, identical or different, substituents R¹², or when R⁵ is linked to R⁸ or R¹¹, then R⁵ is a bond or C₁₋₃ alkanediyl;
   R⁶ is independently selected from the group consisting of H, deuterium, F, C₁₋₃ alkyl substituted with R¹⁴ and C₁₋₃ alkyl optionally substituted with one or more, identical or different, substituents R¹²;
   R⁷ is independently selected from the group consisting of H, deuterium, F and C₁₋₃ alkyl optionally substituted with one or more, identical or different, substituents R¹²;
   R^{a} is selected from the group consisting of H, deuterium, F and C₁₋₃ alkyl optionally substituted with one or more, identical or different, substituents R¹², or when R^{a} is linked to R⁵ or R⁹, then R^{a} is a bond or C₁₋₃ alkanediyl;
   R⁹ is selected from the group consisting of H, C₁₋₅ alkyl optionally substituted with one or more, identical or different, substituents R¹², and C₁₋₃ alkyl substituted with R¹⁴, or when R⁹ is linked to R⁸, then R⁹ is a bond or C₁₋₃ alkanediyl;
   R¹⁰ is independently selected from benzyl optionally substituted with one or more, identical or different, substituents R¹³;
   R¹¹ is selected from the group consisting of H, C₁₋₅ alkyl optionally substituted with one or more, identical or different, substituents R¹², and C₁₋₃ alkyl substituted with R¹⁵, or when R¹¹ is linked to R⁵, then R¹¹ is a bond or C₁₋₃ alkanediyl;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F;
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; and
   R¹⁵ is independently selected from the group consisting of C₃₋₆ cycloalkyl optionally substituted with one or more, identical or different, substituents R¹², C₆₋₁₀ aryl optionally substituted with one or more, identical or different, substituents R¹³ and 5- to 10-membered heteroaryl optionally substituted with one or more, identical or different, substituents R¹³;
or a pharmaceutically acceptable salt, hydrate, polymorph, tautomer, or solvate thereof, provided that the compound is not a compound selected from the group consisting of:
10-chloro-9-methoxybicyclo[5.4.0]undeca-1(7),8,10-triene-8-carboxylic acid; methyl 10-chloro-9-methoxybicyclo[5.4.0]undeca-1(7),8,10-triene-8-carboxylate;
ethyl 10-chloro-9-methoxybicyclo[5.4.0]undeca-1(7),8,10-triene-8-carboxylate; 3-chloro-2-methoxy-5,6,7,8-tetrahydro-1-naphthoic acid; methyl 3-chloro-2-methoxy-5,6,7,8-tetrahydro-1-naphthoate; ethyl 3-chloro-2-methoxy-5,6,7,8-tetrahydro-1-naphthoate; methyl 2-methoxy-3-(trifluoromethyl)-5,6,7,8-tetrahydro-1-naphthoate; 2,3-dimethyl-5,6,7,8-tetrahydro-1-naphthoic acid; 2,3,4-trichloro-5,6,7,8-tetrahydro-1-naphthoic acid; 5,6-dimethyl-4-indancarboxylic acid; methyl 5,6-dimethyl-4-indancarboxylate; 6-chloro-5-methoxy-1,3-dihydro-4-isobenzofuroic acid; methyl 6-chloro-5-methoxy-1,3-dihydro-4-isobenzofuroate; ethyl 6-chloro-5-methoxy-1,3-dihydro-4-isobenzofuroate; 5,6-dichloro-1,3-dihydro-4-isobenzofuroic acid; 5-methoxy-6-methyl-1,3-dihydro-4-isobenzofuroic acid; 5-methoxy-6-methyl-2,3-dihydro-1-benzofuran-4-carboxylic acid; methyl 5-methoxy-6-methyl-2,3-dihydro-1-benzofuran-4-carboxylate; ethyl 5-methoxy-6-methyl-2,3-dihydro-1-benzofuran-4-carboxylate; 5-ethyl-6-methoxy-2,3-dihydro-1-benzofuran-7-carboxylic acid; 5,6-difluoro-2,2-dimethyl-2,3-dihydro-1-benzofuran-7-carboxylic acid; 5-fluoro-6-methyl-2,3-dihydro-1-benzothiophene-4-carboxylic acid; 5-chloro-6-methyl-2,3-dihydro-1-benzothiophene-4-carboxylic acid; 5,6-dichloro-2,3-dihydro-1-benzothiophene-4-carboxylic acid; 6-chloro-5-methyl-2,3-dihydro-1-benzothiophene-7-carboxylic acid; and 5,6-dimethyl-2,3-dihydro-1-benzothiophene-7-carboxylic acid.

In another aspect, the disclosure concerns a compound as defined herein for use in treating, ameliorating and/or preventing a neuromuscular disorder, and/or for use in reversing and/or ameliorating a neuromuscular blockade. In yet another aspect, the disclosure concerns a composition comprising a compound as defined herein.

### Definitions

The terms “C₁₋₃ alkyl” and “C₁₋₅ alkyl” refer to a branched or unbranched alkyl group having from one to three or one to five carbon atoms respectively, including but not limited to methyl, ethyl, prop-1-yl, prop-2-yl, 2-methyl-prop-1-yl, 2-methyl-prop-2-yl, 2,2-dimethyl-prop-1-yl, but-1-yl, but-2-yl, 3-methyl-but-1-yl, 3-methyl-but-2-yl, pent-1-yl, pent-2-yl and pent-3-yl.

The term “alkanediyl” refers to the corresponding derivative of an alkyl group having two bonding sites. Thus, a C₁₋₃ alkanediyl refers to —(CH₂)ₙ—, wherein n is a positive integer from 1 to 3, i.e. including —CH₂—, —CH₂CH₂—, and —CH₂CH₂CH₂-.

The terms “C₂₋₃ alkenyl” and “C₂₋₅ alkenyl” refer to a branched or unbranched alkenyl group having from two to three or two to five carbon atoms respectively, two of which are connected by a double bond, including but not limited to ethenyl, propenyl, isopropenyl, butenyl, isobutenyl, pentenyl and isopentenyl.

The term “C₂₋₅ alkynyl” refers to a branched or unbranched alkynyl group having from two to five carbon atoms, two of which are connected by a triple bond, including but not limited to ethynyl, prop-1-ynyl, prop-2-ynyl, but-1-ynyl, but-2-ynyl, but-3-ynyl, buta-1,3-diynyl, pent-1-ynyl, pent-2-ynyl, pent-3-ynyl, pent-4-ynyl, penta-2,4-diynyl and penta-1,3-diynyl.

The terms “C₃ cycloalkyl”, “C₃₋₅ cycloalkyl” and “C₃₋₆ cycloalkyl” refer to carbocycle groups having three, three to five or three to six carbon atoms respectively including monocyclic or bicyclic carbocycles, including but not limited to cyclopropyl, cyclobutyl, cyclopentyl and cyclohexyl.

The term “5- to 10-membered heteroaryl” refers to a monovalent, aromatic heterocyclic group having one or more heteroatoms, preferably one to three heteroatoms, selected from the group consisting of a nitrogen atom, an oxygen atom, and a sulfur atom. The term “5- to 10-membered heteroaryl” encompasses an aromatic heterocyclic group condensed with an aromatic or non-aromatic carbocyclic ring or an aromatic or non-aromatic heterocyclic ring to form a bicyclic group, and also encompasses an aromatic carbocyclic group condensed with an aromatic or non-aromatic heterocyclic ring to form a bicyclic group. Binding to the heteroaryl may be via a heteroatom or via a carbon atom of the heteroaryl.

In some embodiments, the 5- to 10-membered heteroaryl is a 5-membered heteroaryl. The term “5-membered heteroaryl” refers to a monovalent, aromatic ring system having 5 ring atoms and which contains at least one heteroatom which may be identical or different, said heteroatom being oxygen, nitrogen or sulfur. 5-membered heteroaryls include but are not limited to oxazolyl, thiazolyl, thiadiazolyl, oxadiazolyl, triazolyl, pyrrolyl, thienyl, furanyl, diazolyl, imidazolyl, pyrazolyl, isoxazolyl, isothiazolyl, and tetrazolyl. In one embodiment, the 5-membered heteroaryl is furanyl, such as furan-2-yl. In one embodiment, the 5-membered heteroaryl is thienyl, such as thien-2-yl or thien-3-yl. In one embodiment, the 5-membered heteroaryl is thiazolyl, such as thiazol-2-yl. In one embodiment, the 5-membered heteroaryl is oxazolyl, such as oxazol-2-yl.

In some embodiments, the 5- to 10-membered heteroaryl is a 6-membered heteroaryl. The term “6-membered heteroaryl” refers to a monovalent, aromatic ring system having 6 ring atoms and which contains at least one heteroatom which may be identical or different, said heteroatom being oxygen, nitrogen or sulfur. 6-membered heteroaryls include but are not limited to pyridinyl, pyridazinyl, pyrimidinyl, pyrazinyl, and triazinyl.

In some embodiments, the 5- to 10-membered heteroaryl is a 8-membered heteroaryl. The term “8-membered heteroaryl” refers to a monovalent, aromatic ring system having 8 ring atoms and which contains at least one heteroatom which may be identical or different, said heteroatom being oxygen, nitrogen or sulfur. 8-membered heteroaryls include but are not limited to groups encompassing an aromatic heterocyclic group condensed with an aromatic or non-aromatic carbocyclic ring or an aromatic or non-aromatic heterocyclic ring to form a bicyclic group, and also groups encompassing an aromatic carbocyclic group condensed with an aromatic or non-aromatic heterocyclic ring to form a bicyclic group. 8-membered heteroaryls include but are not limited to 1,4-dihydropyrrolo[3,2-b]pyrrolyl, 1,6-dihydropyrrolo[2,3-b]pyrrolyl, 6H-furo[2,3-b]pyrrole, 4H-furo[2,3-b]pyrrolyl, 6H-thieno[2,3-b]pyrrolyl and 4H-thieno[2,3-b]pyrrolyl.

In some embodiments, the 5- to 10-membered heteroaryl is a 9-membered heteroaryl. The term “9-membered heteroaryl” refers to a monovalent, aromatic ring system having 9 ring atoms and which contains at least one heteroatom which may be identical or different, said heteroatom being oxygen, nitrogen or sulfur. 9-membered heteroaryls include but are not limited to groups encompassing an aromatic heterocyclic group condensed with an aromatic or non-aromatic carbocyclic ring or an aromatic or non-aromatic heterocyclic ring to form a bicyclic group, and also groups encompassing an aromatic carbocyclic group condensed with an aromatic or non-aromatic heterocyclic ring to form a bicyclic group. 9-membered heteroaryls include but are not limited to indolyl, isoindolyl, indolizinyl, indazolyl, benzimidazolyl, azaindolyl, azaindazolyl, pyrazolo[1,5-a]pyrimidinyl, purinyl, benzofuranyl, benzo[b]thiophenyl, benzisoxazolyl, benzthiazolyl, benzisothiazolyl, and benzo[c][1,2,5]thiadiazolyl.

In some embodiments, the 5- to 10-membered heteroaryl is a 10-membered heteroaryl. The term “10-membered heteroaryl” refers to a monovalent, aromatic ring system having 10 ring atoms and which contains at least one heteroatom which may be identical or different, said heteroatom being oxygen, nitrogen or sulfur. 10-membered heteroaryls include but are not limited to groups encompassing an aromatic heterocyclic group condensed with an aromatic or non-aromatic carbocyclic ring or an aromatic or non-aromatic heterocyclic ring to form a bicyclic group, and also groups encompassing an aromatic carbocyclic group condensed with an aromatic or non-aromatic heterocyclic ring to form a bicyclic group. 10-membered heteroaryls include but are not limited to quinolinyl, isoquinolinyl, 4H-quinolizinyl, quinoxalinyl, phthalazinyl, quinazolinyl, cinnolinyl, 1,8-naphthyridinyl, pyrido[3,2-d]pyrimidinyl, pyrido[4,3-dlpyrimidinyl, pyrido[3,4-b]pyrazinyl, pyrido[2,3-b]pyrazinyl and pteridinyl.

The term “aryl” as used herein represents a mono-, bicyclic, or multicyclic carbocyclic ring system having one or two aromatic rings. The term “C₆-.o aryl” refers to an aryl group having 6 to 10 carbon atoms, for example a phenyl group or a naphthyl group.

The term “half-life” as used herein is the time it takes for the compound to lose one-half of its pharmacologic activity. The term “plasma half-life” is the time that it takes the compound to lose one-half of its pharmacologic activity in the blood plasma.

The term “treatment” refers to the combating of a disease or disorder. “Treatment” or “treating,” as used herein, includes any desirable effect on the symptoms or pathology of a disease or condition as described herein, and may include even minimal changes or improvements in one or more measurable markers of the disease or condition being treated. “Treatment” or “treating” does not necessarily indicate complete eradication or cure of the disease or condition, or associated symptoms thereof. In some embodiments, the term “treatment” encompasses amelioration and prevention.

The term “amelioration” refers to moderation in the severity of the symptoms of a disease or condition. Improvement in a patient's condition, or the activity of making an effort to correct, or at least make more acceptable, conditions that are difficult to endure related to patient's conditions is considered “ameliorative” treatment.

The term “prevent” or “preventing” refers to precluding, averting, obviating, forestalling, stopping, or hindering something from happening, especially by advance action.

The term “reversal” or “reversing” refers to the ability of a compound to restore nerve-stimulated force in skeletal muscle exposed either ex vivo or in vivo to a non-depolarizing neuromuscular blocking agent or another pharmaceutical that is able to depress neuromuscular transmission

The term “non-depolarizing blockers” refers to pharmaceutical agents that antagonize the activation of acetylcholine receptors at the post-synaptic muscle fibre membrane by blocking the acetylcholine binding site on the receptor. These agents are used to block neuromuscular transmission and induce muscle paralysis in connection with surgery.

The term “ester hydrolysing reagent” refers to a chemical reagent which is capable of converting an ester functional group to a carboxylic acid with elimination of the alcohol moiety of the original ester, including but not limited to acid, base, a fluoride source, PBr₃, PCl₃ and lipase enzymes.

The term “total membrane conductance (Gm)” is the electrophysiological measure of the ability of ions to cross the muscle fibre surface membrane. It reflects the function of ion channels that are active in resting muscle fibres of which CIC-1 is known to contribute around 80% in most animal species.

### DETAILED DESCRIPTION

### Compounds

It is within the scope of the present disclosure to provide a compound for use in treating, ameliorating and/or preventing neuromuscular disorders that reduce neuromuscular function. As disclosed herein, inhibition of CIC-1 improves or restores neuromuscular function. The compounds of the present disclosure comprise compounds capable of inhibiting the CIC-1 channel thereby improving or restoring neuromuscular function.

In one aspect, the disclosure concerns a compound of Formula (I):

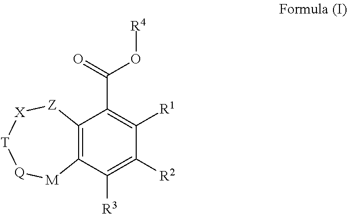

wherein:
   M is CR⁵R⁶, NR⁹, O or S;
   Q is CR⁷R⁶, NR¹⁰, O or S;
   T is absent, CR⁷R⁶, NR¹⁰, O or S;
   X is absent, CR⁷R⁶, NR¹⁰, 0 or S;
   Z is CR⁸R⁶, NR¹¹, O or S;
wherein no more than one of M, Q, T, X or Z is selected from the group consisting of NR⁹, NR¹⁰, NR¹¹, O or S;
wherein R⁵ and R⁸, or R⁵ and R¹¹, or R⁹ and R⁸ are optionally joined together to form a ring by —CH₂—, —CH₂CH₂- or —CH₂CH₂CH₂—;
   R¹ is selected from the group consisting of H; F; Cl; Br; I; C₁₋₃ alkyl optionally substituted with one or more, identical or different, substituents R¹²; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of H; F; Cl; Br; I; C₁₋₃ alkyl optionally substituted with one or more, identical or different, substituents R¹²; —OC₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R⁵ is selected from the group consisting of H, deuterium, F, and C₁₋₃ alkyl optionally substituted with one or more, identical or different, substituents R¹², or when R⁵ is linked to R^{a} or R¹¹, then R⁵ is a bond or C₁₋₃ alkanediyl;
   R⁶ is independently selected from the group consisting of H, deuterium, F, C₁₋₃ alkyl substituted with R¹⁴ and C₁₋₃ alkyl optionally substituted with one or more, identical or different, substituents R¹²;
   R⁷ is independently selected from the group consisting of H, deuterium, F and C₁₋₃ alkyl optionally substituted with one or more, identical or different, substituents R¹²;
   R^{a} is selected from the group consisting of H, deuterium, F and C₁₋₃ alkyl optionally substituted with one or more, identical or different, substituents R¹², or when R^{a} is linked to R⁵ or R⁹, then R^{a} is a bond or C₁₋₃ alkanediyl;
   R⁹ is selected from the group consisting of H, C₁₋₅ alkyl optionally substituted with one or more, identical or different, substituents R¹², and C₁₋₃ alkyl substituted with R¹⁴, or when R⁹ is linked to R⁸, then R⁹ is a bond or C₁₋₃ alkanediyl;
   R¹⁰ is independently selected from benzyl optionally substituted with one or more, identical or different, substituents R¹³;
   R¹¹ is selected from the group consisting of H, C₁₋₅ alkyl optionally substituted with one or more, identical or different, substituents R¹², and C₁₋₃ alkyl substituted with R¹⁵, or when R¹¹ is linked to R⁵, then R¹¹ is a bond or C₁₋₃ alkanediyl;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F;
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; and
   R¹⁵ is independently selected from the group consisting of C₃₋₆ cycloalkyl optionally substituted with one or more, identical or different, substituents R¹², aryl optionally substituted with one or more, identical or different, substituents R¹³ and 5- to 10-membered heteroaryl optionally substituted with one or more, identical or different, substituents R¹³; or a pharmaceutically acceptable salt, hydrate, polymorph, tautomer, or solvate thereof.

In one aspect, the present invention relates to a compound of Formula (I):

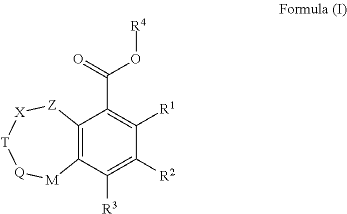

M is CR⁵R⁶, NR⁹, O or S;
Q is CR⁷R⁶, NR¹⁰, O or S;
T is absent, CR⁷R⁶, NR¹⁰, O or S;
X is absent, CR⁷R⁶, NR¹⁰, O or S;
Z is CR⁸R⁶, NR¹, O or S;
wherein no more than one of M, Q, T, X or Z is selected from the group consisting of NR⁹, NR¹⁰, NR¹¹, O or S;
wherein R⁵ and R⁸, or R⁵ and R¹¹, or R⁹ and R⁸ are optionally joined together to form a ring by —CH₂—, —CH₂CH₂- or —CH₂CH₂CH₂—;
   R¹ is selected from the group consisting of H; F; Cl; Br; I; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of H; F; Cl; Br; I; C₁₋₃ alkyl optionally substituted with one or more, identical or different, substituents R¹²; —OC₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R⁵ is selected from the group consisting of H, deuterium, F, a bond and C₁₋₃ alkyl optionally substituted with one or more, identical or different, substituents R¹²;
   R⁶ is independently selected from the group consisting of H, deuterium, F and C₁₋₃ alkyl optionally substituted with one or more, identical or different, substituents R¹²;
   R⁷ is independently selected from the group consisting of H, deuterium, F and C₁₋₃ alkyl optionally substituted with one or more, identical or different, substituents R¹²;
   R^{a} is selected from the group consisting of H, deuterium, a bond, F and C₁₋₃ alkyl optionally substituted with one or more, identical or different, substituents R¹²;
   R⁹ is selected from the group consisting of H, C₁₋₅ alkyl optionally substituted with one or more, identical or different, substituents R¹², a bond and benzyl optionally substituted with one or more, identical or different, substituents R¹³;
   R¹⁰ is independently selected from benzyl optionally substituted with one or more, identical or different, substituents R¹³;
   R¹¹ is selected from the group consisting of H, C₁₋₅ alkyl optionally substituted with one or more, identical or different, substituents R¹², a bond and benzyl optionally substituted with one or more, identical or different, substituents R¹³;
   R¹² is independently selected from the group consisting of deuterium, F and OMe; and
   R¹³ is independently selected from the group consisting of deuterium, methoxy, nitro, cyano, Cl, Br, I, and F;
or a pharmaceutically acceptable salt, hydrate, polymorph, tautomer, or solvate thereof.

In one aspect, the present invention relates to a compound of Formula (I):

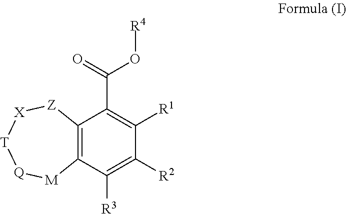

wherein:
   M is CR⁵R⁶, NR⁹, O or S;
   Q is CR⁷R⁶, NR¹⁰, O or S;
   T is absent, CR⁷R⁶, NR¹⁰, O or S;
   X is absent, CR⁷R⁶, NR¹⁰, O or S;
   Z is CR⁸R⁶, NR¹, O or S;
wherein no more than one of M, Q, T, X or Z is selected from the group consisting of NR⁹, NR¹⁰, NR¹¹, O or S;
wherein R⁵ and R⁸, or R⁵ and R¹¹, or R⁹ and R⁸ are optionally joined together to form a ring by —CH₂—, —CH₂CH₂- or —CH₂CH₂CH₂—;
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R⁵ is selected from the group consisting of H, deuterium, F, a bond and C₁₋₃ alkyl optionally substituted with one or more, identical or different, substituents R¹²;
   R⁶ is independently selected from the group consisting of H, deuterium, F and C₁₋₃ alkyl optionally substituted with one or more, identical or different, substituents R¹²;
   R⁷ is independently selected from the group consisting of H, deuterium, F and C₁₋₃ alkyl optionally substituted with one or more, identical or different, substituents R¹²;
   R^{a} is selected from the group consisting of H, deuterium, a bond, F and C₁₋₃ alkyl optionally substituted with one or more, identical or different, substituents R¹²;
   R⁹ is selected from the group consisting of H, C₁₋₅ alkyl optionally substituted with one or more, identical or different, substituents R¹², a bond and benzyl optionally substituted with one or more, identical or different, substituents R¹³;
   R¹⁰ is independently selected from benzyl optionally substituted with one or more, identical or different, substituents R¹³;
   R¹¹ is selected from the group consisting of H, C₁₅ alkyl optionally substituted with one or more, identical or different, substituents R¹², a bond and benzyl optionally substituted with one or more, identical or different, substituents R¹³;
   R¹² is independently selected from the group consisting of deuterium, F and OMe; and
   R¹³ is independently selected from the group consisting of deuterium, methoxy, nitro, cyano, Cl, Br, I, and F;
or a pharmaceutically acceptable salt, hydrate, polymorph, tautomer, or solvate thereof provided that:
   when M, Q, T, X and Z are CH₂, R¹ is OMe, R² is Cl and R³ is H then R⁴ is not H, Me or Et;
   when M, Q, T and Z are CH₂, X is absent, R¹ is OMe, R² is Cl and R³ is H then R⁴ is not H, Me or Et;
   when M, Q, T and Z are CH₂, X is absent, R¹ is OMe, R² is CF₃ and R³ is H then R⁴ is not Me;
   when M, Q, T and Z are CH₂, X is absent, R¹ is Me, R² is Me and R³ is H then R⁴ is not H;
   when M, Q, T and Z are CH₂, X is absent, R¹ is Cl, R² is Cl and R³ is Cl then R⁴ is not H;
   when M, Q and Z are CH₂, T and X are absent, R¹ is Me, R² is Me and R³ is H then R⁴ is not H or Me;
   when M and Z are CH₂, Q is 0, T and X are absent, R¹ is OMe, R² is Cl and R³ is H then R⁴ is not H, Me or Et;
   when M and Z are CH₂, Q is 0, T and X are absent, R¹ is Cl, R² is C, and R³ is H then R⁴ is not H;
   when M and Z are CH₂, Q is 0, T and X are absent, R¹ is OMe, R² is Me, and R³ is H then R⁴ is not H;
   when Q and Z are CH₂, M is 0, T and X are absent, R¹ is OMe, R² is Me and R³ is H then R⁴ is not H, Me or Et;
   when M and Q are CH₂, Z is 0, T and X are absent, R¹ is OMe, R² is Et and R³ is H then R⁴ is not H;
   when M is CH₂, Q is CMe₂, Z is 0, T and X are absent, R¹ and R² are F and R³ is H then R⁴ is not H;
   when Q and Z are CH₂, M is S, T and X are absent, R¹ is Cl or F, R² is Me and R³ is H then R⁴ is not H;
   when Q and Z are CH₂, M is S, T and X are absent, R¹ is Cl, R² is Cl and R³ is H then R⁴ is not H; and
   when Q and M are CH₂, Z is S, T and X are absent, R¹ is Cl or Me, R² is Me and R³ is H then R⁴ is not H.

In one embodiment, R¹ is selected from the group consisting of F; Cl; Br; I; C₁₋₃ alkyl optionally substituted with one or more, identical or different, substituents R¹²; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹².

In one embodiment, R² is selected from the group consisting of H; F; Cl; Br; I; C₁₋₃ alkyl optionally substituted with one or more, identical or different, substituents R¹² and C₃ cycloalkyl optionally substituted with one or more, identical or different, substituents R¹².

In one embodiment, when R¹ is OMe then R² is not OMe. In one embodiment, when M, Q, T, X and Z are CH₂, R¹ is OMe, R² is Cl and R³ is H then R⁴ is not H, Me or Et. In one embodiment, the compound is not 10-chloro-9-methoxybicyclo[5.4.0]undeca-1(7),8,10-triene-8-carboxylic acid, methyl 10-chloro-9-methoxybicyclo[5.4.0]undeca-1(7),8,10-triene-8-carboxylate, or ethyl 10-chloro-9-methoxybicyclo[5.4.0]undeca-1(7),8,10-triene-8-carboxylate. In one embodiment, when M, Q, T and Z are CH₂, X is absent, R¹ is OMe, R² is Cl and R³ is H then R⁴ is not H, Me or Et. In one embodiment, the compound is not 3-chloro-2-methoxy-5,6,7,8-tetrahydro-1-naphthoic acid, methyl 3-chloro-2-methoxy-5,6,7,8-tetrahydro-1-naphthoate, or ethyl 3-chloro-2-methoxy-5,6,7,8-tetrahydro-1-naphthoate. In one embodiment, when M, Q, T and Z are CH₂, X is absent, R¹ is OMe, R² is CF₃ and R³ is H then R⁴ is not Me. In one embodiment, the compound is not methyl 2-methoxy-3-(trifluoromethyl)-5,6,7,8-tetrahydro-1-naphthoate. In one embodiment, when M, Q, T and Z are CH₂, X is absent, R¹ is Me, R² is Me and R³ is H then R⁴ is not H. In one embodiment, the compound is not 2,3-dimethyl-5,6,7,8-tetrahydro-1-naphthoic acid. In one embodiment, when M, Q and Z are CH₂, T and X are absent, R¹ is Me, R² is Me and R³ is H then R⁴ is not H or Me. In one embodiment, the compound is not 5,6-dimethyl-4-indancarboxylic acid, or methyl 5,6-dimethyl-4-indancarboxylate. In one embodiment, when M and Z are CH₂, Q is 0, T and X are absent, R¹ is OMe, R² is Cl and R³ is H then R⁴ is not H, Me or Et. In one embodiment, the compound is not 6-chloro-5-methoxy-1,3-dihydro-4-isobenzofuroic acid, methyl 6-chloro-5-methoxy-1,3-dihydro-4-isobenzofuroate, or ethyl 6-chloro-5-methoxy-1,3-dihydro-4-isobenzofuroate. In one embodiment, when M and Z are CH₂, Q is 0, T and X are absent, R¹ is Cl, R² is Cl, and R³ is H then R⁴ is not H. In one embodiment, the compound is not 5,6-dichloro-1,3-dihydro-4-isobenzofuroic acid. In one embodiment, when M, Q, T and Z are CH₂, X is absent, R¹ is Cl, R² is Cl and R³ is Cl then R⁴ is not H. In one embodiment, the compound is not 2,3,4-trichloro-5,6,7,8-tetrahydro-1-naphthoic acid. In one embodiment, when M and Z are CH₂, Q is 0, T and X are absent, R¹ is OMe, R² is Me, and R³ is H then R⁴ is not H. In one embodiment, the compound is not 5-methoxy-6-methyl-1,3-dihydro-4-isobenzofuroic acid. In one embodiment, when Q and Z are CH₂, M is 0, T and X are absent, R¹ is OMe, R² is Me and R³ is H then R⁴ is not H, Me or Et. In one embodiment, the compound is not 5-methoxy-6-methyl-2,3-dihydro-1-benzofuran-4-carboxylic acid, methyl 5-methoxy-6-methyl-2,3-dihydro-1-benzofuran-4-carboxylate, or ethyl 5-methoxy-6-methyl-2,3-dihydro-1-benzofuran-4-carboxylate. In one embodiment, when M and Q are CH₂, Z is 0, T and X are absent, R¹ is OMe, R² is Et and R³ is H then R⁴ is not H. In one embodiment, the compound is not 5-ethyl-6-methoxy-2,3-dihydro-1-benzofuran-7-carboxylic acid. In one embodiment, when M is CH₂, Q is CMe₂, Z is 0, T and X are absent, R¹ and R² are F and R³ is H then R⁴ is not H. In one embodiment, the compound is not 5,6-difluoro-2,2-dimethyl-2,3-dihydro-1-benzofuran-7-carboxylic acid. In one embodiment, when Q and Z are CH₂, M is S, T and X are absent, R¹ is C or F, R² is Me and R³ is H then R⁴ is not H. In one embodiment, the compound is not 5-fluoro-6-methyl-2,3-dihydro-1-benzothiophene-4-carboxylic acid, or 5-chloro-6-methyl-2,3-dihydro-1-benzothiophene-4-carboxylic acid. In one embodiment, when Q and Z are CH₂, M is S, T and X are absent, R¹ is Cl, R² is Cl and R³ is H then R⁴ is not H. In one embodiment, the compound is not 5,6-dichloro-2,3-dihydro-1-benzothiophene-4-carboxylic acid. In one embodiment, when Q and M are CH₂, Z is S, T and X are absent, R¹ is Cl or Me, R² is Me and R³ is H then R⁴ is not H. In one embodiment, the compound is not 6-chloro-5-methyl-2,3-dihydro-1-benzothiophene-7-carboxylic acid, or 5,6-dimethyl-2,3-dihydro-1-benzothiophene-7-carboxylic acid.

In one aspect, the disclosure concerns a compound of Formula (I):

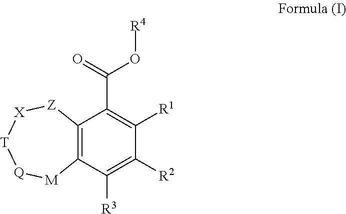

wherein:
   M is CR⁵R⁶, NR⁹, O or S;
   Q is CR⁷R⁶, NR¹⁰, O or S;
   T is absent, CR⁷R⁶, NR¹⁰, O or S;
   X is absent, CR⁷R⁶, NR¹⁰, O or S;
   Z is CR⁸R⁶, NR¹¹, O or S;
wherein no more than one of M, Q, T, X or Z is selected from the group consisting of NR⁹, NR¹⁰, NR¹¹, O or S;
wherein R⁵ and R⁸, or R⁵ and R¹¹, or R⁹ and R⁸ are optionally joined together to form a ring by —CH₂—, —CH₂CH₂- or —CH₂CH₂CH₂—;
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R⁵ is selected from the group consisting of H, deuterium, F, and C₁₋₃ alkyl optionally substituted with one or more, identical or different, substituents R¹² or when R⁵ is linked to R^{a} or R¹¹, then R⁵ is a bond or C₁₋₃ alkanediyl;
   R⁶ is independently selected from the group consisting of H, deuterium, F, C₁₋₃ alkyl substituted with R¹⁴ and C₁₋₃ alkyl optionally substituted with one or more, identical or different, substituents R¹²;
   R⁷ is independently selected from the group consisting of H, deuterium, F and C₁₋₃ alkyl optionally substituted with one or more, identical or different, substituents R¹²;
   R^{a} is selected from the group consisting of H, deuterium, F and C₁₋₃ alkyl optionally substituted with one or more, identical or different, substituents R¹², or when R^{a} is linked to R⁵ or R⁹, then R^{a} is a bond or C₁₋₃ alkanediyl;
   R⁹ is selected from the group consisting of H, C₁₋₅ alkyl optionally substituted with one or more, identical or different, substituents R¹², and C₁₋₃ alkyl substituted with R¹⁴, or when R⁹ is linked to R⁸, then R⁹ is a bond or C₁₋₃ alkanediyl;
   R¹⁰ is independently selected from benzyl optionally substituted with one or more, identical or different, substituents R¹³;
   R¹¹ is selected from the group consisting of H, C₁₋₅ alkyl optionally substituted with one or more, identical or different, substituents R¹², and C₁₋₃ alkyl substituted with R¹⁵, or when R¹¹ is linked to R⁵, then R¹¹ is a bond or C₁₋₃ alkanediyl;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F;
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; and
   R¹⁵ is independently selected from the group consisting of C₃₋₆ cycloalkyl optionally substituted with one or more, identical or different, substituents R¹², C₆10 aryl optionally substituted with one or more, identical or different, substituents R¹³ and 5- to 10-membered heteroaryl optionally substituted with one or more, identical or different, substituents R¹³;
or a pharmaceutically acceptable salt, hydrate, polymorph, tautomer, or solvate thereof provided that:
a) when M, Q, T, X and Z are CH₂, R¹ is OMe, R² is Cl and R³ is H then R⁴ is not H, Me or Et;
when M, Q, T and Z are CH₂, X is absent, R¹ is OMe, R² is Cl and R³ is H then R⁴ is not H, Me or Et;
when M, Q, T and Z are CH₂, X is absent, R¹ is OMe, R² is CF₃ and R³ is H then R⁴ is not Me;
when M, Q, T and Z are CH₂, X is absent, R¹ is Me, R² is Me and R³ is H then R⁴ is not H;
when M, Q and Z are CH₂, T and X are absent, R¹ is Me, R² is Me and R³ is H then R⁴ is not H or Me;
when M and Z are CH₂, Q is 0, T and X are absent, R¹ is OMe, R² is C and R³ is H then R⁴ is not H, Me or Et;
when M and Z are CH₂, Q is 0, T and X are absent, R¹ is Cl, R² is Cl, and R³ is H then R⁴ is not H;
when M and Z are CH₂, Q is 0, T and X are absent, R¹ is OMe, R² is Me, and R³ is H then R⁴ is not H;
when Q and Z are CH₂, M is 0, T and X are absent, R¹ is OMe, R² is Me and R³ is H then R⁴ is not H, Me or Et;
when M and Q are CH₂, Z is 0, T and X are absent, R¹ is OMe, R² is Et and R³ is H then R⁴ is not H;
when M is CH₂, Q is CMe₂, Z is 0, T and X are absent, R¹ and R² are F and R³ is H then R⁴ is not H;
when Q and Z are CH₂, M is S, T and X are absent, R¹ is Cl or F, R² is Me and R³ is H then R⁴ is not H;
when Q and Z are CH₂, M is S, T and X are absent, R¹ is Cl, R² is Cl and R³ is H then R⁴ is not H; and when Q and M are CH₂, Z is S, T and X are absent, R¹ is Cl or Me, R² is Me and R³ is H then R⁴ is not H; or
b) the compound is not a compound selected from the group consisting of:
10-chloro-9-methoxybicyclo[5.4.0]undeca-1(7),8,10-triene-8-carboxylic acid;
methyl 10-chloro-9-methoxybicyclo[5.4.0]undeca-1(7),8,10-triene-8-carboxylate;
ethyl 10-chloro-9-methoxybicyclo[5.4.0]undeca-1(7),8,10-triene-8-carboxylate;
3-chloro-2-methoxy-5,6,7,8-tetrahydro-1-naphthoic acid;
methyl 3-chloro-2-methoxy-5,6,7,8-tetrahydro-1-naphthoate;
ethyl 3-chloro-2-methoxy-5,6,7,8-tetrahydro-1-naphthoate;
methyl 2-methoxy-3-(trifluoromethyl)-5,6,7,8-tetrahydro-1-naphthoate;
2,3-dimethyl-5,6,7,8-tetrahydro-1-naphthoic acid;
2,3,4-trichloro-5,6,7,8-tetrahydro-1-naphthoic acid;
5,6-dimethyl-4-indancarboxylic acid;
methyl 5,6-dimethyl-4-indancarboxylate;
6-chloro-5-methoxy-1,3-dihydro-4-isobenzofuroic acid;
methyl 6-chloro-5-methoxy-1,3-dihydro-4-isobenzofuroate;
ethyl 6-chloro-5-methoxy-1,3-dihydro-4-isobenzofuroate;
5,6-dichloro-1,3-dihydro-4-isobenzofuroic acid;
5-methoxy-6-methyl-1,3-dihydro-4-isobenzofuroic acid;
5-methoxy-6-methyl-2,3-dihydro-1-benzofuran-4-carboxylic acid;
methyl 5-methoxy-6-methyl-2,3-dihydro-1-benzofuran-4-carboxylate;
ethyl 5-methoxy-6-methyl-2,3-dihydro-1-benzofuran-4-carboxylate;
5-ethyl-6-methoxy-2,3-dihydro-1-benzofuran-7-carboxylic acid;
5,6-difluoro-2,2-dimethyl-2,3-dihydro-1-benzofuran-7-carboxylic acid;
5-fluoro-6-methyl-2,3-dihydro-1-benzothiophene-4-carboxylic acid;
5-chloro-6-methyl-2,3-dihydro-1-benzothiophene-4-carboxylic acid;
5,6-dichloro-2,3-dihydro-1-benzothiophene-4-carboxylic acid;
6-chloro-5-methyl-2,3-dihydro-1-benzothiophene-7-carboxylic acid; and
5,6-dimethyl-2,3-dihydro-1-benzothiophene-7-carboxylic acid.

In one aspect, the disclosure concerns a compound of Formula (I):

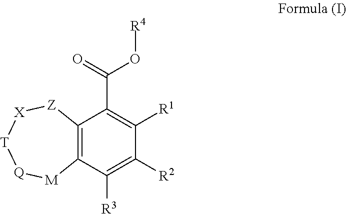

wherein:
M is CR⁵R⁶, NR⁹, O or S;
Q is CR⁷R⁶ or NR¹⁰;
T is absent, CR⁷R⁶ or NR¹⁰;
X is absent, CR⁷R⁶ or NR¹⁰;
Z is CR⁸R⁶, NR¹¹, O or S;
wherein no more than one of M, Q, T, X or Z is selected from the group consisting of NR⁹, NR¹⁰, NR¹¹, O or S;
wherein R⁵ and R⁸, or R⁵ and R¹¹, or R⁹ and R⁸ are optionally joined together to form a ring by —CH₂—, —CH₂CH₂- or —CH₂CH₂CH₂—;
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R⁵ is selected from the group consisting of H, deuterium, F, and C₁₋₃ alkyl optionally substituted with one or more, identical or different, substituents R¹², or when R⁵ is linked to R⁸ or R¹¹, then R⁵ is a bond or C₁₋₃ alkanediyl;
   R⁶ is independently selected from the group consisting of H, deuterium, F, C₁₋₃ alkyl substituted with R¹⁴ and C₁₋₃ alkyl optionally substituted with one or more, identical or different, substituents R¹²;
   R⁷ is independently selected from the group consisting of H, deuterium, F and C₁₋₃ alkyl optionally substituted with one or more, identical or different, substituents R¹²;
   R⁸ is selected from the group consisting of H, deuterium, F and C₁₋₃ alkyl optionally substituted with one or more, identical or different, substituents R¹², or when R⁸ is linked to R⁵ or R⁹, then R⁸ is a bond or C₁₋₃ alkanediyl;
   R⁹ is selected from the group consisting of H, C₁₋₅ alkyl optionally substituted with one or more, identical or different, substituents R¹², and C₁₋₃ alkyl substituted with R¹⁴, or when R⁹ is linked to R⁸, then R⁹ is a bond or C₁₋₃ alkanediyl;
   R¹⁰ is independently selected from benzyl optionally substituted with one or more, identical or different, substituents R¹³:
   R¹¹ is selected from the group consisting of H, C₁₋₅ alkyl optionally substituted with one or more, identical or different, substituents R¹², and C₁₋₃ alkyl substituted with R¹⁵, or when R¹¹ is linked to R⁵, then R¹¹ is a bond or C₁₋₃ alkanediyl;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F;
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; and
   R¹⁵ is independently selected from the group consisting of C₃₋₆ cycloalkyl optionally substituted with one or more, identical or different, substituents R¹², C₆10 aryl optionally substituted with one or more, identical or different, substituents R¹³ and 5- to 10-membered heteroaryl optionally substituted with one or more, identical or different, substituents R¹³;
or a pharmaceutically acceptable salt, hydrate, polymorph, tautomer, or solvate thereof provided that:
   when M, Q, T, X and Z are CH₂, R¹ is OMe, R² is Cl and R³ is H then R⁴ is not H, Me or Et;
   when M, Q, T and Z are CH₂, X is absent, R¹ is OMe, R² is Cl and R³ is H then R⁴ is not H, Me or Et;
   when M, Q, T and Z are CH₂, X is absent, R¹ is OMe, R² is CF₃ and R³ is H then R⁴ is not Me;
   when M, Q, T and Z are CH₂, X is absent, R¹ is Me, R² is Me and R³ is H then R⁴ is not H;
   when M, Q and Z are CH₂, T and X are absent, R¹ is Me, R² is Me and R³ is H then R⁴ is not H or Me;
   when Q and Z are CH₂, M is 0, T and X are absent, R¹ is OMe, R² is Me and R³ is H then R⁴ is not H, Me or Et;
   when M and Q are CH₂, Z is 0, T and X are absent, R¹ is OMe, R² is Et and R³ is H then R⁴ is not H;
   when M is CH₂, Q is CMe₂, Z is 0, T and X are absent, R¹ and R² are F and R³ is H then R⁴ is not H;
   when Q and Z are CH₂, M is S, T and X are absent, R¹ is C or F, R² is Me and R³ is H then R⁴ is not H;
   when Q and Z are CH₂, M is S, T and X are absent, R¹ is Cl, R² is Cl and R³ is H then R⁴ is not H; and
   when Q and M are CH₂, Z is S, T and X are absent, R¹ is Cl or Me, R² is Me and R³ is H then R⁴ is not H.

In one aspect, the disclosure concerns a compound of Formula (I):

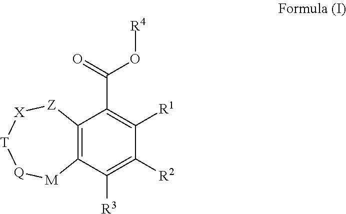

wherein:
   M is CR⁵R⁶, NR⁹, O or S;
   Q is CR⁷R⁶, NR¹⁰, O or S;
   T is absent, CR⁷R⁶, NR¹⁰, O or S;
   X is absent, CR⁷R⁶, NR¹⁰, O or S;
   Z is CR⁸R⁶, NR¹¹, O or S;
wherein no more than one of M, Q, T, X or Z is selected from the group consisting of NR⁹, NR¹⁰, NR¹¹, O or S;
wherein R⁵ and R⁸, or R⁵ and R¹¹, or R⁹ and R⁸ are optionally joined together to form a ring by —CH₂—, —CH₂CH₂- or —CH₂CH₂CH₂—;
   R¹ is selected from the group consisting of F; CI; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R⁵ is selected from the group consisting of H, deuterium, F, and C₁₋₃ alkyl optionally substituted with one or more, identical or different, substituents R¹², or when R⁵ is linked to R^{a} or R¹¹, then R⁵ is a bond or C₁₋₃ alkanediyl;
   R⁶ is independently selected from the group consisting of H, deuterium, F, C₁₋₃ alkyl substituted with R¹⁴ and C₁₋₃ alkyl optionally substituted with one or more, identical or different, substituents R¹²;
   R⁷ is independently selected from the group consisting of H, deuterium, F and C₁₋₃ alkyl optionally substituted with one or more, identical or different, substituents R¹²;
   R^{a} is selected from the group consisting of H, deuterium, F and C₁₋₃ alkyl optionally substituted with one or more, identical or different, substituents R¹², or when R^{a} is linked to R⁵ or R⁹, then R^{a} is a bond or C₁₋₃ alkanediyl;
   R⁹ is selected from the group consisting of H, C₁₋₅ alkyl optionally substituted with one or more, identical or different, substituents R¹², and C₁₃ alkyl substituted with R¹⁴, or when R⁹ is linked to R⁸, then R⁹ is a bond or C₁₋₃ alkanediyl;
   R¹⁰ is independently selected from benzyl optionally substituted with one or more, identical or different, substituents R¹³;
   R¹¹ is selected from the group consisting of H, C₁₋₅ alkyl optionally substituted with one or more, identical or different, substituents R¹², and C₁₋₃ alkyl substituted with R¹⁵, or when R¹¹ is linked to R⁵, then R¹¹ is a bond or C₁₋₃ alkanediyl;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F;
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; and
   R¹⁵ is independently selected from the group consisting of C₃₋₆ cycloalkyl optionally substituted with one or more, identical or different, substituents R¹², C₆10 aryl optionally substituted with one or more, identical or different, substituents R¹³ and 5- to 10-membered heteroaryl optionally substituted with one or more, identical or different, substituents R¹³;
or a pharmaceutically acceptable salt, hydrate, polymorph, tautomer, or solvate thereof, provided that the compound is not a compound selected from the group consisting of:
10-chloro-9-methoxybicyclo[5.4.0]undeca-1(7),8,10-triene-8-carboxylic acid;
methyl 10-chloro-9-methoxybicyclo[5.4.0]undeca-1(7),8,10-triene-8-carboxylate;
ethyl 10-chloro-9-methoxybicyclo[5.4.0]undeca-1(7),8,10-triene-8-carboxylate;
3-chloro-2-methoxy-5,6,7,8-tetrahydro-1-naphthoic acid;
methyl 3-chloro-2-methoxy-5,6,7,8-tetrahydro-1-naphthoate;
ethyl 3-chloro-2-methoxy-5,6,7,8-tetrahydro-1-naphthoate;
methyl 2-methoxy-3-(trifluoromethyl)-5,6,7,8-tetrahydro-1-naphthoate;
2,3-dimethyl-5,6,7,8-tetrahydro-1-naphthoic acid;
2,3,4-trichloro-5,6,7,8-tetrahydro-1-naphthoic acid;
5,6-dimethyl-4-indancarboxylic acid;
methyl 5,6-dimethyl-4-indancarboxylate;
6-chloro-5-methoxy-1,3-dihydro-4-isobenzofuroic acid;
methyl 6-chloro-5-methoxy-1,3-dihydro-4-isobenzofuroate;
ethyl 6-chloro-5-methoxy-1,3-dihydro-4-isobenzofuroate;
5,6-dichloro-1,3-dihydro-4-isobenzofuroic acid;
5-methoxy-6-methyl-1,3-dihydro-4-isobenzofuroic acid;
5-methoxy-6-methyl-2,3-dihydro-1-benzofuran-4-carboxylic acid;
methyl 5-methoxy-6-methyl-2,3-dihydro-1-benzofuran-4-carboxylate;
ethyl 5-methoxy-6-methyl-2,3-dihydro-1-benzofuran-4-carboxylate;
5-ethyl-6-methoxy-2,3-dihydro-1-benzofuran-7-carboxylic acid;
5,6-difluoro-2,2-dimethyl-2,3-dihydro-1-benzofuran-7-carboxylic acid;
5-fluoro-6-methyl-2,3-dihydro-1-benzothiophene-4-carboxylic acid;
5-chloro-6-methyl-2,3-dihydro-1-benzothiophene-4-carboxylic acid;
5,6-dichloro-2,3-dihydro-1-benzothiophene-4-carboxylic acid;
6-chloro-5-methyl-2,3-dihydro-1-benzothiophene-7-carboxylic acid; and
5,6-dimethyl-2,3-dihydro-1-benzothiophene-7-carboxylic acid.

In one embodiment, the compound is of Formula (I) wherein T and X are absent. In one embodiment, the compound is of formula (II):

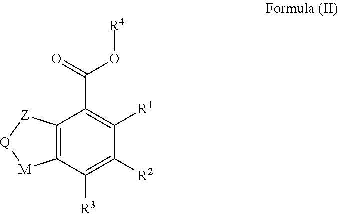

wherein:
   M is CR^{5M}R^{6M}, NR⁹, O or S;
   Q is CR^{7Q}R^{6Q}, NR^{10Q}, O or S;
   Z is CR^{8Z}R^{6Z}, NR¹¹, O or S;
wherein no more than one of M, Q or Z is selected from the group consisting of NR⁹, NR¹⁰, NR¹¹, O or S;
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
R^{5M} is selected from the group consisting of H, deuterium, F and C₁₋₃ alkyl optionally substituted with one or more, identical or different, substituents R¹²;
   R^{6M}, R^{6Q} and R^{6Z} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q} is selected from the group consisting of H, deuterium, F and C₁₋₃ alkyl optionally substituted with one or more, identical or different, substituents R¹²;
R^{8Z} is selected from the group consisting of H, deuterium, F and C₁₋₃ alkyl optionally substituted with one or more, identical or different, substituents R¹²;
   R⁹ is selected from the group consisting of H, C₁₋₃ alkyl optionally substituted with one or more, identical or different, substituents R¹² and C₁₋₃ alkyl substituted with R¹⁴;
   R^{10Q} is selected from benzyl optionally substituted with one or more, identical or different, substituents R¹³;
   R¹¹ is selected from the group consisting of H, C₁₋₅ alkyl optionally substituted with one or more, identical or different, substituents R¹² and C₁₋₃ alkyl substituted with R¹⁵;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F;
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; and
   R¹⁵ is independently selected from the group consisting of C₃₋₆ cycloalkyl optionally substituted with one or more, identical or different, substituents R¹², C₆10 aryl optionally substituted with one or more, identical or different, substituents R¹³ and 5- to 10-membered heteroaryl optionally substituted with one or more, identical or different, substituents R¹³;
or a pharmaceutically acceptable salt, hydrate, polymorph, tautomer, or solvate thereof provided that:
a) when M, Q and Z are CH₂, R¹ is Me, R² is Me and R³ is H then R⁴ is not H or Me;
   when M and Z are CH₂, Q is 0, R¹ is OMe, R² is Cl and R³ is H then R⁴ is not H, Me or Et;
   when M and Z are CH₂, Q is 0, R¹ is Cl, R² is Cl, and R³ is H then R⁴ is not H;
   when M and Z are CH₂, Q is 0, R¹ is OMe, R² is Me, and R³ is H then R⁴ is not H;
   when Q and Z are CH₂, M is 0, R¹ is OMe, R² is Me and R³ is H then R⁴ is not H, Me or Et;
   when M and Q are CH₂, Z is 0, R¹ is OMe, R² is Et and R³ is H then R⁴ is not H;
   when M is CH₂, Q is CMe₂, Z is O, R¹ and R² are F and R³ is H then R⁴ is not H;
   when Q and Z are CH₂, M is S, R¹ is Cl or F, R² is Me and R³ is H then R⁴ is not H;
   when Q and Z are CH₂, M is S, R¹ is Cl, R² is Cl and R³ is H then R⁴ is not H; and
   when Q and M are CH₂, Z is S, R¹ is Cl or Me, R² is Me and R³ is H then R⁴ is not H; or
b) the compound is not a compound selected from the group consisting of:
5,6-dimethyl-4-indancarboxylic acid;
methyl 5,6-dimethyl-4-indancarboxylate;
6-chloro-5-methoxy-1,3-dihydro-4-isobenzofuroic acid;
methyl 6-chloro-5-methoxy-1,3-dihydro-4-isobenzofuroate;
ethyl 6-chloro-5-methoxy-1,3-dihydro-4-isobenzofuroate;
5,6-dichloro-1,3-dihydro-4-isobenzofuroic acid;
5-methoxy-6-methyl-1,3-dihydro-4-isobenzofuroic acid;
5-methoxy-6-methyl-2,3-dihydro-1-benzofuran-4-carboxylic acid;
methyl 5-methoxy-6-methyl-2,3-dihydro-1-benzofuran-4-carboxylate;
ethyl 5-methoxy-6-methyl-2,3-dihydro-1-benzofuran-4-carboxylate;
5-ethyl-6-methoxy-2,3-dihydro-1-benzofuran-7-carboxylic acid;
5,6-difluoro-2,2-dimethyl-2,3-dihydro-1-benzofuran-7-carboxylic acid;
5-fluoro-6-methyl-2,3-dihydro-1-benzothiophene-4-carboxylic acid;
5-chloro-6-methyl-2,3-dihydro-1-benzothiophene-4-carboxylic acid;
5,6-dichloro-2,3-dihydro-1-benzothiophene-4-carboxylic acid;
6-chloro-5-methyl-2,3-dihydro-1-benzothiophene-7-carboxylic acid; and
5,6-dimethyl-2,3-dihydro-1-benzothiophene-7-carboxylic acid.

In one embodiment, the compound is of formula (II):

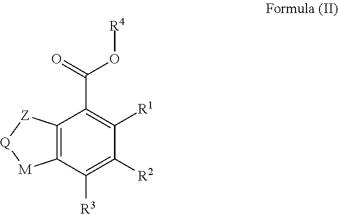

wherein:
   M is CR^{5M}R^{6M}, NR⁹, O or S;
   Q is CR^{7Q}R^{6Q} or NR^{10Q};
   Z is CR^{8Z}R^{6Z}, NR¹¹, O or S;
wherein no more than one of M, Q or Z is selected from the group consisting of NR⁹, NR¹⁰, NR¹¹, O or S;
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{5M} is selected from the group consisting of H, deuterium, F and C₁₋₃ alkyl optionally substituted with one or more, identical or different, substituents R¹²;
   R^{6M}, R^{6Q} and R^{6Z} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q} is selected from the group consisting of H, deuterium, F and C₁₋₃ alkyl optionally substituted with one or more, identical or different, substituents R¹²;
   R^{8Z} is selected from the group consisting of H, deuterium, F and C₁₋₃ alkyl optionally substituted with one or more, identical or different, substituents R¹²;
   R⁹ is selected from the group consisting of H, C₁₋₃ alkyl optionally substituted with one or more, identical or different, substituents R¹² and C₁₋₃ alkyl substituted with R¹⁴;
   R^{10Q} is selected from benzyl optionally substituted with one or more, identical or different, substituents R¹³;
   R¹¹ is selected from the group consisting of H, C₁₋₅ alkyl optionally substituted with one or more, identical or different, substituents R¹² and C₁₋₃ alkyl substituted with R¹⁵;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F;
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; and
   R¹⁵ is independently selected from the group consisting of C₃₋₆ cycloalkyl optionally substituted with one or more, identical or different, substituents R¹², C₆₋₁₀ aryl optionally substituted with one or more, identical or different, substituents R¹³ and 5- to 10-membered heteroaryl optionally substituted with one or more, identical or different, substituents R¹³;
or a pharmaceutically acceptable salt, hydrate, polymorph, tautomer, or solvate thereof provided that:
a) when M, Q and Z are CH₂, R¹ is Me, R² is Me and R³ is H then R⁴ is not H or Me;
   when Q and Z are CH₂, M is 0, R¹ is OMe, R² is Me and R³ is H then R⁴ is not H, Me or Et;
   when M and Q are CH₂, Z is O, R¹ is OMe, R² is Et and R³ is H then R⁴ is not H;
   when M is CH₂, Q is CMe₂, Z is 0, R¹ and R² are F and R³ is H then R⁴ is not H;
   when Q and Z are CH₂, M is S, R¹ is Cl or F, R² is Me and R³ is H then R⁴ is not H;
   when Q and Z are CH₂, M is S, R¹ is Cl, R² is Cl and R³ is H then R⁴ is not H; and
   when Q and M are CH₂, Z is S, R¹ is Cl or Me, R² is Me and R³ is H then R⁴ is not H; or
b) the compound is not a compound selected from the group consisting of:
5,6-dimethyl-4-indancarboxylic acid;
methyl 5,6-dimethyl-4-indancarboxylate;
5-methoxy-6-methyl-2,3-dihydro-1-benzofuran-4-carboxylic acid;
methyl 5-methoxy-6-methyl-2,3-dihydro-1-benzofuran-4-carboxylate;
ethyl 5-methoxy-6-methyl-2,3-dihydro-1-benzofuran-4-carboxylate;
5-ethyl-6-methoxy-2,3-dihydro-1-benzofuran-7-carboxylic acid;
5,6-difluoro-2,2-dimethyl-2,3-dihydro-1-benzofuran-7-carboxylic acid;
5-fluoro-6-methyl-2,3-dihydro-1-benzothiophene-4-carboxylic acid;
5-chloro-6-methyl-2,3-dihydro-1-benzothiophene-4-carboxylic acid;
5,6-dichloro-2,3-dihydro-1-benzothiophene-4-carboxylic acid;
6-chloro-5-methyl-2,3-dihydro-1-benzothiophene-7-carboxylic acid; and
5,6-dimethyl-2,3-dihydro-1-benzothiophene-7-carboxylic acid.

In one embodiment, the compound is of Formula (I) wherein X is absent. In one embodiment, the compound is of formula (III):

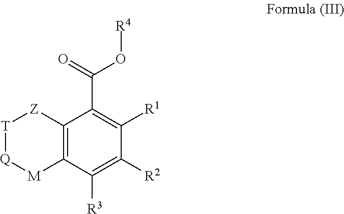

wherein:
   M is CR^{5M}R^{6M}, NR⁹, O or S;
   Q is CR^{7Q}R^{6Q}, NR^{10Q}, O or S;
   T is CR^{7T}R^{6T}, NR^{10T}, O or S;
   Z is CR^{8Z}R^{6Z}, NR¹¹, O or S;
wherein no more than one of M, Q, T or Z is selected from the group consisting of NR^{9M}, NR^{10Q}, NR^{10T}, NR¹¹, O or S;
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{5M} is selected from the group consisting of H, deuterium, F and C₁₋₃ alkyl optionally substituted with one or more, identical or different, substituents R¹²;
   R^{6M}, R^{6Q}, R^{6T} and R^{6Z} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q} and R^{7T} are independently selected from the group consisting of H, deuterium, F and C₁₋₃ alkyl optionally substituted with one or more, identical or different, substituents R¹²;
   R^{8Z} is selected from the group consisting of H, deuterium, F and C₁₋₃ alkyl optionally substituted with one or more, identical or different, substituents R¹²;
   R⁹ is selected from the group consisting of H, C₁₋₃ alkyl optionally substituted with one or more, identical or different, substituents R¹² and C₁₋₃ alkyl substituted with R¹⁴;
   R¹⁰ and R^{10T} are independently selected from benzyl optionally substituted with one or more, identical or different, substituents R¹³;
   R¹¹ is selected from the group consisting of H, C₁₋₅ alkyl optionally substituted with one or more, identical or different, substituents R¹² and C₁₋₃ alkyl substituted with R¹⁵;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F;
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; and
   R¹⁵ is independently selected from the group consisting of C₃₋₆ cycloalkyl optionally substituted with one or more, identical or different, substituents R¹², C₆10 aryl optionally substituted with one or more, identical or different, substituents R¹³ and 5- to 10-membered heteroaryl optionally substituted with one or more, identical or different, substituents R¹³;
or a pharmaceutically acceptable salt, hydrate, polymorph, tautomer, or solvate thereof provided that:
a) when M, Q, T and Z are CH₂, R¹ is OMe, R² is Cl and R³ is H then R⁴ is not H, Me or Et;
   when M, Q, T and Z are CH₂, R¹ is OMe, R² is CF₃ and R³ is H then R⁴ is not Me; and
   when M, Q, T and Z are CH₂, R¹ is Me, R² is Me and R³ is H then R⁴ is not H; or
b) the compound is not a compound selected from the group consisting of:
3-chloro-2-methoxy-5,6,7,8-tetrahydro-1-naphthoic acid;
methyl 3-chloro-2-methoxy-5,6,7,8-tetrahydro-1-naphthoate;
ethyl 3-chloro-2-methoxy-5,6,7,8-tetrahydro-1-naphthoate;
methyl 2-methoxy-3-(trifluoromethyl)-5,6,7,8-tetrahydro-1-naphthoate; and
2,3-dimethyl-5,6,7,8-tetrahydro-1-naphthoic acid.

In one embodiment, the compound is of formula (III):

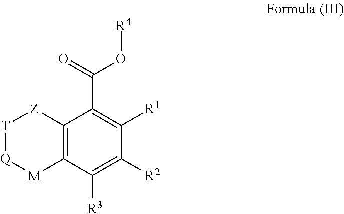

wherein:
   M is CR^{5M}R^{6M}, NR⁹, O or S;
   Q is CR^{7Q}R^{6Q} or NR^{10Q};
   T is CR^{7T}R^{6T} or NR^{10T};
   Z is CR^{8Z}R^{6Z}, NR¹¹, O or S;
wherein no more than one of M, Q, T or Z is selected from the group consisting of NR⁹M, NR^{10Q}, NR^{10T}, NR¹¹, O or S;
   R¹ is selected from the group consisting of F; Cl; C₁₋₃ alkyl optionally substituted with one or more deuterium; C₂₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{5M} is selected from the group consisting of H, deuterium, F and C₁₋₃ alkyl optionally substituted with one or more, identical or different, substituents R¹²;
   R^{6M} R^{6Q} R^{6T} and R^{6Z} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q} and R^{7T} are independently selected from the group consisting of H, deuterium, F and C₁₋₃ alkyl optionally substituted with one or more, identical or different, substituents R¹²;
   R^{8Z} is selected from the group consisting of H, deuterium, F and C₁₋₃ alkyl optionally substituted with one or more, identical or different, substituents R¹²;
   R⁹ is selected from the group consisting of H, C₁₋₃ alkyl optionally substituted with one or more, identical or different, substituents R¹² and C₁₋₃ alkyl substituted with R¹⁴;
   R^{10Q} and R^{10T} are independently selected from benzyl optionally substituted with one or more, identical or different, substituents R¹³;
   R¹¹ is selected from the group consisting of H, C₁₋₅ alkyl optionally substituted with one or more, identical or different, substituents R¹² and C₁₋₃ alkyl substituted with R¹⁵;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F;
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; and
   R¹⁵ is independently selected from the group consisting of C₃₋₆ cycloalkyl optionally substituted with one or more, identical or different, substituents R¹², C₆10 aryl optionally substituted with one or more, identical or different, substituents R¹³ and 5- to 10-membered heteroaryl optionally substituted with one or more, identical or different, substituents R¹³;
or a pharmaceutically acceptable salt, hydrate, polymorph, tautomer, or solvate thereof provided that:
a) when M, Q, T and Z are CH₂, R¹ is OMe, R² is Cl and R³ is H then R⁴ is not H, Me or Et;
   when M, Q, T and Z are CH₂, R¹ is OMe, R² is CF₃ and R³ is H then R⁴ is not Me; and
   when M, Q, T and Z are CH₂, R¹ is Me, R² is Me and R³ is H then R⁴ is not H; or
b) the compound is not a compound selected from the group consisting of:
3-chloro-2-methoxy-5,6,7,8-tetrahydro-1-naphthoic acid;
methyl 3-chloro-2-methoxy-5,6,7,8-tetrahydro-1-naphthoate;
ethyl 3-chloro-2-methoxy-5,6,7,8-tetrahydro-1-naphthoate;
methyl 2-methoxy-3-(trifluoromethyl)-5,6,7,8-tetrahydro-1-naphthoate; and
2,3-dimethyl-5,6,7,8-tetrahydro-1-naphthoic acid.

In one embodiment, the compound is of formula (I):

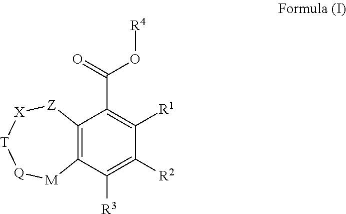

wherein:
   M is CR^{5M}R^{6M}, NR⁹, O or S;
   Q is CR^{7Q}R^{6Q}, NR^{10Q}, O or S;
   T is CR^{7T}R^{6T}, NR^{10T}, O or S;
   X is CR^{7X}R^{6X}, NR^{10X}, O or S;
   Z is CR^{8Z}R^{6Z}, NR¹¹, O or S;
wherein no more than one of M, Q, T, X or Z is selected from the group consisting of NR⁹M, NR^{10Q}, NR^{10T}, NR^{10X}, NR¹¹, O or S;
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{5M} is selected from the group consisting of H, deuterium, F and C₁₋₃ alkyl optionally substituted with one or more, identical or different, substituents R¹²;
   R^{6M} R^{6Q} R^{6T} R^{6X} and R^{6Z} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q}, R^{7T} and R^{7X} are independently selected from the group consisting of H, deuterium, F and C₁₋₃ alkyl optionally substituted with one or more, identical or different, substituents R¹²;
R^{8Z} is selected from the group consisting of H, deuterium, F and C₁₋₃ alkyl optionally substituted with one or more, identical or different, substituents R¹²;
   R⁹ is selected from the group consisting of H, C₁₋₃ alkyl optionally substituted with one or more, identical or different, substituents R¹² and C₁₋₃ alkyl substituted with R¹⁴;
   R¹⁰, R^{10T} and R^{10X} are independently selected from benzyl optionally substituted with one or more, identical or different, substituents R¹³;
   R¹¹ is selected from the group consisting of H, C₁₋₃ alkyl optionally substituted with one or more, identical or different, substituents R¹² and C₁₋₃ alkyl substituted with R¹⁵;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F;
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; and
   R¹⁵ is independently selected from the group consisting of C₃₋₆ cycloalkyl optionally substituted with one or more, identical or different, substituents R¹², C₆₋₁₀ aryl optionally substituted with one or more, identical or different, substituents R¹³ and 5- to 10-membered heteroaryl optionally substituted with one or more, identical or different, substituents R¹³;
or a pharmaceutically acceptable salt, hydrate, polymorph, tautomer, or solvate thereof provided that:
a) when M, Q, T, X and Z are CH₂, R¹ is OMe, R² is Cl and R³ is H then R⁴ is not H, Me or Et; or
b) the compound is not a compound selected from the group consisting of:
10-chloro-9-methoxybicyclo[5.4.0]undeca-1(7),8,10-triene-8-carboxylic acid;
methyl 10-chloro-9-methoxybicyclo[5.4.0]undeca-1(7),8,10-triene-8-carboxylate; and
ethyl 10-chloro-9-methoxybicyclo[5.4.0]undeca-1(7),8,10-triene-8-carboxylate.

In one embodiment, the compound is of formula (I):

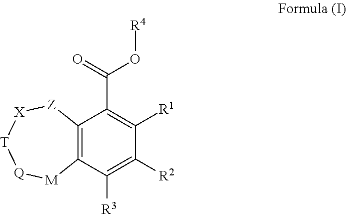

wherein:
   M is CR^{5M}R^{6M}, NR⁹, O or S;
   Q is CR^{7Q}R^{6Q} or NR^{10Q};
   T is CR^{7T}R^{6T} or NR^{10T};
   X is CR^{7X}R^{6X} or NR^{10X};
   Z is CR^{8Z}R^{6Z}, NR¹¹, O or S;
wherein no more than one of M, Q, T, X or Z is selected from the group consisting of NR^{9M}, NR^{10Q}, NR^{10T}, NR^{10X}, NR¹¹, O or S;
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{5M} is selected from the group consisting of H, deuterium, F and C₁₋₃ alkyl optionally substituted with one or more, identical or different, substituents R¹²;
   R^{6M} R^{6Q} R^{6T} R^{6X} and R^{6Z} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q}, R^{7T} and R^{7X} are independently selected from the group consisting of H, deuterium, F and C₁₋₃ alkyl optionally substituted with one or more, identical or different, substituents R¹²;
   R^{8Z} is selected from the group consisting of H, deuterium, F and C₁₋₃ alkyl optionally substituted with one or more, identical or different, substituents R¹²;
   R⁹ is selected from the group consisting of H, C₁₋₃ alkyl optionally substituted with one or more, identical or different, substituents R¹² and C₁₋₃ alkyl substituted with R¹⁴;
   R¹⁰, R^{10T} and R^{10X} are independently selected from benzyl optionally substituted with one or more, identical or different, substituents R¹³;
   R¹¹ is selected from the group consisting of H, C₁₋₃ alkyl optionally substituted with one or more, identical or different, substituents R¹² and C₁₋₃ alkyl substituted with R¹⁵;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F;
   R¹⁴ is independently selected from the group consisting of C₃₋₅ cycloalkyl optionally substituted with one or more, identical or different, substituents R¹², phenyl optionally substituted with one or more, identical or different, substituents R¹³ 5-membered heteroaryl optionally substituted with one or more, identical or different, substituents R¹³; and
   R¹⁵ is independently selected from the group consisting of C₃₋₆ cycloalkyl optionally substituted with one or more, identical or different, substituents R¹², C₆10 aryl optionally substituted with one or more, identical or different, substituents R¹³ and 5- to 10-membered heteroaryl optionally substituted with one or more, identical or different, substituents R¹³;
or a pharmaceutically acceptable salt, hydrate, polymorph, tautomer, or solvate thereof provided that:
a) when M, Q, T, X and Z are CH₂, R¹ is OMe, R² is Cl and R³ is H then R⁴ is not H, Me or Et; or
b) the compound is not a compound selected from the group consisting of:
10-chloro-9-methoxybicyclo[5.4.0]undeca-1(7),8,10-triene-8-carboxylic acid;
methyl 10-chloro-9-methoxybicyclo[5.4.0]undeca-1(7),8,10-triene-8-carboxylate; and
ethyl 10-chloro-9-methoxybicyclo[5.4.0]undeca-1(7),8,10-triene-8-carboxylate.

In one embodiment, the compound is of Formula (I) and M is CR⁵R⁶. In one embodiment, the compound is of Formula (I), Formula (II) or Formula (III) and M is CR^{5M}R^{6M}. In one embodiment, M is NR⁹. In one embodiment, M is O. In one embodiment, M is S.

In one embodiment, the compound is of Formula (I) and Q is CR⁷R⁶. In one embodiment, the compound is of Formula (I), Formula (II) or Formula (III) and Q is CR^{7Q}R⁶⁰. In one embodiment, the compound is of Formula (I) and Q is NR¹⁰. In one embodiment, the compound is of Formula (I), Formula (II) or Formula (III) and Q is NR^{10Q}. In one embodiment, Q is O. In one embodiment, Q is S.

In one embodiment, the compound is of Formula (I) or Formula (III) and T is absent. In one embodiment, the compound is of Formula (I) and T is CR⁷R⁶. In one embodiment, the compound is of Formula (I) or Formula (III) and T is CR^{7T}R^{6T}. In one embodiment, the compound is of Formula (I) and T is NR¹⁰. In one embodiment, the compound is of Formula (I) or Formula (III) and T is NR^{10T}. In one embodiment, T is O. In one embodiment, T is S.

In one embodiment, the compound is of Formula (I) and X is absent. In one embodiment, the compound is of Formula (I) and X is CR⁷R⁶. In one embodiment, the compound is of Formula (I) and X is CR^{7X}R^{6X}. In one embodiment, the compound is of Formula (I) and X is NR¹⁰. In one embodiment, the compound is of Formula (I) and X is NR^{10X}. In one embodiment, X is O. In one embodiment, X is S.

In one embodiment, the compound is of Formula (I) and Z is CR⁸R⁶. In one embodiment, the compound is of Formula (I), Formula (II) or (Ill) and Z is CR^{8Z}R^{6Z}. In one embodiment, Z is NR¹¹. In one embodiment, Z is O. In one embodiment, Z is S. In some embodiments, two groups are linked together to form a ring together with their intervening atoms. Thus, in some embodiments, one hydrogen from each of said two groups is replaced with a joint bond. In some embodiments, R⁵ and R⁸, or R⁵ and R¹¹, or R⁹ and R⁸ are optionally joined together to form a ring by —CH₂—, —CH₂CH₂- or —CH₂CH₂CH₂-. When R⁵ and R⁸, or R⁵ and R¹¹, or R⁹ and R⁸ are joined together by —CH₂— to form a ring, then i) R⁵ or R⁹ is a bond and R⁸ or R¹¹ is C, alkandediyl, or ii) R⁵ or R⁹ is C, alkandediyl and R⁸ or R¹¹ is a bond. When R⁵ and R⁸, or R⁵ and R¹¹, or R⁹ and R⁸ are joined together by —CH₂CH₂- to form a ring, then i) R⁵ or R⁹ is a bond and R⁸ or R¹¹ is C₂ alkandediyl, or ii) R⁵ or R⁹ is C₂ alkandediyl and R⁸ or R¹¹ is a bond, or iii) R⁵ or R⁹ is C, alkandediyl and R⁸ or R¹¹ is C, alkandediyl. When R⁵ and R⁸, or R⁵ and R¹¹, or R⁹ and R⁸ are joined together by —CH₂CH₂CH₂- to form a ring, then i) R⁵ or R⁹ is a bond and R⁸ or R¹¹ is C₃ alkandediyl, or ii) R⁵ or R⁹ is C₃ alkandediyl and R⁸ or R¹¹ is a bond, or iii) R⁵ or R⁹ is C, alkandediyl and R⁸ or R¹¹ is C₂ alkandediyl, or iv) R⁵ or R⁹ is C₂ alkandediyl and R⁸ or R¹¹ is C, alkandediyl, In one embodiment, R⁵ and R⁸ are joined together by —CH₂—, —CH₂CH₂- or —CH₂CH₂CH₂— to form a ring.

In one embodiment, R⁵ and R¹¹ are joined together by —CH₂—, —CH₂CH₂- or —CH₂CH₂CH₂- to form a ring.

In one embodiment, R⁹ and R⁸ are joined together by —CH₂—, —CH₂CH₂- or —CH₂CH₂CH₂— to form a ring.

In one embodiment, M is CR⁵R⁶; Q is CR⁷R⁶; T is absent; X is absent; and Z is CR⁸R⁶.

In one embodiment, the compound is of formula (IV):

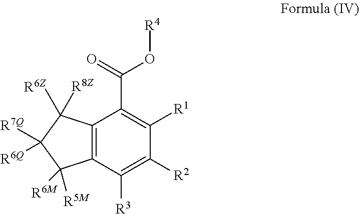

wherein:
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{5M} is selected from the group consisting of H, deuterium, F and C₁₋₃ alkyl optionally substituted with one or more, identical or different, substituents R¹²;
   R^{6M}, R^{6Q} and R^{6Z} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q} is selected from the group consisting of H, deuterium, F and C₁₋₃ alkyl optionally substituted with one or more, identical or different, substituents R¹²;
   R^{8Z} is selected from the group consisting of H, deuterium, F and C₁₋₃ alkyl optionally substituted with one or more, identical or different, substituents R¹²;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F; and
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³;
or a pharmaceutically acceptable salt, hydrate, polymorph, tautomer, or solvate thereof provided that:
a) when R¹ is Me, R² is Me, R³, R^{5M}, R^{6M}, R^{6Q} R^{6Z} R^{7Q} and R^{8Z} are H then R⁴ is not H or Me; or
b) the compound is not 5,6-dimethyl-4-indancarboxylic acid or methyl 5,6-dimethyl-4-indancarboxylate.

In one embodiment, M is CR⁵R⁶; Q is CR⁷R⁶; T is CR⁷R⁶; X is absent; and Z is CR⁸R⁶.

In one embodiment, the compound is of formula (V):

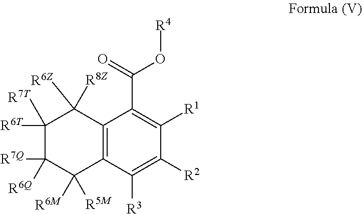

wherein:
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{5M} is selected from the group consisting of H, deuterium, F and C₁₋₃ alkyl optionally substituted with one or more, identical or different, substituents R¹²;
   R^{6M}, R^{6Q} R^{6T} and R^{6Z} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q} and R^{7T} are independently selected from the group consisting of H, deuterium, F and C₁₋₃ alkyl optionally substituted with one or more, identical or different, substituents R¹²;
   R^{8Z} is selected from the group consisting of H, deuterium, F and C₁₋₃ alkyl optionally substituted with one or more, identical or different, substituents R¹²;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F; and
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³;
or a pharmaceutically acceptable salt, hydrate, polymorph, tautomer, or solvate thereof provided that:
a) when R¹ is OMe, R² is Cl, R³, R^{5M} R^{6M}R^{6Q} R^{6T} R^{6Z} R^{7Q}, R^{7T}, and R^{8Z} are H then R⁴ is not H, Me or Et;
when R¹ is OMe, R² is CF₃, R³, R^{5M} R^{6M} R^{6Q} R^{6T} R^{6Z} R7Q, R^{7T}, and R^{8Z} are H then R⁴ is not Me; and when R¹ is Me, R² is Me, R³, R^{5M} R^{6M} R^{6Q} R^{6T} R^{6Z} R7Q, R^{7T}, and R^{8Z} are H then R⁴ is not H; or
b) the compound is not a compound selected from the group consisting of:
3-chloro-2-methoxy-5,6,7,8-tetrahydro-1-naphthoic acid;
methyl 3-chloro-2-methoxy-5,6,7,8-tetrahydro-1-naphthoate;
ethyl 3-chloro-2-methoxy-5,6,7,8-tetrahydro-1-naphthoate;
methyl 2-methoxy-3-(trifluoromethyl)-5,6,7,8-tetrahydro-1-naphthoate; and
2,3-dimethyl-5,6,7,8-tetrahydro-1-naphthoic acid.

In one embodiment, M is CR⁵R⁶; Q is CR⁷R⁶; T is CR⁷R⁶; X is CR⁷R⁶; and Z is CR⁸R⁶.

In one embodiment, the compound is of formula (VI):

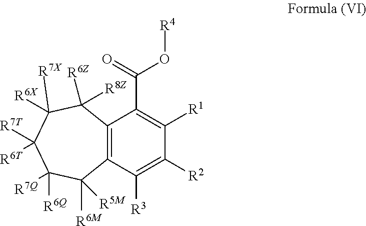

wherein:
   R¹ is selected from the group consisting of F; Cl; C₁₋₃ alkyl optionally substituted with one or more deuterium; C₂₋₃ alkenyl optionally substituted with one or more, identical or different, substituents R¹². —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{5M} is selected from the group consisting of H, deuterium, F and C₁₋₃ alkyl optionally substituted with one or more, identical or different, substituents R¹²;
   R^{6M} R^{6Q} R^{6T} R^{6X} and R^{6Z} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q}, R^{7T} and R^{7X} are independently selected from the group consisting of H, deuterium, F and C₁₋₃ alkyl optionally substituted with one or more, identical or different, substituents R¹²;
   R^{8Z} is selected from the group consisting of H, deuterium, F and C₁₋₃ alkyl optionally substituted with one or more, identical or different, substituents R¹²;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F; and
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; or a pharmaceutically acceptable salt, hydrate, polymorph, tautomer, or solvate thereof provided that:
a) when R¹ is OMe, R² is Cl, R³, R^{5M} R^{6M} R^{6Q} R^{6T} R^{6X} R^{6Z} R^{7Q} R^{7T} R^{7X} and R^{8Z} are H then R⁴ is not H, Me or Et; or
b) the compound is not 10-chloro-9-methoxybicyclo[5.4.0]undeca-1(7),8,10-triene-8-carboxylic acid, methyl 10-chloro-9-methoxybicyclo[5.4.0]undeca-1(7),8,10-triene-8-carboxylate, or ethyl 10-chloro-9-methoxybicyclo[5.4.0]undeca-1(7),8,10-triene-8-carboxylate.

In one embodiment, M is CR⁵R⁶; Q is CR⁷R⁶; T is absent; X is absent; and Z is O. In one embodiment, the compound is of formula (VII):

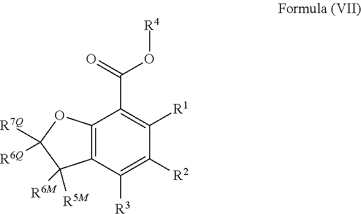

wherein:
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{5M} is selected from the group consisting of H, deuterium, F and C₁₋₃ alkyl optionally substituted with one or more, identical or different, substituents R¹²;
   R^{6M} and R^{6Q} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q} is selected from the group consisting of H, deuterium, F and C₁₋₃ alkyl optionally substituted with one or more, identical or different, substituents R¹²;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F; and
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; or a pharmaceutically acceptable salt, hydrate, polymorph, tautomer, or solvate thereof provided that:
a) when R¹ is OMe, R² is Et, R³, R^{5M} R^{6M}R^{6Q} and R^{7Q} are H then R⁴ is not H; and when R¹ and R² are F, R³, R^{5M}, R^{6M} are H, and R^{6Q} and R^{7Q} are Me then R⁴ is not H; or
b) the compound is not 5-ethyl-6-methoxy-2,3-dihydro-1-benzofuran-7-carboxylic acid, or 5,6-difluoro-2,2-dimethyl-2,3-dihydro-1-benzofuran-7-carboxylic acid.

In one embodiment, M is CR⁵R⁶; Q is CR⁷R⁶; T is CR⁷R⁶; X is absent; and Z is O. In one embodiment, the compound is of formula (VIII):

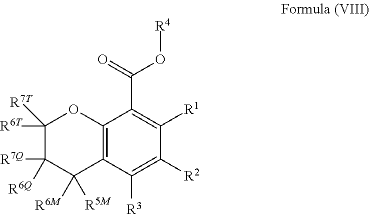

wherein:
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{5M} is selected from the group consisting of H, deuterium, F and C₁₋₃ alkyl optionally substituted with one or more, identical or different, substituents R¹²;
   R^{6M}, R^{6Q} and R^{6T} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q} and R^{7T} are independently selected from the group consisting of H, deuterium, F and C₁₋₃ alkyl optionally substituted with one or more, identical or different, substituents R¹²;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F; and
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; or a pharmaceutically acceptable salt, hydrate, polymorph, tautomer, or solvate thereof.

In one embodiment, wherein M is CR⁵R⁶; Q is CR⁷R⁶; T is CR⁷R⁶; X is absent; and Z is S. In one embodiment, the compound is of formula (IX):

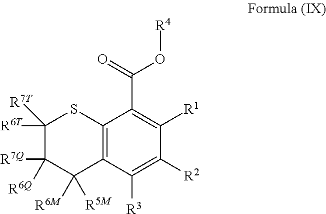

wherein:
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{5M} is selected from the group consisting of H, deuterium, F and C₁₋₃ alkyl optionally substituted with one or more, identical or different, substituents R¹²;
   R^{6M}, R^{6Q} and R^{6T} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q} and R^{7T} are independently selected from the group consisting of H, deuterium, F and C₁₋₃ alkyl optionally substituted with one or more, identical or different, substituents R¹²;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F; and
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³;
or a pharmaceutically acceptable salt, hydrate, polymorph, tautomer, or solvate thereof.

In one embodiment, M is CR⁵R⁶; Q is CR⁷R⁶; T is CR⁷R⁶; X is absent; and Z is NR¹¹. In one embodiment, the compound is of formula (X):

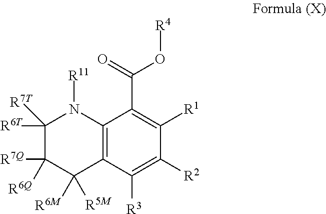

wherein:
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{5M} is selected from the group consisting of H, deuterium, F and C₁₋₃ alkyl optionally substituted with one or more, identical or different, substituents R¹²;
   R^{6M}, R^{6Q} and R^{6T} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q} and R^{7T} are independently selected from the group consisting of H, deuterium, F and C₁₋₃ alkyl optionally substituted with one or more, identical or different, substituents R¹²;
   R¹¹ is selected from the group consisting of H, C₁₋₃ alkyl optionally substituted with one or more, identical or different, substituents R¹² and C₁₋₃ alkyl substituted with R¹⁵;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F;
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; and
   R¹⁵ is independently selected from the group consisting of C₃₋₆ cycloalkyl optionally substituted with one or more, identical or different, substituents R¹², C₆10 aryl optionally substituted with one or more, identical or different, substituents R¹³ and 5- to 10-membered heteroaryl optionally substituted with one or more, identical or different, substituents R¹³;
or a pharmaceutically acceptable salt, hydrate, polymorph, tautomer, or solvate thereof.

In one embodiment, M is 0; Q is CR⁷R⁶; T is CR⁷R⁶; X is absent; and Z is CR⁸R⁶. In one embodiment, the compound is of formula (XI):

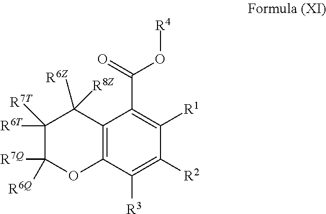

wherein:
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{6Q}, R^{6T} and R^{6Z} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q} and R^{7T} are independently selected from the group consisting of H, deuterium, F and C₁₋₃ alkyl optionally substituted with one or more, identical or different, substituents R¹²;
   R^{8Z} is selected from the group consisting of H, deuterium, F and C₁₋₃ alkyl optionally substituted with one or more, identical or different, substituents R¹²;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F; and
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; or a pharmaceutically acceptable salt, hydrate, polymorph, tautomer, or solvate thereof.

In one embodiment, M is S; Q is CR⁷R⁶; T is CR⁷R⁶; X is absent; and Z is CR⁸R⁶. In one embodiment, the compound is of formula (XII):

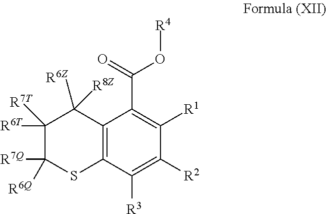

wherein:
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{6Q}, R^{6T} and R^{6Z} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q} and R^{7T} are independently selected from the group consisting of H, deuterium, F and C₁₋₃ alkyl optionally substituted with one or more, identical or different, substituents R¹²;
   R^{8Z} is selected from the group consisting of H, deuterium, F and C₁₋₃ alkyl optionally substituted with one or more, identical or different, substituents R¹²;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F; and
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; or a pharmaceutically acceptable salt, hydrate, polymorph, tautomer, or solvate thereof.

In one embodiment, M is NR⁹; Q is CR⁷R⁶; T is CR⁷R⁶; X is absent; and Z is CR⁸R⁶. In one embodiment, the compound is of formula (XIII):

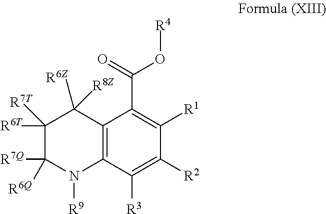

wherein:
   R¹ is selected from the group consisting of F; Cl; C₁₋₃ alkyl optionally substituted with one or more deuterium; C₂₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{6Q}, R^{6T} and R^{6Z} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q} and R^{7T} are independently selected from the group consisting of H, deuterium, F and C₁₋₃ alkyl optionally substituted with one or more, identical or different, substituents R¹²;
   R^{8Z} is selected from the group consisting of H, deuterium, F and C₁₋₃ alkyl optionally substituted with one or more, identical or different, substituents R¹²;
   R⁹ is selected from the group consisting of H, C₁₋₃ alkyl optionally substituted with one or more, identical or different, substituents R¹² and C₁₋₃ alkyl substituted with R¹⁴;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F; and
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; or a pharmaceutically acceptable salt, hydrate, polymorph, tautomer, or solvate thereof.

In one embodiment, M is CR⁵R⁶; Q is CR⁷R⁶; T is CR⁷R⁶; X is absent; Z is CR⁸R⁶; and R⁵ and R^{a} are joined together to form a ring by —CH₂—. In one embodiment, the compound is of formula (XIV):

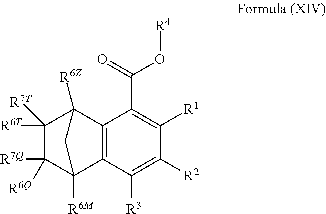

wherein:
   R¹ is selected from the group consisting of F; C; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; C; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{6M}, R^{6Q} R^{6T} and R^{6Z} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q} and R^{7T} are independently selected from the group consisting of H, deuterium, F and C₁₋₃ alkyl optionally substituted with one or more, identical or different, substituents R¹²;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F; and
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; or a pharmaceutically acceptable salt, hydrate, polymorph, tautomer, or solvate thereof.

In one embodiment, M is CR⁵R⁶; Q is CR⁷R⁶; T is CR⁷R⁶; X is absent; Z is CR⁸R⁶; and R⁵ and R^{a} are joined together to form a ring by-CH₂CH₂—. In one embodiment, the compound is of formula (XV):

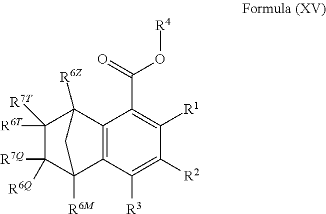

wherein:
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{6M}, R^{6Q} R^{6T} and R^{6Z} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q} and R^{7T} are independently selected from the group consisting of H, deuterium, F and C₁₋₃ alkyl optionally substituted with one or more, identical or different, substituents R¹²;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F; and
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; or a pharmaceutically acceptable salt, hydrate, polymorph, tautomer, or solvate thereof.

In one embodiment, M is CR⁵R⁶; Q is CR⁷R⁶; T is absent; X is absent; and Z is S. In one embodiment, the compound is of formula (XVI):

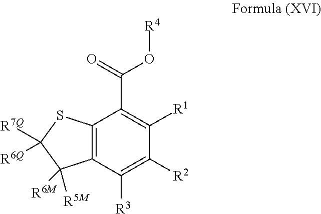

wherein:
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{5M} is selected from the group consisting of H, deuterium, F and C₁₋₃ alkyl optionally substituted with one or more, identical or different, substituents R¹²;
   R^{6M} and R^{6Q} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q} is selected from the group consisting of H, deuterium, F and C₁₋₃ alkyl optionally substituted with one or more, identical or different, substituents R¹²;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F; and
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³;
or a pharmaceutically acceptable salt, hydrate, polymorph, tautomer, or solvate thereof provided that:
a) when R¹ is Cl or Me, R² is Me, R³, R^{5M}, R^{6M}, R^{6Q} and R^{7Q} are H then R⁴ is not H; or
b) the compound is not 6-chloro-5-methyl-2,3-dihydro-1-benzothiophene-7-carboxylic acid, or 5,6-dimethyl-2,3-dihydro-1-benzothiophene-7-carboxylic acid.

In one embodiment, M is CR⁵R⁶; Q is CR⁷R⁶; T is absent; X is absent; and Z is NR¹¹. In one embodiment, the compound is of formula (XVII):

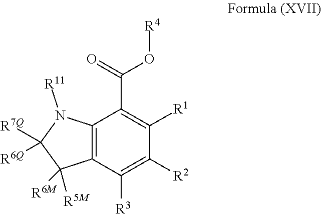

wherein:
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{5M} is selected from the group consisting of H, deuterium, F and C₁₋₃ alkyl optionally substituted with one or more, identical or different, substituents R¹²;
   R^{6M} and R^{6Q} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q} is selected from the group consisting of H, deuterium, F and C₁₋₃ alkyl optionally substituted with one or more, identical or different, substituents R¹²;
   R¹¹ is selected from the group consisting of H, C₁₋₃ alkyl optionally substituted with one or more, identical or different, substituents R¹² and C₁₋₃ alkyl substituted with R¹⁵;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F;
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; and
   R¹⁵ is independently selected from the group consisting of C₃₋₆ cycloalkyl optionally substituted with one or more, identical or different, substituents R¹², C₆10 aryl optionally substituted with one or more, identical or different, substituents R¹³ and 5- to 10-membered heteroaryl optionally substituted with one or more, identical or different, substituents R¹³;
or a pharmaceutically acceptable salt, hydrate, polymorph, tautomer, or solvate thereof.

In one embodiment, M is 0; Q is CR⁷R⁶; T is absent; X is absent; and Z is CR⁸R⁶. In one embodiment, the compound is of formula (XVIII):

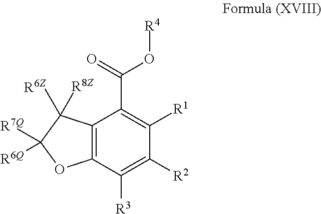

wherein:
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{6Q} and R^{6Z} are independently selected from the group consisting of H, deuterium, F, C₁₋₃ alkyl substituted with R¹⁴ and C₁₋₃ alkyl optionally substituted with one or more, identical or different, substituents R¹²;
R^{7Q} is selected from the group consisting of H, deuterium, F and C₁₋₃ alkyl optionally substituted with one or more, identical or different, substituents R¹²;
   R^{8Z} is selected from the group consisting of H, deuterium, F and C₁₋₃ alkyl optionally substituted with one or more, identical or different, substituents R¹²;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F; and
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; or a pharmaceutically acceptable salt, hydrate, polymorph, tautomer, or solvate thereof provided that:
a) when R¹ is OMe or Me, R² is Me, R³, R^{6Q}, R^{6Z}, R^{7Q} and R^{8Z} are H then R⁴ is not H, Me or Et; or
b) the compound is not 5-methoxy-6-methyl-2,3-dihydro-1-benzofuran-4-carboxylic acid; methyl 5-methoxy-6-methyl-2,3-dihydro-1-benzofuran-4-carboxylate; or ethyl 5-methoxy-6-methyl-2,3-dihydro-1-benzofuran-4-carboxylate.

In one embodiment, M is S; Q is CR⁷R⁶; T is absent; X is absent; and Z is CR⁸R⁶. In one embodiment, the compound is of formula (XIX):

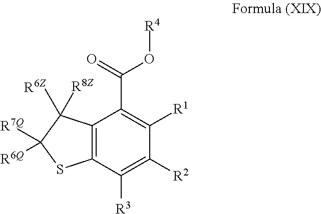

wherein:
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{6Q} and Rz are independently selected from the group consisting of H, deuterium, F, C₁₋₃ alkyl substituted with R¹⁴ and C₁₋₃ alkyl optionally substituted with one or more, identical or different, substituents R¹²;
   R^{7Q} is selected from the group consisting of H, deuterium, F and C₁₋₃ alkyl optionally substituted with one or more, identical or different, substituents R¹²;
   R^{8Z} is selected from the group consisting of H, deuterium, F and C₁₋₃ alkyl optionally substituted with one or more, identical or different, substituents R¹²;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F; and
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; or a pharmaceutically acceptable salt, hydrate, polymorph, tautomer, or solvate thereof provided that:
a) when R¹ is Cl or F, R² is Me, R³, R^{6Q}, R^{6Z}, R^{7Q} and Raz are H then R⁴ is not H; and when R¹ is Cl, R² is Cl, R³, R^{6Q}, R^{6Z}, R^{7Q} and Raz are H then R⁴ is not H; or b) the compound is not 5-fluoro-6-methyl-2,3-dihydro-1-benzothiophene-4-carboxylic acid, 5-chloro-6-methyl-2,3-dihydro-1-benzothiophene-4-carboxylic acid, or 5,6-dichloro-2,3-dihydro-1-benzothiophene-4-carboxylic acid.

In one embodiment, M is NR⁹; Q is CR⁷R⁶; T is absent; X is absent; and Z is CR⁸R⁶. In one embodiment, the compound is of formula (XX):

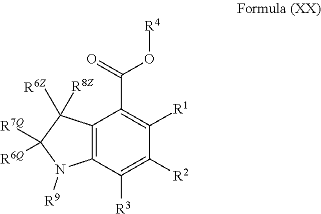

wherein:
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{6Q} and R^{6Z} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q} is selected from the group consisting of H, deuterium, F and C₁₋₃ alkyl optionally substituted with one or more, identical or different, substituents R¹²;
   R^{8Z} is selected from the group consisting of H, deuterium, F and C₁₋₃ alkyl optionally substituted with one or more, identical or different, substituents R¹²;
   R⁹ is selected from the group consisting of H, C₁₋₃ alkyl optionally substituted with one or more, identical or different, substituents R¹² and C₁₋₃ alkyl substituted with R¹⁴;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F; and
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; or a pharmaceutically acceptable salt, hydrate, polymorph, tautomer, or solvate thereof.

In one embodiment, M is CR⁵R⁶; Q is CR⁷R⁶; T is CR⁷R⁶; X is CR⁷R⁶; and Z is O. In one embodiment, the compound is of formula (XXI):

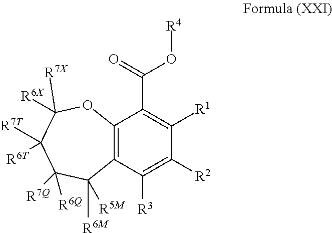

wherein:
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{5M} is selected from the group consisting of H, deuterium, F and C₁₋₃ alkyl optionally substituted with one or more, identical or different, substituents R¹²;
   R^{6M}, R^{6Q} R^{6T} and R^{6X} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q}, R^{7T} and R^{7X} are independently selected from the group consisting of H, deuterium, F and C₁₋₃ alkyl optionally substituted with one or more, identical or different, substituents R¹²;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F; and
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; or a pharmaceutically acceptable salt, hydrate, polymorph, tautomer, or solvate thereof.

In one embodiment, M is CR⁵R⁶; Q is CR⁷R⁶; T is CR⁷R⁶; X is CR⁷R⁶; and Z is S. In one embodiment, the compound is of formula (XXII):

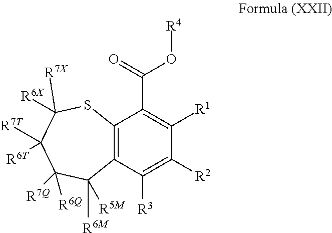

wherein:
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{5M} is selected from the group consisting of H, deuterium, F and C₁₋₃ alkyl optionally substituted with one or more, identical or different, substituents R¹²;
   R^{6M}, R^{6Q} R^{6T} and R^{6X} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q}, R^{7T} and R^{7X} are independently selected from the group consisting of H, deuterium, F and C₁₋₃ alkyl optionally substituted with one or more, identical or different, substituents R¹²;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F; and
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; or a pharmaceutically acceptable salt, hydrate, polymorph, tautomer, or solvate thereof.

In one embodiment, M is CR⁵R⁶; Q is CR⁷R⁶; T is CR⁷R⁶; X is CR⁷R⁶; and Z is NR¹¹. In one embodiment, the compound is of formula (XXIII):

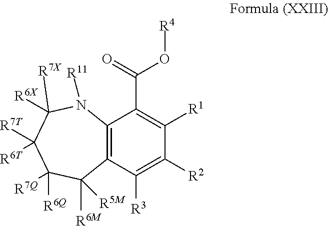

wherein:
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{5M} is selected from the group consisting of H, deuterium, F and C₁₋₃ alkyl optionally substituted with one or more, identical or different, substituents R¹²;
   R^{6M}, R^{6Q} R^{6T} and R^{6X} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q}, R^{7T} and R^{7X} are independently selected from the group consisting of H, deuterium, F and C₁₋₃ alkyl optionally substituted with one or more, identical or different, substituents R¹²;
   R¹¹ is selected from the group consisting of H, C₁₋₃ alkyl optionally substituted with one or more, identical or different, substituents R¹² and C₁₋₃ alkyl substituted with R¹⁵;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F;
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; and
   R¹⁵ is independently selected from the group consisting of C₃₋₆ cycloalkyl optionally substituted with one or more, identical or different, substituents R¹², C₆10 aryl optionally substituted with one or more, identical or different, substituents R¹³ and 5- to 10-membered heteroaryl optionally substituted with one or more, identical or different, substituents R¹³;
or a pharmaceutically acceptable salt, hydrate, polymorph, tautomer, or solvate thereof.

In one embodiment, M is 0; Q is CR⁷R⁶; T is CR⁷R⁶; X is CR⁷R⁶; and Z is CR⁸R⁶. In one embodiment, the compound is of formula (XXIV):

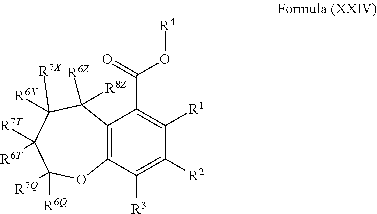

wherein:
   R¹ is selected from the group consisting of F; Cl; C₁₋₃ alkyl optionally substituted with one or more deuterium; C₂₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{6Q}, R^{6T} R^{6X} and R^{6Z} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q}, R^{7T} and R^{7X} are independently selected from the group consisting of H, deuterium, F and C₁₋₃ alkyl optionally substituted with one or more, identical or different, substituents R¹²;
   R^{8Z} is selected from the group consisting of H, deuterium, F and C₁₋₃ alkyl optionally substituted with one or more, identical or different, substituents R¹²;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F; and
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; or a pharmaceutically acceptable salt, hydrate, polymorph, tautomer, or solvate thereof.

M is S; Q is CR⁷R⁶; T is CR⁷R⁶; X is CR⁷R⁶; and Z is CR⁸R⁶. In one embodiment, the compound is of formula (XXV):

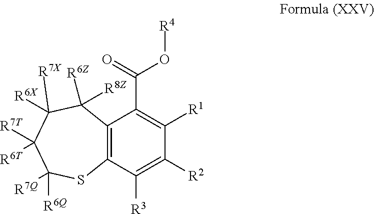

wherein:
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{6Q}, R^{6T} R^{6X} and R^{6Z} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q}, R^{7T} and R^{7X} are independently selected from the group consisting of H, deuterium, F and C₁₋₃ alkyl optionally substituted with one or more, identical or different, substituents R¹²;
   R^{8Z} is selected from the group consisting of H, deuterium, F and C₁₋₃ alkyl optionally substituted with one or more, identical or different, substituents R¹²;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F; and
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; or a pharmaceutically acceptable salt, hydrate, polymorph, tautomer, or solvate thereof.

In one embodiment, M is NR⁹; Q is CR⁷R⁶; T is CR⁷R⁶; X is CR⁷R⁶; and Z is CR⁸R⁶. In one embodiment, the compound is of formula (XXVI):

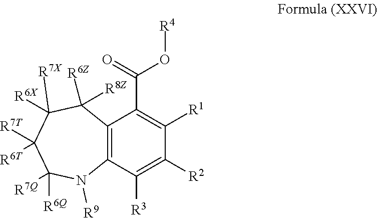

wherein:
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{6Q}, R^{6T} R^{6X} and R^{6Z} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q}, R^{7T} and R^{7X} are independently selected from the group consisting of H, deuterium, F and C₁₋₃ alkyl optionally substituted with one or more, identical or different, substituents R¹²;
   R^{8Z} is selected from the group consisting of H, deuterium, F and C₁₋₃ alkyl optionally substituted with one or more, identical or different, substituents R¹²;
   R⁹ is selected from the group consisting of H, C₁₋₃ alkyl optionally substituted with one or more, identical or different, substituents R¹² and C₁₋₃ alkyl substituted with R¹⁴;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F; and
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; or a pharmaceutically acceptable salt, hydrate, polymorph, tautomer, or solvate thereof.

In one embodiment, M is CR⁵R⁶; Q is NR¹⁰; T is absent; X is absent; and Z is CR⁸R⁶. In one embodiment, the compound is of formula (XXVII):

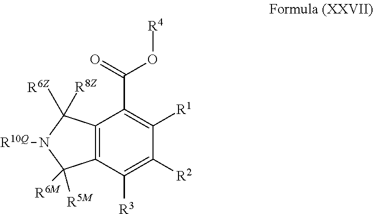

wherein:
   R¹ is selected from the group consisting of F; Cl; C₁₋₃ alkyl optionally substituted with one or more deuterium; C₂₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{5M} is selected from the group consisting of H, deuterium, F and C₁₋₃ alkyl optionally substituted with one or more, identical or different, substituents R¹²;
   R^{6M} and R^{6Z} are independently selected from the group consisting of H, deuterium, F, C₁₋₃ alkyl substituted with R¹⁴ and C₁₋₃ alkyl optionally substituted with one or more, identical or different, substituents R¹²;
   R^{8Z} is selected from the group consisting of H, deuterium, F and C₁₋₃ alkyl optionally substituted with one or more, identical or different, substituents R¹²;
   R^{10Q} is selected from benzyl optionally substituted with one or more, identical or different, substituents R¹³;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F; and
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³;
or a pharmaceutically acceptable salt, hydrate, polymorph, tautomer, or solvate thereof.

In one embodiment, R^{5M} is R⁵. In one embodiment, R^{5M} is H. In one embodiment, R^{6M} is R⁶. In one embodiment, R^{6M} is H. In one embodiment, R^{6Q} is R⁶. In one embodiment, R^{6Q} is H. In one embodiment, R^{6T} is R⁶. In one embodiment, R^{6T} is H. In one embodiment, R^{6X} is R⁶. In one embodiment, R^{6X} is H. In one embodiment, R^{6Z} is R⁶. In one embodiment, R^{6Z} is H. In one embodiment, R^{7Q} is R⁷. In one embodiment, R^{7Q} is H.

In one embodiment, R^{7T} is R⁷. In one embodiment, In one embodiment, R^{7T} is H. In one embodiment, R^{6X} is R⁷. In one embodiment, R^{6X} is H. In one embodiment, R^{8Z} is R⁸. In one embodiment, R^{8Z} is H. In one embodiment, R⁹ is C₁₋₃ alkyl, such as Me. In one embodiment, R^{10Q} is R¹⁰. In one embodiment, R^{10Q} is benzyl. In one embodiment, R10T is R¹⁰. In one embodiment, R^{10T} is benzyl. In one embodiment, Rx is R¹⁰. In one embodiment, R^{10X} is benzyl. In one embodiment, R¹¹ is C₁₋₃ alkyl, such as Me.

In one embodiment, R¹ is F. In one embodiment, R¹ is Cl. In one embodiment, R¹ is C₁. 3 alkyl, such as Me or Et. In one embodiment, R¹ is —C₁₋₃ alkyl substituted with one or more deuterium. In one embodiment, R¹ is —OC₁₋₃ alkyl, such as OMe. In one embodiment, R¹ is —OC₁₋₃ alkyl substituted with one or more, identical or different substituents R¹², wherein R¹² is independently selected from the group consisting of deuterium, F and OMe. In one embodiment, R¹ is —OCH₂F, —OCHF₂, or —OCF₃. In one embodiment, R¹ is —SC₁₋₃ alkyl, such as SMe. In one embodiment, R¹ is —SC₁₋₃ alkyl substituted with one or more, identical or different substituents R¹², wherein R¹² is independently selected from the group consisting of deuterium, F and OMe. In one embodiment, R¹ is selected from the group consisting of F, Cl, Me, OMe, OCH₂F, OCHF₂, OCF₃ and SMe. In one embodiment, R¹ is selected from the group consisting of F, Cl, OMe, OCH₂F, OCHF₂ and OCF₃. In one embodiment, R¹ is selected from the group consisting of F, Cl, OMe and OCHF₂. In one embodiment, R¹ is C₂₋₃ alkenyl, such as C₃ alkenyl. In one embodiment, R¹ is C₂₋₃ alkenyl, such as ethenyl. In one embodiment, R¹ is C₂₋₃ alkenyl substituted with one or more, identical or different substituents R¹², wherein R¹² is independently selected from the group consisting of deuterium, F and OMe. In one embodiment, R¹ is —OC₃₋₅ cycloalkyl. In one embodiment, R¹ is —OC₃₋₅ cycloalkyl substituted with one or more, identical or different substituents R¹², wherein R¹² is independently selected from the group consisting of deuterium, F and OMe. In one embodiment, R¹ is —SC₃₋₅ cycloalkyl. In one embodiment, R¹ is —SC₃₋₅ cycloalkyl substituted with one or more, identical or different substituents R¹², wherein R¹² is independently selected from the group consisting of deuterium, F and OMe.

In one embodiment, R² is F. In one embodiment, R² is Cl. In one embodiment, R² is Br.

In one embodiment, R² is I. In one embodiment, R² is C₁₋₃ alkyl, such as Me or Et. In one embodiment, R² is C₁₋₃ alkyl substituted with one or more, identical or different substituents R¹², wherein R¹² is independently selected from the group consisting of deuterium, F and OMe. In one embodiment, R² is C₃ cycloalkyl, such as cyclopropyl. In one embodiment, R² is C₃ cycloalkyl substituted with one or more, identical or different substituents R¹², wherein R¹² is independently selected from the group consisting of deuterium, F and OMe. In one embodiment, R² is selected from the group consisting of F, Cl and Me. In one embodiment, R² is selected from the group consisting of F and Cl.

In one embodiment, R³ is H. In one embodiment, R³ is F. In one embodiment, R³ is Cl.

In one embodiment, R⁴ is H. In one embodiment, R⁴ is C₁₋₅ alkyl. In one embodiment, R⁴ is C₁₋₅ alkyl substituted with one or more, identical or different substituents R¹²; wherein R¹² is independently selected from the group consisting of deuterium, F and Cl. In one embodiment, R⁴ is C₂₋₅ alkenyl. In one embodiment, R⁴ is C₂₋₅ alkenyl substituted with one or more, identical or different substituents R¹², wherein R¹² is independently selected from the group consisting of deuterium, F and Cl. In one embodiment, R⁴ is C₂₋₅ alkynyl. In one embodiment, R⁴ is C₂₋₅ alkynyl substituted with one or more, identical or different substituents R¹², wherein R¹² is independently selected from the group consisting of deuterium, F and Cl. In one embodiment, R⁴ is C₃₋₆ cycloalkyl. In one embodiment, R⁴ is C₃₋₆ cycloalkyl substituted with one or more, identical or different substituents R¹², wherein R¹² is independently selected from the group consisting of deuterium, F and Cl. In one embodiment, R⁴ is phenyl. In one embodiment, R⁴ is phenyl substituted with one or more, identical or different substituents R¹³, wherein R¹³ is independently selected from the group consisting of deuterium, methoxy, nitro, cyano, Cl, Br, I, and F. In one embodiment, R⁴ is benzyl. In one embodiment, R⁴ is benzyl substituted with one or more, identical or different substituents R¹³, wherein R¹³ is independently selected from the group consisting of deuterium, methoxy, nitro, cyano, Cl, Br, I, and F.

In one embodiment, R⁵ is H. In one embodiment, R⁵ is deuterium. In one embodiment, R⁵ is F. In one embodiment, R⁵ is a bond. When R⁵ is a bond, then R⁵ is linked to R⁸ or R¹¹ to form a ring. In one embodiment, R⁵ is linked to R^{a} or R¹¹ to form a ring, and R⁵ is C₁₋₃ alkanediyl. In one embodiment, R⁵ is C₁₋₃ alkyl. In one embodiment, R⁵ is C₁₋₃ alkyl substituted with one or more, identical or different, substituents R¹², wherein R¹² is independently selected from the group consisting of deuterium, F and OMe.

In one embodiment, R⁶ is H. In one embodiment, R⁶ is deuterium. In one embodiment, R⁶ is F. In one embodiment, R⁶ is C₁₋₃ alkyl substituted with R¹⁴. In one embodiment, R⁶ is C₁₋₃ alkyl substituted with R¹⁴, wherein R¹⁴ is C₃₋₅ cycloalkyl optionally substituted with one or more, identical or different, substituents R¹², and R¹² is independently selected from the group consisting of deuterium, F and OMe. In one embodiment, R⁶ is C₁₋₃ alkyl substituted with R¹⁴, wherein R¹⁴ is phenyl optionally substituted with one or more, identical or different, substituents R¹³, and R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F. In one embodiment, R⁶ is C₁₋₃ alkyl substituted with R¹⁴, wherein R¹⁴ is 5-membered heteroaryl optionally substituted with one or more, identical or different, substituents R¹³, and R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F. In one embodiment, R⁶ is C₁ alkyl substituted with R¹⁴; R¹⁴ is phenyl optionally substituted with one or more, identical or different, substituents R¹³; and R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F. In one embodiment, R⁶ is benzyl. In one embodiment, R⁶ is C₁₋₃ alkyl. In one embodiment, R⁶ is C₁₋₃ alkyl substituted with one or more, identical or different, substituents R¹², wherein R¹² is independently selected from the group consisting of deuterium, F and OMe.

In one embodiment, R⁷ is H. In one embodiment, R⁷ is deuterium. In one embodiment, R⁷ is F. In one embodiment, R⁷ is C₁₋₃ alkyl. In one embodiment, R⁷ is C₁₋₃ alkyl substituted with one or more, identical or different, substituents R¹², wherein R¹² is independently selected from the group consisting of deuterium, F and OMe.

In one embodiment, R^{a} is H. In one embodiment, R^{a} is deuterium. In one embodiment, R^{a} is F. In one embodiment, R^{a} is a bond. When R^{a} is a bond, then R^{a} is linked to R⁵ or R⁹ to form a ring. In one embodiment, R^{a} is linked to R⁵ or R⁹ to form a ring, and R^{a} is C₁₋₃ alkanediyl. In one embodiment, R^{a} is C₁₋₃ alkyl. In one embodiment, R^{a} is C₁₋₃ alkyl substituted with one or more, identical or different, substituents R¹², wherein R¹² is independently selected from the group consisting of deuterium, F and OMe.

In one embodiment, R⁹ is H. In one embodiment, R⁹ is C₁₋₅ alkyl. In one embodiment, R⁹ is C₁₋₅ alkyl substituted with one or more, identical or different, substituents R¹², wherein R¹² is independently selected from the group consisting of deuterium, F and OMe. In one embodiment, R⁹ is a bond. When R⁹ is a bond, then R⁹ is linked to R⁸ to form a ring. In one embodiment, R⁹ is linked to R^{a} to form a ring, and R⁹ is C₁.3 alkanediyl. In one embodiment, R⁹ is C₁₋₃ alkyl substituted with R¹⁴. In one embodiment, R⁹ is C₁₋₃ alkyl substituted with R¹⁴, wherein R¹⁴ is C₃₋₅ cycloalkyl optionally substituted with one or more, identical or different, substituents R¹², and R¹²; is independently selected from the group consisting of deuterium, F and OMe. In one embodiment, R⁹ is C₁₋₃ alkyl substituted with R¹⁴, wherein R¹⁴ is phenyl optionally substituted with one or more, identical or different, substituents R¹³, and wherein R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F. In one embodiment, R⁹ is C₁₋₃ alkyl substituted with R¹⁴, wherein R¹⁴ is 5-membered heteroaryl optionally substituted with one or more, identical or different, substituents R¹³, and wherein R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F. In one embodiment, R⁹ is benzyl. In one embodiment, R⁹ is benzyl optionally substituted with one or more, identical or different, substituents R¹³, wherein R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F. In one embodiment, R⁹ is benzyl optionally substituted with one or more, identical or different, substituents R¹³, wherein R¹³ is independently selected from the group consisting of deuterium, methoxy, nitro, cyano, Cl, Br, I, and F.

In one embodiment, R¹⁰ is benzyl. In one embodiment, R¹⁰ is benzyl optionally substituted with one or more, identical or different, substituents R¹³, wherein R¹³ is independently selected from the group consisting of deuterium, methoxy, nitro, cyano, Cl, Br, I, and F. In one embodiment, R¹⁰ is benzyl optionally substituted with one or more, identical or different, substituents R¹³, wherein R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F.

In one embodiment, R¹¹ is H. In one embodiment, R¹¹ is C₁₋₅ alkyl. In one embodiment, R¹¹ is C₁₋₅ alkyl substituted with one or more, identical or different, substituents R¹², wherein R¹² is independently selected from the group consisting of deuterium, F and OMe. In one embodiment, R¹¹ is a bond. When R¹¹ is a bond, then R¹¹ is linked to R⁵ to form a ring. In one embodiment, R¹¹ is linked to R⁵ to form a ring, and R¹¹ is C₁₋₃ alkanediyl. In one embodiment, R¹¹ is C₁₋₃ alkyl substituted with R¹⁵. In one embodiment, R¹¹ is C₁₋₃ alkyl substituted with R¹⁵, wherein R¹⁵ is C₃₋₆ cycloalkyl optionally substituted with one or more, identical or different, substituents R¹², and wherein R¹² is independently selected from the group consisting of deuterium, F and OMe. In one embodiment, R¹¹ is C₁₋₃ alkyl substituted with R¹⁵, wherein R¹⁵ is C₆-1o aryl optionally substituted with one or more, identical or different, substituents R¹³, and wherein R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F. In one embodiment, R¹¹ is C₁₋₃ alkyl substituted with R¹⁵, wherein R¹⁵ is 5- to 10-membered heteroaryl optionally substituted with one or more, identical or different, substituents R¹³, and wherein R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F. In one embodiment, R¹¹ is benzyl. In one embodiment, R¹¹ is benzyl optionally substituted with one or more, identical or different, substituents R¹³, wherein R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F.

In one embodiment, R¹¹ is benzyl optionally substituted with one or more, identical or different, substituents R¹³, wherein R¹³ is independently selected from the group consisting of deuterium, methoxy, nitro, cyano, Cl, Br, I, and F.

In one embodiment, R⁵, R⁶ R⁷ and R⁸ are H.

In one embodiment, R¹² is F. In one embodiment, R¹² is OMe. In one embodiment, R¹²; is deuterium.

In one embodiment, R¹³ is deuterium. In one embodiment, R¹³ is methoxy. In one embodiment, R¹³ is —OCF₃. In one embodiment, R¹³ is Me. In one embodiment, R¹³ is CF₃. In one embodiment, R¹³ is CF₂Cl. In one embodiment, R¹³ is CF₂H. In one embodiment, R¹³ is CFH₂. In one embodiment, R¹³ is CD₃. In one embodiment, R¹³ is cyclopropyl. In one embodiment, R¹³ is NH₂. In one embodiment, R¹³ is —NHAc. In one embodiment, R¹³ is —C(═O)—NH₂. In one embodiment, R¹³ is nitro. In one embodiment, R¹³ is cyano. In one embodiment, R¹³ is Cl. In one embodiment, R¹³ is Br. In one embodiment, R¹³ is I. In one embodiment, R¹³ is F.

In one embodiment, R¹⁴ is C₃₋₅ cycloalkyl, such as cyclopropyl. In one embodiment, R¹⁴ is C₃₋₅ cycloalkyl substituted with one or more, identical or different substituents R¹²; wherein R¹² is independently selected from the group consisting of deuterium, F and OMe. In one embodiment, R¹⁴ is phenyl. In one embodiment, R¹⁴ is phenyl substituted with one or more, identical or different, substituents R¹³, wherein R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F.

In one embodiment, R¹⁴ is 5-membered heteroaryl. In one embodiment, R¹⁴ is 5-membered heteroaryl substituted with one or more, identical or different, substituents R¹³, wherein R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F.

In one embodiment, R¹⁴ is selected from the group consisting of oxazolyl, thiazolyl, thiadiazolyl, oxadiazolyl, triazolyl, pyrrolyl, thienyl, furanyl, diazolyl, imidazolyl, pyrazolyl, isoxazolyl, isothiazolyl, and tetrazolyl. In one embodiment, R¹⁴ is selected from the group consisting of oxazolyl, thiazolyl, thiadiazolyl, oxadiazolyl, triazolyl, pyrrolyl, thienyl, furanyl, diazolyl, imidazolyl, pyrazolyl, isoxazolyl, isothiazolyl, and tetrazolyl, each of which is substituted with one or more, identical or different, substituents R¹³, wherein R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F. In one embodiment, R¹⁴ is furanyl, such as furan-2-yl, optionally substituted with one or more, identical or different, substituents R¹³. In one embodiment, R¹⁴ is thiazolyl, such as thiazol-2-yl, optionally substituted with one or more, identical or different, substituents R¹³. In one embodiment, R¹⁴ is oxazolyl, such as oxazol-2-yl, optionally substituted with one or more, identical or different, substituents R¹³.

In one embodiment, R¹⁵ is C₃₋₆ cycloalkyl, such as cyclobutyl, optionally substituted with one or more, identical or different, substituents R¹², wherein R¹² is independently selected from the group consisting of deuterium, F and OMe. In one embodiment, R¹⁵ is C₆10 aryl optionally substituted with one or more, identical or different, substituents R¹³, wherein R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F. In one embodiment, R¹⁵ is 5- to 10-membered heteroaryl optionally substituted with one or more, identical or different, substituents R¹³, wherein R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F. In one embodiment, R¹⁵ is selected from the group consisting of oxazolyl, thiazolyl, thiadiazolyl, oxadiazolyl, triazolyl, pyrrolyl, thienyl, furanyl, diazolyl, imidazolyl, pyrazolyl, isoxazolyl, isothiazolyl, and tetrazolyl, pyridinyl, pyridazinyl, pyrimidinyl, pyrazinyl, triazinyl, 1,4-dihydropyrrolo[3,2-b]pyrrolyl, 1,6-dihydropyrrolo[2,3-b]pyrrolyl, 6H-furo[2,3-b]pyrrole, 4H-furo[2,3-b]pyrrolyl, 6H-thieno[2,3-b]pyrrolyl, 4H-thieno[2,3-b]pyrrolyl, indolyl, isoindolyl, indolizinyl, indazolyl, benzimidazolyl, azaindolyl, azaindazolyl, pyrazolo[1,5-a]pyrimidinyl, purinyl, benzofuranyl, benzo[b]thiophenyl, benzisoxazolyl, benzthiazolyl, benzisothiazolyl, benzo[c][1,2,5]thiadiazolyl, quinolinyl, isoquinolinyl, 4H-quinolizinyl, quinoxalinyl, phthalazinyl, quinazolinyl, cinnolinyl, 1,8-naphthyridinyl, pyrido[3,2-d]pyrimidinyl, pyrido[4,3-d]pyrimidinyl, pyrido[3,4-b]pyrazinyl, pyrido[2,3-b]pyrazinyl and pteridinyl. In one embodiment, R¹⁵ is selected from the group consisting of oxazolyl, thiazolyl, thiadiazolyl, oxadiazolyl, triazolyl, pyrrolyl, thienyl, furanyl, diazolyl, imidazolyl, pyrazolyl, isoxazolyl, isothiazolyl, and tetrazolyl, pyridinyl, pyridazinyl, pyrimidinyl, pyrazinyl, triazinyl, 1,4-dihydropyrrolo[3,2-b]pyrrolyl, 1,6-dihydropyrrolo[2,3-b]pyrrolyl, 6H-furo[2,3-b]pyrrole, 4H-furo[2,3-b]pyrrolyl, 6H-thieno[2,3-b]pyrrolyl, 4H-thieno[2,3-b]pyrrolyl, indolyl, isoindolyl, indolizinyl, indazolyl, benzimidazolyl, azaindolyl, azaindazolyl, pyrazolo[1,5-a]pyrimidinyl, purinyl, benzofuranyl, benzo[b]thiophenyl, benzisoxazolyl, benzthiazolyl, benzisothiazolyl, benzo[c][1,2,5]thiadiazolyl, quinolinyl, isoquinolinyl, 4H-quinolizinyl, quinoxalinyl, phthalazinyl, quinazolinyl, cinnolinyl, 1,8-naphthyridinyl, pyrido[3,2-d]pyrimidinyl, pyrido[4,3-d]pyrimidinyl, pyrido[3,4-b]pyrazinyl, pyrido[2,3-b]pyrazinyl and pteridinyl, each of which is substituted with one or more, identical or different, substituents R¹³, wherein R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F. In one embodiment, R¹⁵ is thienyl, such as thien-3-yl, optionally substituted with one or more, identical or different, substituents R¹³. In one embodiment, R¹⁵ is naphthyl, such as naphth-1-yl, optionally substituted with one or more, identical or different, substituents R¹³.

In one embodiment, R¹ is selected from the group consisting of F, Cl, Me, OMe, OCH₂F, OCHF₂, OCF₃ and SMe, and R² is selected from the group consisting of F, Cl and Me. In one embodiment, R¹ is selected from the group consisting of F, Cl, Me, OMe, OCH₂F, OCHF₂, OCF₃ and SMe; R² is selected from the group consisting of F, Cl and Me; and R⁴ is H. In one embodiment, R¹ is selected from the group consisting of F, Cl, Me, OMe, OCH₂F, OCHF₂, OCF₃ and SMe; R² is selected from the group consisting of F, Cl and Me; and R³ is H. In one embodiment, R¹ is selected from the group consisting of F, Cl, Me, OMe, OCH₂F, OCHF₂, OCF₃ and SMe; R² is selected from the group consisting of F, Cl and Me; R³ is H; and R⁴ is H. In one embodiment, R³, R⁴, R⁵, R⁶, R⁷ and R⁸ are H. In one embodiment, R¹ is selected from the group consisting of F, Cl, Me, OMe, OCH₂F, OCHF₂, OCF₃ and SMe; R² is selected from the group consisting of F, Cl and Me; and R³, R⁴, R⁵, R⁶, R⁷ and R^{a} are H.

In one embodiment, the compound is selected from the group consisting of:
6-chloro-5-methyl-2,3-dihydro-1H-indene-4-carboxylic acid;
6-chloro-5-methoxy-2,3-dihydro-1H-indene-4-carboxylic acid;
6-chloro-5-(fluoromethoxy)-2,3-dihydro-1H-indene-4-carboxylic acid;
3-chloro-2-methyl-5,6,7,8-tetrahydronaphthalene-1-carboxylic acid;
6-chloro-5-(difluoromethoxy)-2,3-dihydro-1H-indene-4-carboxylic acid;
6-chloro-5-(trifluoromethoxy)-2,3-dihydro-1H-indene-4-carboxylic acid;
5-chloro-6-methyl-2,3-dihydro-1-benzofuran-7-carboxylic acid;
6-chloro-5-ethyl-2,3-dihydro-1H-indene-4-carboxylic acid;
6-chloro-7-methyl-3,4-dihydro-2H-1-benzopyran-8-carboxylic acid;
5-chloro-6-methoxy-2,3-dihydro-1-benzofuran-7-carboxylic acid;
5-chloro-4-methoxytricyclo[6.2.1.0^{2,7}]undeca-2(7),3,5-triene-3-carboxylic acid;
3-fluoro-2-methoxy-5,6,7,8-tetrahydronaphthalene-1-carboxylic acid;
3-chloro-4-fluoro-2-methoxy-5,6,7,8-tetrahydronaphthalene-1-carboxylic acid;
4-chloro-5-methyltricyclo[6.2.1.0^{2,7}]undeca-2,4,6-triene-3-carboxylic acid;
5-chloro-4-methyltricyclo[6.2.1.0^{2,7}]undeca-2(7),3,5-triene-3-carboxylic acid;
6-chloro-5-(methylsulfanyl)-2,3-dihydro-1H-indene-4-carboxylic acid;
3-chloro-2-(difluoromethoxy)-5,6,7,8-tetrahydronaphthalene-1-carboxylic acid;
3-chloro-2-fluoro-5,6,7,8-tetrahydronaphthalene-1-carboxylic acid;
2-chloro-3-fluoro-5,6,7,8-tetrahydronaphthalene-1-carboxylic acid;
2-chloro-3-methyl-5,6,7,8-tetrahydronaphthalene-1-carboxylic acid;
5-chloro-4-methoxytricyclo[6.2.2.0^{2,7}]dodeca-2,4,6-triene-3-carboxylic acid;
2,3-dichloro-5,6,7,8-tetrahydronaphthalene-1-carboxylic acid;
6-chloro-7-methoxy-3,4-dihydro-2H-1-benzothiopyran-8-carboxylic acid;
3-chloro-2-(methylsulfanyl)-5,6,7,8-tetrahydronaphthalene-1-carboxylic acid;
3-chloro-2-methyl-6,7,8,9-tetrahydro-5H-benzo[7]annulene-1-carboxylic acid;
2-chloro-3-methyl-6,7,8,9-tetrahydro-5H-benzo[7]annulene-1-carboxylic acid;
3-chloro-2-ethenyl-5,6,7,8-tetrahydronaphthalene-1-carboxylic acid;
6-chloro-7-fluoro-3,4-dihydro-2H-1-benzopyran-8-carboxylic acid;
7-chloro-6-fluoro-3,4-dihydro-2H-1-benzopyran-8-carboxylic acid;
6-chloro-7-fluoro-3,4-dihydro-2H-1-benzothiopyran-8-carboxylic acid;
7-chloro-6-fluoro-3,4-dihydro-2H-1-benzopyran-5-carboxylic acid;
6-chloro-7-fluoro-1-methyl-1,2,3,4-tetrahydroquinoline-8-carboxylic acid;
6-chloro-7-fluoro-1,2,3,4-tetrahydroquinoline-8-carboxylic acid;
6,7-difluoro-3,4-dihydro-2H-1-benzothiopyran-8-carboxylic acid;
5-chloro-6-fluoro-2,3-dihydro-1-benzofuran-7-carboxylic acid;
2,3-difluoro-5,6,7,8-tetrahydronaphthalene-1-carboxylic acid;
7-chloro-6-fluoro-1-methyl-1,2,3,4-tetrahydroquinoline-5-carboxylic acid;
7-chloro-6-fluoro-1-methyl-1,2,3,4-tetrahydroquinoline-8-carboxylic acid;
5-methoxy-6-methyl-2,3-dihydro-1H-indene-4-carboxylic acid;
7-fluoro-6-methyl-3,4-dihydro-2H-1-benzothiopyran-8-carboxylic acid;
2-fluoro-3-methyl-5,6,7,8-tetrahydronaphthalene-1-carboxylic acid;
7-fluoro-6-methyl-3,4-dihydro-2H-1-benzopyran-8-carboxylic acid;
3-ethyl-2-fluoro-5,6,7,8-tetrahydronaphthalene-1-carboxylic acid;
3-fluoro-2-methyl-5,6,7,8-tetrahydronaphthalene-1-carboxylic acid;
3-cyclopropyl-2-fluoro-5,6,7,8-tetrahydronaphthalene-1-carboxylic acid;
8-chloro-7-fluoro-2,3,4,5-tetrahydro-1-benzoxepine-9-carboxylic acid;
6-fluoro-7-methyl-3,4-dihydro-2H-1-benzopyran-5-carboxylic acid;
8-chloro-7-fluoro-2,3,4,5-tetrahydro-1-benzoxepine-6-carboxylic acid;
3-chloro-8,8-difluoro-2-methoxy-5,6,7,8-tetrahydronaphthalene-1-carboxylic acid;
2-fluoro-3-(trifluoromethyl)-5,6,7,8-tetrahydronaphthalene-1-carboxylic acid;
6-chloro-7-fluoro-2,2-dimethyl-3,4-dihydro-2H-1-benzopyran-8-carboxylic acid;
5-chloro-6-fluoro-2,3-dihydro-1H-indene-4-carboxylic acid;
6-chloro-5-fluoro-2,3-dihydro-1H-indene-4-carboxylic acid;
7-chloro-6-fluoro-3,4-dihydro-2H-1-benzothiopyran-8-carboxylic acid;
7-chloro-6-fluoro-3,4-dihydro-2H-1-benzothiopyran-5-carboxylic acid;
2-benzyl-5,6-dichloro-2,3-dihydro-1H-isoindole-4-carboxylic acid;
6-chloro-7-methoxy-3,4-dihydro-2H-1-benzopyran-8-carboxylic acid;
6-chloro-7-fluoro-1-[(3-thienyl)methyl]-1,2,3,4-tetrahydro-8-quinolinecarboxylic acid;
1-benzyl-6-chloro-7-fluoro-1,2,3,4-tetrahydro-8-quinolinecarboxylic acid;
6-chloro-7-fluoro-1-[(p-fluorophenyl)methyl]-1,2,3,4-tetrahydro-8-quinolinecarboxylic acid;
3-chloro-5,5-difluoro-2-methoxy-5,6,7,8-tetrahydro-1-naphthoic acid;
1-benzyl-7-chloro-6-fluoro-1,2,3,4-tetrahydro-8-quinolinecarboxylic acid;
7-chloro-6-fluoro-2,2-dimethyl-8-chromancarboxylic acid;
1-benzyl-6-chloro-5-fluoro-4-indolinecarboxylic acid;
7-chloro-6-fluoro-1-methyl-1,2,3,4-tetrahydro-8-quinolinecarboxylic acid;
4-benzyl-6-chloro-7-fluoro-8-chromancarboxylic acid;
7-chloro-6-methoxy-5-chromancarboxylic acid;
4-benzyl-7-fluoro-6-methoxy-8-chromancarboxylic acid;
4-benzyl-7-fluoro-6-methoxy-5-chromancarboxylic acid;
6-chloro-7-fluoro-5-chromancarboxylic acid; and
3-chloro-2-ethoxy-5,6,7,8-tetrahydro-1-naphthoic acid.

In one embodiment, the compound or the compound for use according to the present disclosure has been modified in order to increase its half-life when administered to a patient, in particular its plasma half-life.

In one embodiment, the compound or the compound for use according to the present disclosure further comprises a moiety conjugated to said compound, thus generating a moiety-conjugated compound. In one embodiment, said moiety-conjugated compound has a plasma and/or serum half-life being longer than the plasma and/or serum half-life of the non-moiety conjugated compound.

In one embodiment, the moiety conjugated to the compound or compound for use according to the present disclosure, is one or more type(s) of moieties selected from the group consisting of albumin, fatty acids, polyethylene glycol (PEG), acylation groups, antibodies and antibody fragments.

In one embodiment, the compound has activity on the CIC-1 receptor. In one embodiment, the compound is an inhibitor of the CIC-1 ion channel.

In one embodiment, the compound is capable of improving the recovered force in isolated rat soleus muscles after exposure to tubocurarine. In one embodiment, the recovery of force in muscles with neuromuscular dysfunction is >5%, for example >10%, for example >15%, for example >20%, for example >25%, for example >30% and for example >35%.

### Neuromuscular Disorders

The compound or compound for use of the present disclosure may be used for treating, ameliorating and/or preventing a neuromuscular disorder, or reversing neuromuscular blockade.

The inventors of the present disclosure have shown that inhibition of CIC-1 channels strengthens neuromuscular transmission. CIC-1 function may therefore contribute to muscle weakness in conditions of compromised neuromuscular transmission. Thus, in one embodiment, the compound as described herein inhibits CIC-1 channels.

Thus, it is appreciated that compounds and/or compounds for use of Formula (I) inhibit CIC-1 channels.

The neuromuscular disorder may also include neuromuscular dysfunctions.

Neuromuscular disorders include for example disorders with symptoms of muscle weakness and fatigue. Such disorders may include conditions with reduced neuromuscular transmission safety factor. In one embodiment the neuromuscular disorders are motor neuron disorders. Motor neuron disorders are disorders with reduced safety in the neuromuscular transmission. In one embodiment motor neuron disorders are selected from the group consisting of amyotrophic lateral sclerosis (ALS) (Killian J M, Wilfong A A, Burnett L, Appel S H, Boland D. Decremental motor responses to repetitive nerve stimulation in ALS. Muscle Nerve, 1994, 17, 747-754), spinal muscular atrophy (SMA) (Wadman R I, Vrancken A F, van den Berg L H, van der Pol WL. Dysfunction of the neuromuscular junction in spinal muscular atrophy types 2 and 3. Neurology, 2012, 79, 2050-2055), Charcot-Marie Tooth disease (Bansagi B, Griffin H, Whittaker R G, Antoniadi T, Evangelista T, Miller J, Greenslade M, Forester N, Duff J, Bradshaw A, Kleinle S, Boczonadi V, Steele H, Ramesh V, Franko E, Pyle A, Lochmuller H, Chinnery P F, Horvath R. Genetic heterogeneity of motor neuropathies. Neurology, 2017, 28; 88(13):1226-1234), X-linked spinal and bulbar muscular atrophy (Yamada, M., Inaba, A., Shiojiri, T. X-linked spinal and bulbar muscular atrophy with myasthenic symptoms. Journal of the Neurological Sciences, 1997, 146, 183-185), Kennedy's disorder (Stevic, Z., Peric, S., Pavlovic, S., Basta, I., Lavrnic, D., Myasthenic symptoms in a patient with Kennedy's disorder. Acta Neurologica Belgica, 2014, 114, 71-73), multifocal motor neuropathy (Roberts, M., Willison, H. J., Vincent, A., Newsom-Davis, J. Multifocal motor neuropathy human sera block distal motor nerve conduction in mice. Ann Neurol. 1995, 38, 111-118), Guillain-Barre syndrome (Ansar, V., Valadi, N. Guillain-Barré Syndrome Prim. Care, 2015, 42, 189-193); poliomyelitis (Trojan, D. A., Gendron, D., Cashman, N. R. Electrophysiology and electrodiagnosis of the post-polio motor unit. Orthopedics, 1991, 14, 1353-1361, and Birk T. J. Poliomyelitis and the post-polio syndrome: exercise capacities and adaptation—current research, future directions, and widespread applicability. Med. Sci. Sports Exerc., 1993, 25, 466-472), post-polio syndrome (Garcia, C. C., Potian, J. G., Hognason, K., Thyagarajan, B., Sultatos, L. G., Souayah, N., Routh, V. H., McArdle, J. J. Acetylcholinesterase deficiency contributes to neuromuscular junction dysfunction in type 1 diabetic neuropathy. Am. J. Physiol. Endocrinol. Metab., 2012, 15, E551-561) and sarcopenia (Gilmore K. J., Morat T., Doherty T. J., Rice C. L., Motor unit number estimation and neuromuscular fidelity in 3 stages of sarcopenia. 2017 55(5):676-684). In one embodiment, the neuromuscular disorder is diabetic polyneuropathy. In one embodiment, the neuromuscular disorder is sarcopenia. In one embodiment, the neuromuscular disorder is Kennedy's disorder. In one embodiment, the neuromuscular disorder is multifocal motor neuropathy.

Thus, in one embodiment of the present disclosure the neuromuscular disorder is amyotrophic lateral sclerosis (ALS). In another embodiment the neuromuscular disorder is spinal muscular atrophy (SMA). In another embodiment the neuromuscular disorder is Charcot-Marie tooth disease (CMT). In another embodiment the neuromuscular disorder is sarcopenia. In yet another embodiment, the neuromuscular disorder is critical illness myopathy (CIM).

As stated above the neuromuscular disorders include for example disorders with symptoms of muscle weakness and fatigue. Such disorder may for example include diabetes (Am. J. Physiol. Endocrinol. Metab., 2012, 15, E551-561).

In another embodiment the neuromuscular disorders is chronic fatigue syndrome. Chronic fatigue syndrome (CFS) (Fletcher, S. N., Kennedy, D. D., Ghosh, I. R., Misra, V. P., Kiff, K., Coakley, J. H., Hinds, C. J. Persistent neuromuscular and neurophysiologic abnormalities in long-term survivors of prolonged critical illness. Crit. Care Med. 2003, 31, 1012-1016) is the common name for a medical condition characterized by debilitating symptoms, including fatigue that lasts for a minimum of six months in adults. CFS may also be referred to as systemic exertion intolerance disorder (SEID), myalgic encephalomyelitis (ME), post-viral fatigue syndrome (PVFS), chronic fatigue immune dysfunction syndrome (CFIDS), or by several other terms. Symptoms of CFS include malaise after exertion; unrefreshing sleep, widespread muscle and joint pain, physical exhaustion, and muscle weakness.

In a further embodiment the neuromuscular disorder is a critical illness polyneuropathy (Angelini C. Spectrum of metabolic myopathies. Biochim. Biophys. Acta., 2015, 1852, 615-621) or CIM (Latronico, N., Bolton, C. F. Critical illness polyneuropathy and myopathy: a major cause of muscle weakness and paralysis. Lancet Neurol. 2011, 10, 931-941). Critical illness polyneuropathy and CIM are overlapping syndromes of widespread muscle weakness and neurological dysfunction developing in critically ill patients.

The neuromuscular disorder may also include metabolic myopathy (Milone, M., Wong, L. J. Diagnosis of mitochondrial myopathies. Mol. Genet. Metab., 2013, 110, 35-41) and mitochondrial myopathy (Srivastava, A., Hunter, J. M. Reversal of neuromuscular block. Br. J. Anaesth. 2009, 103, 115-129). Metabolic myopathies result from defects in biochemical metabolism that primarily affects muscle. These may include glycogen storage disorders, lipid storage disorder and 3-phosphocreatine stores disorder. Mitochondrial myopathy is a type of myopathy associated with mitochondrial disorder. Symptoms of mitochondrial myopathies include muscular and neurological problems such as muscle weakness, exercise intolerance, hearing loss and trouble with balance and coordination. Thus, in one embodiment of the present disclosure the neuromuscular disorder is metabolic myopathy.

In another embodiment the neuromuscular disorder is periodic paralysis, in particular hypokalemic periodic paralysis which is a disorder of skeletal muscle excitability that presents with recurrent episodes of weakness, often triggered by exercise, stress, or carbohydrate-rich meals (Wu, F., Mi, W., Cannon, S. C., Neurology, 2013, 80, 1110-1116 and Suetterlin, K. et at, Current Opinion Neurology, 2014, 27, 583-590) or hyperkalemic periodic paralysis which is an inherited autosomal dominant disorder that affects sodium channels in muscle cells and the ability to regulate potassium levels in the blood (Ammat, T. et at, Journal of General Physiology, 2015, 146, 509-525).

In an embodiment the neuromuscular disorder is a myasthenic condition. Myasthenic conditions are characterized by muscle weakness and neuromuscular transmission failure. Congenital myasthenic syndrome (Finlayson, S., Beeson, D., Palace, J. Congenital myasthenic syndromes: an update. Pract. Neurol., 2013, 13, 80-91) is an inherited neuromuscular disorder caused by defects of several types at the neuromuscular junction.

Myasthenia gravis and Lambert-Eaton syndrome (Titulaer M J, Lang B, Verschuuren J J. Lambert-Eaton myasthenic syndrome: from clinical characteristics to therapeutic strategies. Lancet Neurol. 2011, 10, 1098-107) are examples of myasthenic conditions. Myasthenia gravis is either an autoimmune or congenital neuromuscular disorder that leads to fluctuating muscle weakness and fatigue. In the most common cases, muscle weakness is caused by circulating antibodies that block ACh receptors at the postsynaptic neuromuscular junction, inhibiting the excitatory effects of the neurotransmitter ACh on nicotinic ACh-receptors at neuromuscular junctions (Gilhus, N. E., Owe, J. F., Hoff, J. M., Romi, F., Skeie, G. O., Aarli, J. A. Myasthenia Gravis: A Review of Available Treatment Approaches, Autoimmune Diseases, 2011, Article ID 84739). Lambert-Eaton myasthenic syndrome (also known as LEMS, Lambert-Eaton syndrome, or Eaton-Lambert syndrome) is a rare autoimmune disorder that is characterized by muscle weakness of the limbs. It is the result of an autoimmune reaction in which antibodies are formed against presynaptic voltage-gated calcium channels, and likely other nerve terminal proteins, in the neuromuscular junction.

Thirty to fifty percent of patients with acetylcholine receptor (AChR) antibody-negative myasthenia gravis (MG) have antibodies to muscle specific kinase (MuSK) and are referred to as having MuSK-MG (Borges, L. S., Richman, D. P., Muscle-Specific Kinase Myasthenia Gravis, Frontiers in Immunology, 2020, 11:707). In one embodiment of the present disclosure the neuromuscular disorder is myasthenia gravis. In another embodiment the neuromuscular disorder is autoimmune myasthenia gravis. In another embodiment the neuromuscular disorder is MuSK-MG. In another embodiment the neuromuscular disorder is Lambert-Eaton syndrome. In another embodiment the neuromuscular disorder is seronegative myasthenia gravis.

X-linked myotubular myopathy is a part of a group of centronuclear myopathies where cell nuclei are abnormally located in the centre of muscle cells instead of their normal location at the periphery. It is one of the severest congenital muscle diseases and is characterized by marked muscle weakness, hypotonia and feeding and breathing difficulties (Dowling J J, Lawlor M W, Das S. X-Linked Myotubular Myopathy. 2002 Feb. 25 [Updated 2018 Aug. 23]. In: Adam M P, Ardinger H H, Pagon R A, et al., editors. GeneReviews® [Internet]. Seattle (WA): University of Washington, Seattle; 1993-2022. Available from: https://www.ncbi.nlm.nih.gov/books/NBK1432/). In one embodiment the neuromuscular disorder is myotubular myopathy.

Duchenne muscular dystrophy is a severe type of muscular dystrophy that primarily affects boys resulting initially in fatigue and muscle weakness (Angelini C, Tasca E, Fatigue in muscular dystrophies, Neuromuscular Disorders, 2012, 22 Suppl 3: S214; 20. doi:10.1016/j.nmd.2012.10.010). In one embodiment the neuromuscular disorder is Duchenne muscular dystrophy.

Multiple sclerosis (MS) is the most common demyelinating disease, in which the insulating covers of nerve cells in the brain and spinal cord are damaged. This damage disrupts the ability of parts of the nervous system to transmit signals, resulting in a range of signs and symptoms, including physical, mental, and sometimes psychiatric problems. It has been shown that people with multiple sclerosis typically experience greater levels of exercise-induced fatigue compared with healthy individuals (Brotherton et al, J. Neuorophysiol. 2022, 128, 105-117). There are four types of MS in what is known as the Lublin classification: clinically isolated syndrome (CIS), relapsing-remitting MS (RRMS), primary progressive MS (PPMS) and secondary progressive MS (SPMS). In one embodiment, the neuromuscular disorder is selected from the group consisting of multiple sclerosis (MS), clinically isolated syndrome (CIS), relapsing-remitting MS (RRMS), primary progressive MS (PPMS) and secondary progressive MS (SPMS).

Neuromuscular blockade is used in connection with surgery under general anaesthesia. Reversing agents are used for more rapid and safer recovery of muscle function after such blockade. Complications with excessive muscle weakness after blockade during surgery can result in delayed weaning from mechanical ventilation and respiratory complications after the surgery. These complications can have pronounced effects on outcome of the surgery and future quality of life of patients, there is a need for improved reversing agents (Murphy GS, Brull S J. Residual neuromuscular block: lessons unlearned. Part I: definitions, incidence, and adverse physiologic effects of residual neuromuscular block. Anesth Analg. 2010 111(1):120-8). Thus, in one embodiment, the neuromuscular disorder has been induced by a neuromuscular blocking agent. In one particular embodiment the neuromuscular disorder is muscle weakness caused by neuromuscular blockade after surgery. In another embodiment of the present disclosure the compound or the compound for use is used for reversing and/or ameliorating neuromuscular blockade after surgery. In one embodiment, the neuromuscular blockade is drug induced. In one embodiment, the neuromuscular blockade is caused by non-depolarizing neuromuscular blocker or antibiotic agent. In one embodiment the neuromuscular blockade is induced by an antibiotic. In one embodiment the neuromuscular blockade is induced by a non-depolarizing neuromuscular blocker.

In one embodiment the compound or the compound for use of the present disclosure is used to prevent a neuromuscular disorder. The compound or the compound for use may for example be used prophylactically against nerve gas that is known to cause symptoms of muscle weakness and fatigue (Kawamura, Y., Kihara, M., Nishimoto, K., Taki, M. Efficacy of a half dose of oral pyridostigmine in the treatment of chronic fatigue syndrome: three case reports. Pathophysiology, 2003, 9, 189-194). In one embodiment the compound or the compound for use of the present disclosure may be used in the treatment of muscle weakness (such as drooping eyelids, loss of facial expression, constipation, muscle weakness in arms, muscle weakness in legs and dyspnoea) caused by botulism poisoning. In one embodiment the compound or the compound for use of the present disclosure may be used in the treatment of snake bites where the snake toxin, such as a-neurotoxin or myotoxin, is known to cause symptoms of muscle weakness and fatigue.

In one embodiment, the neuromuscular disorder is selected from the group consisting of myasthenia gravis, autoimmune myasthenia gravis, congenital myasthenic syndrome, seronegative myasthenia gravis, muscle specific kinase myasthenia gravis (MuSK-MG), Lambert-Eaton Syndrome, amyotrophic lateral sclerosis (ALS), spinal muscular atrophy (SMA), critical illness myopathy (CIM), Charcot-Marie Tooth disease, diabetic polyneuropathy, periodic paralysis, hypokalemic periodic paralysis, hyperkalemic periodic paralysis, myotubular myopathy, Duchenne muscular dystrophy, Guillain-Barre syndrome, poliomyelitis, post-polio syndrome, chronic fatigue syndrome, critical illness polyneuropathy, metabolic myopathy, Kennedy's disorder, multiple sclerosis and multifocal motor neuropathy.

### Pharmaceutical Formulations

In one aspect, the present invention relates to a composition comprising the compound as disclosed herein. The composition according to the present disclosure may be used for treating, ameliorating and/or preventing a neuromuscular disorder, and/or for use in reversing and/or ameliorating a neuromuscular blockade. Thus, the compositions and compounds described herein can be pharmaceutically acceptable. In one embodiment the composition as described herein is in the form of a pharmaceutical formulation. In one embodiment, the composition as described herein further comprises a pharmaceutically acceptable carrier. In one aspect, the present invention concerns a composition comprising the compound as defined herein and a pharmaceutically acceptable carrier.

### Combination Therapy

The composition of the present disclosure may comprise further active ingredients/agents or other components to increase the efficiency of the composition. Thus, in one embodiment the composition further comprises at least one further active agent. It is appreciated that the active agent can be suitable for treating, preventing or ameliorating said neuromuscular disorder.

The active agent in certain embodiments can be an acetylcholine esterase inhibitor. Said acetylcholine esterase inhibitor may for example be selected from the group consisting of delta-9-tetrahydrocannabinol, carbamates, physostigmine, neostigmine, pyridostigmine, ambenonium, demecarium, rivastigmine, phenanthrene derivatives, galantamine, piperidines, donepezil, tacrine, edrophonium, huperzine, ladostigil, ungeremine and lactucopicrin.

In certain embodiments, the acetylcholine esterase inhibitor is selected from the group consisting of neostigmine, physostigmine and pyridostigmine. In certain embodiments, the acetylcholine esterase inhibitor is neostigmine or pyridostigmine.

The active agent may also be an immunosuppressive drug. Immunosuppressive drugs are drugs that suppress or reduce the strength of the body's immune system. Immunosuppressive drugs include but are not limited to glucocorticoids, corticosteroids, cytostatics, antibodies and drugs acting on immunophilins. In one embodiment the active agent is prednisone.

The active agent may also be an agent that is used in anti-myotonic treatment. Such agents include for example blockers of voltage gated Na⁺ channels, and aminoglycosides.

The active agent may also be an agent for reversing a neuromuscular blockade after surgery. Such agents include for example neostigmine or sugammadex (Org 25969, tradename Bridion).

The active agent may also be an agent for increasing ACh release by blocking voltage-gated K⁺ channels in the pre-synaptic terminal. Such agent includes 3,4-diaminopyridine (Amifampridine; tradename Firdapse).

The active agent may also be an agent for increasing the levels of survival motor neuron (SMN) protein that are produced. For example, by alternating the splicing of the SMN2 gene in order to increase the expression of full-length SMN protein from SMN2 (Zanetta C, Nizzardo M, Simone C, Monguzzi E, Bresolin N, Comi GP, Corti S, “Molecular therapeutic strategies for spinal muscular atrophies: current and future clinical trials”. Clinical Therapeutics, 2014, 36 (1): 128-40). Such agents include antisense oligonucleotides such as Nusinersen (tradename Spinraza) or small molecules such as Risdiplam (tradename Evrysdi).

The active agent may be a gene therapy, for example by using viral vectors to deliver the SMN1 transgene to the affected motor neurons, where it leads to an increase in SMN protein production. Such gene therapies include onasemnogene abeparvovec (tradename Zolgensma). Such gene therapies include nusinersen (tradename Spinraza), risdiplam (tradename Evrysdi) and Branaplam.

The active agent may be a small molecule that increases expression of the SMN2 gene, thus increasing the amount of full-length SMN protein available. Such therapies include salbutamol (also, called albuterol; tradename Ventolin), The active agent may also be an agent for increasing muscle reactivity. Such agents include skeletal troponin activators such as Tirasemtiv and Reldesemtiv (CK-2127107) (Hwee, D. T., Kennedy, A. R., Hartman, J. J., Ryans, J., Durham, N., Malik, F. I., Jasper, J. R. The small-molecule fast skeletal troponin activator, CK-2127107, improves exercise tolerance in a rat model of heart failure. Journal of Pharmacology and Experimental Therapeutics, 2015, 353, 159-168). Such agents may also be antibodies that block the activation of the skeletal muscle protein myostatin, such as Apitegromab (SRK-015) or GYM329 (RO7204239), The active agent may also be an agent that disrupts or blocks the IgG-FcRn interaction thereby reducing the overall IgG recycling. Such agents may be antibodies, such as the aglycosylated immunoglobulin (Ig)G1 monoclonal antibody Nipocalimab, or IgG1 Fc fragment such as Efgartigimod alfa (tradename Vyvgart).

The active agent may also be an agent that is an inhibitor of the complement component C5a. The active agent may also be an agent that downregulates the overexpression of PMP22 protein, leading to improvement of neuronal signalling in dysfunctional peripheral nerves. Such agents may be combination drugs such as PXT3003. Alternatively, the active agent may also be an agent that binds to the protein complement component 5 (C5) and inhibits its cleavage into C5a and C5b. Such agents may be Zilucoplan (RA101495).

### Methods

In one aspect, the present invention relates to a compound as defined herein for use as a medicament.

In one aspect, the present invention relates to a compound as defined herein for use in treating, ameliorating and/or preventing a neuromuscular disorder.

In one aspect, the present invention related to a compound as defined herein for use in the treatment of an indication selected from the group consisting of myasthenia gravis, autoimmune myasthenia gravis, congenital myasthenic syndrome, seronegative myasthenia gravis, muscle specific kinase myasthenia gravis (MuSK-MG), Lambert-Eaton Syndrome, amyotrophic lateral sclerosis (ALS), spinal muscular atrophy (SMA), critical illness myopathy (CIM), Charcot-Marie Tooth disease, diabetic polyneuropathy, periodic paralysis, hypokalemic periodic paralysis, hyperkalemic periodic paralysis, myotubular myopathy, Duchenne muscular dystrophy, Guillain-Barre syndrome, poliomyelitis, post-polio syndrome, chronic fatigue syndrome, critical illness polyneuropathy, metabolic myopathy, Kennedy's disorder, multiple sclerosis and multifocal motor neuropathy.

In one aspect, the present invention relates to a compound as defined herein for use in the symptomatic treatment of sarcopenia.

In one aspect, the present invention relates to a compound as defined herein for use in reversing and/or ameliorating a neuromuscular blockade.

In one aspect, the present disclosure relates to a compound of Formula (I):

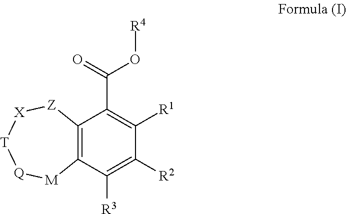

wherein:
   M is CR⁵R⁶, NR⁹, O or S;
   Q is CR⁷R⁶, NR¹⁰, O or S;
   T is absent, CR⁷R⁶, NR¹⁰, O or S;
   X is absent, CR⁷R⁶, NR¹⁰, O or S;
   Z is CR⁸R⁶, NR¹¹, O or S;
wherein no more than one of M, Q, T, X or Z is selected from the group consisting of NR⁹, NR¹⁰, NR¹¹, O or S;
wherein R⁵ and R⁸, or R⁵ and R¹¹, or R⁹ and R⁸ are optionally joined together to form a ring by —CH₂—, —CH₂CH₂- or —CH₂CH₂CH₂—;
   R¹ is selected from the group consisting of H; F; Cl; Br; I; C₁₋₃ alkyl optionally substituted with one or more, identical or different, substituents R¹²; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
R² is selected from the group consisting of H; F; Cl; Br; I; C₁₋₃ alkyl optionally substituted with one or more, identical or different, substituents R¹²; —OC₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R⁵ is selected from the group consisting of H, deuterium, F, and C₁₋₃ alkyl optionally substituted with one or more, identical or different, substituents R¹², or when R⁵ is linked to R^{a} or R¹¹, then R⁵ is a bond or C₁₋₃ alkanediyl;
   R⁶ is independently selected from the group consisting of H, deuterium, F, C₁₋₃ alkyl substituted with R¹⁴ and C₁₋₃ alkyl optionally substituted with one or more, identical or different, substituents R¹²;
   R⁷ is independently selected from the group consisting of H, deuterium, F and C₁₋₃ alkyl optionally substituted with one or more, identical or different, substituents R¹²;
   R^{a} is selected from the group consisting of H, deuterium, F and C₁₋₃ alkyl optionally substituted with one or more, identical or different, substituents R¹², or when R^{a} is linked to R⁵ or R⁹, then R^{a} is a bond or C₁₋₃ alkanediyl;
   R⁹ is selected from the group consisting of H, C₁₋₅ alkyl optionally substituted with one or more, identical or different, substituents R¹², and C₁₋₃ alkyl substituted with R¹⁴, or when R⁹ is linked to R⁸, then R⁹ is a bond or C₁₋₃ alkanediyl;
   R¹⁰ is independently selected from benzyl optionally substituted with one or more, identical or different, substituents R¹³;
   R¹¹ is selected from the group consisting of H, C₁₋₅ alkyl optionally substituted with one or more, identical or different, substituents R¹², and C₁₃ alkyl substituted with R¹⁵, or when R¹¹ is linked to R⁵, then R¹¹ is a bond or C₁₋₃ alkanediyl;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F;
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; and
   R¹⁵ is independently selected from the group consisting of C₃₋₆ cycloalkyl optionally substituted with one or more, identical or different, substituents R¹², C₆10 aryl optionally substituted with one or more, identical or different, substituents R¹³ and 5- to 10-membered heteroaryl optionally substituted with one or more, identical or different, substituents R¹³;
or a pharmaceutically acceptable salt, hydrate, polymorph, tautomer, or solvate thereof for use in treating, ameliorating and/or preventing a neuromuscular disorder, and/or for use in reversing and/or ameliorating a neuromuscular blockade.

In one embodiment, the compound for use is selected from the group consisting of:
6-chloro-5-methyl-2,3-dihydro-1H-indene-4-carboxylic acid;
6-bromo-5-methyl-2,3-dihydro-1H-indene-4-carboxylic acid;
6-chloro-5-(fluoromethyl)-2,3-dihydro-1H-indene-4-carboxylic acid;
3-chloro-5,6,7,8-tetrahydronaphthalene-1-carboxylic acid;
5,6-dimethyl-2,3-dihydro-1H-indene-4-carboxylic acid;
2,3-dimethyl-5,6,7,8-tetrahydronaphthalene-1-carboxylic acid;
6-chloro-2,3-dihydro-1H-indene-4-carboxylic acid;
6-chloro-5-methoxy-2,3-dihydro-1H-indene-4-carboxylic acid;
6-chloro-5-(fluoromethoxy)-2,3-dihydro-1H-indene-4-carboxylic acid;
3-chloro-2-methoxy-5,6,7,8-tetrahydronaphthalene-1-carboxylic acid;
3-chloro-2-methyl-5,6,7,8-tetrahydronaphthalene-1-carboxylic acid;
6-chloro-5-(difluoromethoxy)-2,3-dihydro-1H-indene-4-carboxylic acid;
6-chloro-5-(trifluoromethoxy)-2,3-dihydro-1H-indene-4-carboxylic acid;
5-chloro-6-methyl-2,3-dihydro-1-benzofuran-7-carboxylic acid;
6-chloro-5-ethyl-2,3-dihydro-1H-indene-4-carboxylic acid;
6-chloro-7-methyl-3,4-dihydro-2H-1-benzopyran-8-carboxylic acid;
5-chloro-6-methoxy-2,3-dihydro-1-benzofuran-7-carboxylic acid;
5-chloro-4-methoxytricyclo[6.2.1.0^{2,7}]undeca-2(7),3,5-triene-3-carboxylic acid;
3-chloro-4-fluoro-5,6,7,8-tetrahydronaphthalene-1-carboxylic acid;
3-fluoro-2-methoxy-5,6,7,8-tetrahydronaphthalene-1-carboxylic acid;
3-chloro-4-fluoro-2-methoxy-5,6,7,8-tetrahydronaphthalene-1-carboxylic acid;
4-chloro-5-methyltricyclo[6.2.1.0^{2,7}]undeca-2,4,6-triene-3-carboxylic acid;
5-chloro-4-methyltricyclo[6.2.1.0^{2,7}]undeca-2(7),3,5-triene-3-carboxylic acid;
3-chloro-2-methoxy-6,7,8,9-tetrahydro-5H-benzo[7]annulene-1-carboxylic acid;
6-chloro-5-(methylsulfanyl)-2,3-dihydro-1H-indene-4-carboxylic acid;
2-chloro-3-methoxy-6,7,8,9-tetrahydro-5H-benzo[7]annulene-1-carboxylic acid;
3-chloro-2-(difluoromethoxy)-5,6,7,8-tetrahydronaphthalene-1-carboxylic acid;
3-chloro-2-fluoro-5,6,7,8-tetrahydronaphthalene-1-carboxylic acid;
2-chloro-3-fluoro-5,6,7,8-tetrahydronaphthalene-1-carboxylic acid;
2-chloro-3-methyl-5,6,7,8-tetrahydronaphthalene-1-carboxylic acid;
5-chloro-4-methoxytricyclo[6.2.2.0^{2,7}]dodeca-2,4,6-triene-3-carboxylic acid;
6-chloro-5-(trifluoromethyl)-2,3-dihydro-1H-indene-4-carboxylic acid;
2,3-dichloro-5,6,7,8-tetrahydronaphthalene-1-carboxylic acid;
6-chloro-7-methoxy-3,4-dihydro-2H-1-benzothiopyran-8-carboxylic acid;
3-chloro-2-(methylsulfanyl)-5,6,7,8-tetrahydronaphthalene-1-carboxylic acid;
3-chloro-2-methyl-6,7,8,9-tetrahydro-5H-benzo[7]annulene-1-carboxylic acid;
2-chloro-3-methyl-6,7,8,9-tetrahydro-5H-benzo[7]annulene-1-carboxylic acid;
3-chloro-2-ethenyl-5,6,7,8-tetrahydronaphthalene-1-carboxylic acid;
6-chloro-7-fluoro-3,4-dihydro-2H-1-benzopyran-8-carboxylic acid;
7-chloro-6-fluoro-3,4-dihydro-2H-1-benzopyran-8-carboxylic acid;
6-chloro-7-fluoro-3,4-dihydro-2H-1-benzothiopyran-8-carboxylic acid;
7-chloro-6-fluoro-3,4-dihydro-2H-1-benzopyran-5-carboxylic acid;
6-chloro-7-fluoro-1-methyl-1,2,3,4-tetrahydroquinoline-8-carboxylic acid;
6-chloro-7-fluoro-1,2,3,4-tetrahydroquinoline-8-carboxylic acid;
6,7-difluoro-3,4-dihydro-2H-1-benzothiopyran-8-carboxylic acid;
5-chloro-6-fluoro-2,3-dihydro-1-benzofuran-7-carboxylic acid;
2,3-difluoro-5,6,7,8-tetrahydronaphthalene-1-carboxylic acid;
7-chloro-6-fluoro-1-methyl-1,2,3,4-tetrahydroquinoline-5-carboxylic acid;
7-chloro-6-fluoro-1-methyl-1,2,3,4-tetrahydroquinoline-8-carboxylic acid;
5-methoxy-6-methyl-2,3-dihydro-1H-indene-4-carboxylic acid;
7-fluoro-6-methyl-3,4-dihydro-2H-1-benzothiopyran-8-carboxylic acid;
2-fluoro-3-methyl-5,6,7,8-tetrahydronaphthalene-1-carboxylic acid;
7-fluoro-6-methyl-3,4-dihydro-2H-1-benzopyran-8-carboxylic acid;
3-ethyl-2-fluoro-5,6,7,8-tetrahydronaphthalene-1-carboxylic acid;
3-fluoro-2-methyl-5,6,7,8-tetrahydronaphthalene-1-carboxylic acid;
6-chloro-2,2-dimethyl-3,4-dihydro-2H-1-benzopyran-8-carboxylic acid;
3-cyclopropyl-2-fluoro-5,6,7,8-tetrahydronaphthalene-1-carboxylic acid;
2-fluoro-3-iodo-5,6,7,8-tetrahydronaphthalene-1-carboxylic acid;
8-chloro-7-fluoro-2,3,4,5-tetrahydro-1-benzoxepine-9-carboxylic acid;
6-chloro-7-fluoro-2,3,4,5-tetrahydro-1-benzoxepine-9-carboxylic acid;
6-fluoro-7-methyl-3,4-dihydro-2H-1-benzopyran-5-carboxylic acid;
8-chloro-7-fluoro-2,3,4,5-tetrahydro-1-benzoxepine-6-carboxylic acid;
3-chloro-8,8-difluoro-2-methoxy-5,6,7,8-tetrahydronaphthalene-1-carboxylic acid;
2-fluoro-3-(trifluoromethyl)-5,6,7,8-tetrahydronaphthalene-1-carboxylic acid;
6-chloro-7-fluoro-2,2-dimethyl-3,4-dihydro-2H-1-benzopyran-8-carboxylic acid;
5-chloro-6-fluoro-2,3-dihydro-1H-indene-4-carboxylic acid;
6-chloro-5-fluoro-2,3-dihydro-1H-indene-4-carboxylic acid;
7-chloro-6-fluoro-3,4-dihydro-2H-1-benzothiopyran-8-carboxylic acid;
7-chloro-6-fluoro-3,4-dihydro-2H-1-benzothiopyran-5-carboxylic acid;
2-bromo-3-chloro-5,6,7,8-tetrahydronaphthalene-1-carboxylic acid;
3-bromo-2-chloro-5,6,7,8-tetrahydronaphthalene-1-carboxylic acid;
2-benzyl-5,6-dichloro-2,3-dihydro-1H-isoindole-4-carboxylic acid;
6-chloro-7-methoxy-3,4-dihydro-2H-1-benzopyran-8-carboxylic acid;
6-chloro-7-fluoro-1-[(3-thienyl)methyl]-1,2,3,4-tetrahydro-8-quinolinecarboxylic acid;
1-benzyl-6-chloro-7-fluoro-1,2,3,4-tetrahydro-8-quinolinecarboxylic acid;
6-chloro-7-fluoro-1-[(p-fluorophenyl)methyl]-1,2,3,4-tetrahydro-8-quinolinecarboxylic acid;
3-chloro-5,5-difluoro-2-methoxy-5,6,7,8-tetrahydro-1-naphthoic acid;
1-benzyl-7-chloro-6-fluoro-1,2,3,4-tetrahydro-8-quinolinecarboxylic acid;
7-chloro-6-fluoro-2,2-dimethyl-8-chromancarboxylic acid;
1-benzyl-6-chloro-5-fluoro-4-indolinecarboxylic acid;
7-chloro-6-fluoro-1-methyl-1,2,3,4-tetrahydro-8-quinolinecarboxylic acid;
4-benzyl-6-chloro-7-fluoro-8-chromancarboxylic acid;
7-chloro-6-methoxy-5-chromancarboxylic acid;
4-benzyl-7-fluoro-6-methoxy-8-chromancarboxylic acid;
4-benzyl-7-fluoro-6-methoxy-5-chromancarboxylic acid;
6-chloro-7-fluoro-5-chromancarboxylic acid;
7-chloro-6-methoxy-3,4-dihydro-2H-1-benzothiin-8-carboxylic acid;
3-chloro-2-ethoxy-5,6,7,8-tetrahydro-1-naphthoic acid;
6-chloro-1,2,3,4-tetrahydro-8-quinolinecarboxylic acid; and
7-chloro-6-methoxy-8-chromancarboxylic acid.

In one aspect, the present disclosure relates to a method of treating, preventing and/or ameliorating a neuromuscular disorder, said method comprising administering a therapeutically effective amount of the compound or the compound for use as defined herein to a person in need thereof.

In one aspect, the present disclosure relates to a method of reversing and/or ameliorating a neuromuscular blockade, said method comprising administering a therapeutically effective amount of the compound or the compound for use as defined herein to a person in need thereof.

In one aspect, the present disclosure relates to a method for recovery of neuromuscular transmission, said method comprising administering a therapeutically effective amount of the compound or the compound for use as defined herein to a person in need thereof.

The person in need thereof may be a person having a neuromuscular disorder or a person at risk of developing a neuromuscular disorder or a person having symptoms of muscle weakness and/or fatigue. In another embodiment the person in need thereof is a person with reduced neuromuscular transmission safety with prolonged recovery after neuromuscular blockade. Types of neuromuscular disorders are defined herein above. In an embodiment the person has amyotrophic lateral sclerosis, spinal muscular atrophy, myasthenia gravis or Lambert-Eaton syndrome.

A therapeutically effective amount is an amount that produces a therapeutic response or desired effect in the person taking it. Administration routes, formulations and dosages can be optimized by persons of skill in the art.

The method of treatment may be combined with other methods that are known to treat, prevent and/or ameliorate neuromuscular disorders. The treatment method may for example be combined with administration of any of the agents mentioned herein above. In one embodiment the treatment is combined with administration of acetylcholine esterase inhibitor such as for example neostigmine or pyridostigmine.

In one aspect, the invention relates to a method for recovery of force in muscles with neuromuscular dysfunction, said method comprising administering a compound or a composition as defined herein to a subject in need thereof. The term “recovery of force in muscles with neuromuscular dysfunction” as used herein refers to the ability of a compound to recover contractile force in nerve-stimulated healthy rat muscle after exposure to submaximal concentration (115 nM) of tubocurarine for 90 minutes.

Recovery of force is quantified as the percentage of the force prior to tubocurarine that is recovered after addition of the compound. In one embodiment, said recovery of force is >5%, such as >10%, such as >15%, such as >20%, such as >25%, such as >30%, such as >35%.

In one aspect, the present invention relates to a method of treating botulism poisoning, snake bites or nerve gas poisoning or preventing nerve gas poisoning, said method comprising administering a therapeutically effective amount of the compound as defined herein to a person in need thereof.

Another aspect of the disclosure relates to use of a compound as defined herein, for the manufacture of a medicament for the treatment, prevention and/or amelioration of a neuromuscular disorder.

Another aspect relates to use of a compound as defined herein, for the manufacture of a medicament or a reversal agent for reversing and/or ameliorating a neuromuscular blockade after surgery.

In one aspect, the present invention related to use of a compound as defined herein for the manufacture of a medicament for the treatment of botulism poisoning, snake bites or nerve gas poisoning or prevention of nerve gas poisoning.

### Method of Manufacturing

In one aspect, the present disclosure relates to methods of manufacturing compounds or compounds for use according to formula (I).

Compounds according to the present invention may be prepared according to any conventional methods of chemical synthesis known by the skilled person, e.g. those described in the working examples. The starting materials for the processes described in the present application are known or may readily be prepared by conventional methods known by the skilled artisan from commercially available chemicals.

The end products of the reactions described herein may be isolated by conventional technique such as extraction, crystallisation, distillation, chromatography etc.

The compounds of this invention may exist in unsolvated as well as in solvated forms with pharmaceutically acceptable solvents such as water, ethanol and the like. In general, the solvated forms are considered equivalent to the unsolvated forms for the purposes of this invention.

### Items

1. A compound of Formula (I):

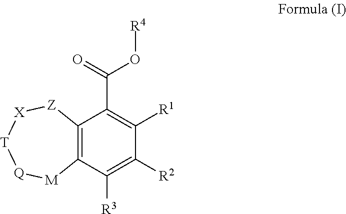

M is CR⁵R⁶, NR⁹, 0 or S;
Q is CR⁷R⁶, NR¹⁰, O or S;
T is absent, CR⁷R⁶, NR¹⁰, O or S;
X is absent, CR⁷R⁶, NR¹⁰, O or S;
Z is CR⁸R⁶, NR¹¹, O or S;
wherein no more than one of M, Q, T, X or Z is selected from the group consisting of NR⁹, NR¹⁰, NR¹¹, O or S;
wherein R⁵ and R⁸, or R⁵ and R¹¹, or R⁹ and R⁸ are optionally joined together to form a ring by —CH₂—, —CH₂CH₂- or —CH₂CH₂CH₂—;
   R¹ is selected from the group consisting of H; F; Cl; Br; I; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of H; F; Cl; Br; I; C₁₋₃ alkyl optionally substituted with one or more, identical or different, substituents R¹²; —OC₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹². phenyl optionally substituted with one or more, identical or different, substituents R¹³; and benzyl optionally substituted with one or more, identical or different, substituents R¹³;
   R⁵ is selected from the group consisting of H, deuterium, F, and C₁₋₃ alkyl optionally substituted with one or more, identical or different, substituents R¹², or when R⁵ is linked to R⁸ or R¹¹, then R⁵ is a bond or C₁₋₃ alkanediyl; -R⁶ is independently selected from the group consisting of H, deuterium, F, C₁₋₃ alkyl substituted with R¹⁴ and C₁₋₃ alkyl optionally substituted with one or more, identical or different, substituents R¹²;
   R⁷ is independently selected from the group consisting of H, deuterium, F and C₁₋₃ alkyl optionally substituted with one or more, identical or different, substituents R¹²;
   R^{a} is selected from the group consisting of H, deuterium, F and C₁₋₃ alkyl optionally substituted with one or more, identical or different, substituents R¹², or when R^{a} is linked to R⁵ or R⁹, then R^{a} is a bond or C₁₋₃ alkanediyl;
   R⁹ is selected from the group consisting of H, C₁₋₅ alkyl optionally substituted with one or more, identical or different, substituents R¹², and C₁₋₃ alkyl substituted with R¹⁴, or when R⁹ is linked to R⁸, then R⁹ is a bond or C₁₋₃ alkanediyl; -R¹⁰ is independently selected from benzyl optionally substituted with one or more, identical or different, substituents R¹³;
   R¹¹ is selected from the group consisting of H, C₁₋₅ alkyl optionally substituted with one or more, identical or different, substituents R¹², and C₁₋₃ alkyl substituted with R¹⁵, or when R¹¹ is linked to R⁵, then R¹¹ is a bond or C₁₋₃ alkanediyl;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F;
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; and
   R¹⁵ is independently selected from the group consisting of C₃₋₆ cycloalkyl optionally substituted with one or more, identical or different, substituents R¹², C₆10 aryl optionally substituted with one or more, identical or different, substituents R¹³ and 5- to 10-membered heteroaryl optionally substituted with one or more, identical or different, substituents R¹³;
or a pharmaceutically acceptable salt, hydrate, polymorph, tautomer, or solvate thereof.

2. A compound of Formula (I):

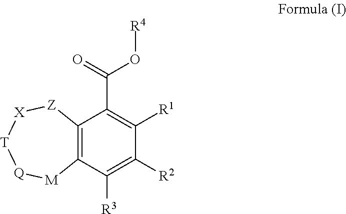

M is CR⁵R⁶, NR⁹, O or S;
Q is CR⁷R⁶, NR¹⁰, O or S;
T is absent, CR⁷R⁶, NR¹⁰, O or S;
X is absent, CR⁷R⁶, NR¹⁰, O or S;
Z is CR⁸R⁶, NR¹¹, O or S;
wherein no more than one of M, Q, T, X or Z is selected from the group consisting of NR⁹, NR¹⁰, NR¹¹, O or S;
wherein R⁵ and R⁸, or R⁵ and R¹¹, or R⁹ and R⁸ are optionally joined together to form a ring by —CH₂—, —CH₂CH₂- or —CH₂CH₂CH₂—;
   R¹ is selected from the group consisting of H; F; Cl; Br; I; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of H; F; Cl; Br; I; C₁₋₃ alkyl optionally substituted with one or more, identical or different, substituents R¹²; —OC₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R⁵ is selected from the group consisting of H, deuterium, F, a bond and C₁₋₃ alkyl optionally substituted with one or more, identical or different, substituents R¹²;
   R⁶ is independently selected from the group consisting of H, deuterium, F and C₁₋₃ alkyl optionally substituted with one or more, identical or different, substituents R¹²;
   R⁷ is independently selected from the group consisting of H, deuterium, F and C₁₋₃ alkyl optionally substituted with one or more, identical or different, substituents R¹²;
   R^{a} is selected from the group consisting of H, deuterium, a bond, F and C₁₋₃ alkyl optionally substituted with one or more, identical or different, substituents R¹²;
   R⁹ is selected from the group consisting of H, C₁₋₅ alkyl optionally substituted with one or more, identical or different, substituents R¹², a bond and benzyl optionally substituted with one or more, identical or different, substituents R¹³;
   R¹⁰ is independently selected from benzyl optionally substituted with one or more, identical or different, substituents R¹³;
   R¹¹ is selected from the group consisting of H, C₁₋₅ alkyl optionally substituted with one or more, identical or different, substituents R¹², a bond and benzyl optionally substituted with one or more, identical or different, substituents R¹³;
   R¹² is independently selected from the group consisting of deuterium, F and OMe; and
   R¹³ is independently selected from the group consisting of deuterium, methoxy, nitro, cyano, Cl, Br, I, and F;
or a pharmaceutically acceptable salt, hydrate, polymorph, tautomer, or solvate thereof.

3. The compound according to any one of items 1 or 2, wherein R¹ is selected from the group consisting of F; Cl; Br; I; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;

4. The compound according to item 1, wherein the compound is of Formula (I):

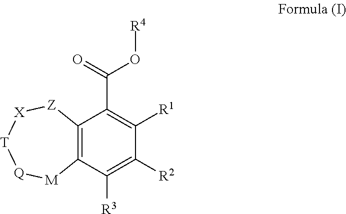

wherein:
   M is CR⁵R⁶, NR⁹, O or S;
   Q is CR⁷R⁶, NR¹⁰, O or S;
   T is absent, CR⁷R⁶, NR¹⁰, O or S;
   X is absent, CR⁷R⁶, NR¹⁰, O or S;
   Z is CR⁸R⁶, NR¹¹, O or S;
wherein no more than one of M, Q, T, X or Z is selected from the group consisting of NR⁹, NR¹⁰, NR¹¹, O or S;
wherein R⁵ and R⁸, or R⁵ and R¹¹, or R⁹ and R⁸ are optionally joined together to form a ring by —CH₂—, —CH₂CH₂- or —CH₂CH₂CH₂—;
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R⁵ is selected from the group consisting of H, deuterium, F, a bond and C₁₋₃ alkyl optionally substituted with one or more, identical or different, substituents R¹²;
   R⁶ is independently selected from the group consisting of H, deuterium, F and C₁₋₃ alkyl optionally substituted with one or more, identical or different, substituents R¹²;
   R⁷ is independently selected from the group consisting of H, deuterium, F and C₁₋₃ alkyl optionally substituted with one or more, identical or different, substituents R¹²;
   R^{a} is selected from the group consisting of H, deuterium, a bond, F and C₁₋₃ alkyl optionally substituted with one or more, identical or different, substituents R¹²;
   R⁹ is selected from the group consisting of H, C₁₋₅ alkyl optionally substituted with one or more, identical or different, substituents R¹², a bond and benzyl optionally substituted with one or more, identical or different, substituents R¹³;
   R¹⁰ is independently selected from benzyl optionally substituted with one or more, identical or different, substituents R¹³;
   R¹¹ is selected from the group consisting of H, C₁₋₅ alkyl optionally substituted with one or more, identical or different, substituents R¹², a bond and benzyl optionally substituted with one or more, identical or different, substituents R¹³;
   R¹² is independently selected from the group consisting of deuterium, F and OMe; and
   R¹³ is independently selected from the group consisting of deuterium, methoxy, nitro, cyano, Cl, Br, I, and F;
or a pharmaceutically acceptable salt, hydrate, polymorph, tautomer, or solvate thereof provided that:
when M, Q, T, X and Z are CH₂, R¹ is OMe, R² is Cl and R³ is H then R⁴ is not H, Me or Et;
when M, Q, T and Z are CH₂, X is absent, R¹ is OMe, R² is Cl and R³ is H then R⁴ is not H, Me or Et;
when M, Q, T and Z are CH₂, X is absent, R¹ is OMe, R² is CF₃ and R³ is H then R⁴ is not Me;
when M, Q, T and Z are CH₂, X is absent, R¹ is Me, R² is Me and R³ is H then R⁴ is not H;
when M, Q, T and Z are CH₂, X is absent, R¹ is Cl, R² is Cl and R³ is Cl then R⁴ is not H;
when M, Q and Z are CH₂, T and X are absent, R¹ is Me, R² is Me and R³ is H then R⁴ is not H or Me;
when M and Z are CH₂, Q is 0, T and X are absent, R¹ is OMe, R² is Cl and R³ is H then R⁴ is not H, Me or Et;
when M and Z are CH₂, Q is 0, T and X are absent, R¹ is Cl, R² is Cl, and R³ is H then R⁴ is not H;
when M and Z are CH₂, Q is 0, T and X are absent, R¹ is OMe, R² is Me, and R³ is H then R⁴ is not H;
when Q and Z are CH₂, M is 0, T and X are absent, R¹ is OMe, R² is Me and R³ is H then R⁴ is not H, Me or Et;
when M and Q are CH₂, Z is 0, T and X are absent, R¹ is OMe, R² is Et and R³ is H then R⁴ is not H;
when M is CH₂, Q is CMe₂, Z is 0, T and X are absent, R¹ and R² are F and R³ is H then R⁴ is not H;
when Q and Z are CH₂, M is S, T and X are absent, R¹ is Cl or F, R² is Me and R³ is H then R⁴ is not H;
when Q and Z are CH₂, M is S, T and X are absent, R¹ is Cl, R² is Cl and R³ is H then R⁴ is not H; and when Q and M are CH₂, Z is S, T and X are absent, R¹ is Cl or Me, R² is Me and R³ is H then R⁴ is not H.

5. The compound according to item 1, wherein the compound is of Formula (I):

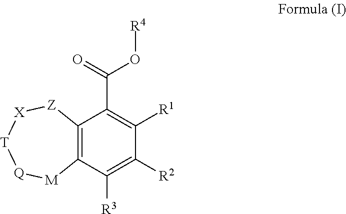

wherein:
   M is CR⁵R⁶, NR⁹, O or S;
   Q is CR⁷R⁶, NR¹⁰, O or S;
   T is absent, CR⁷R⁶, NR¹⁰, O or S;
   X is absent, CR⁷R⁶, NR¹⁰, O or S;
   Z is CR⁸R⁶, NR¹¹, O or S;
wherein no more than one of M, Q, T, X or Z is selected from the group consisting of NR⁹, NR¹⁰, NR¹¹, O or S;
wherein R⁵ and R⁸, or R⁵ and R¹¹, or R⁹ and R⁸ are optionally joined together to form a ring by —CH₂—, —CH₂CH₂- or —CH₂CH₂CH₂—;
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R⁵ is selected from the group consisting of H, deuterium, F, and C₁₋₃ alkyl optionally substituted with one or more, identical or different, substituents R¹², or when R⁵ is linked to R^{a} or R¹¹, then R⁵ is a bond or C₁₋₃ alkanediyl;
   R⁶ is independently selected from the group consisting of H, deuterium, F, C₁₋₃ alkyl substituted with R¹⁴ and C₁₋₃ alkyl optionally substituted with one or more, identical or different, substituents R¹²;
   R⁷ is independently selected from the group consisting of H, deuterium, F and C₁₋₃ alkyl optionally substituted with one or more, identical or different, substituents R¹²;
   R^{a} is selected from the group consisting of H, deuterium, F and C₁₋₃ alkyl optionally substituted with one or more, identical or different, substituents R¹², or when R^{a} is linked to R⁵ or R⁹, then R^{a} is a bond or C₁₃ alkanediyl;
   R⁹ is selected from the group consisting of H, C₁₋₅ alkyl optionally substituted with one or more, identical or different, substituents R¹², and C₁₋₃ alkyl substituted with R¹⁴, or when R⁹ is linked to R⁸, then R⁹ is a bond or C₁₋₃ alkanediyl;
   R¹⁰ is independently selected from benzyl optionally substituted with one or more, identical or different, substituents R¹³;
   R¹¹ is selected from the group consisting of H, C₁₋₅ alkyl optionally substituted with one or more, identical or different, substituents R¹², and C₁₋₃ alkyl substituted with R¹⁵, or when R¹¹ is linked to R⁵, then R¹¹ is a bond or C₁₋₃ alkanediyl;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F;
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; and
   R¹⁵ is independently selected from the group consisting of C₃₋₆ cycloalkyl optionally substituted with one or more, identical or different, substituents R¹², C₆-1o aryl optionally substituted with one or more, identical or different, substituents R¹³ and 5- to 10-membered heteroaryl optionally substituted with one or more, identical or different, substituents R¹³;
or a pharmaceutically acceptable salt, hydrate, polymorph, tautomer, or solvate thereof provided that:
when M, Q, T, X and Z are CH₂, R¹ is OMe, R² is Cl and R³ is H then R⁴ is not H, Me or Et;
when M, Q, T and Z are CH₂, X is absent, R¹ is OMe, R² is Cl and R³ is H then R⁴ is not H, Me or Et;
when M, Q, T and Z are CH₂, X is absent, R¹ is OMe, R² is CF₃ and R³ is H then R⁴ is not Me;
when M, Q, T and Z are CH₂, X is absent, R¹ is Cl, R² is Cl and R³ is Cl then R⁴ is not H;
when M, Q, T and Z are CH₂, X is absent, R¹ is Me, R² is Me and R³ is H then R⁴ is not H;
when M, Q and Z are CH₂, T and X are absent, R¹ is Me, R² is Me and R³ is H then R⁴ is not H or Me;
when M and Z are CH₂, Q is 0, T and X are absent, R¹ is OMe, R² is Cl and R³ is H then R⁴ is not H, Me or Et;
when M and Z are CH₂, Q is 0, T and X are absent, R¹ is Cl, R² is Cl, and R³ is H then R⁴ is not H;
when M and Z are CH₂, Q is 0, T and X are absent, R¹ is OMe, R² is Me, and R³ is H then R⁴ is not H;
when Q and Z are CH₂, M is 0, T and X are absent, R¹ is OMe, R² is Me and R³ is H then R⁴ is not H, Me or Et;
when M and Q are CH₂, Z is 0, T and X are absent, R¹ is OMe, R² is Et and R³ is H then R⁴ is not H;
when M is CH₂, Q is CMe₂, Z is 0, T and X are absent, R¹ and R² are F and R³ is H then R⁴ is not H;
when Q and Z are CH₂, M is S, T and X are absent, R¹ is C₁ or F, R² is Me and R³ is H then R⁴ is not H;
when Q and Z are CH₂, M is S, T and X are absent, R¹ is Cl, R² is C₁ and R³ is H then R⁴ is not H; and when Q and M are CH₂, Z is S, T and X are absent, R¹ is C₁ or Me, R² is Me and R³ is H then R⁴ is not H.

6. The compound according to any one of items 1 to 5, wherein the compound is of formula (1):

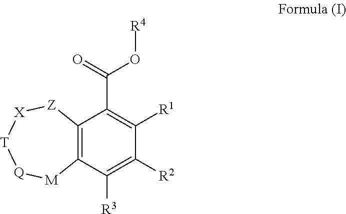

wherein:
   M is CR^{5M}R^{6M}, NR⁹, O or S;
   Q is CR^{7Q}R^{6Q}, NR^{10Q}, O or S;
   T is CR^{7T}R6T, NR^{10T}, O or S;
   X is CR^{7X}R^{6X}, NR^{10X}, O or S;
   Z is CR^{8Z}R^{6Z}, NR¹¹, O or S;
wherein no more than one of M, Q, T, X or Z is selected from the group consisting of NR^{9M}, NR^{10Q}, NR^{10T}, NR^{10X}, NR¹¹, O or S;
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{5M} is selected from the group consisting of H, deuterium, F and C₁₋₃ alkyl optionally substituted with one or more, identical or different, substituents R¹²;
   R^{6M} R^{6Q} R^{6T} R^{6X} and R^{6Z} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q}, R^{7T} and R^{7X} are independently selected from the group consisting of H, deuterium, F and C₁₋₃ alkyl optionally substituted with one or more, identical or different, substituents R¹²;
   R^{8Z} is selected from the group consisting of H, deuterium, F and C₁₋₃ alkyl optionally substituted with one or more, identical or different, substituents R¹²;
   R⁹ is selected from the group consisting of H, C₁₋₃ alkyl optionally substituted with one or more, identical or different, substituents R¹² and C₁₋₃ alkyl substituted with R¹⁴;
   R¹⁰, R^{10T} and R^{10X} are independently selected from benzyl optionally substituted with one or more, identical or different, substituents R¹³;
   R¹¹ is selected from the group consisting of H, C₁₋₃ alkyl optionally substituted with one or more, identical or different, substituents R¹² and C₁₋₃ alkyl substituted with R¹⁵;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F;
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; and
   R¹⁵ is independently selected from the group consisting of C₃₋₆ cycloalkyl optionally substituted with one or more, identical or different, substituents R¹², C-10 aryl optionally substituted with one or more, identical or different, substituents R¹³ and 5- to 10-membered heteroaryl optionally substituted with one or more, identical or different, substituents R¹³;
or a pharmaceutically acceptable salt, hydrate, polymorph, tautomer, or solvate thereof.

7. The compound according to any one of items 1 to 7, wherein compound is of formula (III):

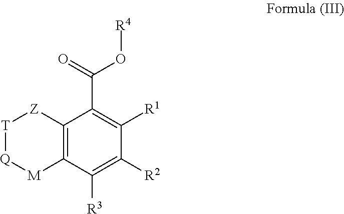

wherein:
   M is CR^{5M}R^{6M}, NR⁹, O or S;
   Q is CR^{7Q}R^{6Q}, NR^{10Q}, O or S;
   T is CR^{7T}R^{6T}, NR^{10T}, O or S;
   Z is CR^{8Z}R^{6Z}, NR¹¹, O or S;
wherein no more than one of M, Q, T or Z is selected from the group consisting of NR⁹M, NR^{10Q}, NR^{10T}, NR¹¹, O or S;
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{5M} is selected from the group consisting of H, deuterium, F and C₁₋₃ alkyl optionally substituted with one or more, identical or different, substituents R¹²;
   R^{6M}, R^{6Q} R^{6T} and R^{6Z} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q} and R^{7T} are independently selected from the group consisting of H, deuterium, F and C₁₋₃ alkyl optionally substituted with one or more, identical or different, substituents R¹²;
   R^{8Z} is selected from the group consisting of H, deuterium, F and C₁₋₃ alkyl optionally substituted with one or more, identical or different, substituents R¹²;
   R⁹ is selected from the group consisting of H, C₁₋₃ alkyl optionally substituted with one or more, identical or different, substituents R¹² and C₁₋₃ alkyl substituted with R¹⁴;
   R¹⁰ and R^{10T} are independently selected from benzyl optionally substituted with one or more, identical or different, substituents R¹³;
   R¹¹ is selected from the group consisting of H, C₁₋₅ alkyl optionally substituted with one or more, identical or different, substituents R¹² and C₁₋₃ alkyl substituted with R¹⁵;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F;
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; and
   R¹⁵ is independently selected from the group consisting of C₃₋₆ cycloalkyl optionally substituted with one or more, identical or different, substituents R¹², C₆10 aryl optionally substituted with one or more, identical or different, substituents R¹³ and 5- to 10-membered heteroaryl optionally substituted with one or more, identical or different, substituents R¹³;
or a pharmaceutically acceptable salt, hydrate, polymorph, tautomer, or solvate thereof.

8. The compound according to any one of items 1 to 8, wherein the compound is of formula (II):

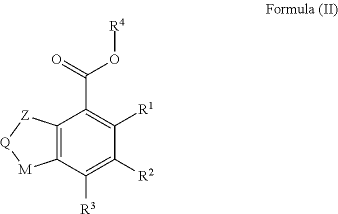

wherein:
   M is CR^{5M}R^{6M}, NR⁹, O or S;
   Q is CR^{7Q}R^{6Q}, NR^{10Q}, O or S;
   Z is CR^{8Z}R^{6Z}, NR¹¹, O or S;
wherein no more than one of M, Q or Z is selected from the group consisting of NR⁹, NR¹⁰, NR¹¹, O or S;
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{5M} is selected from the group consisting of H, deuterium, F and C₁₋₃ alkyl optionally substituted with one or more, identical or different, substituents R¹²;
   R^{6M}, R^{6Q} and R^{6Z} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q} is selected from the group consisting of H, deuterium, F and C₁₋₃ alkyl optionally substituted with one or more, identical or different, substituents R¹²;
   R^{8Z} is selected from the group consisting of H, deuterium, F and C₁₋₃ alkyl optionally substituted with one or more, identical or different, substituents R¹²;
   R⁹ is selected from the group consisting of H, C₁₋₃ alkyl optionally substituted with one or more, identical or different, substituents R¹² and C₁₋₃ alkyl substituted with R¹⁴;
   R^{10Q} is selected from benzyl optionally substituted with one or more, identical or different, substituents R¹³;
   R¹¹ is selected from the group consisting of H, C₁₋₅ alkyl optionally substituted with one or more, identical or different, substituents R¹² and C₁₋₃ alkyl substituted with R¹⁵;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F;
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; and
   R¹⁵ is independently selected from the group consisting of C₃₋₆ cycloalkyl optionally substituted with one or more, identical or different, substituents R¹², C₆10 aryl optionally substituted with one or more, identical or different, substituents R¹³ and 5- to 10-membered heteroaryl optionally substituted with one or more, identical or different, substituents R¹³;
or a pharmaceutically acceptable salt, hydrate, polymorph, tautomer, or solvate thereof.

9. The compound according to any one of items 1 to 5, wherein M is CR⁵R⁶.

10. The compound according to any one of items 6 to 8, wherein M is CR^{5M}R^{6M}.

11. The compound according to any one of items 1 to 8, wherein M is NR⁹.

12. The compound according to any one of items 1 to 8, wherein M is O.

13. The compound according to any one of items 1 to 8, wherein M is S.

14. The compound according to any one of items 1 to 5, wherein Q is CR⁷R⁶.

15. The compound according to any one of items 6 to 8, wherein Q is CR^{7Q}R^{6Q}.

16. The compound according to any one of items 1 to 13, wherein Q is NR¹⁰.

17. The compound according to any one of items 1 to 13, wherein Q is NR^{10Q}.

18. The compound according to any one of items 1 to 13, wherein Q is O.

19. The compound according to any one of items 1 to 13, wherein Q is S.

20. The compound according to any one of items 1 to 7, wherein T is absent.

21. The compound according to any one of items 1 to 5, wherein T is CR⁷R⁶.

22. The compound according to any one of items 6 or 7, wherein T is CR^{7T}R^{6T}.

23. The compound according to any one of items 1 to 19, wherein T is NR¹⁰.

24. The compound according to any one of items 1 to 19, wherein T is NR^{10T}

25. The compound according to any one of items 1 to 19, wherein T is O.

26. The compound according to any one of items 1 to 19, wherein T is S.

27. The compound according to any one of items 1 to 6, wherein X is absent.

28. The compound according to any one of items 1 to 5, wherein X is CR⁷R⁶.

29. The compound according to item, wherein X is CR^{7X}R^{6X}.

30. The compound according to any one of items 1 to 26, wherein X is NR¹⁰.

31. The compound according to any one of items 1 to 26, wherein X is NR^{10X}.

32. The compound according to any one of items 1 to 26, wherein X is O.

33. The compound according to any one of items 1 to 26, wherein X is S.

34. The compound according to any one of items 1 to 5, wherein Z is CR⁸R⁶.

35. The compound according to any one of items 6 to 8, wherein Z is CR^{8Z}R^{6Z}.

36. The compound according to any one of items 1 to 33, wherein Z is NR¹¹.

37. The compound according to any one of items 1 to 33, wherein Z is O.

38. The compound according to any one of items 1 to 33, wherein Z is S.

39. The compound according to any one of items 1 to 8, wherein Q is CR^{7Q}R^{6Q} or NR⁹.

40. The compound according to any one of items 1 to 7, wherein Q is CR^{7Q}R^{6Q} or NR⁹ and T is CR^{7T}R^{6T} or NR^{10T}

41. The compound according to any one of items 1 to 6, wherein Q is CR^{7Q}R^{6Q} or NR⁹, T is CR^{7T}R^{6T} or NR^{10T} and X is CR^{7X}R^{6X} or NR^{10X}.

42. The compound according to any one of the preceding items, wherein R⁵ and R⁸ are joined together to form a ring by —CH₂—, —CH₂CH₂- or —CH₂CH₂CH₂-.

43. The compound according to any one of the preceding items, wherein R⁵ and R¹¹ are joined together to form a ring by —CH₂—, —CH₂CH₂- or —CH₂CH₂CH₂-.

44. The compound according to any one of the preceding items, wherein R⁹ and R⁸ are joined together to form a ring by —CH₂—, —CH₂CH₂- or —CH₂CH₂CH₂-.

45. The compound according to any one of items 1 to 5, wherein M is CR⁵R⁶; Q is CR⁷R⁶; T is absent; X is absent; and Z is CR⁸R⁶.

46. The compound according to any one of items 1 to 5, wherein M is CR⁵R⁶; Q is CR⁷R⁶; T is CR⁷R⁶; X is absent; and Z is CR⁸R⁶.

47. The compound according to any one of items 1 to 5, wherein M is CR⁵R⁶; Q is CR⁷R⁶; T is CR⁷R⁶; X is CR⁷R⁶; and Z is CR⁸R⁶.

48. The compound according to any one of items 1 to 5, wherein M is CR⁵R⁶; Q is CR⁷R⁶; T is absent; X is absent; and Z is O.

49. The compound according to any one of items 1 to 5, wherein M is CR⁵R⁶; Q is CR⁷R⁶; T is CR⁷R⁶; X is absent; and Z is O.

50. The compound according to any one of items 1 to 5, wherein M is CR⁵R⁶; Q is CR⁷R⁶; T is CR⁷R⁶; X is absent; and Z is S.

51. The compound according to any one of items 1 to 5, wherein M is CR⁵R⁶; Q is CR⁷R⁶; T is CR⁷R⁶; X is absent; and Z is NR¹¹.

52. The compound according to any one of items 1 to 5, wherein M is 0; Q is CR⁷R⁶; T is CR⁷R⁶; X is absent; and Z is CR⁸R⁶.

53. The compound according to any one of items 1 to 5, wherein M is S; Q is CR⁷R⁶; T is CR⁷R⁶; X is absent; and Z is CR⁸R⁶.

54. The compound according to any one of items 1 to 5, wherein M is NR⁹; Q is CR⁷R⁶; T is CR⁷R⁶; X is absent; and Z is CR⁸R⁶.

55. The compound according to any one of items 1 to 5, wherein M is CR⁵R⁶; Q is CR⁷R⁶; T is CR⁷R⁶; X is absent; Z is CR⁸R⁶; and R⁵ and R^{a} are joined together to form a ring by —CH₂-.

56. The compound according to any one of items 1 to 5, wherein M is CR⁵R⁶; Q is CR⁷R⁶; T is CR⁷R⁶; X is absent; Z is CR⁸R⁶; and R⁵ and R⁸ are joined together to form a ring by —CH₂CH₂-.

57. The compound according to any one of items 1 to 5, wherein M is CR⁵R⁶; Q is CR⁷R⁶; T is absent; X is absent; and Z is S.

58. The compound according to any one of items 1 to 5, wherein M is CR⁵R⁶; Q is CR⁷R⁶; T is absent; X is absent; and Z is NR¹¹.

59. The compound according to any one of items 1 to 5, wherein M is 0; Q is CR⁷R⁶; T is absent; X is absent; and Z is CR⁸R⁶.

60. The compound according to any one of items 1 to 5, wherein M is S; Q is CR⁷R⁶; T is absent; X is absent; and Z is CR⁸R⁶.

61. The compound according to any one of items 1 to 5, wherein M is NR⁹; Q is CR⁷R⁶; T is absent; X is absent; and Z is CR⁸R⁶.

62. The compound according to any one of items 1 to 5, wherein M is CR⁵R⁶; Q is CR⁷R⁶; T is CR⁷R⁶; X is CR⁷R⁶; and Z is O.

63. The compound according to any one of items 1 to 5, wherein M is CR⁵R⁶; Q is CR⁷R⁶; T is CR⁷R⁶; X is CR⁷R⁶; and Z is S.

64. The compound according to any one of items 1 to 5, wherein M is CR⁵R⁶; Q is CR⁷R⁶; T is CR⁷R⁶; X is CR⁷R⁶; and Z is NR¹¹.

65. The compound according to any one of items 1 to 5, wherein M is 0; Q is CR⁷R⁶; T is CR⁷R⁶; X is CR⁷R⁶; and Z is CR⁸R⁶.

66. The compound according to any one of items 1 to 5, wherein M is S; Q is CR⁷R⁶; T is CR⁷R⁶; X is CR⁷R⁶; and Z is CR⁸R⁶.

67. The compound according to any one of items 1 to 5, wherein M is NR⁹; Q is CR⁷R⁶; T is CR⁷R⁶; X is CR⁷R⁶; and Z is CR⁸R⁶.

68. The compound according to any one of items 1 to 5, wherein M is CR⁵R⁶; Q is NR¹⁰; T is absent; X is absent; and Z is CR⁶R⁶.

69. The compound according to item 1 or 2, wherein the compound is selected from the group consisting of Formula (IV), Formula (V), Formula (VI), Formula (VII), Formula (VIII), Formula (IX), Formula (X), Formula (XI), Formula (XII), Formula (XIII), Formula (XIV), Formula (XV), Formula (XVI), Formula (XVII), Formula (XVIII), Formula (XIX), Formula (XX), Formula (XXI), Formula (XXII), Formula (XXIII), Formula (XXIV), Formula (XXV), Formula (XXVI) and Formula (XXVII):

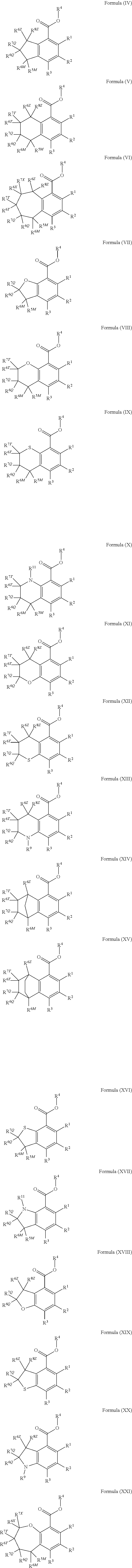

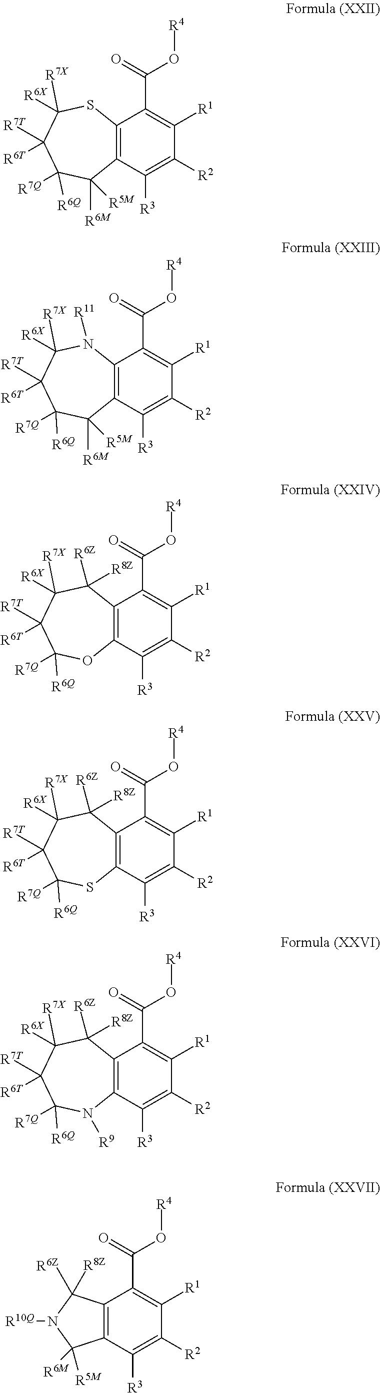

wherein:
   R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
   R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
   R³ is selected from the group consisting of H; F and Cl;
   R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
   R^{5M} is selected from the group consisting of H, deuterium, F and C₁₋₃ alkyl optionally substituted with one or more, identical or different, substituents R¹²;
   R^{6M} R^{6Q} R^{6T} R^{6X} and R^{6Z} are independently selected from the group consisting of H, deuterium, F, C₁₋₃ alkyl substituted with R¹⁴ and C₁₋₃ alkyl optionally substituted with one or more, identical or different, substituents R¹²;
   R^{7Q}, R^{7T} and R^{7X} are independently selected from the group consisting of H, deuterium, F and C₁₋₃ alkyl optionally substituted with one or more, identical or different, substituents R¹²;
   R^{8Z} is selected from the group consisting of H, deuterium, F and C₁₋₃ alkyl optionally substituted with one or more, identical or different, substituents R¹²;
   R⁹ is selected from the group consisting of H, C₁₋₃ alkyl optionally substituted with one or more, identical or different, substituents R¹² and C₁₋₃ alkyl substituted with R¹⁴;
   R¹⁰ is selected from benzyl optionally substituted with one or more, identical or different, substituents R¹³;
   R¹¹ is selected from the group consisting of H, C₁₋₃ alkyl optionally substituted with one or more, identical or different, substituents R¹² and C₁₋₃ alkyl substituted with R¹⁵;
   R¹² is independently selected from the group consisting of deuterium, F and OMe;
   R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F;
   R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; and
   R¹⁵ is independently selected from the group consisting of C₃₋₆ cycloalkyl optionally substituted with one or more, identical or different, substituents R¹², C₆₋₁₀ aryl optionally substituted with one or more, identical or different, substituents R¹³ and 5- to 10-membered heteroaryl optionally substituted with one or more, identical or different, substituents R¹³;
or a pharmaceutically acceptable salt, hydrate, polymorph, tautomer, or solvate thereof.

70. The compound according to item 69, wherein the compound is of Formula (IV).

71. The compound according to item 69, wherein the compound is of Formula (V).

72. The compound according to item 69, wherein the compound is of Formula (VI).

73. The compound according to item 69, wherein the compound is of Formula (VII).

74. The compound according to item 69, wherein the compound is of Formula (VIII).

75. The compound according to item 69, wherein the compound is of Formula (IX).

76. The compound according to item 69, wherein the compound is of Formula (X).

77. The compound according to item 69, wherein the compound is of Formula (XI).

78. The compound according to item 69, wherein the compound is of Formula (XII).

79. The compound according to item 69, wherein the compound is of Formula (XIII).

80. The compound according to item 69, wherein the compound is of Formula (XIV).

81. The compound according to item 69, wherein the compound is of Formula (XV).

82. The compound according to item 69, wherein the compound is of Formula (XVI).

83. The compound according to item 69, wherein the compound is of Formula (XVII).

84. The compound according to item 69, wherein the compound is of Formula (XVIII).

85. The compound according to item 69, wherein the compound is of Formula (XIX).

86. The compound according to item 69, wherein the compound is of Formula (XX).

87. The compound according to item 69, wherein the compound is of Formula (XXI).

88. The compound according to item 69, wherein the compound is of Formula (XXII).

89. The compound according to item 69, wherein the compound is of Formula (XXIII).

90. The compound according to item 69, wherein the compound is of Formula (XXIV).

91. The compound according to item 69, wherein the compound is of Formula (XXV).

92. The compound according to item 69, wherein the compound is of Formula (XXVI).

93. The compound according to item 69, wherein the compound is of Formula (XXVII).

94. The compound according to any one of items 69 to 93, wherein R^{5M} is R⁵.

95. The compound according to any one of items 69 to 93, wherein R^{5M} is H.

96. The compound according to any one of items 69 to 95, wherein R^{6M} is R⁶.

97. The compound according to any one of items 69 to 95, wherein R^{6M} is H.

98. The compound according to any one of items 69 to 97, wherein R^{6Q} is R⁶.

99. The compound according to any one of items 69 to 97, wherein R^{6Q} is H.

100. The compound according to any one of items 69 to 99, wherein R^{6T} is R⁶.

101. The compound according to any one of items 69 to 99, wherein R^{6T} is H.

102. The compound according to any one of items 69 to 101, wherein R^{6X} is R⁶.

103. The compound according to any one of items 69 to 101, wherein R^{6X} is H.

104. The compound according to any one of items 69 to 103, wherein R^{6Z} is R⁶.

105. The compound according to any one of items 69 to 103, wherein R^{6Z} is H.

106. The compound according to any one of items 69 to 105, wherein R^{7Q} is R⁷.

107. The compound according to any one of items 69 to 105, wherein R^{7Q} is H.

108. The compound according to any one of items 69 to 107, wherein R^{7T} is R⁷.

109. The compound according to any one of items 69 to 107, wherein R^{7T} is H.

110. The compound according to any one of items 69 to 109, wherein R^{7X} is R⁷.

111. The compound according to any one of items 69 to 109, wherein R^{7X} is H.

112. The compound according to any one of items 69 to 111, wherein R^{8Z} is R⁸.

113. The compound according to any one of items 69 to 111, wherein R^{8Z} is H.

114. The compound according to any one of items 69 to 113, wherein R⁹ is C₁₋₃ alkyl, such as Me.

115. The compound according to any one of items 69 to 114, wherein R^{10Q} is R¹⁰.

116. The compound according to any one of items 69 to 115, wherein R^{10Q} is benzyl.

117. The compound according to any one of items 69 to 115, wherein R^{10T} is R¹⁰

118. The compound according to any one of items 69 to 115, wherein R^{10T} is benzyl.

119. The compound according to any one of items 69 to 118, wherein R^{1O}X is R¹⁰.

120. The compound according to any one of items 69 to 118, wherein R¹ox is benzyl.

121. The compound according to any one of items 69 to 120, wherein R¹¹ is C₁₋₃ alkyl, such as Me.

122. The compound according to any one of items 1 to 121, wherein R¹ is F.

123. The compound according to any one of items 1 to 121, wherein R¹ is Cl.

124. The compound according to any one of items 1 to 121, wherein R¹ is C₁₋₃ alkyl, such as Me or Et.

125. The compound according to any one of items 1 to 121, wherein R¹ is —C₁₋₃ alkyl substituted with one or more deuterium.

126. The compound according to any one of items 1 to 121, wherein R¹ is —OC₁₋₃ alkyl, such as OMe.

127. The compound according to any one of items 1 to 121, wherein R¹ is —OC₁₋₃ alkyl substituted with one or more, identical or different substituents R¹², wherein R¹² is independently selected from the group consisting of deuterium, F and OMe.

128. The compound according to any one of items 1 to 121, wherein R¹ is —OCH₂F, —OCHF₂, or —OCF₃.

129. The compound according to any one of items 1 to 121, wherein R¹ is —SC₁₋₃ alkyl, such as SMe.

130. The compound according to any one of items 1 to 121, wherein R¹ is —SC₁₋₃ alkyl substituted with one or more, identical or different substituents R¹², wherein R¹² is independently selected from the group consisting of deuterium, F and OMe.

131. The compound according to any of items 1 to 121, wherein R¹ is selected from the group consisting of F, Cl, Me, OMe, OCH₂F, OCHF₂, OCF₃ and SMe.

132. The compound according to any of items 1 to 121, wherein R¹ is C₂₋₃ alkenyl, such as ethenyl.

133. The compound according to any one of items 1 to 121, wherein R¹ is C₂₋₃ alkenyl substituted with one or more, identical or different substituents R¹², wherein R¹² is independently selected from the group consisting of deuterium, F and OMe.

134. The compound according to any one of items 1 to 121, wherein R¹ is —OC₃₋₅ cycloalkyl.

135. The compound according to any one of items 1 to 121, wherein R¹ is —OC₃₋₅ cycloalkyl substituted with one or more, identical or different substituents R¹², wherein R¹² is independently selected from the group consisting of deuterium, F and OMe.

136. The compound according to any one of items 1 to 121, wherein R¹ is —SC₃₋₅ cycloalkyl.

137. The compound according to any one of items 1 to 121, wherein R¹ is —SC₃₋₅ cycloalkyl substituted with one or more, identical or different substituents R¹², wherein R¹² is independently selected from the group consisting of deuterium, F and OMe.

138. The compound according to any of items 1 to 137, wherein R² is F.

139. The compound according to any of items 1 to 137, wherein R² is Cl.

140. The compound according to any of items 1 to 137, wherein R² is Br.

141. The compound according to any of items 1 to 137, wherein R² is I.

142. The compound according to any of items 1 to 137, wherein R² is C₁₋₃ alkyl, such as Me or Et.

143. The compound according to any of items 1 to 137, wherein R² is C₁₋₃ alkyl substituted with one or more, identical or different substituents R¹², wherein R¹² is independently selected from the group consisting of deuterium, F and OMe.

144. The compound according to any of items 1 to 137, wherein R² is C₃ cycloalkyl.

145. The compound according to any of items 1 to 137, wherein R² is C₃ cycloalkyl substituted with one or more, identical or different substituents R¹², wherein R¹² is independently selected from the group consisting of deuterium, F and OMe.

146. The compound according to any of items 1 to 137, wherein R² is selected from the group consisting of F, Cl and Me.

147. The compound according to any of items 1 to 146, wherein R³ is H.

148. The compound according to any of items 1 to 146, wherein R³ is F.

149. The compound according to any of items 1 to 146, wherein R³ is Cl.

150. The compound according to any of items 1 to 149, wherein R⁴ is H.

151. The compound according to any of items 1 to 149, wherein R⁴ is C₁₅ alkyl.

152. The compound according to any of items 1 to 149, wherein R⁴ is C₁₋₅ alkyl substituted with one or more, identical or different substituents R¹², wherein R¹² is independently selected from the group consisting of deuterium, F and OMe.

153. The compound according to any of items 1 to 149, wherein R⁴ is C₂₋₅ alkenyl.

154. The compound according to any of items 1 to 149, wherein R⁴ is C₂₋₅ alkenyl substituted with one or more, identical or different substituents R¹², wherein R¹² is independently selected from the group consisting of deuterium, F and OMe.

155. The compound according to any of items 1 to 149, wherein R⁴ is C₂₋₅ alkynyl.

156. The compound according to any of items 1 to 149, wherein R⁴ is C₂₋₅ alkynyl substituted with one or more, identical or different substituents R¹², wherein R¹² is independently selected from the group consisting of deuterium, F and OMe.

157. The compound according to any of items 1 to 149, wherein R⁴ is C₃₋₆ cycloalkyl.

158. The compound according to any of items 1 to 149, wherein R⁴ is C₃₋₆ cycloalkyl substituted with one or more, identical or different substituents R¹², wherein R¹² is independently selected from the group consisting of deuterium, F and OMe.

159. The compound according to any of items 1 to 149, wherein R⁴ is phenyl.

160. The compound according to any of items 1 to 149, wherein R⁴ is phenyl substituted with one or more, identical or different substituents R¹³, wherein R¹³ is independently selected from the group consisting of deuterium, methoxy, nitro, cyano, Cl, Br, I, and F.

161. The compound according to any of items 1 to 149, wherein R⁴ is benzyl.

162. The compound according to any of items 1 to 149, wherein R⁴ is benzyl substituted with one or more, identical or different substituents R¹³, wherein R¹³ is independently selected from the group consisting of deuterium, methoxy, nitro, cyano, Cl, Br, I, and F.

163. The compound according to any one of items 1 to 162, wherein R⁵ is H.

164. The compound according to any one of items 1 to 162, wherein R⁵ is deuterium.

165. The compound according to any one of items 1 to 162, wherein R⁵ is F.

166. The compound according to any one of items 1 to 162, wherein R⁵ is a bond.

167. The compound according to any one of items 1 to 162, wherein R⁵ is C₁.3 alkanediyl.

168. The compound according to any one of items 1 to 162, wherein R⁵ is C₁₋₃ alkyl.

169. The compound according to any one of items 1 to 162, wherein R⁵ is C₁₋₃ alkyl substituted with one or more, identical or different substituents R¹², wherein R¹² is independently selected from the group consisting of deuterium, F and OMe.

170. The compound according to any one of items 1 to 169, wherein R⁶ is H.

171. The compound according to any one of items 1 to 169, wherein R⁶ is deuterium.

172. The compound according to any one of items 1 to 169, wherein R⁶ is F.

173. The compound according to any one of items 1 to 169, wherein R⁶ is C₁₋₃ alkyl.

174. The compound according to any one of items 1 to 169, wherein R⁶ is C₁₋₃ alkyl substituted with one or more, identical or different substituents R¹², wherein R¹² is independently selected from the group consisting of deuterium, F and OMe.

175. The compound according to any one of items 1 to 169, wherein R⁶ is C₁₋₃ alkyl substituted with R¹⁴.

176. The compound according to any one of items 1 to 169, wherein R⁶ is C₁₋₃ alkyl substituted with R¹⁴; R¹⁴ is C₃₋₅ cycloalkyl optionally substituted with one or more, identical or different, substituents R¹²; and R¹² is independently selected from the group consisting of deuterium, F and OMe.

177. The compound according to any one of items 1 to 169, wherein R⁶ is C₁₋₃ alkyl substituted with R¹⁴; R¹⁴ is phenyl optionally substituted with one or more, identical or different, substituents R¹³; and R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F.

178. The compound according to any one of items 1 to 169, wherein R⁶ is C, alkyl substituted with R¹⁴; R¹⁴ is phenyl optionally substituted with one or more, identical or different, substituents R¹³; and R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F.

179. The compound according to any one of items 1 to 169, wherein R⁶ is C₁₋₃ alkyl substituted with R¹⁴; R¹⁴ is 5-membered heteroaryl optionally substituted with one or more, identical or different, substituents R¹³; and R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F.

180. The compound according to any one of items 1 to 169, wherein R⁶ is benzyl.

181. The compound according to any one of items 1 to 180, wherein R⁷ is H.

182. The compound according to any one of items 1 to 180, wherein R⁷ is deuterium.

183. The compound according to any one of items 1 to 180, wherein R⁷ is F.

184. The compound according to any one of items 1 to 180, wherein R⁷ is C₁₋₃ alkyl.

185. The compound according to any one of items 1 to 180, wherein R⁷ is C₁₋₃ alkyl substituted with one or more, identical or different substituents R¹², wherein R¹² is independently selected from the group consisting of deuterium, F and OMe.

186. The compound according to any one of items 1 to 185, wherein R^{a} is H.

187. The compound according to any one of items 1 to 185, wherein R^{a} is deuterium.

188. The compound according to any one of items 1 to 185, wherein R^{a} is F.

189. The compound according to any one of items 1 to 185, wherein R^{a} is a bond.

190. The compound according to any one of items 1 to 185, wherein R^{a} is C₁₋₃ alkanediyl.

191. The compound according to any one of items 1 to 185, wherein R^{a} is C₁₃ alkyl.

192. The compound according to any one of items 1 to 185, wherein R^{a} is C₁₋₃ alkyl substituted with one or more, identical or different substituents R¹², wherein R¹² is independently selected from the group consisting of deuterium, F and OMe.

193. The compound according to any one of items 1 to 192, wherein R⁹ is H.

194. The compound according to any one of items 1 to 192, wherein R⁹ is C₁₋₅ alkyl.

195. The compound according to any one of items 1 to 192, wherein R⁹ is C₁₋₅ alkyl substituted with one or more, identical or different substituents R¹², wherein R¹² is independently selected from the group consisting of deuterium, F and OMe.

196. The compound according to any one of items 1 to 192, wherein R⁹ is C₁₋₅ alkyl substituted with R¹⁴.

197. The compound according to any one of items 1 to 192, wherein R⁹ is C₁₋₃ alkyl substituted with R¹⁴, wherein R¹⁴ is C₃₋₅ cycloalkyl optionally substituted with one or more, identical or different, substituents R¹², and wherein R¹² is independently selected from the group consisting of deuterium, F and OMe.

198. The compound according to any one of items 1 to 192, wherein R⁹ is C₁₋₃ alkyl substituted with R¹⁴, wherein R¹⁴ is phenyl optionally substituted with one or more, identical or different, substituents R¹³, and wherein R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F.

199. The compound according to any one of items 1 to 192, wherein R⁹ is C₁₋₃ alkyl substituted with R¹⁴, wherein R¹⁴ is 5-membered heteroaryl optionally substituted with one or more, identical or different, substituents R¹³, and wherein R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F.

200. The compound according to any one of items 1 to 192, wherein R⁹ is a bond.

201. The compound according to any one of items 1 to 192, wherein R⁹ is C₁.3 alkanediyl.

202. The compound according to any one of items 1 to 192, wherein R⁹ is benzyl.

203. The compound according to any one of items 1 to 183, wherein R⁹ is benzyl optionally substituted with one or more, identical or different, substituents R¹³, wherein R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F.

204. The compound according to any one of items 1 to 192, wherein R⁹ is benzyl optionally substituted with one or more, identical or different, substituents R¹³, wherein R¹³ is independently selected from the group consisting of deuterium, methoxy, nitro, cyano, Cl, Br, I, and F.

205. The compound according to any one of items 1 to 204, wherein R¹⁰ is benzyl.

206. The compound according to any one of items 1 to 204, wherein R¹⁰ is benzyl optionally substituted with one or more, identical or different, substituents R¹³, wherein R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F.

207. The compound according to any one of items 1 to 204, wherein R¹⁰ is benzyl optionally substituted with one or more, identical or different, substituents R¹³, wherein R¹³ is independently selected from the group consisting of deuterium, methoxy, nitro, cyano, Cl, Br, I, and F.

208. The compound according to any one of items 1 to 207, wherein R¹¹ is H.

209. The compound according to any one of items 1 to 207, wherein R¹¹ is C₁₋₅ alkyl.

210. The compound according to any one of items 1 to 207, wherein R¹¹ is C₁₋₅ alkyl substituted with one or more, identical or different substituents R¹², wherein R¹² is independently selected from the group consisting of deuterium, F and OMe.

211. The compound according to any one of items 1 to 207, wherein R¹¹ is a bond.

212. The compound according to any one of items 1 to 207, wherein R¹¹ is C₁.3 alkanediyl.

213. The compound according to any one of items 1 to 207, wherein R¹¹ is C₁₋₃ alkyl substituted with one or more, identical or different substituents R¹⁵, wherein R¹⁵ is C₃₋₆ cycloalkyl optionally substituted with one or more, identical or different, substituents R¹², and wherein R¹² is independently selected from the group consisting of deuterium, F and OMe.

214. The compound according to any one of items 1 to 207, wherein R¹¹ is C₁₋₃ alkyl substituted with one or more, identical or different substituents R¹⁵, wherein R¹⁵ is C₆10 aryl optionally substituted with one or more, identical or different, substituents R¹³, and wherein R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F.

215. The compound according to any one of items 1 to 207, wherein R¹¹ is 5- to 10-membered heteroaryl optionally substituted with one or more, identical or different, substituents R¹³, and wherein R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F.

216. The compound according to any one of items 1 to 207, wherein R¹¹ is benzyl.

217. The compound according to any one of items 1 to 207, wherein R¹¹ is benzyl optionally substituted with one or more, identical or different, substituents R¹³, wherein R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F.

218. The compound according to any one of items 1 to 207, wherein R¹¹ is benzyl optionally substituted with one or more, identical or different, substituents R¹³, wherein R¹³ is independently selected from the group consisting of deuterium, methoxy, nitro, cyano, Cl, Br, I, and F.

219. The compound according to any of items 1 to 162, wherein R⁵, R⁶ R⁷ and R⁸ are H.

220. The compound according to any one of items 1 to 219, wherein R¹⁴ is C₃₋₅ cycloalkyl.

221. The compound according to any one of items 1 to 219, wherein R¹⁴ is C₃₋₅ cycloalkyl substituted with one or more, identical or different substituents R¹²; wherein R¹² is independently selected from the group consisting of deuterium, F and OMe.

222. The compound according to any one of items 1 to 219, wherein R¹⁴ is phenyl.

223. The compound according to any one of items 1 to 219, wherein R¹⁴ is phenyl substituted with one or more, identical or different, substituents R¹³, wherein R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F.

224. The compound according to any one of items 1 to 219, wherein R¹⁴ is 5-membered heteroaryl.

225. The compound according to any one of items 1 to 219, wherein R¹⁴ is 5-membered heteroaryl substituted with one or more, identical or different, substituents R¹³, wherein R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F.

226. The compound according to any one of items 1 to 219, wherein R¹⁴ is selected from the group consisting of oxazolyl, thiazolyl, thiadiazolyl, oxadiazolyl, triazolyl, pyrrolyl, thienyl, furanyl, diazolyl, imidazolyl, pyrazolyl, isoxazolyl, isothiazolyl, and tetrazolyl.

227. The compound according to any one of items 1 to 219, wherein R¹⁴ is selected from the group consisting of oxazolyl, thiazolyl, thiadiazolyl, oxadiazolyl, triazolyl, pyrrolyl, thienyl, furanyl, diazolyl, imidazolyl, pyrazolyl, isoxazolyl, isothiazolyl, and tetrazolyl, each of which is substituted with one or more, identical or different substituents R¹³, wherein R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F.

228. The compound according to any one of items 1 to 219, wherein R¹⁴ is furanyl, such as furan-2-yl, optionally substituted with one or more, identical or different, substituents R¹³.

229. The compound according to any one of items 1 to 219, wherein R¹⁴ is thiazolyl, such as thiazol-2-yl, optionally substituted with one or more, identical or different, substituents R¹³.

230. The compound according to any one of items 1 to 219, wherein R¹⁴ is oxazolyl, such as oxazol-2-yl, optionally substituted with one or more, identical or different, substituents R¹³.

231. The compound according to any one of items 1 to 230, wherein R¹⁵ is C₃₋₆ cycloalkyl optionally substituted with one or more, identical or different, substituents R¹², wherein R¹² is independently selected from the group consisting of deuterium, F and OMe.

232. The compound according to any one of items 1 to 230, wherein R¹⁵ is cyclobutyl.

233. The compound according to any one of items 1 to 230, wherein R¹⁵ is C₆-1o aryl optionally substituted with one or more, identical or different, substituents R¹³, wherein R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F.

234. The compound according to any one of items 1 to 230, wherein R¹⁵ is 5- to 10-membered heteroaryl optionally substituted with one or more, identical or different, substituents R¹³, wherein R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F.

235. The compound according to any one of items 1 to 230, wherein R¹⁵ is selected from the group consisting of oxazolyl, thiazolyl, thiadiazolyl, oxadiazolyl, triazolyl, pyrrolyl, thienyl, furanyl, diazolyl, imidazolyl, pyrazolyl, isoxazolyl, isothiazolyl, and tetrazolyl, pyridinyl, pyridazinyl, pyrimidinyl, pyrazinyl, triazinyl, 1,4-dihydropyrrolo[3,2-b]pyrrolyl, 1,6-dihydropyrrolo[2,3-b]pyrrolyl, 6H-furo[2,3-b]pyrrole, 4H-furo[2,3-b]pyrrolyl, 6H-thieno[2,3-b]pyrrolyl, 4H-thieno[2,3-b]pyrrolyl, indolyl, isoindolyl, indolizinyl, indazolyl, benzimidazolyl, azaindolyl, azaindazolyl, pyrazolo[1,5-a]pyrimidinyl, purinyl, benzofuranyl, benzo[b]thiophenyl, benzisoxazolyl, benzthiazolyl, benzisothiazolyl, benzo[c][1,2,5]thiadiazolyl, quinolinyl, isoquinolinyl, 4H-quinolizinyl, quinoxalinyl, phthalazinyl, quinazolinyl, cinnolinyl, 1,8-naphthyridinyl, pyrido[3,2-d]pyrimidinyl, pyrido[4,3-d]pyrimidinyl, pyrido[3,4-b]pyrazinyl, pyrido[2,3-b]pyrazinyl and pteridinyl.

236. The compound according to any one of items 1 to 230, wherein R¹⁵ is selected from the group consisting of oxazolyl, thiazolyl, thiadiazolyl, oxadiazolyl, triazolyl, pyrrolyl, thienyl, furanyl, diazolyl, imidazolyl, pyrazolyl, isoxazolyl, isothiazolyl, and tetrazolyl, pyridinyl, pyridazinyl, pyrimidinyl, pyrazinyl, triazinyl, 1,4-dihydropyrrolo[3,2-b]pyrrolyl, 1,6-dihydropyrrolo[2,3-b]pyrrolyl, 6H-furo[2,3-b]pyrrole, 4H-furo[2,3-b]pyrrolyl, 6H-thieno[2,3-b]pyrrolyl, 4H-thieno[2,3-b]pyrrolyl, indolyl, isoindolyl, indolizinyl, indazolyl, benzimidazolyl, azaindolyl, azaindazolyl, pyrazolo[1,5-a]pyrimidinyl, purinyl, benzofuranyl, benzo[b]thiophenyl, benzisoxazolyl, benzthiazolyl, benzisothiazolyl, benzo[c][1,2,5]thiadiazolyl, quinolinyl, isoquinolinyl, 4H-quinolizinyl, quinoxalinyl, phthalazinyl, quinazolinyl, cinnolinyl, 1,8-naphthyridinyl, pyrido[3,2-d]pyrimidinyl, pyrido[4,3-d]pyrimidinyl, pyrido[3,4-b]pyrazinyl, pyrido[2,3-b]pyrazinyl and pteridinyl, each of which is substituted with one or more, identical or different, substituents R¹³, wherein R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F.

237. The compound according to any one of items 1 to 230, wherein R¹⁵ is thienyl, such as thien-3-yl, optionally substituted with one or more, identical or different, substituents R¹³.

238. The compound according to any one of items 1 to 230, wherein R¹⁵ is naphthyl, such as naphth-1-yl, optionally substituted with one or more, identical or different, substituents R¹³.

239. The compound according to any of items 1 to 238, wherein R¹² is F.

240. The compound according to any one of items 1 to 238, wherein R¹² is OMe.

241. The compound according to any one of items 1 to 238, wherein R¹² is deuterium.

242. The compound according to any one of items 1 to 241, wherein R¹³ is deuterium.

243. The compound according to any one of items 1 to 241, wherein R¹³ is methoxy.

244. The compound according to any one of items 1 to 241, wherein R¹³ is nitro.

245. The compound according to any one of items 1 to 241, wherein R¹³ is cyano.

246. The compound according to any one of items 1 to 241, wherein R¹³ is Cl.

247. The compound according to any one of items 1 to 241, wherein R¹³ is Br.

248. The compound according to any one of items 1 to 241, wherein R¹³ is I.

249. The compound according to any one of items 1 to 241, wherein R¹³ is F.

250. The compound according to any one of items 1 to 241, wherein R¹³ is —OCF₃.

251. The compound according to any one of items 1 to 241, wherein R¹³ is Me.

252. The compound according to any one of items 1 to 241, wherein R¹³ is CF₃. 253. The compound according to any one of items 1 to 241, wherein R¹³ is CF₂Cl.

254. The compound according to any one of items 1 to 241, wherein R¹³ is CF₂H.

255. The compound according to any one of items 1 to 241, wherein R¹³ is CFH₂. 256. The compound according to any one of items 1 to 241, wherein R¹³ is CD₃. 257. The compound according to any one of items 1 to 241, wherein R¹³ is cyclopropyl.

258. The compound according to any one of items 1 to 241, wherein R¹³ is NH₂. 259. The compound according to any one of items 1 to 241, wherein R¹³ is —NHAc.

260. The compound according to any one of items 1 to 241, wherein R¹³ is —C(═O)—NH₂.

261. The compound according to any one of the preceding items, provided that:
when R¹ is OMe then R² is not OMe
when M, Q, T, X and Z are CH₂, R¹ is OMe, R² is Cl and R³ is H then R⁴ is not H, Me or Et;
when M, Q, T and Z are CH₂, X is absent, R¹ is OMe, R² is Cl and R³ is H then R⁴ is not H, Me or Et;
when M, Q, T and Z are CH₂, X is absent, R¹ is OMe, R² is CF₃ and R³ is H then R⁴ is not Me;
when M, Q, T and Z are CH₂, X is absent, R¹ is Me, R² is Me and R³ is H then R⁴ is not H;
when M, Q, T and Z are CH₂, X is absent, R¹ is Cl, R² is Cl and R³ is Cl then R⁴ is not H;
when M, Q and Z are CH₂, T and X are absent, R¹ is Me, R² is Me and R³ is H then R⁴ is not H or Me;
when M and Z are CH₂, Q is 0, T and X are absent, R¹ is OMe, R² is Cl and R³ is H then R⁴ is not H, Me or Et;
when M and Z are CH₂, Q is 0, T and X are absent, R¹ is Cl, R² is Cl, and R³ is H then R⁴ is not H;
when M and Z are CH₂, Q is 0, T and X are absent, R¹ is OMe, R² is Me, and R³ is H then R⁴ is not H;
when Q and Z are CH₂, M is 0, T and X are absent, R¹ is OMe, R² is Me and R³ is H then R⁴ is not H, Me or Et;
when M and Q are CH₂, Z is 0, T and X are absent, R¹ is OMe, R² is Et and R³ is H then R⁴ is not H;
when M is CH₂, Q is CMe₂, Z is 0, T and X are absent, R¹ and R² are F and R³ is H then R⁴ is not H;
when Q and Z are CH₂, M is S, T and X are absent, R¹ is C₁ or F, R² is Me and R³ is H then R⁴ is not H;
when Q and Z are CH₂, M is S, T and X are absent, R¹ is Cl, R² is C₁ and R³ is H then R⁴ is not H; and when Q and M are CH₂, Z is S, T and X are absent, R¹ is C₁ or Me, R² is Me and R³ is H then R⁴ is not H.

262. The compound according to any one of the preceding items, provided that the compound is not a compound selected from the group consisting of:
10-chloro-9-methoxybicyclo[5.4.0]undeca-1(7),8,10-triene-8-carboxylic acid; methyl 10-chloro-9-methoxybicyclo[5.4.0]undeca-1(7),8,10-triene-8-carboxylate;
ethyl 10-chloro-9-methoxybicyclo[5.4.0]undeca-1(7),8,10-triene-8-carboxylate;
3-chloro-2-methoxy-5,6,7,8-tetrahydro-1-naphthoic acid;
methyl 3-chloro-2-methoxy-5,6,7,8-tetrahydro-1-naphthoate;
ethyl 3-chloro-2-methoxy-5,6,7,8-tetrahydro-1-naphthoate;
methyl 2-methoxy-3-(trifluoromethyl)-5,6,7,8-tetrahydro-1-naphthoate;
2,3-dimethyl-5,6,7,8-tetrahydro-1-naphthoic acid;
2,3,4-trichloro-5,6,7,8-tetrahydro-1-naphthoic acid;
5,6-dimethyl-4-indancarboxylic acid;
methyl 5,6-dimethyl-4-indancarboxylate;
6-chloro-5-methoxy-1,3-dihydro-4-isobenzofuroic acid;
methyl 6-chloro-5-methoxy-1,3-dihydro-4-isobenzofuroate;
ethyl 6-chloro-5-methoxy-1,3-dihydro-4-isobenzofuroate;
5,6-dichloro-1,3-dihydro-4-isobenzofuroic acid;
5-methoxy-6-methyl-1,3-dihydro-4-isobenzofuroic acid;
5-methoxy-6-methyl-2,3-dihydro-1-benzofuran-4-carboxylic acid;
methyl 5-methoxy-6-methyl-2,3-dihydro-1-benzofuran-4-carboxylate;
ethyl 5-methoxy-6-methyl-2,3-dihydro-1-benzofuran-4-carboxylate;
5-ethyl-6-methoxy-2,3-dihydro-1-benzofuran-7-carboxylic acid;
5,6-difluoro-2,2-dimethyl-2,3-dihydro-1-benzofuran-7-carboxylic acid;
5-fluoro-6-methyl-2,3-dihydro-1-benzothiophene-4-carboxylic acid;
5-chloro-6-methyl-2,3-dihydro-1-benzothiophene-4-carboxylic acid;
5,6-dichloro-2,3-dihydro-1-benzothiophene-4-carboxylic acid;
6-chloro-5-methyl-2,3-dihydro-1-benzothiophene-7-carboxylic acid; and
5,6-dimethyl-2,3-dihydro-1-benzothiophene-7-carboxylic acid.

263. The compound according to any one of the preceding items, wherein the compound is selected from the group consisting of:
6-chloro-5-methyl-2,3-dihydro-1H-indene-4-carboxylic acid;
6-bromo-5-methyl-2,3-dihydro-1H-indene-4-carboxylic acid;
6-chloro-5-(fluoromethyl)-2,3-dihydro-1H-indene-4-carboxylic acid;
3-chloro-5,6,7,8-tetrahydronaphthalene-1-carboxylic acid;
5,6-dimethyl-2,3-dihydro-1H-indene-4-carboxylic acid;
2,3-dimethyl-5,6,7,8-tetrahydronaphthalene-1-carboxylic acid;
6-chloro-2,3-dihydro-1H-indene-4-carboxylic acid;
6-chloro-5-methoxy-2,3-dihydro-1H-indene-4-carboxylic acid;
6-chloro-5-(fluoromethoxy)-2,3-dihydro-1H-indene-4-carboxylic acid;
3-chloro-2-methoxy-5,6,7,8-tetrahydronaphthalene-1-carboxylic acid;
3-chloro-2-methyl-5,6,7,8-tetrahydronaphthalene-1-carboxylic acid;
6-chloro-5-(difluoromethoxy)-2,3-dihydro-1H-indene-4-carboxylic acid;
6-chloro-5-(trifluoromethoxy)-2,3-dihydro-1H-indene-4-carboxylic acid;
5-chloro-6-methyl-2,3-dihydro-1-benzofuran-7-carboxylic acid;
6-chloro-5-ethyl-2,3-dihydro-1H-indene-4-carboxylic acid;
6-chloro-7-methyl-3,4-dihydro-2H-1-benzopyran-8-carboxylic acid;
5-chloro-6-methoxy-2,3-dihydro-1-benzofuran-7-carboxylic acid;
5-chloro-4-methoxytricyclo[6.2.1.0^{2,7}]undeca-2(7),3,5-triene-3-carboxylic acid;
3-chloro-4-fluoro-5,6,7,8-tetrahydronaphthalene-1-carboxylic acid;
3-fluoro-2-methoxy-5,6,7,8-tetrahydronaphthalene-1-carboxylic acid;
3-chloro-4-fluoro-2-methoxy-5,6,7,8-tetrahydronaphthalene-1-carboxylic acid;
4-chloro-5-methyltricyclo[6.2.1.0^{2,7}]undeca-2,4,6-triene-3-carboxylic acid;
5-chloro-4-methyltricyclo[6.2.1.0^{2,7}]undeca-2(7),3,5-triene-3-carboxylic acid;
3-chloro-2-methoxy-6,7,8,9-tetrahydro-5H-benzo[7]annulene-1-carboxylic acid;
6-chloro-5-(methylsulfanyl)-2,3-dihydro-1H-indene-4-carboxylic acid;
2-chloro-3-methoxy-6,7,8,9-tetrahydro-5H-benzo[7]annulene-1-carboxylic acid;
3-chloro-2-(difluoromethoxy)-5,6,7,8-tetrahydronaphthalene-1-carboxylic acid;
3-chloro-2-fluoro-5,6,7,8-tetrahydronaphthalene-1-carboxylic acid;
2-chloro-3-fluoro-5,6,7,8-tetrahydronaphthalene-1-carboxylic acid;
2-chloro-3-methyl-5,6,7,8-tetrahydronaphthalene-1-carboxylic acid;
5-chloro-4-methoxytricyclo[6.2.2.0^{2,7}]dodeca-2,4,6-triene-3-carboxylic acid;
6-chloro-5-(trifluoromethyl)-2,3-dihydro-1H-indene-4-carboxylic acid;
2,3-dichloro-5,6,7,8-tetrahydronaphthalene-1-carboxylic acid;
6-chloro-7-methoxy-3,4-dihydro-2H-1-benzothiopyran-8-carboxylic acid;
3-chloro-2-(methylsulfanyl)-5,6,7,8-tetrahydronaphthalene-1-carboxylic acid;
3-chloro-2-methyl-6,7,8,9-tetrahydro-5H-benzo[7]annulene-1-carboxylic acid;
2-chloro-3-methyl-6,7,8,9-tetrahydro-5H-benzo[7]annulene-1-carboxylic acid;
3-chloro-2-ethenyl-5,6,7,8-tetrahydronaphthalene-1-carboxylic acid;
6-chloro-7-fluoro-3,4-dihydro-2H-1-benzopyran-8-carboxylic acid;
7-chloro-6-fluoro-3,4-dihydro-2H-1-benzopyran-8-carboxylic acid;
6-chloro-7-fluoro-3,4-dihydro-2H-1-benzothiopyran-8-carboxylic acid;
7-chloro-6-fluoro-3,4-dihydro-2H-1-benzopyran-5-carboxylic acid;
6-chloro-7-fluoro-1-methyl-1,2,3,4-tetrahydroquinoline-8-carboxylic acid;
6-chloro-7-fluoro-1,2,3,4-tetrahydroquinoline-8-carboxylic acid;
6,7-difluoro-3,4-dihydro-2H-1-benzothiopyran-8-carboxylic acid;
5-chloro-6-fluoro-2,3-dihydro-1-benzofuran-7-carboxylic acid;
2,3-difluoro-5,6,7,8-tetrahydronaphthalene-1-carboxylic acid;
7-chloro-6-fluoro-1-methyl-1,2,3,4-tetrahydroquinoline-5-carboxylic acid;
7-chloro-6-fluoro-1-methyl-1,2,3,4-tetrahydroquinoline-8-carboxylic acid;
5-methoxy-6-methyl-2,3-dihydro-1H-indene-4-carboxylic acid;
7-fluoro-6-methyl-3,4-dihydro-2H-1-benzothiopyran-8-carboxylic acid;
2-fluoro-3-methyl-5,6,7,8-tetrahydronaphthalene-1-carboxylic acid;
7-fluoro-6-methyl-3,4-dihydro-2H-1-benzopyran-8-carboxylic acid;
3-ethyl-2-fluoro-5,6,7,8-tetrahydronaphthalene-1-carboxylic acid;
3-fluoro-2-methyl-5,6,7,8-tetrahydronaphthalene-1-carboxylic acid;
6-chloro-2,2-dimethyl-3,4-dihydro-2H-1-benzopyran-8-carboxylic acid;
3-cyclopropyl-2-fluoro-5,6,7,8-tetrahydronaphthalene-1-carboxylic acid;
2-fluoro-3-iodo-5,6,7,8-tetrahydronaphthalene-1-carboxylic acid;
8-chloro-7-fluoro-2,3,4,5-tetrahydro-1-benzoxepine-9-carboxylic acid;
6-chloro-7-fluoro-2,3,4,5-tetrahydro-1-benzoxepine-9-carboxylic acid;
6-fluoro-7-methyl-3,4-dihydro-2H-1-benzopyran-5-carboxylic acid;
8-chloro-7-fluoro-2,3,4,5-tetrahydro-1-benzoxepine-6-carboxylic acid;
3-chloro-8,8-difluoro-2-methoxy-5,6,7,8-tetrahydronaphthalene-1-carboxylic acid;
2-fluoro-3-(trifluoromethyl)-5,6,7,8-tetrahydronaphthalene-1-carboxylic acid;
6-chloro-7-fluoro-2,2-dimethyl-3,4-dihydro-2H-1-benzopyran-8-carboxylic acid;
5-chloro-6-fluoro-2,3-dihydro-1H-indene-4-carboxylic acid;
6-chloro-5-fluoro-2,3-dihydro-1H-indene-4-carboxylic acid;
7-chloro-6-fluoro-3,4-dihydro-2H-1-benzothiopyran-8-carboxylic acid;
7-chloro-6-fluoro-3,4-dihydro-2H-1-benzothiopyran-5-carboxylic acid;
2-bromo-3-chloro-5,6,7,8-tetrahydronaphthalene-1-carboxylic acid;
3-bromo-2-chloro-5,6,7,8-tetrahydronaphthalene-1-carboxylic acid;
2-benzyl-5,6-dichloro-2,3-dihydro-1H-isoindole-4-carboxylic acid;
6-chloro-7-methoxy-3,4-dihydro-2H-1-benzopyran-8-carboxylic acid;
6-chloro-7-fluoro-1-[(3-thienyl)methyl]-1,2,3,4-tetrahydro-8-quinolinecarboxylic acid;
1-benzyl-6-chloro-7-fluoro-1,2,3,4-tetrahydro-8-quinolinecarboxylic acid;
6-chloro-7-fluoro-1-[(p-fluorophenyl)methyl]-1,2,3,4-tetrahydro-8-quinolinecarboxylic acid;
3-chloro-5,5-difluoro-2-methoxy-5,6,7,8-tetrahydro-1-naphthoic acid;
1-benzyl-7-chloro-6-fluoro-1,2,3,4-tetrahydro-8-quinolinecarboxylic acid;
7-chloro-6-fluoro-2,2-dimethyl-8-chromancarboxylic acid;
1-benzyl-6-chloro-5-fluoro-4-indolinecarboxylic acid;
7-chloro-6-fluoro-1-methyl-1,2,3,4-tetrahydro-8-quinolinecarboxylic acid;
4-benzyl-6-chloro-7-fluoro-8-chromancarboxylic acid;
7-chloro-6-methoxy-5-chromancarboxylic acid;
4-benzyl-7-fluoro-6-methoxy-8-chromancarboxylic acid;
4-benzyl-7-fluoro-6-methoxy-5-chromancarboxylic acid;
6-chloro-7-fluoro-5-chromancarboxylic acid;
7-chloro-6-methoxy-3,4-dihydro-2H-1-benzothiin-8-carboxylic acid;
3-chloro-2-ethoxy-5,6,7,8-tetrahydro-1-naphthoic acid;
6-chloro-1,2,3,4-tetrahydro-8-quinolinecarboxylic acid; and
7-chloro-6-methoxy-8-chromancarboxylic acid.

264. The compound according to any one of the preceding items, wherein the compound is selected from the group consisting of:
6-chloro-5-methyl-2,3-dihydro-1H-indene-4-carboxylic acid;
6-chloro-5-methoxy-2,3-dihydro-1H-indene-4-carboxylic acid;
6-chloro-5-(fluoromethoxy)-2,3-dihydro-1H-indene-4-carboxylic acid;
3-chloro-2-methyl-5,6,7,8-tetrahydronaphthalene-1-carboxylic acid;
6-chloro-5-(difluoromethoxy)-2,3-dihydro-1H-indene-4-carboxylic acid;
6-chloro-5-(trifluoromethoxy)-2,3-dihydro-1H-indene-4-carboxylic acid;
5-chloro-6-methyl-2,3-dihydro-1-benzofuran-7-carboxylic acid;
6-chloro-5-ethyl-2,3-dihydro-1H-indene-4-carboxylic acid;
6-chloro-7-methyl-3,4-dihydro-2H-1-benzopyran-8-carboxylic acid;
5-chloro-6-methoxy-2,3-dihydro-1-benzofuran-7-carboxylic acid;
5-chloro-4-methoxytricyclo[6.2.1.0^{2,7}]undeca-2(7),3,5-triene-3-carboxylic acid;
3-fluoro-2-methoxy-5,6,7,8-tetrahydronaphthalene-1-carboxylic acid;
3-chloro-4-fluoro-2-methoxy-5,6,7,8-tetrahydronaphthalene-1-carboxylic acid;
4-chloro-5-methyltricyclo[6.2.1.0^{2,7}]undeca-2,4,6-triene-3-carboxylic acid;
5-chloro-4-methyltricyclo[6.2.1.0^{2,7}]undeca-2(7),3,5-triene-3-carboxylic acid;
6-chloro-5-(methylsulfanyl)-2,3-dihydro-1H-indene-4-carboxylic acid;
3-chloro-2-(difluoromethoxy)-5,6,7,8-tetrahydronaphthalene-1-carboxylic acid;
3-chloro-2-fluoro-5,6,7,8-tetrahydronaphthalene-1-carboxylic acid;
2-chloro-3-fluoro-5,6,7,8-tetrahydronaphthalene-1-carboxylic acid;
2-chloro-3-methyl-5,6,7,8-tetrahydronaphthalene-1-carboxylic acid;
5-chloro-4-methoxytricyclo[6.2.2.0^{2,7}]dodeca-2,4,6-triene-3-carboxylic acid;
2,3-dichloro-5,6,7,8-tetrahydronaphthalene-1-carboxylic acid;
6-chloro-7-methoxy-3,4-dihydro-2H-1-benzothiopyran-8-carboxylic acid;
3-chloro-2-(methylsulfanyl)-5,6,7,8-tetrahydronaphthalene-1-carboxylic acid;
3-chloro-2-methyl-6,7,8,9-tetrahydro-5H-benzo[7]annulene-1-carboxylic acid;
2-chloro-3-methyl-6,7,8,9-tetrahydro-5H-benzo[7]annulene-1-carboxylic acid;
3-chloro-2-ethenyl-5,6,7,8-tetrahydronaphthalene-1-carboxylic acid;
6-chloro-7-fluoro-3,4-dihydro-2H-1-benzopyran-8-carboxylic acid;
7-chloro-6-fluoro-3,4-dihydro-2H-1-benzopyran-8-carboxylic acid;
6-chloro-7-fluoro-3,4-dihydro-2H-1-benzothiopyran-8-carboxylic acid;
7-chloro-6-fluoro-3,4-dihydro-2H-1-benzopyran-5-carboxylic acid;
6-chloro-7-fluoro-1-methyl-1,2,3,4-tetrahydroquinoline-8-carboxylic acid;
6-chloro-7-fluoro-1,2,3,4-tetrahydroquinoline-8-carboxylic acid;
6,7-difluoro-3,4-dihydro-2H-1-benzothiopyran-8-carboxylic acid;
5-chloro-6-fluoro-2,3-dihydro-1-benzofuran-7-carboxylic acid;
2,3-difluoro-5,6,7,8-tetrahydronaphthalene-1-carboxylic acid;
7-chloro-6-fluoro-1-methyl-1,2,3,4-tetrahydroquinoline-5-carboxylic acid;
7-chloro-6-fluoro-1-methyl-1,2,3,4-tetrahydroquinoline-8-carboxylic acid;
5-methoxy-6-methyl-2,3-dihydro-1H-indene-4-carboxylic acid;
7-fluoro-6-methyl-3,4-dihydro-2H-1-benzothiopyran-8-carboxylic acid;
2-fluoro-3-methyl-5,6,7,8-tetrahydronaphthalene-1-carboxylic acid;
7-fluoro-6-methyl-3,4-dihydro-2H-1-benzopyran-8-carboxylic acid;
3-ethyl-2-fluoro-5,6,7,8-tetrahydronaphthalene-1-carboxylic acid;
3-fluoro-2-methyl-5,6,7,8-tetrahydronaphthalene-1-carboxylic acid;
3-cyclopropyl-2-fluoro-5,6,7,8-tetrahydronaphthalene-1-carboxylic acid;
8-chloro-7-fluoro-2,3,4,5-tetrahydro-1-benzoxepine-9-carboxylic acid;
6-fluoro-7-methyl-3,4-dihydro-2H-1-benzopyran-5-carboxylic acid;
8-chloro-7-fluoro-2,3,4,5-tetrahydro-1-benzoxepine-6-carboxylic acid;
3-chloro-8,8-difluoro-2-methoxy-5,6,7,8-tetrahydronaphthalene-1-carboxylic acid;
2-fluoro-3-(trifluoromethyl)-5,6,7,8-tetrahydronaphthalene-1-carboxylic acid;
6-chloro-7-fluoro-2,2-dimethyl-3,4-dihydro-2H-1-benzopyran-8-carboxylic acid;
5-chloro-6-fluoro-2,3-dihydro-1H-indene-4-carboxylic acid;
6-chloro-5-fluoro-2,3-dihydro-1H-indene-4-carboxylic acid;
7-chloro-6-fluoro-3,4-dihydro-2H-1-benzothiopyran-8-carboxylic acid;
7-chloro-6-fluoro-3,4-dihydro-2H-1-benzothiopyran-5-carboxylic acid;
2-benzyl-5,6-dichloro-2,3-dihydro-1H-isoindole-4-carboxylic acid;
6-chloro-7-methoxy-3,4-dihydro-2H-1-benzopyran-8-carboxylic acid;
6-chloro-7-fluoro-1-[(3-thienyl)methyl]-1,2,3,4-tetrahydro-8-quinolinecarboxylic acid;
1-benzyl-6-chloro-7-fluoro-1,2,3,4-tetrahydro-8-quinolinecarboxylic acid;
6-chloro-7-fluoro-1-[(p-fluorophenyl)methyl]-1,2,3,4-tetrahydro-8-quinolinecarboxylic acid;
3-chloro-5,5-difluoro-2-methoxy-5,6,7,8-tetrahydro-1-naphthoic acid;
1-benzyl-7-chloro-6-fluoro-1,2,3,4-tetrahydro-8-quinolinecarboxylic acid;
7-chloro-6-fluoro-2,2-dimethyl-8-chromancarboxylic acid;
1-benzyl-6-chloro-5-fluoro-4-indolinecarboxylic acid;
7-chloro-6-fluoro-1-methyl-1,2,3,4-tetrahydro-8-quinolinecarboxylic acid;
4-benzyl-6-chloro-7-fluoro-8-chromancarboxylic acid;
7-chloro-6-methoxy-5-chromancarboxylic acid;
4-benzyl-7-fluoro-6-methoxy-8-chromancarboxylic acid;
4-benzyl-7-fluoro-6-methoxy-5-chromancarboxylic acid;
6-chloro-7-fluoro-5-chromancarboxylic acid; and
3-chloro-2-ethoxy-5,6,7,8-tetrahydro-1-naphthoic acid.

265. The compound according to any one of the preceding items, wherein the compound has activity on the CIC-1 receptor.

266. The compound according to any one of the preceding items, wherein the compound is an inhibitor of the CIC-1 ion channel.

267. The compound according to any one of the preceding items, wherein the recovery of force in muscles with neuromuscular dysfunction is >5%, for example >10%, for example >15%, for example >20%, for example >25%, for example >30% and for example >35%. 268. The compound according to any one of the preceding items, wherein the compound improves the recovered force in isolated rat soleus muscles after exposure to tubocurarine.

269. A composition comprising the compound according to any one of the preceding items.

270. The composition according to item 269, wherein the composition further comprises a pharmaceutically acceptable carrier.

271. The composition according to any one of items 269 or 270, wherein the composition further comprises at least one further active agent.

272. The composition according to item 271, wherein said further active agent is suitable for treating, preventing or ameliorating said neuromuscular disorder.

273. The composition according to item 271, wherein said further active agent is an acetylcholine esterase inhibitor.

274. The composition according to item 273, wherein said acetylcholine esterase inhibitor is selected from the group consisting of delta-9-tetrahydrocannabinol, carbamates, physostigmine, neostigmine, pyridostigmine, ambenonium, demecarium, rivastigmine, phenanthrene derivatives, galantamine, piperidines, donepezil, tacrine, edrophonium, huperzine, ladostigil, ungeremine and lactucopicrin.

275. The composition according to item 273, wherein said acetylcholine esterase inhibitor is neostigmine or pyridostigmine.

276. The composition according to item 271, wherein said further active agent is sugammadex.

277. The composition according to item 271, wherein said further active agent is 3,4-diaminopyridine.

278. The compound according to any one of items 1 to 268 or composition according to any one of items 269 to 277 for use as a medicament.

279. The compound according to any one of items 1 to 268 or composition according to any one of items 269 to 277 for use in treating, ameliorating and/or preventing a neuromuscular disorder, and/or for use in reversing and/or ameliorating a neuromuscular blockade.

280. The compound for use according to item 279, wherein the neuromuscular disorder is one selected from the group consisting of myasthenia gravis, autoimmune myasthenia gravis, congenital myasthenic syndrome, seronegative myasthenia gravis, muscle specific kinase myasthenia gravis (MuSK-MG), Lambert-Eaton Syndrome, amyotrophic lateral sclerosis (ALS), spinal muscular atrophy (SMA), critical illness myopathy (CIM), Charcot-Marie Tooth disease, diabetic polyneuropathy, periodic paralysis, hypokalemic periodic paralysis, hyperkalemic periodic paralysis, myotubular myopathy, Duchenne muscular dystrophy, Guillain-Barre syndrome, poliomyelitis, post-polio syndrome, chronic fatigue syndrome, critical illness polyneuropathy, metabolic myopathy, Kennedy's disorder, multiple sclerosis and multifocal motor neuropathy.

281. The compound for use according to item 279, wherein the neuromuscular disorder is sarcopenia.

282. The compound for use according to item 279, wherein the neuromuscular blockade has been induced by a neuromuscular blocking agent.

283. The compound according to any one of items 1 to 268 or composition according to any one of items 269 to 277 for use in the treatment of an indication selected from the group consisting of myasthenia gravis, autoimmune myasthenia gravis, congenital myasthenic syndrome, seronegative myasthenia gravis, muscle specific kinase myasthenia gravis (MuSK-MG), Lambert-Eaton Syndrome, amyotrophic lateral sclerosis (ALS), spinal muscular atrophy (SMA), critical illness myopathy (CIM), Charcot-Marie Tooth disease, diabetic polyneuropathy, periodic paralysis, hypokalemic periodic paralysis, hyperkalemic periodic paralysis, myotubular myopathy, Duchenne muscular dystrophy, Guillain-Barre syndrome, poliomyelitis, post-polio syndrome, chronic fatigue syndrome, critical illness polyneuropathy, metabolic myopathy, Kennedy's disorder, multiple sclerosis and multifocal motor neuropathy.

284. The compound according to any one of items 1 to 268 or composition according to any one of items 269 to 277 for use in the symptomatic treatment of sarcopenia.

285. The compound according to any one of items 1 to 268 or composition according to any one of items 269 to 277 for use in reversing and/or ameliorating a neuromuscular blockade.

286. The compound according to any one of items 1 to 268 or composition according to any one of items 269 to 277 for use in the treatment of botulism poisoning, in the treatment of snake bites, in the treatment of nerve gas poisoning or prophylactically against nerve gas poisoning.

287. A method of treating, preventing and/or ameliorating a neuromuscular disorder, said method comprising administering a therapeutically effective amount of the compound according to any one of items 1 to 268 or the composition according to any one of items 269 to 277 to a person in need thereof.

288. A method of reversing and/or ameliorating a neuromuscular blockade, said method comprising administering a therapeutically effective amount of the compound according to any one of items 1 to 268 or the composition according to any one of items 269 to 277 to a person in need thereof.

289. Use of a compound according to any one of items 1 to 268 or the composition according to any one of items 269 to 277, for the manufacture of a medicament for the treatment, prevention and/or amelioration of a neuromuscular disorder, and/or for reversing and/or ameliorating of a neuromuscular blockade.

290. A method of treating botulism poisoning, snake bites or nerve gas poisoning or preventing nerve gas poisoning, said method comprising administering a therapeutically effective amount of the compound according to any one of items 1 to 268 or the composition according to any one of items 269 to 277 to a person in need thereof.

291. Use of a compound according to any one of items 1 to 2268 or the composition according to any one of items 269 to 277, for the manufacture of a medicament for the treatment of botulism poisoning, snake bites or nerve gas poisoning or prevention of nerve gas poisoning.

292. A method for recovery of neuromuscular transmission, said method comprising administering a therapeutically effective amount of the compound according to any one of items 1 to 268 or the composition according to any one of items 269 to

277 to a person in need thereof.

293. A method for recovering neuromuscular transmission, the method comprising administering the compound according to any one of items 1 to 268 or the composition according to any one of items 269 to 277 to an individual in need thereof.

### EXAMPLES

General Synthetic Strategies General methods for the synthesis of carboxylic acids and substitution on aromatic rings are featured in literature sources such as: March's Advanced Organic Chemistry: Reactions, Mechanisms, and Structure, 8th Edition, Michael B. Smith, Ed.; ISBN: 978-1-119-37179-3; John Wiley, 2020.

### Materials and Methods

Chemicals Compounds for testing were obtained from different suppliers including Enamine, Vitas, and CanAm Bioresearch. For synthesis of particular compounds please see below.

### NMR Spectra

¹H-NMR and ¹⁹F-NMR spectra were either recorded on a Bruker AM-300 spectrometer and were calibrated using residual nondeuterated solvent as internal reference. Spectra were processed using Spinworks version 4.0 (developed by Dr. Kirk Marat, Department of Chemistry, University of Manitoba).

Otherwise ¹H, ¹³C and ¹⁹F NMR analyses were conducted Jeol 400 using deuterated chloroform or deuterated dimethyl sulfoxide as solvent. The shift (6) of each signal was measured in parts per million (ppm) relative the residual solvent peak, and the multiplicity reported together with the associated coupling constant (J), where applicable.

### LC/MS System

Samples were analysed my direct inject on a Waters Acquity QDa Mass Detector with a Waters 2695 HPLC. Mass spectra were recorded in ESI scan mode (negative/positive).

### HPLC Method

The product was analysed by Waters 2695 HPLC consisting of a Waters 996 photodiode array detector, Kromasil Eternity C18, 5 μm, 4.6×150 mm column. Flow rate: 1 mL/minute, run time 20 minutes. Solvent A: methanol; solvent B: 0.1% formic acid in water. Gradient 0-100% Solvent B over 15 minutes with monitoring at 280 nm.

### UPLC-MS Method

UPLC-MS analysis was carried out on a Waters Acquity UPLC system consisting of an Acquity I-Class Sample Manager-FL, Acquity I-Class Binary Solvent Manager and an Acquity UPLC Column Manager. UV detection was afforded using an Acquity UPLC PDA detector (scanning from 210 to 400 nm), whilst mass detection was achieved using an Acquity QDa detector (mass scanning from 100-1250 Da; positive and negative modes simultaneously), and ELS detection was achieved using an Acquity UPLC ELS Detector.

Samples were prepared by dissolution (with or without sonication) into 1 mL of 50% (v/v) MeCN in water. The resulting solutions were then filtered through a 0.2 m syringe filter before submitting for analysis. All of the solvents, including formic acid and 36% ammonia solution, were purchased as the HPLC grade.

### Acidic 4 min Method

Eluent A: 0.1% v/v formic acid in 10 mM ammonium formate

Eluent B: 0.1% v/v formic acid in MeCN

Flow rate 0.8 mL/min; column oven 50° C.; sample manager 20° C.; injection volume 2 L. 1.5 minutes equilibration time between samples on a Waters Acquity UPLC BEH C₁₈ column (2.1×50 mm, 1.7 m).

Gradient as given in table below.

| Time (min) | Eluent A (%) | Eluent B (%) |
|---|---|---|
| 0.00 | 95 | 5 |
| 0.25 | 95 | 5 |
| 2.75 | 5 | 95 |
| 3.25 | 5 | 95 |
| 3.35 | 95 | 5 |
| 4.00 | 95 | 5 |

### Example 1: 6-Chloro-5-methyl-2,3-dihydro-1H-indene-4-carboxylic acid (A-1)

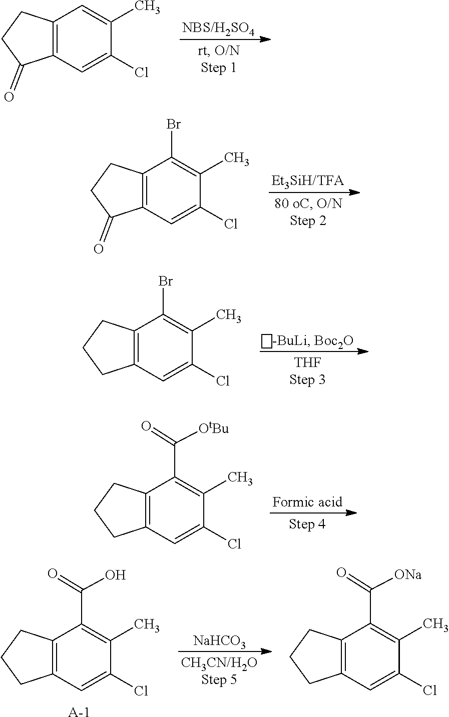

### Step 1: 4-Bromo-6-chloro-5-methyl-2,3-dihydro-1H-inden-1-one

To a solution of 6-chloro-5-methyl-2,3-dihydro-1H-inden-1-one (0.8 g, 4.44 mmol) in sulfuric acid (20 mL) at ambient temperature NBS (0.79 g, 4.44 mmol) was added. The reaction mixture was stirred at ambient temperature for 16 hours. The orange solution was poured into a mixture of crushed ice and water; a solid formed, which was collected by filtration after the ice had melted. The solid was washed with water (15 mL) and purified by column chromatography on silica gel eluting with hexane/EtOAc (0-20%) to provide the title compound (0.60 g, 52%) as a white solid.

¹H NMR (300 MHz, CDCl₃) δ 7.70 (s, 1H), 3.07-3.00 (m, 2H), 2.75-2.70 (m, 2H), 2.61 (s, 3H).

### Step 2: 4-Bromo-6-chloro-5-methyl-2,3-dihydro-1H-indene

A mixture of 4-bromo-6-chloro-5-methyl-2,3-dihydro-1H-inden-1-one (0.60 g, 2.31 mmol), triethylsilane (1.6 g, 13.87 mmol) and TFA (1.58 g, 13.87 mmol) was heated in a sealed tube at 80° C. for 16 h. The reaction mixture was cooled, evaporated and purified by column chromatography on silica gel eluting with EtOAc/hexane (0-10%) to obtain the title compound (0.4 g, 70%) as a colourless oil.

¹H NMR (300 MHz, CDCl₃) δ 7.16 (s, 1H), 3.01-2.87 (m, 4H), 2.47 (s, 3H), 2.13-2.02 (m, 2H).

### Step 3: Tert-butyl 6-chloro-5-methyl-2,3-dihydro-1H-indene-4-carboxylate

To a solution of 4-bromo-6-chloro-5-methyl-2,3-dihydro-1H-indene (0.32 g, 1.30 mmol) in THF (10 mL) at −78° C., n-BuLi (2.5 M in hexane) (0.57 mL, 1.43 mmol) was added. The reaction mixture was stirred at that temperature for 30 min. and di-tert-butyl dicarbonate (0.313 g, 1.43 mmol) in THF (10 mL) was added dropwise. The reaction mixture was stirred at −78° C. temperature for 1 h., slowly brought to ambient temperature and quenched with saturated ammonium chloride solution (5 mL). The reaction mixture was extracted with EtOAc (2×15 mL), washed with water (10 mL), brine (10 mL) before the combined extracts were dried (Na₂SO₄) filtered and concentrated in vacuo. The crude product was purified by column chromatography on silica gel eluting with hexane/EtOAc (0-10%) to afford the title compound (0.21 g, 60%) as a white solid.

¹H NMR (300 MHz, CDC₃) δ 7.31 (s, 1H), 3.01-2.88 (m, 4H), 2.43 (s, 3H), 2.19-2.06 (m, 2H), 1.64 (s, 9H).

### Step 4: 6-Chloro-5-methyl-2,3-dihydro-1H-indene-4-carboxylic acid

A solution of tert-butyl 6-chloro-5-methyl-2,3-dihydro-1H-indene-4-carboxylate (0.21 g, 0.787 mmol) in formic acid (5 mL) was heated at 50° C. for 50 min., cooled and the reaction mixture was concentrated in vacuo. The crude product was purified by column chromatography on silica gel eluting with hexane/EtOAc (0-40%) to provide the title compound (0.10 g, 60%) as a white solid.

¹H NMR (300 MHz, CD₃OD) δ 7.33 (s, 1H), 3.02-2.85 (m, 4H), 2.40 (s, 3H), 2.16-2.01 (m, 2H).

### Step 5: Sodium 6-chloro-5-methyl-2,3-dihydro-1H-indene-4-carboxylate

To a solution of 6-chloro-5-methyl-2,3-dihydro-1H-indene-4-carboxylic acid (0.175 g, 0.83 mmol) in acetonitrile (24 mL), a solution of NaHCO₃ (0.068 g, 0.814 mmol) in water (20 mL) was introduced. The reaction mixture was stirred at ambient temperature for 30 min., the acetonitrile was removed in vacuo before the water layer was washed with DCM (10 mL) to remove any unreacted acid. The aqueous layer was separated and evaporated to obtain the title compound (0.165 g, 85%) as a white solid.

¹H NMR (300 MHz, CD₃OD) δ 7.11 (s, 1H), 2.98-2.82 (m, 4H), 2.37 (s, 3H), 2.15-2.02 (m, 2H).

ES-MS: m/z 209.5 (M-1).

HPLC: Retention time 11.142 min., purity >98% at 280 nm.

### Example 2: 6-Bromo-5-methyl-2,3-dihydro-1H-indene-4-carboxylic acid (A-2)

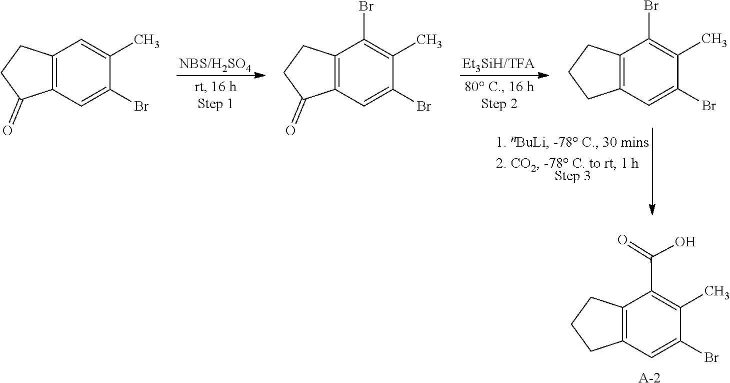

### Step 1: 4,6-Dibromo-5-methyl-1-indanone

6-Bromo-5-methyl-1-indanone (0.50 g, 2.22 mmol) in H₂SO₄ (5.0 mL) was treated with NBS (0.40 g, 2.25 mmol) at ambient temperature and the mixture was stirred for 16 h.

The reaction mixture was poured into cold water (50 mL) and extracted with EtOAc (2×50 mL). The combined extracts were washed with water (50 mL), dried (Na₂SO₄) and concentrated under reduced pressure. The resultant residue was purified by chromatography on silica gel eluting with hexane/EtOAc (5-10%) to provide the title compound (0.31 g, 46%).

¹H NMR (300 MHz, CDCl₃) δ 7.90 (s, 1H), 3.06-3.00 (m, 2H), 2.77-2.70 (m, 2H), 2.68 (s, 3H).

ES-MS: 304 [M+H]

### Step 2: 4,6-Dibromo-5-methylindane

To 4,6-dibromo-5-methyl-1-indanone (0.29 g, 0.95 mmol) was added triethylsilane (0.92 mL, 5.76 mmol) and TFA (0.44 mL, 5.76 mmol) and the reaction mixture was heated at 75° C. over a period of 20 h. The solvent was removed under reduced pressure and the residue was purified by chromatography on silica gel eluting with hexane/EtOAc (0-5%) to provide the title compound (0.22 g, 79%).

¹H NMR (300 MHz, CDCl₃) δ 7.35 (s, 1H), 2.98 (t, 2H), 2.90 (t, 2H), 2.53 (s, 3H), 2.08 (m, 2H).

ES-MS: 290 [M+H]

### Step 3: 6-Bromo-5-methyl-2,3-dihydro-1H-indene-4-carboxylic acid

To a solution of 4,6-dibromo-5-methylindane (0.12 g, 0.41 mmol) in THF (2.0 mL) n-BuLi (0.28 mL, 0.46 mmol) was added at −78° C. The reaction mixture was stirred at ambient temperature for 30 min. The mixture was cooled again to −78° C. and CO₂ was bubbled through it over a period of 30 min. The reaction mixture was warmed to 0° C. and CO₂ was again bubbled over a period of 30 min. and quenched with water (10 mL) prior to extraction with EtOAc (2×15 mL). The aqueous layer was acidified to pH ~2 utilising 1.0 N HCl and extracted with EtOAc (2×15 mL). The combined extracts were dried (Na₂SO₄) and concentrated under reduced pressure. The residue was purified by semi-preparative HPLC eluting 10-100% acetonitrile/0.1% formic acid in water to afford the title compound (6.5 mg, 6.1%).

¹H NMR (300 MHz, CDCl₃) δ 7.60 (s, 1H), 3.11 (t, 2H), 2.97 (t, 2H), 2.60 (s, 3H), 2.68 (m, 2H).

ES-MS: 253 and 255 [M−H]

HPLC Retention Time: 11.4 min.; Purity: >98% @280 nm.

### Example 3: 3-chloro-5,6,7,8-tetrahydronaphthalene-1-carboxylic acid (A-3)

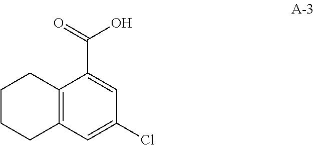 [SMILES: O=C(O)c1cc(Cl)cc2c1CCCC2]

3-Chloro-5,6,7,8-tetrahydronaphthalene-1-carboxylic acid is commercially available from e.g. Enamine (EN300-8778091).

### Example 4: 5,6-dimethyl-2,3-dihydro-1H-indene-4-carboxylic acid (A-4)

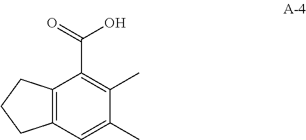

5,6-Dimethyl-2,3-dihydro-1H-indene-4-carboxylic acid is commercially available from e.g. Enamine (EN300-24516099).

### Example 5: 2,3-dimethyl-5,6,7,8-tetrahydronaphthalene-1-carboxylic acid (A-5)

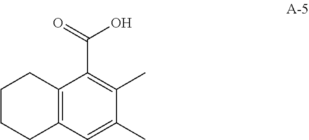

2,3-Dimethyl-5,6,7,8-tetrahydronaphthalene-1-carboxylic acid is commercially available from e.g. Enamine (EN300-9425991).

### Example 6: 6-Chloro-5-methoxy-2,3-dihydro-1H-indene-4-carboxylic acid (A-6)

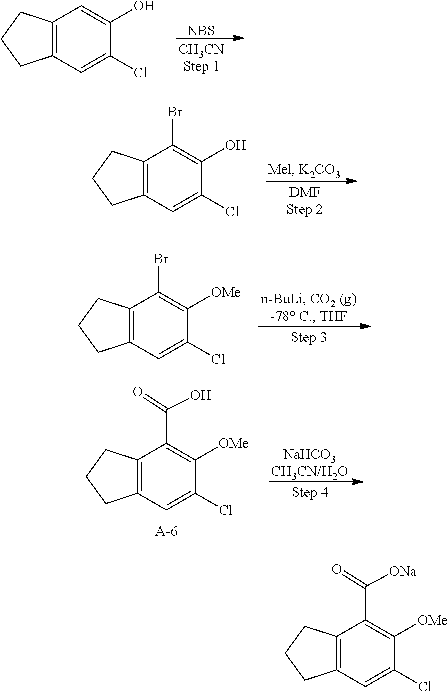

### Step 1: 4-Bromo-6-chloro-2,3-dihydro-1H-inden-5-ol

To a solution of 6-chloro-2,3-dihydro-1H-inden-5-ol (3.7 g, 22.0 mmol) in acetonitrile (80 mL) at ambient temperature NBS (3.12 g, 22.0 mmol) was added. The reaction mixture was stirred at ambient temperature for 16 h, extracted with EtOAc (2×60 mL) and the combined extracts were washed with water (2×30 mL) and brine (20 mL) before being dried (Na₂SO₄) and concentrated in vacuo. The residue was purified by column chromatography on silica gel eluting with hexane/EtOAc (0-10%) to provide the title compound (4.45 g, 81%) as a colourless oil.

¹H NMR (300 MHz, CDCl₃) δ 7.12 (s, 1H), 5.69 (s, 1H), 3.00-2.84 (m, 4H), 2.8-2.01 (m, 2H).

### Step 2: 4-Bromo-6-chloro-5-methoxy-2,3-dihydro-1H-indene

To a solution of 4-bromo-6-chloro-2,3-dihydro-1H-inden-5-ol (1.5 g, 6.06 mmol) in DMF (15 mL) at ambient temperature, K₂CO₃ (1.26 g, 9.09 mmol) was introduced, followed by iodomethane (1.03 g, 7.27 mmol). The reaction mixture was stirred at ambient temperature for 16 h, concentrated in vacuo, extracted with EtOAc (2×30 mL). The combined extracts were washed with water (4×10 mL), brine (2×10 mL) dried (Na₂SO₄) and concentrated to obtain the crude title compound (1.32 g, 83% crude yield) as a colourless oil, which was used in the next step without purification.

¹H NMR (300 MHz, CD₃OD) δ 7.23 (s, 1H), 3.83 (s, 3H), 3.04-2.88 (m, 4H), 2.21-2.08 (m, 2H).

### Step 3: 6-Chloro-5-methoxy-2,3-dihydro-1H-indene-4-carboxylic acid

To a solution of 4-bromo-6-chloro-5-methoxy-2,3-dihydro-1H-indene (0.8 g, 3.05 mmol) in THF (15 mL) at −78° C. and a solution of n-BuLi (2.5 M in hexane) (1.34 mL, 3.35 mmol) was introduced. The reaction mixture was stirred at that temperature for 30 min. and CO₂ (g) was bubbled through it for 20 min. The reaction mixture was stirred for 1 h at −78° C., gradually brought to 0° C. and quenched with 1N NaOH to pH ~11. Following washing with EtOAc (10 mL) the aqueous layer was separated and acidified with 1N HCl to pH ~1 prior to extraction with EtOAc (2×10 mL). The combined extracts were washed with water (5 mL), brine (5 mL), dried (Na₂SO₄) and concentrated to afford the title compound (0.40 g, 57%) as a white solid.

¹H NMR (300 MHz, CD₃OD) δ 7.37 (s, 1H), 3.88 (s, 3H), 3.03-2.86 (m, 4H), 2.18-2.07 (m, 2H).

### Step 4: Sodium 6-chloro-5-methoxy-2,3-dihydro-1H-indene-4-carboxylate

To a solution of 6-chloro-5-methoxy-2,3-dihydro-1H-indene-4-carboxylic acid (0.15 g, 0.662 mmol) in acetonitrile (15 mL), a solution of NaHCO₃ (0.055 g, 0.65 mmol) in water (10 mL) was added. The reaction mixture was stirred at ambient temperature for 30 min. Acetonitrile was removed in vacuo and the aqueous layer was washed with DCM (10 mL). The solution was concentrated in vacuo to provide the title compound (0.160 g, 85%) as a white solid.

¹H NMR (300 MHz, CD₃OD) δ 7.14 (s, 1H), 3.90 (s, 3H), 2.96-2.81 (m, 4H), 2.17-2.01 (m, 2H).

ES-MS: m/z 225.4 (M-1).

HPLC: Retention time 10.281 min., purity >98% at 280 nm.

### Example 7: 6-Chloro-5-(fluoromethoxy)-2,3-dihydro-1H-indene-4-carboxylic acid (A-7)

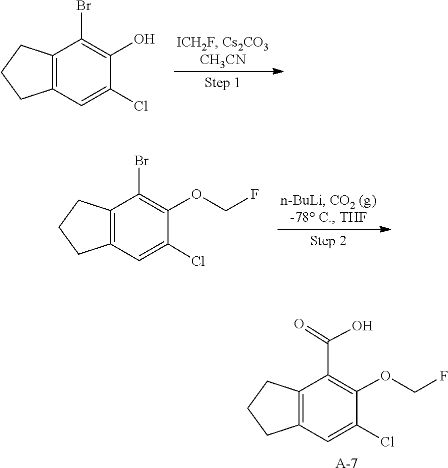

### Step 1: 4-Bromo-6-chloro-5-(fluoromethoxy)-2,3-dihydro-1H-indene

To a solution of 4-bromo-6-chloro-2,3-dihydro-1H-inden-5-ol (480 mg, 1.94 mmol) in acetonitrile (12 mL) at 0° C., Cs₂CO₃ (948 mg, 2.90 mmol) was introduced, followed by fluoroiodomethane (372 mg, 2.33 mmol). The reaction mixture was stirred at ambient temperature for 18 h. and concentrated in vacuo. The residue was purified by column chromatography on silica gel eluting with hexane/EtOAc (0-10%) to provide the title compound (430 mg, 79%) as a white solid.

¹H NMR (300 MHz, CDCl₃) δ 7.21 (s, 1H), 5.78 (s, 1H), 5.59 (s, 1H), 3.04-2.83 (m, 4H), 2.20-2.04 (m, 2H).

¹⁹F NMR (300 MHz, CDCl₃) δ− 149.09 ppm.

### Step 2: 6-chloro-5-(fluoromethoxy)-2,3-dihydro-1H-indene-4-carboxylic acid

n-BuLi (2.5 M in hexane) (0.283 mL, 0.708 mmol) was added to 5-(fluoromethoxy)-2,3-dihydro-1H-indene (180 mg, 0.644 mmol) in THF (15 mL) at −78° C. The reaction mixture was stirred at that temperature for 30 min. and CO₂ (g) was bubbled through it for 20 min. The mixture was stirred for 1 h at −78° C., slowly brought to 0° C. and quenched with 1 N NaOH to pH ~11 before being washed with EtOAc (10 mL). The aqueous layer was acidified with 1 N HCl to pH ~1, extracted with EtOAc (2×10 mL) and the combined extracts were washed with water (5 mL) then brine (5 mL). The organic layer was dried (Na₂SO₄) and evaporated to afford the title compound (40 mg, 24%) as a white solid.

¹H NMR (300 MHz, CD₃OD) δ 7.44 (s, 1H), 5.71 (s, 1H), 5.52 (s, 1H), 3.02 (t, 2H), 2.95 (t, 2H), 2.20-2.07 (m, 2H). ¹⁹F NMR (300 MHz, CD₃OD) δ− 150.45 ppm.

ES-MS: m/z 243.5 (M-1).

HPLC: Retention time 10.088 min., purity 94% at 280 nm.

### Example 8: 3-Chloro-2-methoxy-5,6,7,8-tetrahydronaphthalene-1-carboxylic acid (A-8)

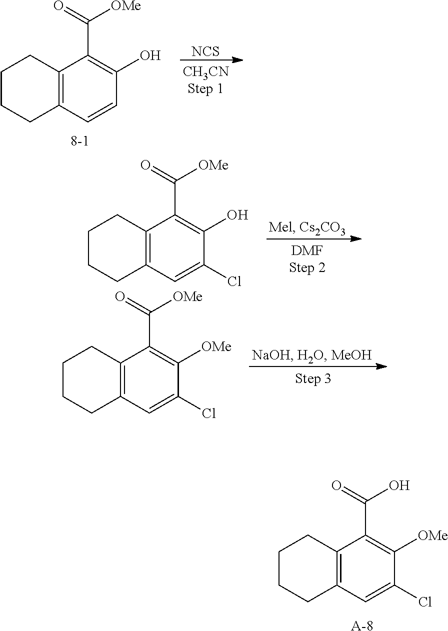

3-Chloro-2-methoxy-5,6,7,8-tetrahydronaphthalene-1-carboxylic acid is commercially available from e.g. Aurora Fine Chemicals (115.237.892) or can be prepared using the route disclosed above.

¹H NMR (300 MHz, CDCl₃) δ 6.97 (s, 1H), 3.88 (s, 3H), 2.78 (m, 4H), 1.78 (m, 4H)) ppm.

ES-MS: m/z 239.5 (M-1).

HPLC: Retention time 10.81 min, purity 94% at 280 nm.

### Example 9: 3-Chloro-2-methyl-5,6,7,8-tetrahydronaphthalene-1-carboxylic acid (A-9)

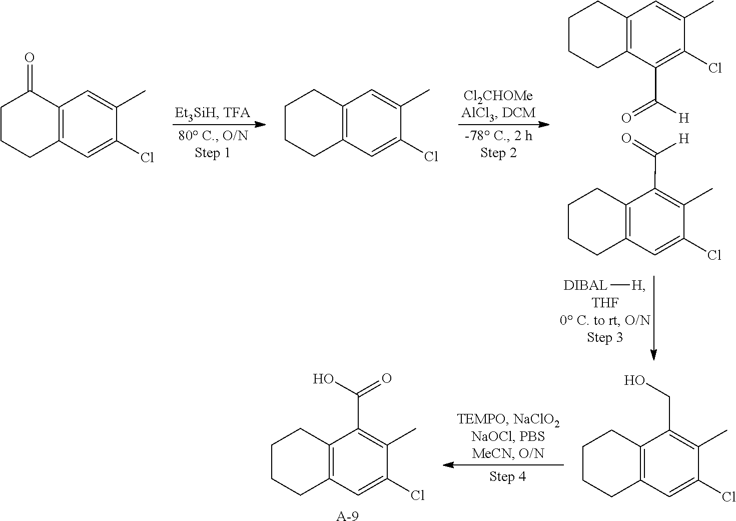

### Step 1: 6-Chloro-7-methyl-1,2,3,4-tetrahydronaphthalene

A mixture of 6-chloro-7-methyl-3,4-dihydro-2H-naphthalen-1-one (1.20 g, 6.18 mmol), triethylsilane (5.93 mL, 37.10 mmol) and TFA (2.84 mL, 37.1 mmol) in a sealed tube was heated at 80° C. for 16 h. The reaction was quenched with water (25 mL), extracted with EtOAc (100 mL) and the extract evaporated. The resultant residue was treated with saturated NaHCO₃ and extracted with DCM (2×20 mL), dried (Na₂SO₄) and evaporated. The resultant residue was purified by chromatography on silica gel eluting with hexane/EtOAc (0-20%) to provide the title compound (0.62 g: 55%).

¹H NMR (300 MHz, CDCl₃) δ 7.26 (s, 1H), 7.12 (s, 1H), 2.96-2.86 (br, 4H), 2.53 (s, 3H), 2.05-1.94 (m, 4H).

### Step 2: 2-Chloro-3-methyl-5,6,7,8-tetrahydronaphthalene-1-carbaldehyde (3) and 3-chloro-2-methyl-5,6,7,8-tetrahydronaphthalene-1-carbaldehyde (4)

To a suspension of 6-chloro-7-methyl-1,2,3,4-tetrahydronaphthalene (0.60 g, 3.33 mmol) and AlCl₃ (0.67 g, 4.99 mmol) in dry DCM (5 mL) a solution of Cl₂CHOMe (0.45 mL, 4.99 mmol) in dry DCM (2 mL) was added at −78° C. under an argon atmosphere.

The reaction mixture was stirred at the same temperature for 30 min. and then at ambient temperature for 2 h. The reaction mixture was quenched with saturated aqueous NaHCO₃ (30 mL) and extracted with DCM (2×30 mL). The DCM layer was separated, washed with brine (30 mL), dried (Na₂SO₄) and concentrated. The resultant residue was purified by chromatography on silica gel eluting with hexane/EtOAc (0-20%) to provide the to provide 2-chloro-3-methyl-5,6,7,8-tetrahydronaphthalene-1-carbaldehyde (3) (234 mg: 33%) and 3-chloro-2-methyl-5,6,7,8-tetrahydronaphthalene-1-carbaldehyde (4) 133 mg: 19%).

¹H NMR of compound 3 (300 MHz, CDC₃) δ 10.76 (s, 1H), 7.26 (s, 1H), 3.20-3.07 (m, 2H), 2.90-2.78 (m, 2H), 2.47 (s, 3H), 1.92-1.80 (m, 4H).

¹H NMR of compound 4 (300 MHz, CDC₃) δ 10.55 (s, 1H), 7.26 (s, 1H), 3.02-2.92 (m, 2H), 2.78-2.66 (m, 2H), 2.52 (s, 3H), 1.84-1.68 (m, 4H).

### Step 3: (3-Chloro-2-methyl-5,6,7,8-tetrahydronaphth-1-yl)methanol (5)

DIBAL-H (1N in toluene, 0.93 mL, 0.93 mmol) was added dropwise over 10 min. to a solution of 3-chloro-2-methyl-5,6,7,8-tetrahydronaphthalene-1-carbaldehyde (130 mg, 0.62 mmol) in THF (5 mL) at 0° C. and the solution was stirred for 15 min. at 0° C. The reaction mixture was allowed to warm to ambient temperature and stirred for 2 h. The reaction was quenched with saturated aqueous sodium potassium tartrate solution (30 mL) and extracted with EtOAc (2×20 mL). The combined extracts were washed with brine (20 mL), dried (Na₂SO₄) and evaporated in vacuo to afford the title compound (130 mg, 99%).

¹H NMR (300 MHz, CDC₃) δ 7.26 (s, 1H), 4.87 (s, 2H), 3.00 (t, 2H), 2.90 (t, 2H), 2.61 (s, 3H), 2.07-1.85 (m, 4H).

### Step 4: 3-Chloro-2-methyl-5,6,7,8-tetrahydronaphthalene-1-carboxylic acid (6)

To a stirred solution of (3-Chloro-2-methyl-5,6,7,8-tetrahydronaphth-1-yl)methanol (130 mg, 0.62 mmol), sodium chlorite (139.91 mg, 1.55 mmol) in acetonitrile (5 mL) and 1N sodium phosphate buffer (pH-6, 5 mL), sodium hypochlorite (0.14 mL, 4-4.99M solution) was added followed by TEMPO (4.83 mg, 0.031 mmol). The reaction mixture was stirred at ambient temperature for 2 h, then further sodium hypochlorite (0.14 mL, 4-4.99M solution) and TEMPO (4.83 mg, 0.031 mmol) were added. The addition of sodium hypochlorite and TEMPO was repeated three more times at 8 h intervals. The reaction mixture was cooled to 0° C. and 1 N NaOH solution was added to adjust pH to ~13 and washed with DCM (40 mL). The aqueous layer was separated, cooled to 0° C., acidified with 1 N HCl to pH ~1, extracted with EtOAc (2×30 mL), washed with water (2×10 mL), brine (10 mL), dried (Na₂SO₄) and evaporated in vacuo. The crude product was purified by semi-prep HPLC utilising 30 to 100% acetonitrile/0.1% formic acid in water to provide the title compound as a white solid (53.2 mg, 38%).

¹H NMR (300 MHz, CD₃OD) δ 7.01 (s, 1H), 2.68-2.53 (m, 4H), 2.18 (s, 3H), 1.74-1.59 (m, 4H).

ES-MS: 223.4 [M−1].

HPLC: Retention time 7.644 min. and purity >98% at 280 nm.

### Example 10: 6-Chloro-5-(difluoromethoxy)-2,3-dihydro-1H-indene-4-carboxylic acid (A-10)

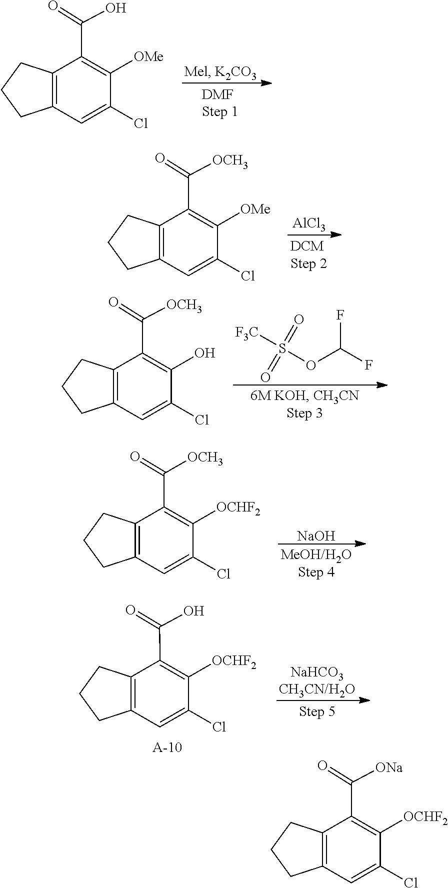

### Step 1: Methyl 6-chloro-5-methoxy-2,3-dihydro-1H-indene-4-carboxylate

To a solution of 6-chloro-5-methoxy-2,3-dihydro-1H-indene-4-carboxylic acid (840 mg, 3.71 mmol) in DMF (15 mL) at ambient temperature, K₂CO₃ (768 mg, 5.56 mmol) was introduced, followed by iodomethane (632 mg, 4.45 mmol). The reaction mixture was stirred at ambient temperature for 18 h. The reaction was concentrated in vacuo, water (20 mL) was added to the residue and the mixture extracted with EtOAc (2×30 mL).

The combined extracts were washed with water (4×10 mL) and brine (2×10 mL). The organic layer was dried (Na₂SO₄) and evaporated in vacuo to provide the title compound (870 mg, 97%) which was used directly in the next step without purification.

¹H NMR (300 MHz, CDC₃) δ 7.30 (s, 1H), 3.91 (s, 3H), 3.88 (s, 3H), 2.94 (t, 2H), 2.85 (t, 2H), 2.13-2.02 (m, 2H).

### Step 2: Methyl 6-chloro-5-hydroxy-2,3-dihydro-1H-indene-4-carboxylate

AlCl₃ (1.930 g, 14.44 mmol) was added to a solution of methyl 6-chloro-5-methoxy-2,3-dihydro-1H-indene-4-carboxylate (870 mg, 3.61 mmol) in DCM (20 mL) at 0° C. The reaction mixture was stirred at ambient temperature for 18 h, quenched with 2N HCl, extracted with EtOAc (3×50 mL) then the combined extracts were washed with water (2×20 mL) and brine (15 mL). The organic layer was dried (Na₂SO₄), concentrated and the residue purified by column chromatography on silica gel eluting with hexane/EtOAc (0-30%) to provide the title compound (750 mg, 91%) as an off-white solid.

¹H NMR (300 MHz, CDC₃) δ 11.60 (s, 1H), 7.38 (s, 1H), 3.96 (s, 3H), 3.15 (t, 2H), 3.82 (t, 2H), 2.11-1.99 (m, 2H).

### Step 3: Methyl 6-chloro-5-(difluoromethoxy)-2,3-dihydro-1H-indene-4-carboxylate

To a solution of methyl 6-chloro-5-hydroxy-2,3-dihydro-1H-indene-4-carboxylate (660 mg, 2.91 mmol) in acetonitrile (16 mL) at 0° C., 6N KOH solution (16 mL) was added, followed by dropwise addition of difluoromethyl trifluoromethanesulfonate (1.2 g, 5.82 mmol). The reaction mixture was stirred at 0° C. for 10 min. The reaction mixture was quenched with water (20 mL), extracted with DCM (3×50 mL) and the combined extracts were washed with water (20 mL) and brine (15 mL). The organic layer was dried (Na₂SO₄), concentrated to a residue which was purified by column chromatography on silica gel eluting with hexane/EtOAc (0-10%) to provide the title compound (410 mg, 50%) as a colourless oil.

¹H NMR (300 MHz, CDC₃) δ 7.38 (s, 1H), 6.52 (t, 1H), 3.90 (s, 3H), 2.99 (t, 2H), 2.90 (t, 2H), 2.17-2.04 (m, 2H). ¹⁹F NMR (300 MHz, CDC₃) δ− 81.49 ppm.

### Step 4: 6-Chloro-5-(difluoromethoxy)-2,3-dihydro-1H-indene-4-carboxylic acid

NaOH (38 mg, 0.94 mmol) was added to a solution of methyl 6-chloro-5-(difluoromethoxy)-2,3-dihydro-1H-indene-4-carboxylate (130 mg, 0.47 mmol) in MeOH (1 mL) and water (3 mL). The reaction mixture was heated under microwave conditions at 130° C. for 50 min. The solvents were removed by evaporation and the crude product was dissolved in water (5 mL) and washed with DCM (10 mL). The aqueous layer was treated with 1 N HCl to pH ~2, extracted with EtOAc (2×20 mL), then the combined extracts were washed with water (10 mL) and brine (10 mL). The organic layer was dried (Na₂SO₄), filtered and concentrated in vacuo to afford the title compound (85 mg, 68%) as a white solid.

¹H NMR (300 MHz, CDCl₃) δ 9.00-7.78 (br s, 1H), 7.44 (s, 1H), 6.57 (t, 1H), 3.13 (t, 2H), 2.93 (t, 2H), 2.20-2.06 (m, 2H). ¹⁹F NMR (300 MHz, CDCl₃) δ −81.88 ppm.

### Step 5: Sodium 6-chloro-5-(difluoromethoxy)-2,3-dihydro-1H-indene-4-carboxylate

To a solution of 6-chloro-5-(difluoromethoxy)-2,3-dihydro-1H-indene-4-carboxylic acid (150 mg, 0.57 mmol) in acetonitrile (6 mL), a solution of NaHCO₃ (45.5 mg, 0.542 mmol) in water (6 mL) was introduced. The reaction mixture was stirred at ambient temperature for 30 min. and the acetonitrile was removed under reduced pressure. The aqueous layer was washed with DCM (10 mL) and concentrated in vacuo to provide the title compound (150 mg, 92%) as a white solid.

¹H NMR (300 MHz, CD₃OD) δ 7.21 (s, 1H), 6.77 (t, 1H), 2.98-2.81 (m, 4H), 2.18-2.03 (m, 2H). ¹⁹F NMR (300 MHz, CD₃OD) δ− 82.08 ppm.

ES-MS: m/z 261.3 (M-1).

HPLC: Retention time 7.243 min., purity >98% at 280 nm.

### Example 11: 6-Chloro-5-(trifluoromethoxy)-2,3-dihydro-1H-indene-4-carboxylic acid (A-11)

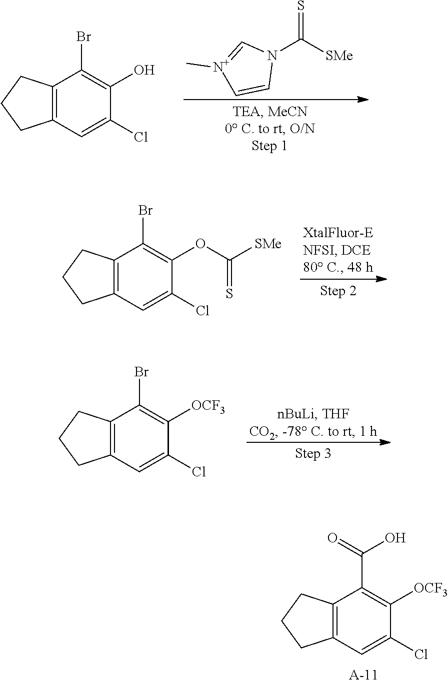

### Step 1: O-Methylthio 4-bromo-6-chloro-5-indancarbothioate

To a solution of 4-bromo-6-chloro-5-indanol (0.40 g, 1.63 mmol) and triethylamine (0.25 mL, 1.79 mmol) in acetonitrile (7 mL) at 0° C., 3-methyl-1-(methylthiocarbonothioyl)-1H-imidazol-3-ium (0.28 g, 1.63 mmol) was added. The reaction mixture was stirred at ambient temperature for 16 h, concentrated in vacuo, treated with EtOAc (50 mL), then the solution was washed with a saturated solution of NaHCO₃ (20 mL) and water (2×30 mL). The organic layer was dried (Na₂SO₄) and concentrated and the resultant residue was purified by chromatography on silica gel eluting with hexane/EtOAc (0-20%) to provide the title compound (0.40 g, 73%).

¹H NMR (300 MHz, CDCl₃) δ 7.29 (s, 1H), 3.14-2.90 (m, 4H), 2.78 (s, 3H), 2.27-2.09 (m, 2H).

### Step 2: 4-Bromo-6-chloro-5-trifluoromethoxyindane

A mixture of O-methylthio 4-bromo-6-chloro-5-indancarbothioate (0.4 g, 1.19 mmol), XtalFluor-E (0.82 g, 3.57 mmol), and N-fluorobenzenesulfonimide (1.13 g, 3.57 mmol) in 1,2-dichloroethane (5 mL) was heated at 80° C. for 48 h. The reaction was quenched with a saturated solution of NaHCO₃ (10 mL), filtered through a pad of Celite® and washed with EtOAc (2×30 mL). The combined extracts were washed with water (2×20 mL), dried (Na₂SO₄) and concentrated. the resultant residue was purified by chromatography on silica gel eluting with hexane/EtOAc (0-10%) to provide the title compound (60 mg, 16%).

¹H NMR (300 MHz, CDCl₃) δ 7.32 (s, 1H), 3.12-2.92 (m, 4H), 2.21 (m, 2H). ¹⁹F NMR (300 MHz, CDCl₃) δ− 55.42 ppm.

### Step 3: 6-Chloro-5-(trifluoromethoxy)-2,3-dihydro-1H-indene-4-carboxylic acid

To a solution of 4-bromo-6-chloro-5-trifluoromethoxyindane (60 mg, 0.19 mmol) in THF (4 mL) at −78° C., n-BuLi (2.5 M in hexane) (0.084 mL, 0.21 mmol) was added. The reaction mixture was stirred at that temperature for 1h. CO₂ was bubbled through the mixture for 10 min. and it was stirred for 1 h at −78° C. The reaction temperature was adjusted to 0° C. and the mixture treated with 1N NaOH to pH ~11. The reaction mixture was washed with DCM (2×10 mL). The water layer was acidified with 1N HCl to pH ~1 and extracted with EtOAc (20 mL) and the solution dried (Na₂SO₄) filtered and concentrated in vacuo. The crude product was purified by semi-prep HPLC utilising 30 to 100% acetonitrile/0.1% formic acid in water to provide 6-chloro-5-(trifluoromethoxy)-2,3-dihydro-1H-indene-4-carboxylic acid (2.6 mg, 4.9%).

¹H NMR (300 MHz, CDCl₃) δ 7.34 (s, 1H), 3.02 (t, 2H), 2.94 (t, 2H), 2.24-1.98 (m, 2H). ¹⁹F NMR (300 MHz, CDCl₃) δ− 58.15 ppm.

ES-MS: 279.49 [M−1].

HPLC: Retention time 7.594 min., purity >98% at 280 nm.

### Example 12: 5-Chloro-6-methyl-2,3-dihydro-1-benzofuran-7-carboxylic acid (A-12)

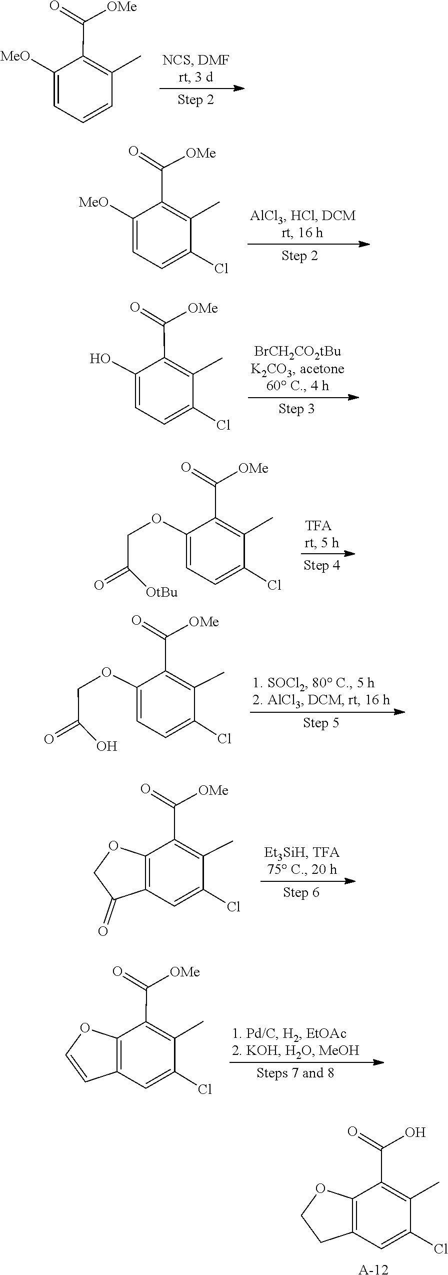

### Step 1: Methyl 6-chloro-3-methoxy-2-toluate

To a solution of methyl 3-methoxy-2-toluate (1.00 g, 5.55 mmol) in DMF (12 mL) NCS (0.89 g, 6.67 mmol) was introduced at ambient temperature and the mixture stirred for 3 days. The reaction was quenched with water (25 mL) and extracted with EtOAc (2×25 mL). The combined organic extracts were washed with 1.0 N HCl (2×25 mL), dried (Na₂SO₄) and concentrated under reduced pressure to give the title compound (1.15 g, 96%).

¹H NMR (300 MHz, CDCl₃) δ 7.32 (d, 1H), 6.72 (d, 1H), 3.92 (s, 3H), 3.80 (s, 3H), 2.28 (s, 3H).

ES-MS: 215 [M+H]

### Step 2: Methyl 6-chloro-3-hydroxy-2-toluate

To a solution of methyl 6-chloro-3-methoxy-2-toluate (1.15 g, 5.35 mmol) in DCM (20 mL) AlCl₃ (2.86 g, 21.4 mmol) was added at 0° C. and the mixture stirred at ambient temperature for 20 h. The reaction mixture was cooled to 0° C. and quenched with 1.0 N HCl (50 mL). The layers were separated and the aqueous layer was extracted with DCM (2×30 mL). The combined organic layers were dried (Na₂SO₄) and concentrated under reduced pressure to obtain the crude title compound (1.03 g, 96%) ¹H NMR (300 MHz, CDCl₃) δ 10.9 (s, 1H), 7.40 (d, 1H), 6.81 (d, 1H), 3.99 (s, 3H), 2.60 (s, 3H).

ES-MS: 199 [M−H]

### Step 3: tert-Butyl (4-chloro-2-methoxycarbonyltolyloxy)acetate

K₂CO₃ (1.03 g, 7.45 mmol) was added to a solution of methyl 6-chloro-3-hydroxy-2-toluate (1.00 g, 4.98 mmol) and tert-butyl bromoacetate (1.27 g, 6.51 mmol) in acetone (15 mL). The reaction mixture was heated at 60° C. for 4 h, then solvent was removed under reduced pressure and the residue diluted with EtOAc (30 mL) and water (30 mL).

The organic layer was dried (Na₂SO₄) and concentrated under reduced pressure. The residue was purified by chromatography on silica gel eluting with hexane/EtOAc (5-10%) to provide the title compound (1.38 g, 88%).

¹H NMR (300 MHz, CDCl₃) δ 7.30 (d, 1H), 6.60 (d, 1H), 4.49 (s, 2H), 3.93 (s, 3H), 2.30 (s, 3H), 1.47 (s, 9H).

ES-MS: 315 [M+H]

### Step 4: (4-Chloro-2-methoxycarbonyltolyloxy)acetic acid

To a solution of tert-butyl (4-chloro-2-methoxycarbonyltolyloxy)acetate (1.38 g, 4.39 mmol) in DCM (12 mL), TFA (5.0 mL) was added and the mixture was stirred at ambient temperature for 5 h. The solvent was removed under reduced pressure and the resultant was triturated with hexane (2×5.0 mL) to provide the title compound (1.00 g, 88%).

¹H NMR (300 MHz, CDCl₃) δ 7.39 (d, 1H), 6.73 (d, 1H), 4.70 (s, 2H), 3.97 (s, 3H), 2.35 (s, 3H).

ES-MS: 257 [M−H]

### Step 5: Methyl 5-chloro-6-methyl-3-oxo-2H-1-benzofuran-7-carboxylate

SOCl₂ (14 mL) was added to a solution of (4-chloro-2-methoxycarbonyltolyloxy)acetic acid (0.80 g, 3.09 mmol) in DCM (12 mL) and the mixture was heated at 40° C. for 5 h. After cooling the solvent was removed under reduced pressure and the residue was dissolved in DCM (4.0 mL) and added slowly to at 0° C. to a suspension of AlCl₃ (0.82 g, 6.15 mmol) in DCM (12 mL). The mixture was stirred at ambient temperature for 16 h, poured onto ice water (30 mL) and diluted with DCM (20 mL) before the layers were allowed to separate. The organic layer was washed with 1.0 N NaOH (40 mL), dried (Na₂SO₄) and concentrated under reduced pressure. The residue was triturated with hexane (2×5.0 mL) to obtain the title ketone (0.43 g, 58%).

¹H NMR (300 MHz, CDC₃) δ 7.74 (s, 1H), 4.70 (s, 2H), 3.99 (s, 3H), 2.52 (s, 3H).

ES-MS: 241 [M+H]

### Step 6: Methyl 5-chloro-6-methyl-1-benzofuran-7-carboxylate

Methyl 5-chloro-6-methyl-3-oxo-2H-1-benzofuran-7-carboxylate (200 mg, 0.83 mmol) was mixed with triethylsilane (0.8 mL, 4.99 mmol) and TFA (0.38 mL, 4.99 mmol) and the reaction was heated at 75° C. for 20 h. The solvent was removed under reduced pressure and the residue was purified by chromatography on silica gel eluting with hexane/EtOAc (0-10%) to provide the title compound (95 mg, 51%).

¹H NMR (300 MHz, CDC₃) δ 7.77 (s, 1H), 7.71 (d, 1H), 6.78 (d, 1H), 4.09 (s, 3H), 2.63 (s, 3H).

ES-MS: 225 [M+H]

### Step 7: Methyl 5-chloro-6-methyl-2,3-dihydro-1-benzofuran-7-carboxylate

To a solution of methyl 5-chloro-6-methyl-1-benzofuran-7-carboxylate (95 mg, 0.42 mmol) in EtOAc (2.0 mL), 10% Pd/C (120 mg) was added and the reaction mixture was subjected to hydrogenation conditions for 24 h. The reaction mixture was filtered through a pad of Celite®, the bed was washed with EtOAc (2×5 mL) and the filtrate evaporated under reduced pressure to provide the title compound (50 mg, 52%).

¹H NMR (300 MHz, CDC₃) δ 7.28 (s, 1H), 4.68 (t, 2H), 3.97 (s, 3H), 3.23 (t, 2H), 2.41 (s, 3H).

ES-MS: 227 [M+H]

### Step 8: 5-Chloro-6-methyl-2,3-dihydro-1-benzofuran-7-carboxylic acid

35% KOH solution (1.0 mL) was added to a solution of methyl 5-chloro-6-methyl-2,3-dihydro-1-benzofuran-7-carboxylate (50 mg, 0.22 mmol) in methanol (3 mL) and the reaction mixture was heated at 80° C. for 4 h. The solvent was removed under reduced pressure and the residue was diluted with water (15 mL) before extraction with EtOAc (2×15 mL). The aqueous layer was acidified to pH ~2 using 1.0 N HCl and extracted with EtOAc (2×15 mL). The combined organic layers were dried (Na₂SO₄) and concentrated under reduced pressure. The resultant residue was purified using semi; preparative HPLC utilising 10-100% acetonitrile/0.1% formic acid in water to afford the title compound (21 mg, 45%).

¹H NMR (300 MHz, CDCl₃) δ 7.42 (s, 1H), 4.82 (t, 2H), 3.32 (t, 2H), 2.67 (s, 3H).

ES-MS: 213 [M+H]

HPLC Retention Time: 11.7 min.; Purity: >98% @280 nm

### Example 13: 6-Chloro-5-ethyl-2,3-dihydro-1H-indene-4-carboxylic acid (A-13)

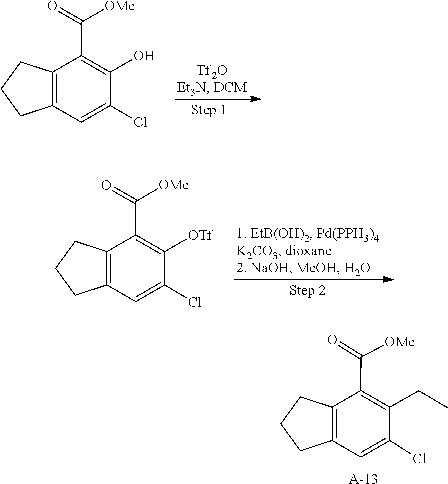

### Step 1: Methyl 6-chloro-5-(trifluoromethylsulfonyloxy)-2,3-dihydro-1H-indene-4-carboxylate

To a solution of methyl 6-chloro-5-hydroxy-2,3-dihydro-1H-indene-4-carboxylate (520 mg, 2.29 mmol) in DCM (20 mL) at 0° C., triethylamine (347 mg, 3.44 mmol) was added, followed by dropwise addition of trifluoromethanesufonic anhydride (712 mg, 2.52 mmol). The reaction mixture was stirred at ambient temperature for 1 h. The reaction mixture was quenched with water (20 mL), extracted with DCM (2×25 mL) then the combined organic extracts were washed with water (3×10 mL) and brine (15 mL). The organic layer was dried (1. Na₂SO₄) and concentrated to a residue which was purified by column chromatography on silica gel eluting with hexane/EtOAc (0-10%) to give the title compound (600 mg, 73.0%) as a colourless oil.

¹H NMR (300 MHz, CDCl₃) δ 7.45 (s, 1H), 3.92 (s, 3H), 3.12 (t, 2H), 2.93 (t, 2H), 2.19-2.06 (m, 2H).

¹⁹F NMR (300 MHz, CDCl₃) δ −73.31 ppm.

### Step 2: 6-Chloro-5-ethyl-2,3-dihydro-1H-indene-4-carboxylic acid

A solution of methyl 6-chloro-5-(trifluoromethylsulfonyloxy)-2,3-dihydro-1H-indene-4-carboxylate (182 mg, 0.507 mmol) in 1,4-dioxane (4 mL) was degassed with Argon gas for 20 min. Ethylboronic acid (60.1 mg, 0.813 mmol), Pd(PPh₃)₄(59 mg, 0.0507 mmol), and K₂CO₃ (112 mg, 0.813 mmol) were introduced. The reaction mixture was subjected to microwave heating at 130° C. for 1 h and treated with EtOAc (30 mL). The mixture was washed with water (2×10 mL) and brine (2×15 mL). The organic layer was dried (Na₂SO₄) The crude product in MeOH (15 mL) and NaOH (24.4 mg, 0.608 mmol) in H₂O (7 mL) was again subjected to microwave conditions at 130° C. for 50 min. The reaction mixture was concentrated, redissolved in water (4 mL), washed with DCM (2×10 mL) and the aqueous solution was acidified with 1N HCl to pH ~1 and extracted with EtOAc (20 mL). The organic layer was dried (Na₂SO₄) and purified by semi-prep HPLC utilising 30-50% acetonitrile/0.1% formic acid in water as eluent to afford the title compound (9.5 mg, 8.3%) as a white solid.

¹H NMR (300 MHz, CD₃OD) δ 7.29 (s, 1H), 3.01-2.78 (m, 6H), 3.17-2.02 (m, 2H), 1.27-1.10 (m, 3H).

ES-MS: m/z 223.6 (M-1).

HPLC: Retention time 7.777 min., purity 98% at 280 nm.

### Example 14: 6-Chloro-7-methyl-3,4-dihydro-2H-1-benzopyran-8-carboxylic acid (A-14)

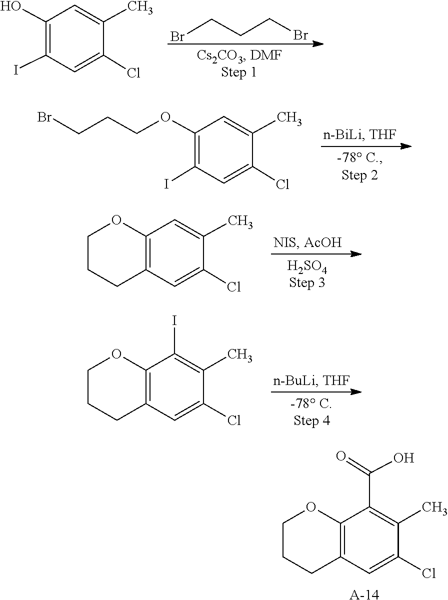

### Step 1: 1-(3-Bromopropoxy)-4-chloro-2-iodo-5-methylbenzene

To a solution of 4-chloro-2-iodo-5-methylphenol (2.5 g, 9.3 mmol) in DMF (15 mL) at ambient temperature, Cs₂CO₃ (9.12 g, 28.0 mmol) was added, followed by 1,3-dibromopropane (5.6 g, 28.0 mmol). The reaction mixture was stirred at 80° C. for 2 h, concentrated in vacuo and water (50 mL) was added. The mixture was extracted with EtOAc (2×50 mL) and the combined extracts were washed with water (3×50 mL), and brine (2×12 mL). The organic layer was dried (Na₂SO₄), evaporated and the residue purified by column chromatography on silica gel eluting with hexane/EtOAc (0-10%) to provide the title compound (1.60 g, 44%) as a colourless oil.

¹H NMR (300 MHz, CDCl₃) δ 7.70 (s, 1H), 6.69 (s, 1H), 4.16-4.08 (m, 2H), 3.75-3.66 (m, 2H), 2.40-2.33 (m, 2H), 2.32 (s, 3H).

### Step 2: 6-Chloro-7-methylchroman

n-BuLi (2.5 M in hexane) (1.8 mL, 4.52 mmol) was added to a solution of 1-(3-bromopropoxy)-4-chloro-2-iodo-5-methylbenzene (1.6 g, 4.1 mmol) in THF/hexane (10/3 mL) at −78° C. The reaction mixture was stirred at that temperature for 1 h., then at ambient temperature for 18 h. The reaction mixture was quenched with water (10 mL) and extracted with EtOAc (2×50 mL). The combined extracts were washed with water (50 mL) and brine (25 mL). The organic layer was dried (Na₂SO₄), evaporated and the residue purified by column chromatography on silica gel eluting with hexane/EtOAc (0-10%) to provide the title compound (0.55 g, 73%) as a colourless oil.

¹H NMR (300 MHz, CDC₃) δ 7.00 (s, 1H), 6.65 (s, 1H), 4.18-4.03 (m, 2H), 2.77-2.62 (m, 2H), 2.27 (s, 3H), 2.09-1.90 (m, 2H).

### Step 3: 6-Chloro-8-iodo-7-methylchroman

To a solution of 6-chloro-7-methylchroman (0.32 g, 1.76 mmol) in AcOH (1.5 mL) at ambient temperature N-iodosuccinimide (0.396 g, 1.76 mmol) was added, followed by H₂SO₄ (0.052 g, 0.53 mmol). The reaction mixture was stirred at ambient temperature for 4 h. quenched with water (10 mL) and then extracted with DCM (2×20 mL). The organic extracts were washed with water (20 mL), 10% sodium metabisulfite solution (2×15 mL) and brine (20 mL) and dried (Na₂SO₄). The residue after evaporation was purified by column chromatography on silica gel eluting with hexane/EtOAc (0-10%) to provide the title compound (0.28 g, 51%).

¹H NMR (300 MHz, CDC₃) δ 7.04 (s, 1H), 4.27 (t, 2H), 2.74 (t, 2H), 2.57 (s, 3H), 2.03-1.91 (m, 2H).

### Step 4: 6-Chloro-7-methyl-3,4-dihydro-2H-1-benzopyran-8-carboxylic acid

To a solution of 6-chloro-8-iodo-7-methylchroman (0.17 g, 0.551 mmol) in dry THF (10 mL) at −78° C. n-BuLi (2.5 M in hexane) (0.243 mL, 0.61 mmol) was added. The reaction mixture was stirred at that temperature for 30 min. and CO₂ (g) was bubbled through it for 10 min. Stirring was continued for 1 h at −78° C. before the mixture was being slowly brought to 0° C. and quenched with 1N NaOH to pH ~11. Washing with EtOAc (10 mL) and separation of the water layer was performed before separation and acidification with 1N HCl to pH ~1. The mixture was extracted with EtOAc (2×10 mL) then washed with water (5 mL) and brine (5 mL). The organic layer was dried (Na₂SO₄) filtered and evaporated to afford the crude product was which was crystallized from DCM/hexane to obtain title compound 40 mg (32%) as a white solid.

¹H NMR (300 MHz, CDCl₃) δ 10.47-9.35 (br s, 1H), 7.10 (s, 1H), 4.25 (t, 2H), 2.76 (t, 2H), 2.39 (s, 3H), 2.09-1.95 (m, 2H).

ES-MS: m/z 225.6 (M-1).

HPLC: Retention time 6.657 min., purity >99% at 280 nm.

### Example 15: 5-Chloro-6-methoxy-2,3-dihydro-1-benzofuran-7-carboxylic acid (A-15)

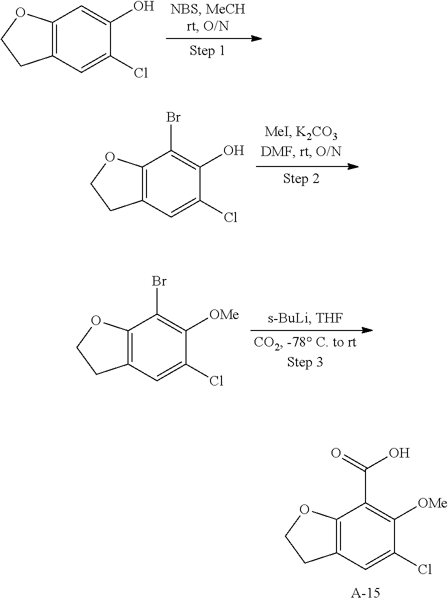

### Step 1: 7-Bromo-5-chloro-2,3-dihydro-1-benzofuran-6-ol

NBS (157 mg, 0.88 mmol) was added to a solution of 5-chloro-2,3-dihydro-1-benzofuran-6-ol (150 mg, 0.88 mmol) in acetonitrile (5 mL) at ambient temperature.

The reaction mixture was stirred for 16 h, concentrated, taken up EtOAc (30 mL) then washed with water (2×20 mL) and brine (2×20 mL). The organic layer was dried (Na₂SO₄), concentrated and the crude product was purified by preparative TLC eluting with hexane/EtOAc (10%) to provide the title compound (161 mg: 73%).

¹H NMR (300 MHz, CDCl₃) δ 7.09 (s, 1H), 5.37 (s, 1H), 4.71 (t, 2H), 3.28 (t, 2H).

### Step 2: 7-Bromo-5-chloro-6-methoxy-2,3-dihydro-1-benzofuran

K₂CO₃ (267.0 mg, 1.94 mmol) and iodomethane (0.06 mL, 0.97 mmol) were added sequentially to a solution of 7-bromo-5-chloro-2,3-dihydro-1-benzofuran-6-ol (160 mg, 0.65 mmol) in DMF (5 mL) at ambient temperature. The reaction mixture was stirred for 16 h, concentrated and the residue treated with EtOAc (20 mL) and the solution washed with water (2×10 mL) and brine (2×10 mL). The organic layer was dried (Na₂SO₄) and concentrated in vacuo to provide the title compound (166 mg, 98%).

¹H NMR (300 MHz, CDCl₃) δ 7.11 (s, 1H), 4.69 (t, 2H), 3.87 (s, 3H), 3.28 (t, 2H).

### Step 3: 5-Chloro-6-methoxy-2,3-dihydro-1-benzofuran-7-carboxylic acid

s-BuLi (2.5 M in hexane, 0.17 mL, 0.42 mmol) was added to a solution of 7-bromo-5-chloro-6-methoxy-2,3-dihydro-1-benzofuran (100 mg, 0.38 mmol) in THF (5 mL) at −78° C. The reaction mixture was stirred at that temperature for 2 h. CO₂ was bubbled through the mixture for 10 min. and it was stirred for 1 h at −78° C. The reaction temperature was adjusted to 0° C., the mixture treated with 1N NaOH to pH ~11 and the reaction mixture was washed with DCM (2×10 mL). The aqueous layer was acidified with 1N HCl to pH ~1 and extracted with EtOAc (20 mL) and the extract dried (Na₂SO₄) and concentrated. The crude product was purified by semi-prep HPLC using 30 to 100% acetonitrile/0.1% formic acid in water to provide the title acid (41 mg, 47%).

¹H NMR (300 MHz, CD₃OD) δ 7.20 (s, 1H), 4.53 (t, 2H), 3.74 (s, 3H), 3.09 (t, 2H).

ES-MS: 249.40 [M−1].

HPLC: Retention time 5.969 min., purity >98% at 280 nm.

### Example 16: 5-Chloro-4-methoxytricyclo[6.2.1.0^{2,7}]undeca-2(7),3,5-triene-3-carboxylic acid (A-16)

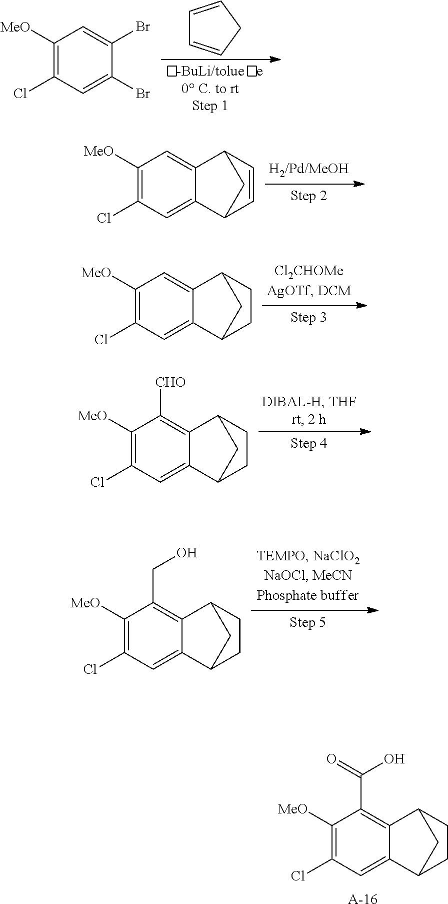

### Step 1: 4-chloro-5-methoxy-tricyclo[6.2.1.0^{2,7}]undeca-2(7),3,5,9-tetraene

1,2-dibromo-4-chloro-5-methoxybenzene (4.12 g, 13.72 mmol) and freshly distilled cyclopentadiene (1.81 mL, 68.6 mmol) were stirred in toluene (60 mL) at 0° C. under an argon atmosphere. n-BuLi (5.76 mL, 2.5 M in hexanes, 14.4 mmol) was introduced dropwise over 30 min. After an additional 10 min. at 0° C. the mixture was stirred at ambient temperature for 18 h, treated with water (30 mL) and extracted with EtOAc (3×50 mL). The combined extracts were dried (Na₂SO₄) and concentrated in vacuo. The residue was purified by chromatography on silica gel eluting with hexane/EtOAc (0-5%) to provide the title compound (2.6 g, 91%) as a colourless liquid.

¹H NMR (300 MHz, CDCl₃) δ 7.26 (s, 1H); 7.02 (s, 1H); 6.84 (br, 2H); 3.97 (s, 1H); 3.95 (s, 1H); 3.93 (s, 3H); 2.41-2.22 (m, 2H); ES-MS: 207 [M+1].

### Step 2: 4-chloro-5-methoxy-tricyclo[6.2.1.0^{2,7}}]undeca-2(7),3,5-triene

4-Chloro-5-methoxy-tricyclo[6.2.1.0^{2,7}]undeca-2(7),3,5,9-tetraene (757 mg, 3.66 mmol) was dissolved in methanol (30 mL), Pd-C catalyst (10%, 385 mg) was added and the reaction mixture was stirred under hydrogen for 4 h. The reaction mixture was filtered and the solvent removed under reduced pressure to give the title compound in quantitative yield as a colourless oil.

¹H NMR (300 MHz, CDCl₃) δ 7.26 (s, 1H); 6.96 (s, 1H); 3.98 (s, 3H); 3.42 (d, 2H); 1.90 (dd, 2H); 1.43 (dd, 2H); ES-MS: 209 [M+1].

### Step 3: 5-chloro-4-methoxy-tricyclo[6.2.1.0^{2,7}]undeca-2(7),3,5-triene-3-carbaldehyde

To a suspension of 4-chloro-5-methoxy-tricyclo[6.2.1.0^{2,7}}]undeca-2(7),3,5-triene (1.51 g, 7.24 mmol) and AgOTf (5.57 g, 21.72 mmol) in dry DCM (11 mL) a solution of Cl₂CHOMe (1.96 mL, 21.72 mmol) in dry DCM (2 mL) was added at −78° C. under an argon atmosphere. After stirring at ambient temperature for 16 h the reaction mixture was quenched with saturated aqueous NaHCO₃. After stirring at ambient temperature for 30 min. the reaction mixture was filtered through a pad of Celite®, the organic layer was separated and the aqueous layer was extracted twice with DCM. The combined extracts were washed with brine, dried (Na₂SO₄) and concentrated in vacuo. The residue was purified by chromatography on silica gel eluting with hexane/EtOAc (0-10%) to provide the title compound (950 mg, 55%) as a colourless oil.

¹H NMR (300 MHz, CDCl₃) δ 10.32 (s, 1H); 7.26 (s, 1H); 4.12 (s, 1H); 3.82 (s, 3H); 3.19 (s, 1H); 1.93-1.73 (m, 2H); 1.58 (d, 1H); 1.40 (d, 1H); 1.06-0.94 (m, 2H); ES-MS: 237 [M+1].

### Step 4: 5-chloro-4-methoxy-3-tricyclo[6.2.1.0^{2,7}]undeca-2(7),3,5-trienyl)methanol

To a solution of 5-chloro-4-methoxy-tricyclo[6.2.1.0^{2,7}]undeca-2(7),3,5-triene-3-carbaldehyde (774 mg, 3.27 mmol) in THF (20 mL) at 0° C., DIBAL-H (1N in toluene, 4.91 mL, 4.91 mmol) was added dropwise over 20 min. and stirring was continued for a further 15 min. at 0° C. The reaction mixture was allowed to warm to ambient temperature and stirred for 2 h, quenched with saturated aqueous sodium potassium tartrate and extracted with EtOAc (2×50 mL). The combined extracts were washed with brine (50 mL), dried (Na₂SO₄) and evaporated. The residue was purified by chromatography on silica gel eluting with hexane/EtOAc (0-10%) to provide the title compound (525 mg, 67%) as a colourless oil.

¹H NMR (300 MHz, CDC₃) δ 7.26 (s, 1H); 4.87 (s, 2H); 4.03 (s, 3H); 3.68 (s, 1H); 3.45 (s, 1H); 2.05 (d, 2H); 1.87 (d, 1H); 1.65 (d, 1H); 1.29 (d, 2H); ES-MS: 239 [M+1].

### Step 5: 5-Chloro-4-methoxytricyclo[6.2.1.0^{2,7}]undeca-2(7),3,5-triene-3-carboxylic acid

To a stirred solution of 5-chloro-4-methoxy-3-tricyclo[6.2.1.0^{2,7}]undeca-2(7),3,5-trienyl)methanol (40 mg, 0.18 mmol), sodium chlorite (41.6 mg, 0.46 mmol) in acetonitrile (1.5 mL) and 1N sodium phosphate buffer (pH-6, 1.5 mL), sodium hypochlorite (5 drops, 4-4.99 M solution) was added followed by TEMPO (1.4 mg, 0.009 mmol). The reaction mixture was stirred at ambient temperature for 2 h. and further sodium hypochlorite (4 drops, 4-4.99 M solution) and TEMPO (1.4 mg, 0.009 mmol) were added, then additions of sodium hypochlorite and TEMPO were repeated three more times at 8 h intervals. After completion of the reaction, the mixture was cooled to 0° C. and 1N NaOH solution was added to pH ~13 followed by washing with DCM (40 mL). The water layer was separated, cooled to 0° C., acidified with 1 N HCl to pH ~1, extracted with EtOAc (2×50 mL) and the crude product was purified by semi-prep HPLC (10 to 100% acetonitrile/0.1% formic acid in water) to afford the title compound (19 mg, 44%).

¹H NMR (300 MHz, CDC₃) δ 7.26 (s, 1H); 4.05 (s, 3H); 3.26 (s, 1H); 1.98-1.79 (m, 2H); 1.65 (d, 1H); 1.45 (d, 1H); 1.22-1.02 (m, 2H); ES-MS: 251 [M−1].

HPLC Retention Time: 11.558 min., 98.9% purity @280 nm.

### Example 17: 3-Fluoro-2-methoxy-5,6,7,8-tetrahydronaphthalene-1-carboxylic acid (A-18)

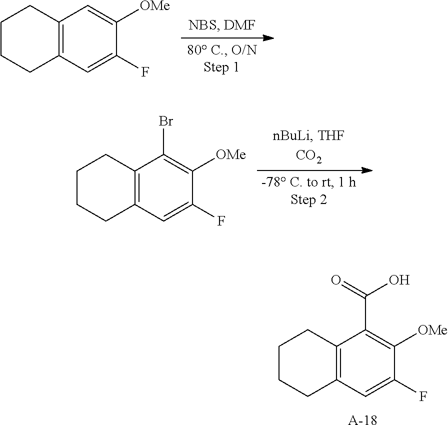

### Step 1: 5-Bromo-7-fluoro-6-methoxy-1,2,3,4-tetrahydronaphthalene

NBS (108.7 mg, 0.61 mmol) was added to a solution of 6-fluoro-7-methoxy-1,2,3,4-tetrahydronaphthalene (100 mg, 0.56 mmol) in DMF (5 mL). The reaction mixture was heated at 80° C. for 16 h, evaporated in vacuo. The residue was treated with EtOAc (30 mL) then the solution washed with water (2×20 mL) and brine (2×20 mL). The EtOAc solution was dried (Na₂SO₄) and concentrated. The resultant residue was purified by chromatography on silica gel eluting with hexane/EtOAc (4%) to provide the title compound (33 mg, 23%).

¹H NMR (300 MHz, CD₃OD) δ 6.76 (d, 1H), 3.73 (d, 3H), 2.65-2.50 (m, 4H), 1.75-1.55 (m, 4H). ¹⁹F NMR (300 MHz, CD₃OD) δ− 133.95 ppm.

### Step 2: 3-Fluoro-2-methoxy-5,6,7,8-tetrahydronaphthalene-1-carboxylic acid

n-BuLi (2.5 M in hexane) (0.05 mL, 0.13 mmol) was added to a solution of 5-bromo-7-fluoro-6-methoxy-1,2,3,4-tetrahydronaphthalene (30 mg, 0.12 mmol) in THF (2 mL) at −78° C. The reaction mixture was stirred at that temperature for 1 h. CO₂ was bubbled through the mixture for 10 min. and it was stirred for 1 h at −78° C. The reaction temperature was adjusted to 0° C. and the mixture treated with 1 N NaOH to pH ~11.

The reaction mixture was extracted with DCM (2×10 mL). The aqueous layer was acidified with 1N HCl to pH ~1 and extracted with EtOAc (20 mL) and the extract dried (Na₂SO₄) and concentrated. The crude product was purified by semi-prep HPLC utilising 30 to 100% acetonitrile/0.1% formic acid in water to provide the title acid (5.7 mg, 21%).

¹H NMR (300 MHz, CD₃OD) δ 6.88 (d, 1H), 3.89 (d, 3H), 2.80-2.62 (m, 4H), 1.86-1.72 (m, 4H).

¹⁹F NMR (300 MHz, CD₃OD) δ− 136.62 ppm.

ES-MS: 223.4 [M−1].

HPLC: Retention time 6.963 min., purity >98% at 280 nm.

### Example 18: 3-Chloro-4-fluoro-2-methoxy-5,6,7,8-tetrahydronaphthalene-1-carboxylic acid (A-19)

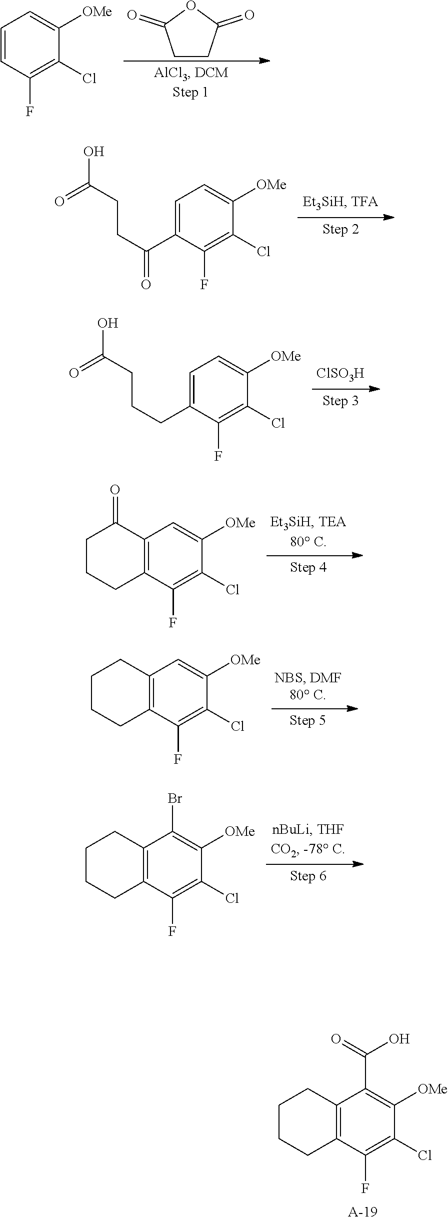

### Step 1: 4-(3-Chloro-2-fluoro-4-methoxyphenyl)-4-oxobutanoic acid

To a solution of 2-chloro-1-fluoro-3-methoxybenzene (3 gm, 18.68 mmol) in dry DCM (30 mL), succinic anhydride (1.68 gm, 16.81 mmol) was added at ambient temperature under an argon atmosphere. AlCl₃ (3.11 gm, 23.35 mmol) was introduced slowly in portions to the reaction mixture and the resulting solution was stirred for 16 h at ambient temperature before being quenched with 1N HCl (25 mL). The solvent was evaporated under reduced pressure, the residue was dissolved in EtOAc (50 mL) and washed with water (2×50 mL). The organic layer was dried (Na₂SO₄), filtered and concentrated under reduced pressure. The residue was purified by column chromatography on silica gel eluting with hexane/EtOAc (30-50%) to provide the title acid (1.70 g, 35%) as a white solid.

¹H NMR (300 MHz, DMSO) δ 7.87-7.82 (m, 1H); 7.16-7.13 (m, 1H); 3.97 (s, 3H); 3.18-3.12 (m, 2H); 2.58-2.54 (m, 2H).

ES-MS: 259.35 [M−1]

### Step 2: 4-(3-Chloro-2-fluoro-4-methoxyphenyl)butanoic acid

4-(3-Chloro-2-fluoro-4-methoxyphenyl)-4-oxobutanoic acid (1.7 gm, 6.45 mmol) in Et₃SiH (6.2 mL, 38.7 mmol) and TFA (3 mL, 38.7 mmol) were placed in a sealed tube and stirred at ambient temperature for 24 h. The solvent was removed under reduced pressure and the residue was purified by column chromatography on silica gel eluting with hexane/EtOAc (30-50%) providing 4-(3-chloro-2-fluoro-4-methoxyphenyl)butanoic acid (1.00 g, 63%) as a white solid.

¹H NMR (300 MHz, CDC₃) δ 10.36 (s, 1H), 7.28-7.23 (m, 1H); 6.92-6.88 (m, 1H); 4.12 (s, 3H); 2.93-2.88 (m, 2H); 2.65-2.60 (m, 2H), 2.22-2.12 (m, 2H).

ES-MS: 245.5 [M−1].

Step 3: 6-Chloro-5-fluoro-7-methoxy-3,4-dihydronapthalen-1(2H)-one 4-(3-chloro-2-fluoro-4-methoxyphenyl)butanoic acid (1.0 gm, 4.06 mmol) and chlorosulfonic acid (6 mL) were mixed and the reaction mixture was stirred for 24 h. at ambient temperature. The reaction mixture was quenched with saturated NaHCO₃ (10 mL) and extracted with EtOAc (2×25 mL). The crude product was purified by column chromatography on silica gel eluting with hexane/EtOAc (0-10%) to afford 6-chloro-5-fluoro-7-methoxy-3,4-dihydronapthalen-1(2H)-one (406 mg, 47%).

¹H NMR (300 MHz, CDC₃) δ 7.26-7.25 (m, 1H); 3.81 (s, 3H); 2.79-2.75 (m, 2H); 2.54-2.49 (m, 2H); 2.05-1.97 (m, 2H).

### Step 4: 6-Chloro-5-fluoro-7-methoxy-1,2,3,4-tetrahydronapthalene

6-Chloro-5-fluoro-7-methoxy-3,4-dihydronapthalen-1(2H)-one (186 mg, 0.813 mmol) in Et₃SiH (0.8 mL, 4.88 mmol) and TFA (0.4 mL, 4.88 mmol) were heated in a sealed tube at 80° C. for 24 h. The solvent was removed under reduced pressure and the residue was purified by column chromatography on silica gel eluting with hexane/EtOAc (0-5%) to obtain 6-chloro-5-fluoro-7-methoxy-1,2,3,4-tetrahydronapthalene (130 mg, 86%) as a yellow oil.

¹H NMR (300 MHz, CDCl₃) δ 6.44 (s, 1H); 3.86 (s, 3H); 2.73-2.63 (m, 4H); 1.79-1.74 (m, 4H).

### Step 5: 5-Bromo-7-chloro-8-fluoro-6-methoxy-1,2,3,4-tetrahydronapthalene

To a solution of 6-chloro-5-fluoro-7-methoxy-1,2,3,4-tetrahydronapthalene (130 mg, 0.61 mmol) in DMF (4 mL), NBS (119.25 mg, 0.67 mmol) was added, and the reaction mixture was stirred at 80° C. for 24 h. The DMF was removed under reduced pressure and the residue was dissolved in EtOAc (100 mL) and washed with water (2×10 mL).

The organic layer was dried (Na₂SO₄), filtered and concentrated under reduced pressure. The residue was purified to column chromatography on silica gel eluting with hexane/EtOAc (0-5%) to afford 5-bromo-7-chloro-8-fluoro-6-methoxy-1,2,3,4-tetrahydronapthalene (110 mg, 86%) as a yellow oil.

¹H NMR (300 MHz, CDCl₃) δ 3.87 (s, 3H); 2.73-2.67 (m, 4H); 1.84-1.70 (m, 4H).

### Step 6: 3-Chloro-4-fluoro-2-methoxy-5,6,7,8-tetrahydronaphthalene-1-carboxylic acid

To a solution of 5-bromo-7-chloro-8-fluoro-6-methoxy-1,2,3,4-tetrahydronapthalene (110 mg, 0.38 mmol) in THF (4 mL) at −78° C., n-BuLi (2.5.M in hexanes, 0.15 mL, 0.38 mmol) was added. The reaction mixture was stirred at −78° C. for 30 min. and CO₂ (g) was bubbled through the reaction mixture for 20 min. and it was stirred for 1 h at −78° C. and slowly bought to 0° C. The reaction mixture was quenched with 1 N NaOH to pH ~11 and washed with EtOAc (10 mL). The aqueous layer was separated and acidified with 1N HCl to pH ~1 and extracted with EtOAc (2×10 mL). The combined organic extracts were washed with water (10 mL) and brine (10 mL) and dried (Na₂SO₄), filtered and concentrated under reduced pressure to obtain 3-chloro-4-fluoro-2-methoxy-5,6,7,8-tetrahydronapthalene-1-carboxylic acid (70 mg, 72%) as a white solid.

¹H NMR (300 MHz, CDCl₃) δ 3.94 (s, 3H), 2.81-2.72 (m, 4H), 1.81-1.75 (m, 4H).

ES-MS: m/z 257.6 (M-1) HPLC: Retention time 14.614 min., purity 92% at 254 nm.

### Example 19: 4-chloro-5-methyltricyclo[6.2.1.0^{2,7}]undeca-2,4,6-triene-3-carboxylic

acid (A-20) and 5-chloro-4-methyltricyclo[6.2.1.0^{2,7}]undeca-2(7),3,5-triene-3-carboxylic acid (A-21)

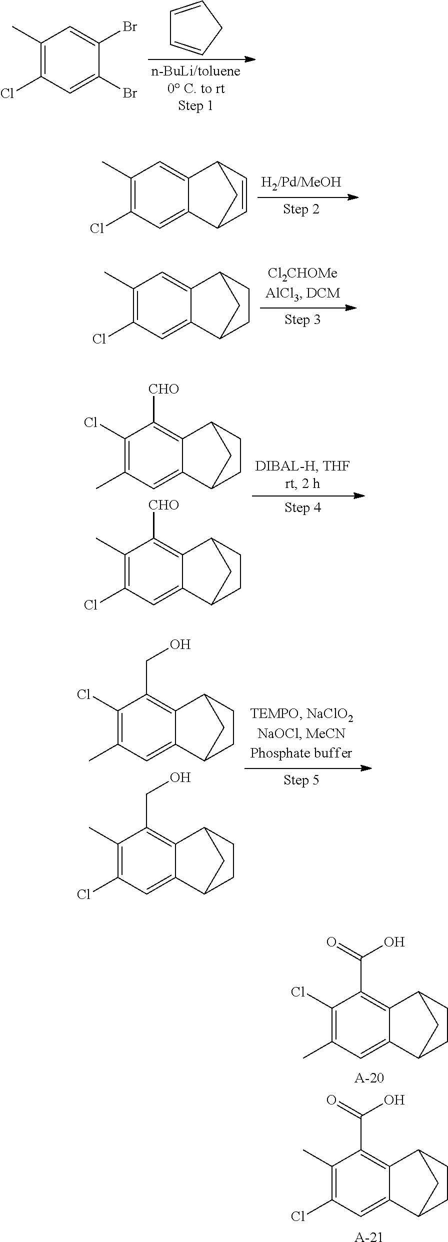

### Step 1: 4-chloro-5-methyl-tricyclo[6.2.1.0^{2,7}]undeca-2(7),3,5,9-tetraene

1,2-Dibromo-4-chloro-5-methylbenzene (2.32 g, 8.16 mmol) and cyclopentadiene (freshly distilled (3.43 mL, 40.8 mmol) were stirred in toluene (30 mL) at 0° C. under an argon atmosphere. n-BuLi (3.43 mL, 2.5M in hexanes, 8.57 mmol) was introduced dropwise over 30 min. After an additional 10 min. at 0° C. the mixture was allowed to warm to ambient temperature, stirred for 18 h, treated with water (30 mL) and extracted with EtOAc (3×50 mL). The combined extracts were dried (Na₂SO₄) and concentrated to a residue which was purified by chromatography on silica gel eluting with hexane/EtOAc (0-5%) to provide the title compound (1.3 g, 83%) as colourless oil.

¹H NMR (300 MHz, CDC₃) δ 7.26 (s, 1H); 7.16 (s, 1H); 6.83 (br, 2H); 3.90 (s, 2H); 2.38 (d, 1H); 2.36 (s, 3H); 2.47 (d, 1H); ES-MS: 191 [M+1].

### Step 2: 4-chloro-5-methyl-tricyclo[6.2.1.0^{2,7}]undeca-2(7),3,5-triene

4-Chloro-5-methyl-tricyclo[6.2.1.0^{2,7}]undeca-2(7),3,5,9-tetraene (853 mg, 4.77 mmol) was dissolved in methanol (20 mL), Pd-C (10%, 285 mg) was added and the reaction mixture was stirred under a hydrogen atmosphere for 4 h. The mixture was filtered and the solution evaporated under reduced pressure to provide the title compound in quantitative yield as a colourless oil.

¹H NMR (300 MHz, CDC₃) δ 7.26 (s, 1H); 7.15 (s, 1H); 3.43 (s, 2H); 2.45 (s, 3H); 2.06-1.97 (m, 2H); 1.88-1.81 (m, 1H); 1.67-1.60 (m, 1H); 1.32-1.22 (m, 2H); ES-MS: 193 [M+1].

### Step 3: 4-chloro-5-methyl-tricyclo[6.2.1.0^{2,7}]undeca-2(7),3,5-triene-3-carbaldehyde and 5-chloro-4-methyl-tricyclo[6.2.1.0^{2,7}]undeca-2(7),3,5-triene-3-carbaldehyde

To a suspension of 4-chloro-5-methyl-tricyclo[6.2.1.0^{A}{2,7}]undeca-2(7),3,5-triene (220 mg, 1.14 mmol) and AlCl₃ (228 mg, 1.71 mmol) in dry DCM (5 mL) a solution of Cl₂CHOMe (0.15 mL, 1.71 mmol) in dry DCM (2 mL) was added at −78° C. under an argon atmosphere and stirred for 1 h at −78° C. The mixture was stirred at 0° C. for 2 h, quenched with saturated aqueous NaHCO₃, stirred at ambient temperature for 30 min. and filtered through a pad of Celite®. The organic layer was separated and the aqueous layer was extracted with DCM (2×25 mL). The combined extracts were washed with brine, dried (Na₂SO₄) and concentrated in vacuo. The residue was purified by chromatography on silica gel eluting with hexane/EtOAc (0-5%) to provide the title compounds (3:1 ratio, 260 mg) as a colourless oil. 4A: ¹H NMR (300 MHz, CDC₃) δ 10.64 (s, 1H); 7.26 (s, 1H); 4.26 (s, 1H); 3.32 (s, 1H);

2.39 (s, 3H); 2.05-1.94 (m, 2H); 1.72-1.64 (m, 1H); 1.56-1.49 (m, 1H); 1.16-1.09 (m, 2H); ES-MS: 221 [M+1]. 4B: ¹H NMR (300 MHz, CDC₃) δ 10.53 (s, 1H); 7.38 (s, 1H); 4.11 (s, 1H); 3.36 (s, 1H); 2.65 (s, 3H); 1.93-1.87 (m, 2H); 1.76-1.69 (m, 1H); 1.61-1.56 (m, 1H); 1.21-1.16 (m, 2H); ES-MS: 221 [M+1].

### Step 4: (4-chloro-5-methyl-3-tricyclo[6.2.1.0^{2,7}]undeca-2(7),3,5-trienyl)methanol and (5-chloro-4-methyl-3-tricyclo[6.2.1.0^{2,7}]undeca-2(7),3,5-trienyl)methanol

To a solution of mixture of aldehydes (260 mg, 1.18 mmol) in THF (7 mL) at 0° C., DIBAL-H (1 N in toluene, 1.78 mL, 1.77 mmol) was added dropwise over 20 min. The solution was stirred for a further 15 min. at 0° C. and allowed to warm to ambient temperature and stirred for 2 h. The reaction was quenched with saturated aqueous sodium potassium tartrate and extracted with EtOAc (2×50 mL). The combined extracts were washed with brine (50 mL), dried (Na₂SO₄) and evaporated. The residue was purified by chromatography on silica gel eluting with hexane/EtOAc (0-10%) to give a mixture of alcohols (220 mg, 83%) as a colourless oil. 5A: ¹H NMR (300 MHz, CDC₃) δ 7.02 (s, 1H); 4.82 (s, 2H); 3.59 (s, 1H); 3.31 (s, 1H);

2.34 (s, 3H); 1.98-1.86 (m, 2H); 1.77-1.66 (m, 1H); 1.56-1.48 (m, 1H); 1.21-1.08 (m, 2H); ES-MS: 223 [M+1]. 5B: ¹H NMR (300 MHz, CDC₃) δ 7.15 (s, 1H); 4.75 (s, 2H); 3.57 (s, 1H); 3.31 (s, 1H);

2.42 (s, 3H); 1.98-1.86 (m, 2H); 1.77-1.66 (m, 1H); 1.56-1.48 (m, 1H); 1.21-1.08 (m, 2H); ES-MS: 223 [M+1].

### Step 5: 4-Chloro-5-methyltricyclo[6.2.1.0^{2,7}]undeca-2,4,6-triene-3-carboxylic acid and 5-chloro-4-methyltricyclo[6.2.1.0^{2,7}]undeca-2(7),3,5-triene-3-carboxylic acid

To a stirred solution of the (4-chloro-5-methyl-3-tricyclo[6.2.1.0^{2,7}]undeca-2(7),3,5-trienyl)methanol and (5-chloro-4-methyl-3-tricyclo[6.2.1.0^{2,7}]undeca-2(7),3,5-trienyl)methanol mixture of alcohols (220 mg, 0.99 mmol), sodium chlorite (223 mg, 2.47 mmol) in acetonitrile (10 mL) and 1N sodium phosphate buffer (pH-6, 10 mL), sodium hypochlorite (5 drops, 4-4.99 M solution) was added, followed by TEMPO (7.7 mg, 0.049 mmol). The reaction mixture was stirred at ambient temperature for 2 h., further sodium hypochlorite (4 drops, 4-4.99 M solution) and TEMPO (7.7 mg, 0.049 mmol) were added, and the reagent additions repeated three more times at 8 h intervals. After cooling to 0° C., 1N NaOH solution was added to pH ~13 and the mixture was washed with DCM (40 mL). The water layer was cooled to 0° C., acidified with 1 N HCl to pH ~1, extracted with EtOAc (2×50 mL), evaporated in vacuo and the two acids were separated by semi-prep HPLC (10 to 100% acetonitrile/0.1% formic acid in water) to afford the two title compounds (A-20: 55 mg, 19.9% & A-21: 18 mg, 6.5%). 4-Chloro-5-methyltricyclo[6.2.1.0^{2,7}]undeca-2,4,6-triene-3-carboxylic acid (A-20) ¹H NMR (300 MHz, CDCl₃) δ 8.91-8.13 (br, 1H); 7.26 (s, 1H); 3.80 (s, 1H); 3.45 (s, 1H);

2.47 (s, 3H); 2.12-1.96 (m, 2H); 1.86 (d, 1H); 1.63 (d, 1H); 1.44-1.21 (m, 2H); ES-MS: 235 [M−1].

HPLC Retention Time: 11.626 min., >98% purity @280 nm. 5-Chloro-4-methyltricyclo[6.2.1.0^{2,7}]undeca-2(7),3,5-triene-3-carboxylic acid (A-21) ¹H NMR (300 MHz, CDCl₃) δ 7.30 (s, 1H); 3.75 (s, 1H); 3.35 (s, 1H); 2.51 (s, 3H); 2.02-1.86 (m, 2H); 1.75 (d, 1H); 1.52 (d, 1H); 1.32-1.11 (m, 2H); ES-MS: 235 [M−1].

HPLC Retention Time: 12.263 min., >98% purity @280 nm.

### Example 20: 3-Chloro-2-methoxy-6,7,8,9-tetrahydro-5H-benzo[7]annulene-1-carboxylic acid (A-22)

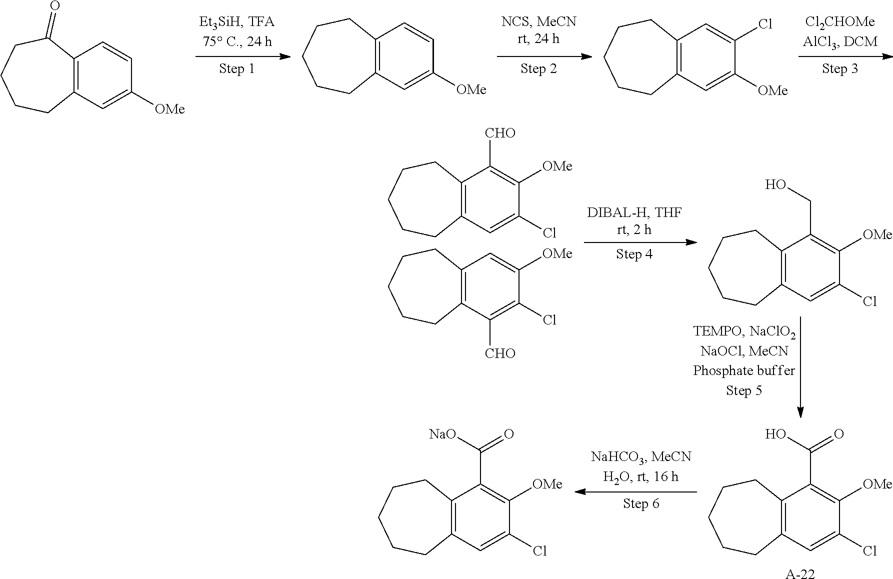

The title compound was prepared from intermediate 4A by the method described in Example 22.

¹H NMR (300 MHz, CD₃OD) δ 7.00 (s, 1H), 3.89 (s, 3H), 2.86-2.72 (m, 4H), 1.91-1.80 (m, 2H), 1.74-1.57 (m, 4H).

ES-MS: 253 [M−H]

HPLC Retention Time: 11.8 min.; Purity: >98% @280 nm

### Example 21: 6-Chloro-5-(methylsulfanyl)-2,3-dihydro-1H-indene-4-carboxylic acid (A-23)

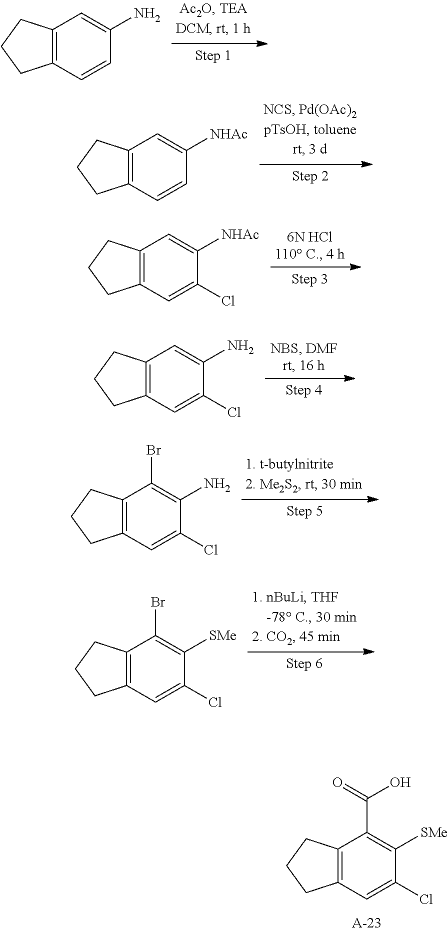

### Step 1: 1-(5-Indanylamino)-1-ethanone

To a solution of 5-indanylamine (5.00 g, 37.5 mmol) in DCM (50 mL) at 0° C., trimethylamine (10.5 mL, 75.1 mmol) was added, followed by slow addition of acetic anhydride (5.3 mL, 56.2 mmol) and the mixture was stirred at ambient temperature for 16 h. The reaction mixture was quenched with 1.0 N HCl (50 mL) and the layers were separated. The aqueous layer was extracted into DCM (2×50 mL), then the combined organic layers were dried (Na₂SO₄) and concentrated under reduced pressure. The residue was triturated with hexanes (2×40 mL) to provide the title compound (4.92 g. 75%).

¹H NMR (300 MHz, CDCl₃) δ 7.56 (s, 1H), 7.37-7.29 (br s, 1H), 7.28-7.23 (m, 2H), 2.99 (q, 4H), 2.27 (s, 3H), 2.18 (m, 2H).

ES-MS: 176 [M+H]

### Step 2: 1-(6-Chloro-5-indanylamino)-1-ethanone

To a solution of 1-(5-indanylamino)-1-ethanone (4.50 g, 25.6 mmol) in toluene (60 mL), p-TsOH·H₂O (2.44 g, 12.8 mmol) and Pd(OAc)₂ (0.29 g, 1.28 mmol) was added followed by NCS (3.60 g, 27.0 mmol) and the mixture was stirred at ambient temperature for 72 h. The solvent was removed under reduced pressure then the residue was dissolved in EtOAc (250 mL) and washed with water (2×150 mL). The organic layer was dried (Na₂SO₄) and concentrated under reduced pressure to a residue that was triturated with ether (3×60 mL) to provide the title compound (3.50 g, 65%).

¹H NMR (300 MHz, CDC₃) δ 8.15 (s, 1H), 7.58-7.45 (br s, 1H), 7.20 (s, 1H), 2.93-2.79 (m, 4H), 2.21 (s, 3H), 2.07 (m, 2H).

ES-MS: 210 [M+H]

### Step 3: 6-Chloro-5-indanylamine

A mixture of 1-(6-chloro-5-indanylamino)-1-ethanone (3.00 g, 14.3 mmol), 6.0 N HCl (150 mL) was heated at 110° C. for 4 h. The reaction mixture was cooled to ambient temperature and washed with EtOAc (2×150 mL). The aqueous layer was basified to pH ~10 utilising NaOH flakes and extracted with EtOAc (2×200 mL). The combined organic layers were dried (Na₂SO₄) and concentrated under reduced pressure to obtain the title compound (1.83 g, 76%).

¹H NMR (300 MHz, CDC₃) δ 7.26 (s, 1H), 6.83 (s, 1H), 4.13-4.01 (br s, 2H), 2.96 (t, 4H), 2.20 (m, 2H).

ES-MS: 168 [M+H]

### Step 4: 4-Bromo-6-chloro-5-indanylamine

To a solution of 6-chloro-5-indanylamine (0.50 g, 2.98 mmol) in DMF (10 mL) at 0° C., NBS (0.58 g, 3.13 mmol) was introduced in several portions and the mixture was stirred at ambient temperature for 16 h. The reaction mixture was quenched with water (20 mL) and extracted with EtOAc (2×30 mL). The combined organic layers were washed with water (2×30 mL), dried (Na₂SO₄,) and concentrated under reduced pressure to obtain the title compound (0.60 g, 81%).

¹H NMR (300 MHz, CDC₃) δ 7.26 (s, 1H), 4.68-4.43 (br s, 2H), 3.17-3.03 (m, 4H), 2.26 (m, 2H).

ES-MS: 246 and 248 [M+H]

### Step 5: 4-Bromo-6-chloro-5-(methylthio)indane

To a stirred solution of 4-bromo-6-chloro-5-indanylamine (0.60 g, 2.43 mmol) in CHCl₃ (3.0 mL), tert-butyl nitrite (0.38 mL, 3.20 mmol) and dimethyl disulphide (0.48 mL, 5.3 mmol) in 5.0 mL of CHCl₃ were introduced, and stirred the mixture at ambient temperature for 30 min. The reaction mixture was quenched with water (12 mL) plus 1.0 N HCl (12 mL) and the layers were separated. The organic layer was dried (Na₂SO₄), concentrated under reduced pressure and the residue was purified by chromatography on silica gel eluting with hexane/EtOAc (0-5%) to provide the title compound (0.17 g, 25%).

¹H NMR (300 MHz, CDC₃) δ 7.27 (s, 1H), 3.04-2.90 (m, 4H), 2.40 (s, 3H), 2.11 (m, 2H).

ES-MS: 278 [M+H]

### Step 6: 6-Chloro-5-(methylsulfanyl)-2,3-dihydro-1H-indene-4-carboxylic acid

To a solution of 4-bromo-6-chloro-5-(methylthio)indane (170 mg, 0.61 mmol) in diethyl ether (4.0 mL) at −78° C., n-BuLi (0.42 mL, 0.67 mmol was added. The reaction mixture was stirred for 30 min. at that temperature and then CO₂ was bubbled through it for 45 min. The reaction mixture was allowed to reach ambient temperature and stirred for 15 min. The reaction mixture was quenched with water (15 mL) and EtOAc (20 mL) added. The layers were separated and the aqueous layer was acidified to pH ~2 with 1.0 N HCl. The aqueous layer was extracted with EtOAc (2×20 mL) and the combined extracts dried (Na₂SO₄), gravity filtered and concentrated under reduced pressure. The residue was purified utilising semi-preparative HPLC with acetonitrile/0.1% formic acid in water (30-50%) to afford the title compound (6.4 mg, 4.3%).

¹H NMR (300 MHz, CDC₃) δ 7.26 (s, 1H), 2.95-2.68 (m, 4H), 2.28 (s, 3H), 2.10-1.88 (m, 2H).

ES-MS: 241 [M−H]

HPLC Retention Time: 11.0 min.; Purity: >98% @280 nm.

### Example 22: 2-Chloro-3-methoxy-6,7,8,9-tetrahydro-5H-benzo[7]annulene-1-carboxylic acid (A-24)

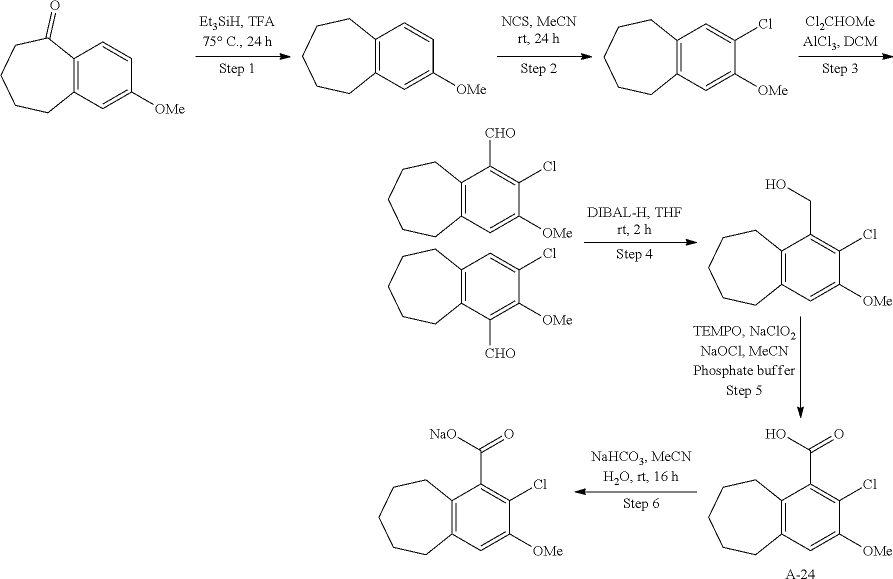

### Step 1: 9-Methoxybicyclo[5.4.0]undeca-1(7),8,10-triene

2-Methoxy-6,7,8,9-tetrahydro-benzocyclohepten-5-one (8.00 g, 42.1 mmol) in a mixture of Et₃SiH (40.3 mL, 252 mmol) and TFA (19.3 mL, 252 mmol) was heated at 75° C. over a period of 24 h. The reaction mixture was evaporated under reduced pressure and the residue was purified by column chromatography on silica gel eluting with hexane/EtOAc (0-5%) to obtain the desired compound (4.90 g, 66%) ¹H NMR (300 MHz, CDCl₃) δ 7.00 (d, 1H), 6.68 (d, 1H), 6.61 (dd, 1H), 3.76 (s, 3H), 2.78-2.68 (m, 4H), 1.88-1.76 (m, 2H), 1.70-1.57 (m, 4H).

ES-MS: 177 [M+H]

### Step 2: 9-Chloro-10-methoxybicyclo[5.4.0]undeca-1(7),8,10-triene

To a solution of 9-methoxybicyclo[5.4.0]undeca-1(7),8,10-triene (4.20 g, 23.8 mmol) in acetonitrile (100 mL), NCS (3.18 g, 23.8 mmol) was added and the reaction mixture was stirred at ambient temperature for 24 h. The solvent was removed under reduced pressure, the residue was dissolved in EtOAc (100 mL) and washed with water (2×100 mL). The organic layer was dried (Na₂SO₄) and concentrated under reduced pressure. The residue was purified by column chromatography on silica gel eluting with hexane/EtOAc (0-5%) to provide the title compound (3.12 g, 62%).

¹H NMR (300 MHz, CDC₃) δ 7.08 (s, 1H), 6.69 (s, 1H), 3.87 (s, 3H), 2.78-2.66 (m, 4H), 1.87-1.76 (m, 2H), 1.68-1.56 (m, 4H).

ES-MS: 211 [M+H]

### Step 3: 10-Chloro-9-methoxybicyclo[5.4.0]undeca-1(7),8,10-triene-8-carbaldehyde (4) and 9-chloro-10-methoxybicyclo[5.4.0]undeca-1(7),8,10-triene-8-carbaldehyde (4a)

To a solution of 9-chloro-10-methoxybicyclo[5.4.0]undeca-1 (7),8,10-triene (1.00 g, 4.75 mmol) in DCM (15 mL), AgOTf (3.66 g, 14.2 mmol) was introduced at −78° C. followed by the addition of dichloromethyl methyl ether (1.29 mL, 14.2 mmol) in DCM (5.0 mL).

The reaction mixture was stirred at ambient temperature for 16 h, cooled to 0° C. and quenched with water (15 mL). The mixture was filtered through a pad of Celite® and the layers were separated. The organic layer was dried (Na₂SO₄) and concentrated under reduced pressure. The residue was purified by column chromatography silica gel eluting with hexane/EtOAc (0-4%) to obtain the title compounds 4 (0.47 g, 41%) and 4A (0.14 g, 12%). 4: ¹H NMR (300 MHz, CDC₃) 610.5 (s, 1H), 7.30 (s, 1H), 3.90 (s, 3H), 3.14-3.06 (m, 2H), 2.82-2.75 (m, 2H), 1.86-1.75 (m, 2H), 1.68-1.57 (m, 4H). 4a: ¹H NMR (300 MHz, CDC₃) 610.3 (s, 1H), 7.49 (s, 1H), 3.96 (s, 3H), 3.14-3.06 (m, 2H), 2.89-2.81 (m, 2H), 1.90-1.78 (m, 2H), 1.70-1.59 (m, 4H).

ES-MS: 239 [M+H]

### Step 4: {9-Chloro-10-methoxybicyclo[5.4.0]undeca-1(7),8,10-trien-8-yl}methanol

To a solution of 9-chloro-10-methoxybicyclo[5.4.0]undeca-1(7),8,10-triene-8-carbaldehyde (4) (0.33 g, 1.57 mmol) in THF (8.0 mL), DIBAL-H (2.4 mL, 2.35 mmol) was added at 0° C. The reaction mixture was stirred at ambient temperature for 2 h, cooled to 0° C., quenched with 1.0 N HCl (50 mL) and extracted with EtOAc (2×50 mL).

The organic layer was dried (Na₂SO₄) and concentrated under reduced pressure. The crude product was utilised in the next step without further purification.

¹H NMR (300 MHz, CDC₃) δ 7.00 (s, 1H), 4.67 (d, 2H), 3.87 (s, 3H), 3.07-2.98 (m, 2H), 2.84-2.75 (m, 2H), 1.87-1.78 (m, 2H), 1.68-1.57 (m, 4H).

ES-MS: 241 [M+H]

### Step 5: 2-Chloro-3-methoxy-6,7,8,9-tetrahydro-5H-benzo[7]annulene-1-carboxylic acid

To a solution of {9-chloro-10-methoxybicyclo[5.4.0]undeca-1(7),8,10-trien-8-yl}methanol (0.35 g, 1.45 mmol) in acetonitrile (4.0 mL), 1.0 M sodium phosphate buffer (pH 6.0, 4.0 mL) was added followed by NaClO₂ (0.272 g, 3.65 mmol), NaOCl (0.2 mL, 4-4.99M solution) and TEMPO (22 mg, 0.14 mmol). The reaction mixture was stirred at ambient temperature for 16 h. To this mixture was added an additional NaOCl (0.2 mL, 4-4.99M solution) and TEMPO (22 mg, 0.14 mmol) and the reaction mixture was stirred for a further 2 h. The reaction mixture was quenched with 1.0 N NaOH (30 mL) and washed with EtOAc (2×30 mL). The aqueous layer was acidified to pH ~2.0 using 1.0 N HCl and extracted with EtOAc (2×50 mL). The combined organic layers were dried (Na₂SO₄) and concentrated under reduced pressure to afford the title compound (0.26 g, 70.4%).

¹H NMR (300 MHz, CDCl₃) δ 7.78 (s, 1H), 4.03 (s, 3H), 3.13-3.05 (m, 2H), 2.90-2.82 (m, 2H), 1.90-1.81 (m, 2H), 1.71-1.58 (m, 4H).

ES-MS: 253 [M−H]

HPLC Retention Time: 12.3; Purity: 97% @280 nm

### Step 6: Sodium 2-chloro-3-methoxy-6,7,8,9-tetrahydro-5H-benzo[7]annulene-1-carboxylate

To a solution of 2-chloro-3-methoxy-6,7,8,9-tetrahydro-5H-benzo[7]annulene-1-carboxylic acid (0.19 g, 0.76 mmol) in acetonitrile (5.0 mL), and water (5.0 mL), NaHCO₃ (64 mg, 0.76 mmol) was added and the reaction mixture was stirred at ambient temperature for 16 h. The solvent was removed under reduced pressure and triturated with diethyl ether (3×5.0 mL) to provide the title compound (150 mg, 71%).

¹H NMR (300 MHz, CD₃OD) δ 7.14 (s, 1H), 3.88 (s, 3H), 3.13-3.03 (m, 2H), 2.87-2.79 (m, 2H), 1.93-1.81 (m, 2H), 1.71-1.56 (m, 4H).

ES-MS: 253 [M−H]

HPLC Retention Time: 12.9 min.; Purity: >98% @254 nm

### Example 23: 3-Chloro-2-(difluoromethoxy)-5,6,7,8-tetrahydronaphthalene-1-carboxylic acid (A-25)

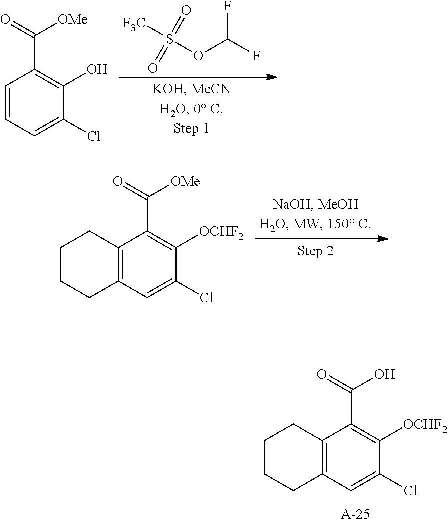

### Step 1: Methyl 3-chloro-2-difluoromethoxy-5,6,7,8-tetrahydronaphthalene-1-carboxylate

To a solution of methyl 3-chloro-2-hydroxy-5,6,7,8-tetrahydronaphthalene-1-carboxylate (550 mg, 2.285 mmol) in acetonitrile (5 mL) at 0° C., 6N KOH solution (5 mL) was added, followed by dropwise addition of (difluoromethoxysulfonyl)trifluoromethane (730 mg, 3.66 mmol). The reaction mixture was stirred at 0° C. for 15 min., quenched with water (20 mL), then the mixture was extracted with DCM (3×50 mL), dried (Na₂SO₄) and concentrated. The resultant residue was purified by chromatography on silica gel eluting with hexane/EtOAc (0-5%) to provide the title compound (350 mg, 52%).

¹H NMR (300 MHz, CDC₃) δ 7.26 (s, 1H), 6.53 (t, 1H), 3.96 (s, 3H), 2.78 (m, 4H), 1.83 (m, 4H) ppm. ¹⁹F NMR (300 MHz, CDC₃) δ 80.57 ppm

### Step 2: 3-Chloro-2-(difluoromethoxy)-5,6,7,8-tetrahydronaphthalene-1-carboxylic acid

A 1 N solution of NaOH (1.2 mL, 1.204 mmol) was added to a solution of methyl 3-chloro-2-difluoromethoxy-5,6,7,8-tetrahydronaphthalene-1-carboxylate (350 mg, 1.20 mmol) in MeOH (1 mL) and water (2 mL) was and heated under microwave conditions at 150° C. for 50 min. The mixture was evaporated in vacuo, the residue was dissolved in water and the solution was washed with DCM (3×10 mL). The aqueous layer was neutralised with 2N HCl, extracted with DCM (3×20 mL), dried (Na₂SO₄) and concentrated to provide the title acid as a white solid (150 mg, 45%).

¹H NMR (300 MHz, CD₃OD) δ 7.36 (s, 1H), 6.70 (t, 1H), 2.78 (m, 4H), 1.81 (m, 4H) ppm.

¹⁹F NMR (300 MHz, CD₃OD) δ 82.03 ppm.

ES-MS: m/z 275.4 (M-1).

HPLC: Retention time 10.51 min., purity >98% at 280 nm.

### Example 24: 3-Chloro-2-fluoro-5,6,7,8-tetrahydronaphthalene-1-carboxylic acid

(A-26) and 2-chloro-3-fluoro-5,6,7,8-tetrahydronaphthalene-1-carboxylic acid (A-27)

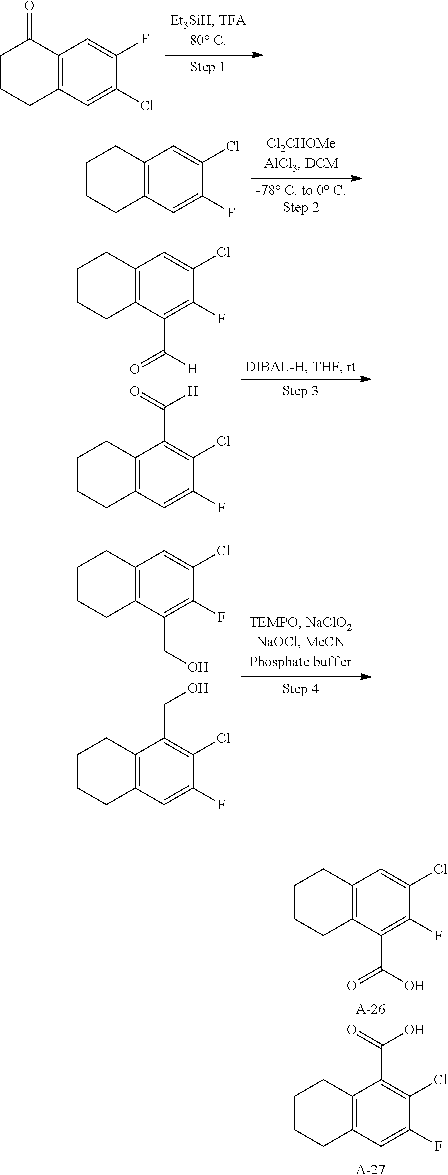

### Step 1: 6-Chloro-7-fluoro-1,2,3,4-tetrahydronapthalene

7-Chloro-6-fluoro-3,4-dihydronaphthalen-1(2H)-one (1.0 g, 5.03 mmol) in Et₃SiH (4.82 mL, 30.18 mmol) and TFA (2.31 mL, 30.18 mmol) were placed in a sealed tube and the mixture stirred at ambient temperature for 24 h. The solvent was removed under reduced pressure and the residue purified by column chromatography on silica gel eluting with hexane/EtOAc (0-5%) to provide 6-chloro-7-fluoro-1,2,3,4-tetrahydronapthalene (680 mg, 73%) as a white solid.

¹H NMR (300 MHz, CDCl₃) δ 7.27 (d, J_{H-F}=7.6 Hz, 1H); 7.03 (d, J_{H-F}=9.9, 1H); 2.94-2.89 (m, 4H); 2.01-1.97 (m, 4H).

### Step 2: 3-Chloro-2-fluoro-5,6,7,8-tetrahydronapthalene-1-carbaldehyde and 2-chloro-3-fluoro-5,6,7,8-tetrahydronapthalene-1-carbaldehyde

To a solution of 6-chloro-7-fluoro-1,2,3,4-tetrahydronapthalene (680 mg, 3.68 mmol) in dry DCM (19 mL), aluminium chloride (736 mg, 5.52 mmol) was introduced at −78° C. under an argon atmosphere. A solution of Cl₂CHOMe (0.5 mL, 5.52 mmol) in dry DCM (5 mL) was slowly added to the reaction mixture at −78° C. The reaction mixture was stirred at that temperature for 1 h and at 0° C. for 2 h, quenched with saturated aqueous NaHCO₃ (10 mL) and stirred at ambient temperature for 30 min. The organic layer was separated and the aqueous layer extracted with DCM (2×30 mL). The combined organic extracts were washed with brine (15 mL) and dried (Na₂SO₄). The filtrate was concentrated, and a mixture of 3-chloro-2-fluoro-5,6,7,8-tetrahydronapthalene-1-carbaldehyde (major isomer) and 2-chloro-3-fluoro-5,6,7,8-tetrahydronapthalene-1-carbaldehyde (minor isomer) (740 mg, 94%) were obtained as a yellow oil. The crude mixture was used for the next step without purification.

Major isomer (3): ¹H NMR (300 MHz, CDC₃) δ 10.42 (s, 1H); 7.25 (d, J_{H-F}=7.8 Hz, 1H); 3.02-2.97 (m, 4H); 2.69-2.59 (m, 4H).

Minor isomer (4): ¹H NMR (300 MHz, CDC₃) δ 10.51 (s, 1H); 6.74 (d, J_{H-F}=10.01 Hz, 1H); 3.02-2.97 (m, 4H); 2.69-2.59 (m, 4H).

### Step 3: 3-Chloro-2-fluoro-5,6,7,8-tetrahydronapthalene-1-yl)methanol and 2-chloro-3-fluoro-5,6,7,8-tetrahydronapthalene-1-yl)methanol

To a solution of the 3-chloro-2-fluoro-5,6,7,8-tetrahydronapthalene-1-carbaldehyde and 2-chloro-3-fluoro-5,6,7,8-tetrahydronapthalene-1-carbaldehyde mixture (740 mg, 3.48 mmol) in THF (10.0 mL) at 0° C. DIBAL-H (1N in toluene, 5.22 mL, 5.22 mmol) was added and the reaction mixture stirred at ambient temperature for 2 h. The mixture was cooled to 0° C., quenched with saturated sodium potassium tartrate (5 mL), stirred for 30 min. and extracted into EtOAc (2×50 mL). The combined extracts were dried (Na₂SO₄) and concentrated under reduced pressure. The resultant mixture of 3-chloro-2-fluoro-5,6,7,8-tetrahydronapthalene-1-yl) methanol (major isomer) and 2-chloro-3-fluoro-5,6,7,8-tetrahydronapthalene-1-yl) methanol (minor isomer) (617 mg, 83% ld) was utilised in the next step without further purification.

Major isomer (5): ¹H NMR (300 MHz, CDC₃) δ 7.27 (d, J_{H-F}=7.8 Hz, 1H); 4.92 (d, 2H); 3.09-2.90 (m, 4H); 2.04-1.95 (m, 4H).

Minor isomer (6): ¹H NMR (300 MHz, CDC₃) δ 7.03 (d, J_{H-F}=9.9 Hz, 1H); 5.02 (s, 2H); 3.09-2.90 (m, 4H); 2.04-1.95 (m, 4H).

### Step 4: 3-Chloro-2-fluoro-5,6,7,8-tetrahydronaphthalene-1-carboxylic acid and 2-chloro-3-fluoro-5,6,7,8-tetrahydronaphthalene-1-carboxylic acid

To a mixture of 3-chloro-2-fluoro-5,6,7,8-tetrahydronapthalene-1-yl) methanol and 2-chloro-3-fluoro-5,6,7,8-tetrahydronapthalene-1-yl) methanol (617 mg, 2.87 mmol) in acetonitrile (30.0 mL), 1.0 M sodium phosphate buffer (pH 6.0, 30.0 mL) was added. This was followed by addition of NaClO₂ (649.81 mg, 7.19 mmol), NaOCl (0.2 mL, 4-4.99 M solution) and TEMPO (23 mg, 0.14 mmol) at ambient temperature and the reaction mixture was stirred for 16 h. Additional NaOCl (0.2 mL, 4-4.99 M solution) and TEMPO (23 mg, 0.14 mmol) were introduced and stirring was continued at ambient temperature for 2 h. The reaction mixture was quenched with 1.0 N NaOH (30 mL) and washed with EtOAc (2×30 mL). The aqueous layer was acidified to pH ~2.0 using 1.0 N HCl and extracted with EtOAc (2×50 mL). The combined organic extracts were dried (Na₂SO₄) and evaporated under reduced pressure to obtain the mixture of 3-chloro-2-fluoro-5,6,7,8-tetrahydronapthalene-1-carboxylic acid (major isomer) and 2-chloro-3-fluoro-5,6,7,8-tetrahydronapthalene-1-carboxylic acid (minor isomer) (453 mg, 69%). The mixture was separated by semi-prep HPLC using 25-100% acetonitrile/0.1% formic acid in water.

3-Chloro-2-fluoro-5,6,7,8-tetrahydronaphthalene-1-carboxylic acid (A-26) ¹H NMR (300 MHz, CDCl₃) δ 7.15 (d, J_{H-F}=6.9 Hz, 1H); 6.55-5.95 (br, 1H); 2.79-2.70 (m, 4H); 1.79-1.69 (m, 4H).

ES-MS: 227.1 [M−1].

HPLC: Retention Time: 11.476 min., purity: 96% @254 nm.

2-Chloro-3-fluoro-5,6,7,8-tetrahydronaphthalene-1-carboxylic acid (A-27)

¹H NMR (300 MHz, CDCl₃) δ 6.89 (d, J_{H-F}=9.4 Hz, 1H); 5.23-4.82 (br, 1H); 2.78-2.70 (m, 4H); 1.80-1.73 (m, 4H).

ES-MS: 227.23 [M−1].

HPLC: Retention Time: 10.898 min., purity: 97% @254 nm.

### Example 25: 2-chloro-3-methyl-5,6,7,8-tetrahydronaphthalene-1-carboxylic acid (A-28)

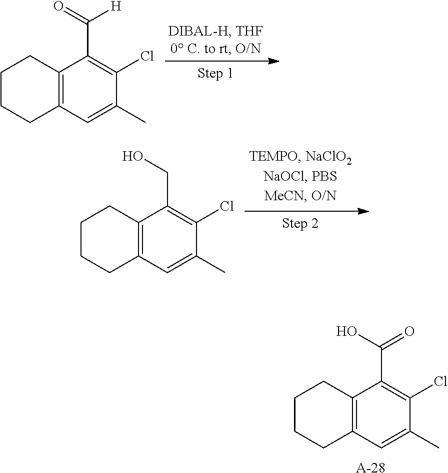

### Step 1: (2-Chloro-3-methyl-5,6,7,8-tetrahydronaphth-1-yl)methanol (2)

DIBAL-H (1N in toluene, 1.64 mL, 1.65 mmol) was added dropwise over 10 min. to a solution of 2-chloro-3-methyl-5,6,7,8-tetrahydronaphthalene-1-carbaldehyde (230 mg, 1.11 mmol) in THF (5 mL) at 0° C. and the solution was stirred for 15 min. at 0° C. The reaction mixture was allowed to warm to ambient temperature and stirred for 2 h. The reaction was quenched with saturated aqueous sodium potassium tartrate solution (30 mL) and extracted with EtOAc (2×20 mL). The combined extracts were washed with brine (20 mL), dried (Na₂SO₄) and evaporated in vacuo to afford the title compound (230 mg, 99%).

¹H NMR (300 MHz, CDC₃) δ 6.93 (s, 1H), 4.81 (s, 2H), 2.87 (t, 2H), 2.71 (t, 2H), 2.33 (s, 3H), 1.88-1.67 (m, 4H).

### Step 2: 2-Chloro-3-methyl-5,6,7,8-tetrahydronaphthalene-1-carboxylic acid (3)

To a stirred solution of (2-chloro-3-methyl-5,6,7,8-tetrahydronaphth-1-yl)methanol (230 mg, 1.09 mmol), sodium chlorite (247.54 mg, 2.74 mmol) in acetonitrile (10 mL) and 1N sodium phosphate buffer (pH-6, 10 mL), sodium hypochlorite (0.24 mL, 4-4.99M solution) was added followed by TEMPO (8.55 mg, 0.055 mmol). The reaction mixture was stirred at ambient temperature for 2 h, then further sodium hypochlorite (0.24 mL, 4-4.99M solution) and TEMPO (8.55 mg, 0.055 mmol) were added. The addition of sodium hypochlorite and TEMPO was repeated three more times at 8 h intervals. The reaction mixture was cooled to 0° C. and 1N NaOH solution was added to adjust pH to ~13 and washed with DCM (40 mL). The aqueous layer was separated, cooled to 0° C., acidified with 1N HCl to pH ~1, extracted with EtOAc (2×30 mL), washed with water (2×10 mL), brine (10 mL), dried (Na₂SO₄) and evaporated in vacuo. The crude solid was recrystallised with acetonitrile to provide the title compound (171 mg, 69%).

¹H NMR (300 MHz, CD₃OD) δ 6.93 (s, 1H), 2.68-2.51 (m, 4H), 2.19 (s, 3H), 1.74-1.58 (m, 4H).

ES-MS: 223.3 [M−1].

HPLC: Retention time 7.460 min. and purity >98% at 280 nm.

### Example 26 5-Chloro-4-methoxytricyclo[6.2.2.0^{2,7}]dodeca-2,4,6-triene-3-carboxylic acid (A-29)

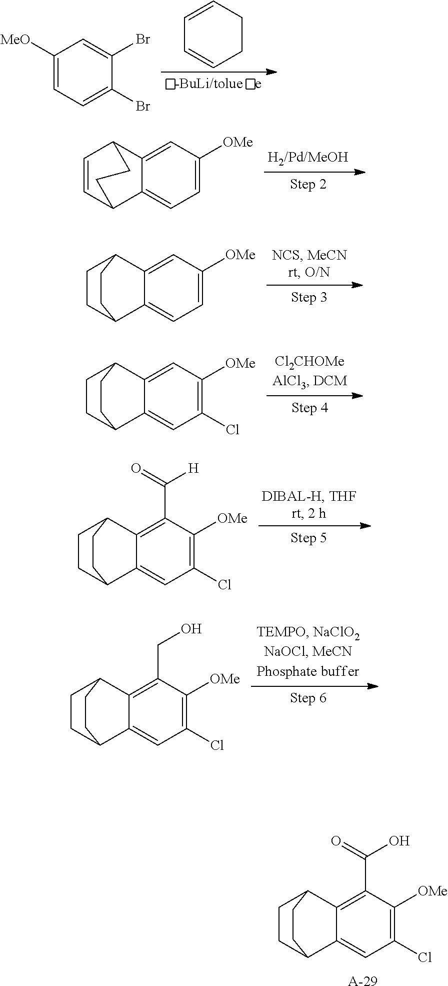

### Step 1: 4-Methoxytricyclo[6.2.2.0^{2, 7}]dodeca-2,4,6,9-tetraene

1,2-Dibromo-4-methoxybenzene (2.0 g, 7.52 mmol, 1.0 eq.) and 1,2-cyclohexadiene (freshly distilled, 2.86 mL, 30.10 mmol) was stirred in toluene (20 mL) at 0° C. under an argon atmosphere. n-BuLi (3.31 mL, 2.5 M in hexanes, 8.27 mmol, 1.1 eq.) was added to the solution dropwise over 20 min. After an additional 10 min. at 0° C. the mixture was allowed to warm to ambient temperature, stirred for 2 h, treated with water (30 mL) and extracted with EtOAc (3×50 mL). The organic layer was dried (Na₂SO₄) and concentrated. The resultant residue was purified by chromatography on silica gel eluting with hexane/EtOAc (0-3%) to provide the title compound (280 mg, 20%) as a colourless oil.

¹H NMR (300 MHz, CDCl₃) δ 7.09 (m, 1H), 7.04 (m, 1H), 6.71 (m, 1H), 6.02 (m, 2H), 4.01 (br, 2H), 3.99 (s, 3H), 1.74 (m, 2H), 1.67 (m, 2H) ppm.

### Step 2: 4-Methoxytricyclo[6.2.2.0^{2, 71}]dodeca-2,4,6-triene

4-Methoxytricyclo[6.2.2.0^{2,7}]dodeca-2,4,6,9-tetraene (280 mg, 1.50 mmol) was dissolved in methanol (20 mL), Pd-C (10%, 140 mg w/w, 0.75 mmol, 0.5 eq.) was added and the reaction mixture was stirred under hydrogen (1 atm. pressure) for 4 h.

The catalyst was filtered off and the solvent was removed under reduced pressure to give the 4-methoxytricyclo[6.2.2.0^{2,7}]dodeca-2,4,6- as a colourless oil, which was used in the next step without purification.

¹H NMR (300 MHz, CDCl₃) δ 7.17 (m, 1H); 6.99 (m, 1H), 6.76 (m, 1H), 4.02 (s, 3H), 3.16 (br, 2H), 1.97 (m, 4H), 1.61 (m, 4H) ppm.

### Step 3: 4-Chloro-5-methoxytricyclo[6.2.2.0^{2, 7}]dodeca-2,4,6-triene

A mixture of 4-methoxytricyclo[6.2.2.0^{2,7}]dodeca-2,4,6-triene (270 mg, 1.43 mmol, 1.0 eq.) and NCS (191 mg, 1.43 mmol) in acetonitrile (5 mL) was stirred at ambient temperature for 48 h. The reaction was quenched with water (20 mL) and extracted with EtOAc (2×30 mL). The combined extracts were washed with brine (30 mL), dried (Na₂SO₄) and concentrated. The resultant residue was purified by column chromatography on silica gel eluting with hexane/EtOAc (0-3%) to provide the title compound (260 mg, 81%).

¹H NMR (300 MHz, CDCl₃) δ 7.26 (s, 1H), 6.90 (s, 1H), 4.06 (s, 3H), 3.06 (m, 2H), 1.86 (m, 4H), 1.45 (m, 4H) ppm.

### Step 4: 5-Chloro-4-methoxytricyclo[6.2.2.0²⁷]dodeca-2,4,6-triene-3-carbaldehyde

To a suspension of 4-chloro-5-methoxytricyclo[6.2.2.0^{2,7}]dodeca-2,4,6-triene (260 mg, 1.167 mmol) and AgOTf (900 mg, 3.502 mmol) in dry DCM (5 mL), a solution of Cl₂CHOMe (0.33 mL, 3.502 mmol) in dry DCM (2 mL) was added at −78° C. under an argon atmosphere. The reaction mixture was stirred at ambient temperature for 16 h and quenched with saturated aqueous NaHCO₃ (20 mL). After stirring at ambient temperature for 30 min., the mixture was filtered through a pad of Celite®. The organic layer was separated, and the aqueous layer was extracted with DCM. (2×20 mL). The combined organic layers were washed with brine (10 mL), dried (Na₂SO₄) and concentrated. The resultant residue was purified by column chromatography on silica gel eluting with hexane/EtOAc (0-5%) to provide the title compound (250 mg, 86%).

¹H NMR (300 MHz, CDCl₃) δ 10.41 (s, 1H), 7.26 (s, 1H), 3.83 (s, 3H), 2.85 (m, 2H), 1.67 (m, 4H), 1.23 (m, 4H) ppm.

### Step 5: (5-Chloro-4-methoxytricyclo[6.2.2.0^{2, 71}]dodeca-2,4,6-trien-3-yl)methanol

To a solution of 3-chloro-2-ethoxy-5,6-xylenecarbaldehyde (250 mg, 0.997 mmol) in THF (5 mL) at 0° C., DIBAL-H (1N in toluene, 2 mL, 0.997 mmol) dropwise over 5 min.

The solution was stirred for a further 15 min. at 0° C. The resulting solution was allowed to warm to RT and stirred for 16 h. The reaction mixture was quenched with saturated aqueous sodium potassium tartrate (10 mL) and stirred for 20 min., then filtered through a pad of Celite® and extracted with EtOAc (2×20 mL). Organics were washed with brine (30 mL) dried over (Na₂SO₄), filtered, and evaporated to provide the title compound (230 mg, 91%).

¹H NMR (300 MHz, CDCl₃) δ 7.26 (s, 1H), 4.81 (m, 2H), 3.92 (s, 3H), 3.41 (br, 1H), 3.03 (br, 1H), 1.85 (m, 4H), 1.42 (m, 4H) ppm.

### Step 6: 5-Chloro-4-methoxytricyclo[6.2.2.0^{2,7}]dodeca-2,4,6-triene-3-carboxylic acid

To a stirred solution of (5-chloro-4-methoxytricyclo[6.2.2.0^{2,7}]dodeca-2,4,6-trien-3-yl)methanol (50 mg, 0.198 mmol), sodium chlorite (45 mg, 0.495 mmol) in acetonitrile (2 mL) and 1 N sodium phosphate buffer (pH ~6, 2 mL) was added sodium hypochlorite (0.2 mL, 4-4.99M solution) followed by TEMPO (6.2 mg, 0.0396 mmol). The reaction mixture was stirred at ambient temperature for 16 h, cooled to 0° C., N NaOH solution was added to adjust pH to ~13 and it was washed with DCM (100 mL). The aqueous layer was separated, cooled to 0° C., acidified with 1N HCl to pH ~1 and extracted with EtOAc (3×20 mL). The combined extracts were washed with water (2×10 mL), brine (10 mL) and dried (Na₂SO₄) and concentrated to provide a residue that was purified by HPLC using acetonitrile/0.1% formic acid solution 10-100% in 40 min. provided product as a colourless solid (4.5 mg, 8.6%).

¹H NMR (300 MHz, CD₃OD) δ 7.06 (s, 1H), 3.78 (s, 3H), 3.05 (br s, 1H), 2.85 (br s, 1H), 1.72 (m, 4H), 1.31 (m, 4H) ppm.

ES-MS: 265 [M−1].

HPLC Retention Time: 12.08 min., >98% purity @280 nm.

### Example 27: 2,3-Dichloro-5,6,7,8-tetrahydronaphthalene-1-carboxylic acid (A-30)

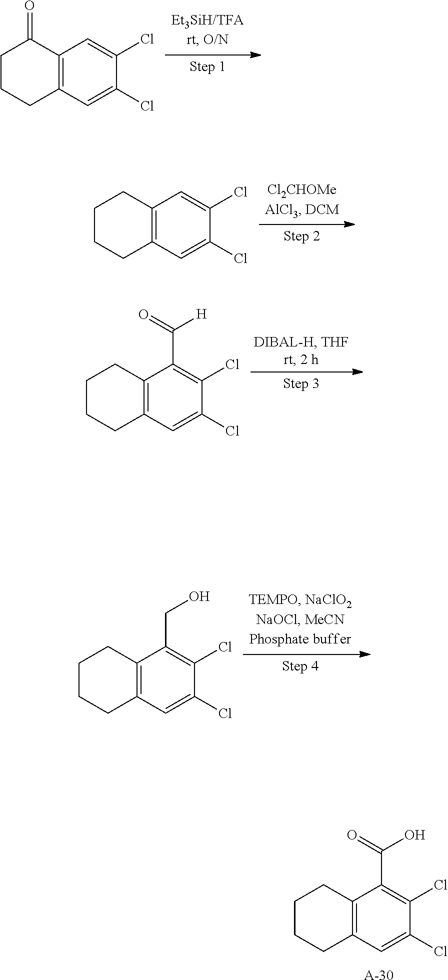

### Step 1: 6,7-Dichloro-1,2,3,4-tetrahydronaphthalene

Triethylsilane (1.48 mL, 9.30 mmol) was added dropwise over 10 min. to a solution of 6,7-dichloro-3,4-dihydronaphthalen-1(2H)-one (667 mg, 3.10 mmol) in TFA (3 mL) at 0° C. and the solution was stirred for 15 min. at 0° C. The resulting solution was allowed to warm to RT and stirred for 16 h. The reaction mixture was evaporated under reduced pressure and the residue was purified by chromatography on silica gel eluting with hexane/EtOAc (0-3%) to provide the title compound (583 mg, 93%) as a yellow solid.

¹H NMR (300 MHz, CDCl₃) δ 7.26 (s, 2H); 2.89-2.75 (m, 4H); 1.96-1.83 (m, 4H); ES-MS: 202 [M+1].

### Step 2: 2,3-Dichloro-5,6,7,8-tetrahydronaphthalene-1-carbaldehyde

To a suspension of 6,7-dichloro-1,2,3,4-tetrahydronaphthalene (359 mg, 1.78 mmol) and AlCl₃ (357 mg, 2.67 mmol) in dry DCM (8 mL) at −78° C., a solution of Cl₂CHOMe (1.24 mL, 2.67 mmol) in dry DCM (4 mL) was added under an argon atmosphere. After stirring at ambient temperature for 16 h, the reaction mixture was quenched with saturated aqueous NaHCO₃ (10 mL) and after a further 30 min., the reaction mixture was filtered through a pad of Celite®. The organic layer was separated and the aqueous layer was extracted twice with DCM (2×20 mL). The combined organic extracts were washed with brine (10 mL), dried (Na₂SO₄) and concentrated in vacuo.

The residue was purified by chromatography on silica gel eluting with hexane/EtOAc (0-5%) to provide the title compound (247 mg, 60%) as an off-white solid.

¹H NMR (300 MHz, CDCl₃) δ 10.46 (s, 1H); 7.27 (s, 1H); 2.97-2.84 (m, 2H); 2.73-2.61 (m, 2H); 1.74-1.58 (m, 4H); ES-MS: 230 [M+1].

### Step 3: (2,3-Dichloro-5,6,7,8-tetrahydronaphthalen-1-yl)methanol

DIBAL-H (1N in toluene, 1.62 mL, 1.62 mmol) was introduced dropwise over 20 min. to a solution of 2,3-dichloro-5,6,7,8-tetrahydro naphthalene-1-carbaldehyde (247 mg, 1.08 mmol) in THF (8 mL) at 0° C. and the solution was stirred for a further 15 min. at 0° C.

The reaction mixture was allowed to warm to RT, stirred for 2 h, quenched with saturated aqueous sodium potassium tartrate and extracted with EtOAc (2×50 mL).

The combined extracts were washed with brine (50 mL), dried (Na₂SO₄) and evaporated. The residue was purified by chromatography on silica gel eluting with hexane/EtOAc (0-10%) to provide the title compound (251 mg, quantitative) as a colourless oil.

¹H NMR (300 MHz, CDCl₃) δ 7.26 (s, 1H); 4.92 (s, 2H); 2.97 (t, 2H); 2.83 (t, 2H); 1.99-1.79 (m, 4H); ES-MS: 232 [M+1].

### Step 4: 2,3-Dichloro-5,6,7,8-tetrahydronaphthalene-1-carboxylic acid

To a stirred solution of (2,3-dichloro-5,6,7,8-tetrahydronaphthalen-1-yl)methanol (251 mg, 1.09 mmol), sodium chlorite (246 mg, 2.73 mmol) in acetonitrile (7 mL) and 1N sodium phosphate buffer (pH ~6, 7 mL), sodium hypochlorite (5 drops, 4-4.99M solution) was added followed by TEMPO (8.5 mg, 0.0545 mmol). The reaction mixture was stirred at ambient temperature for 2 h. and further sodium hypochlorite (4 drops, 4-4.99 M solution) and TEMPO (8.5 mg, 0.054 mmol) were added, and this addition of sodium hypochlorite and TEMPO was repeated three more times in 8 h intervals.

Following completion, the mixture was cooled to 0° C., 1 N NaOH solution was added to adjust the pH to ~13 followed by washing with DCM (40 mL). The water layer was separated, cooled to 0° C., acidified with 1N HCl to pH ~1 and extracted with EtOAc (2×50 mL). The combined extracts were washed with water (2×10 mL), brine (10 mL), dried (Na₂SO₄) and evaporated to provide 2,3-dichloro-5,6,7,8-tetrahydronaphthalene-1-carboxylic acid as a white solid (220 mg, 82%).

¹H NMR (300 MHz, MeOD) δ 7.33 (s, 1H); 2.86-2.68 (m, 4H); 1.91-1.74 (m, 4H); ES-MS: 243 [M−1].

HPLC Retention Time: 13.099 min., 98% purity @280 nm.

### Example 28: 6-chloro-7-methoxy-3,4-dihydro-2H-1-benzothiopyran-8-carboxylic acid (A-31)

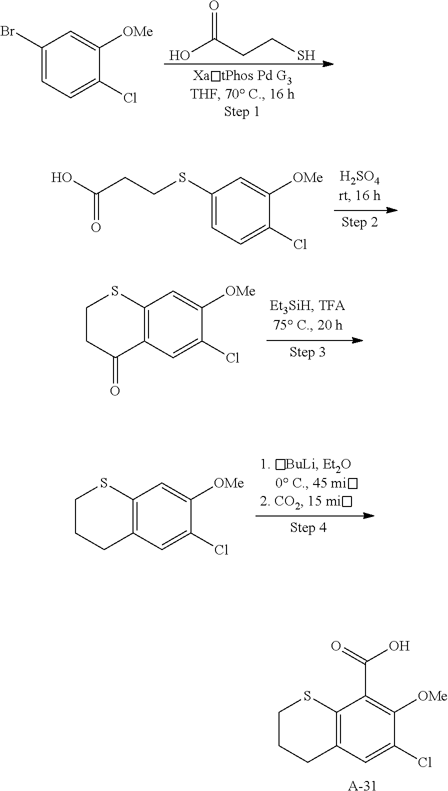

### Step 1: 3-(4-Chloro-3-methoxyphenylthio)propionic acid

To a mixture of 4-bromo-1-chloro-2-methoxybenzene (6.00 g, 27.0 mmol), 3-mercaptopropionic acid (2.6 mL, 29.7 mmol) and XantPhos Pd G3 (1.28 g, 1.35 mmol) in THF (75 mL), triethylamine (7.6 mL, 54.2 mmol) was added in one portion and the reaction mixture was heated at 70° C. for 16 h. The mixture was allowed to cool to ambient temperature then filtered through a pad of Celite® and the filter pad was washed with EtOAc (2×100 mL). The filtrate was evaporated under reduced pressure and the residue was dissolved in EtOAc (200 mL) before washing with 1.0 N HCl (2×150 mL). The organic layer was extracted with 1.0 N NaOH (2×150 mL) and the combined aqueous extracts were acidified to pH ~2 using 3.0 N HCl. Extraction into EtOAc (2×150 mL) and drying (Na₂SO₄) followed by concentration under reduced pressure provided a residue which was triturated with hexane (3×25 mL) to give the title acid (6.20 g, 93%).

¹H NMR (300 MHz, CDC₃) δ 7.29 (d, 1H), 6.97-6.94 (m, 1H), 6.94-6.89 (m, 1H), 3.90 (s, 3H), 3.16 (t, 2H), 2.68 (t, 2H).

ES-MS: 245 [M−H]

### Step 2: 6-chloro-7-methoxy-thiochroman-4-one

To a stirred solution of sulfuric acid (40 mL) at 0° C., 3-(4-chloro-3-methoxyphenylthio)propionic acid (2.70 g, 10.9 mmol) was added in several portions and the reaction mixture was stirred at ambient temperature for 16 h. The mixture was then poured into crushed ice, stirred for 30 min., extracted into EtOAc (2×100 mL) and the organic layer washed with 1.0 M NaOH (2×75 mL). Drying (Na₂SO₄) and concentration under reduced pressure provided the title compound (1.74 g, 69%).

¹H NMR (300 MHz, CDC₃) δ 8.13 (s, 1H), 6.75 (s, 1H), 3.93 (s, 3H), 3.28-3.21 (m, 2H), 2.96-2.89 (m, 2H).

ES-MS: 229 [M+H]

### Step 3: 6-chloro-7-methoxy-thiochromane

A mixture of 6-chloro-7-methoxy-thiochromane (1.55 g, 6.78 mmol) in triethylsilane (6.5 mL, 40.6 mmol) and TFA (3.1 mL, 40.6 mmol) was heated at 75° C. for 20 h. The solvent was removed under reduced pressure and the residue was purified by column chromatography on silica gel eluting with hexane/EtOAc (0-5%) to provide the title compound (1.12 g, 77%).

¹H NMR (300 MHz, CDCl₃) δ 7.02 (s, 1H), 6.64 (s, 1H), 3.84 (s, 3H), 3.04-2.97 (m, 2H), 2.72 (t, 2H), 2.09 (m, 2H).

ES-MS: 215 [M+H]

### Step 4: 6-chloro-7-methoxy-3,4-dihydro-2H-1-benzothiopyran-8-carboxylic acid

To a solution of 6-chloro-7-methoxy-thiochromane (200 mg, 0.93 mmol) in diethyl ether (5.0 mL), n-BuLi (0.7 mL, 1.12 mmol) was added at 0° C. and the mixture was stirred and allowed to reach ambient temperature over 45 min. The reaction mixture was cooled again to 0° C. and CO₂ was bubbled over a period of 15 min., followed by quenching water (25 mL) and washing with EtOAc (2×30 mL). The aqueous layer was acidified to pH ~2 utilising 1.0 N HCl and extracted into EtOAc (2×30 mL). The combined organic layers were dried (Na₂SO₄), filtered and concentrated under reduced pressure to afford the title compound (94 mg, 39%).

¹H NMR (300 MHz, CDCl₃) δ 7.16 (s, 1H), 3.93 (s, 3H), 3.02-2.94 (m, 2H), 2.79 (t, 2H), 2.09 (m, 2H).

ES-MS: 257 [M−H]

HPLC Retention Time: 10.7 min.; Purity: 95% @254 nm

### Example 29: 3-Chloro-2-(methylsulfanyl)-5,6,7,8-tetrahydronaphthalene-1-carboxylic acid (A-32)

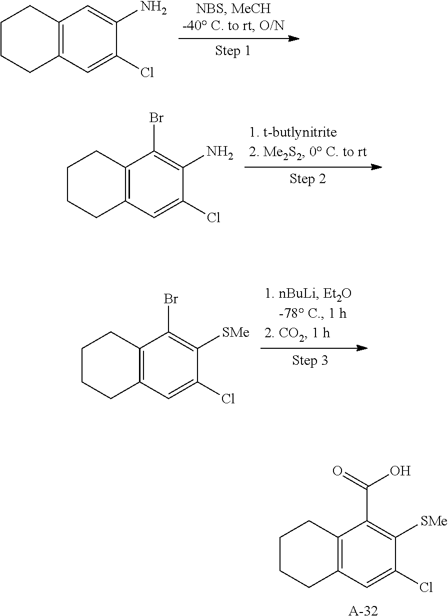

### Step 1: 1-Bromo-3-chloro-5,6,7,8-tetrahydronaphth-2-ylamine

NBS (4.40 g, 24.7 mmol) was added to a solution of 3-chloro-5,6,7,8-tetrahydronaphth-2-ylamine (4.28 g, 23.56 mmol) in acetonitrile (120 mL) at −40° C., and the mixture stirred at ambient temperature for 16 h. The reaction mixture was concentrated in vacuo, dissolved in EtOAc (200 mL), washed with water (2×50 mL) and brine (50 mL). The organic layer was dried (Na₂SO₄) and concentrated to a residue which was purified by chromatography on silica gel eluting with hexane/EtOAc (0-10%) to provide the title compound (5.25 g, 85%).

¹H NMR (300 MHz, CDC₃) δ 7.26 (s, 1H), 4.79-4.57 (br, 2H), 2.95 (q, 4H), 2.14-1.92 (m, 4H).

### Step 2: 5-Bromo-7-chloro-6-(methylthio)-1,2,3,4-tetrahydronaphthalene

A mixture of 1-bromo-3-chloro-5,6,7,8-tetrahydronaphth-2-ylamine (0.9 g, 3.47 mmol) in chloroform (8 mL) and tert-butyl nitrite (0.56 mL, 4.69 mmol) was added to a solution of dimethyl disulfide (0.68 mL, 7.65 mmol) in chloroform (2 mL). The reaction mixture was stirred at ambient temperature for 30 min. and extracted with chloroform (2×20 mL). The combined extracts were washed with water (10 mL), 1N HCl (10 mL) and brine (10 mL). The organic layer was dried (Na₂SO₄), concentrated to a residue which was purified by chromatography on silica gel eluting with hexane/EtOAc (0-10%) to provide the title compound (0.23 g, 22%).

¹H NMR (300 MHz, CDC₃) δ 7.26 (s, 1H), 2.82 (t, 4H), 2.48 (s, 3H), 1.95-1.74 (m, 4H).

### Step 3: 3-chloro-2-(methylsulfanyl)-5,6,7,8-tetrahydronaphthalene-1-carboxylic acid

n-BuLi (2.5 M in hexane) (0.197 mL, 0.49 mmol) was added to a solution of 5-bromo-7-chloro-6-(methylthio)-1,2,3,4-tetrahydronaphthalene (130 mg, 0.45 mmol) in diethyl ether (5 mL) at −78° C. The reaction mixture was stirred at the same temperature for 1 h, CO₂ was bubbled through the mixture for 10 min. and it was stirred for 1 h at −78° C.

The reaction temperature was adjusted to 0° C. and quenched with 1N NaOH to pH ~11.

The reaction mixture was washed with DCM (2×10 mL) and the water layer was acidified with 1N HCl to pH ~1. The solution was extracted with EtOAc (20 mL) then the organic layer dried (Na₂SO₄) and concentrated. The crude product was recrystallized from DCM to afford 3-chloro-2-(methylthio)-5,6,7,8-tetrahydronaphthalene-1-carboxylic acid (65.6 mg, 57%).

¹H NMR (300 MHz, CD₃OD) δ 7.30 (s, 1H), 2.80 (t, 2H), 2.71 (t, 2H), 2.36 (s, 3H), 1.88-1.74 (m, 4H).

ES-MS: m/z 255.26 (M-1).

HPLC: Retention time 8.617 min., purity >98% at 280 nm.

### Example 30: 8-chloro-7-fluoro-2,3,4,5-tetrahydro-1-benzoxepine-9-carboxylic acid (A-56) and 6-chloro-7-fluoro-2,3,4,5-tetrahydro-1-benzoxepine-9-carboxylic acid (A-57)

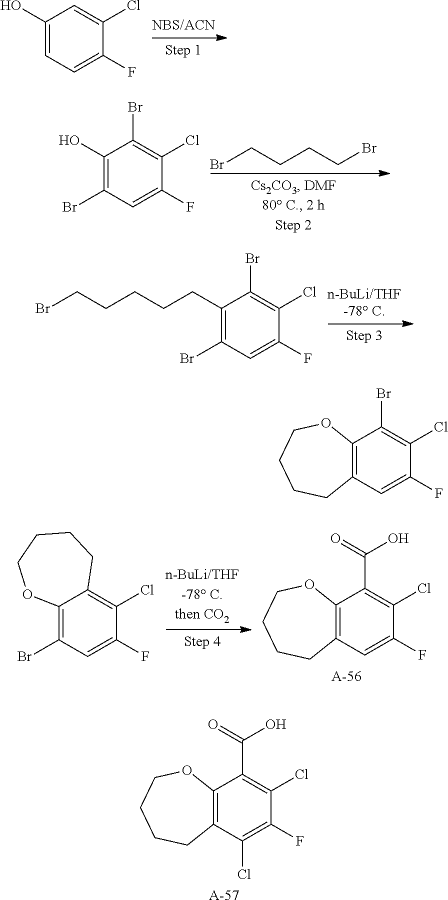

### Step 1: 2,6-Dibromo-3-chloro-4-fluorophenol

NBS (4.7 g, 24.0 mmol) was added to a solution of 3-chloro-4-fluorophenol (3.5 g, 24.0 mmol) in acetonitrile (24 mL) and stirred at ambient temperature for 16 h. The reaction mixture was concentrated in vacuo and the residue was purified by column chromatography on silica gel eluting with hexane/EtOAc (0-10%) to provide the title compound (3.1 g, 42%) as a white solid.

¹H NMR (300 MHz, CDCl₃) δ 7.27 (d, 1H), 5.74 (s, 1H). ¹⁹F NMR (300 MHz, CD₃OD) δ− 117.52 ppm.

ES-MS: m/z 303.5 (M−H).

### Step 2: 1,3-Dibromo-2-(4-bromobutoxy)-4-chloro-5-fluorobenzene

Caesium carbonate (9.8 g, 30.0 mmol) and 1,4-dibromobutane (6.5 g, 30.0 were added to a solution of 2,6-dibromo-3-chloro-4-fluorophenol (3.1 g, 10.0 mmol) in DMF (15 mL). The reaction mixture was heated at 80° C. for 2 h., cooled and evaporated. The resultant residue was purified by column chromatography on silica gel eluting with hexane/EtOAc (0-15%) to obtain the title compound (2.7 g, 61%) as a colourless oil.

¹H NMR (300 MHz, CDCl₃) δ 7.39 (d, 1H), 4.01 (t, 2H), 3.54 (t, 2H), 2.24-2.13 (m, 2H), 2.08-1.96 (m, 2H). ¹⁹F NMR (300 MHz, CD₃OD) δ− 112.07 ppm.

### Step 3: 9-Bromo-8-chloro-7-fluoro-2,3,4,5-tetrahvdrobenzo[b]oxepine

n-BuLi (2.5 M in hexane) (2.7 mL, 6.76 mmol) was added to a solution of 1,3-dibromo-2-(4-bromobutoxy)-4-chloro-5-fluorobenzene (2.7 g, 6.14 mmol) in THF/hexane (20/6 mL) at −78° C. The reaction mixture was stirred at that temperature for 1 h., then at ambient temperature for 16 h. and quenched with saturated NH₄Cl (20 mL). The reaction mixture was extracted with EtOAc (2×50 mL), the combined extracts were evaporated and the residue purified by column chromatography on silica gel eluting with hexane/EtOAc (0-20%) to obtain the regioisomer mixture bromo-8-chloro-7-fluoro-2,3,4,5-tetrahydrobenzo[b]oxepine and 9-bromo-6-chloro-7-fluoro-2,3,4,5-tetrahydrobenzo[b]oxepine (0.73 g, 43%) as a colourless oil.

¹H NMR (300 MHz, CDC₃) δ 7.21 (d, 1H), 4.00 (t, 2H), 3.06 (t, 2H), 2.08-1.89 (m, 2H), 1.77-1.64 (m, 2H). ¹⁹F NMR (300 MHz, CD₃OD) δ− 116.27 and -123.72 ppm.

### Step 4: 8-Chloro-7-fluoro-2,3,4,5-tetrahydro-1-benzoxepine-9-carboxylic acid and 6-chloro-7-fluoro-2,3,4,5-tetrahydro-1-benzoxepine-9-carboxylic acid

n-BuLi (2.5 M in hexane) (0.60 mL, 1.5 mmol) was added to a solution of 9-bromo-8-chloro-7-fluoro-2,3,4,5-tetrahydrobenzo[b]oxepine and 9-bromo-6-chloro-7-fluoro-2,3,4,5-tetrahydro-benzo[b]oxepine (0.38 g, 0.342 mmol) in THF (20 mL) at −78° C. The mixture was stirred at that temperature for 0.5 h., CO₂ gas was bubbled through the solution for 10 min., the reaction stirred at −78° C. for a further 1 h. and slowly brought to ambient temperature before being quenched with 1M NaOH (5 mL). The mixture was washed with EtOAc (2×10 mL), the aqueous layer was acidified to pH ~2.0 using 1.0 N HCl and extracted with EtOAc (2×20 mL). The combined organic layers were dried (Na₂SO₄) and concentrated. The residue was purified by semi-prep HPLC eluting with 30-50% acetonitrile/0.1% formic acid in water to provide 8-chloro-7-fluoro-2,3,4,5-tetrahydro-1-benzoxepine-9-carboxylic acid (5 mg) and 6-chloro-7-fluoro-2,3,4,5-tetrahydro-1-benzoxepine-9-carboxylic acid (7 mg) as white solids with a combined yield of 3.3%.

8-Chloro-7-fluoro-2,3,4,5-tetrahydro-1-benzoxepine-9-carboxylic acid (A-56)

¹H NMR (300 MHz, CD₃OD) δ 7.13 (d, J_{H-F}=9.8 Hz, 1H), 4.00 (t, 2H), 2.82 (t, 2H), 2.04-1.94 (m, 2H), 1.80-1.70 (m, 2H).

¹⁹F NMR (300 MHz, CD₃OD) δ− 123.94 ppm.

ES-MS: 243.60 [M−1].

6-Chloro-7-fluoro-2,3,4,5-tetrahydro-1-benzoxepine-9-carboxylic acid (A-57)

¹H NMR (300 MHz, CD₃OD) δ 7.39 (d, J_{H-F}=9.1 Hz, 1H), 4.08 (t, 2H), 3.10 (t, 2H), 2.08-1.95 (m, 2H), 1.82-1.70 (m, 2H).

¹⁹F NMR (300 MHz, CD₃OD) δ− 119.62 ppm.

ES-MS: 243.60 [M−1].

### Example 31: 3-chloro-8,8-difluoro-2-methoxy-5,6,7,8-tetrahydronaphthalene-1-carboxylic acid (A-59)

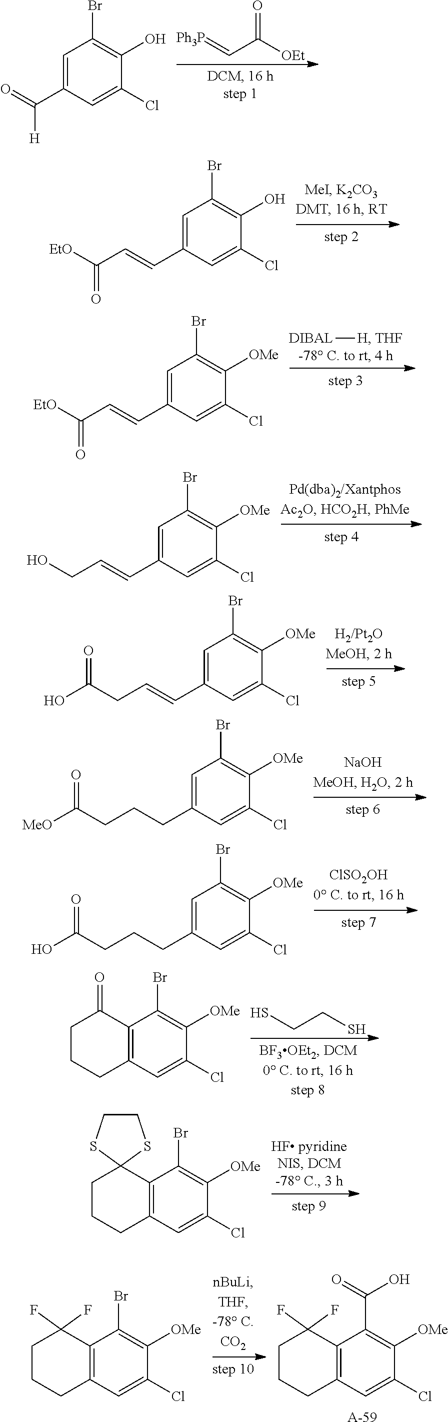

### Step 1: Ethyl (E)-3-(3-bromo-5-chloro-4-hydroxyphenyl)acrylate

Ethyl (triphenylphosphoranylidene)acetate (15.54 g, 44.6 mmol) was added to a solution of 3-bromo-5-chloro-4-hydroxybenzaldehyde (10 g, 42.47 mmol) in CH₂Cl₂ (50 mL) at room temperature and the reaction mixture stirred at ambient temperature for 16 h. The solvent was removed under reduced pressure and the residue purified by column chromatography on silica gel eluting with hexane/EtOAc (0-10%) hexane to provide the [symbol] as a white solid (8.1 g, 62%).

¹H NMR (300 MHz, CDC₃); 6 7.64 (d, 1H), 6 7.56 (d, 1H), 7.52 (d, 1H), 6.39 (d, 1H), 6.30 (s, H), 4.32 (q, 2H), 1.38 (t, 3H) ppm.

### Step 2: Ethyl (E)-3-(3-bromo-5-chloro-4-methoxyphenyl)acrylate

K₂CO₃ (10.5 g, 75.9 mmol) and CH₃I (5.84 mL, 113.9 mmol), was added to a solution of ethyl (E)-3-(3-bromo-5-chloro-4-hydroxyphenyl)acrylate (11.6 g, 38.0 mmol) in DMF (30 mL). The reaction mixture was stirred at ambient temperature for 16 h, diluted with EtOAc (110 mL), washed with 1N HCl solution (3×50 mL), dried (Na₂SO₄) and the solution concentrated under reduced pressure to afford the crude product which was used in the next step without further purification.

¹H NMR (300 MHz, CDC₃); 6 7.62 (d, 1H), 7.52 (d, 1H), 7.48 (d, 1H), 6.35 (d, 1H), 4.24 (q, 2H), 3.90 (s, 3H), 1.35 (t, 3H) ppm.

### Step 3: (E)-3-(3-Bromo-5-chloro-4-methoxyphenyl)-2-propen-1-ol

DIBAL (1M in toluene) (69 mL, 68.8 mmol) was added to a solution of ethyl (E)-3-(3-bromo-5-chloro-4-methoxyphenyl)acrylate (11.0 g, 34.42 mmol) in CH₂Cl₂ (50 mL) at −78° C. The reaction mixture was stirred at the same temperature for 30 min, then at ambient temperature for 2 h. The mixture was cooled to 0° C. and 1N HCl solution was added slowly. The mixture was stirred for 30 min, diluted with CH₂Cl₂ (50 mL) and the organic layer was separated dried (Na₂SO₄) and concentrated under reduced pressure.

The residue was purified by column chromatography on silica gel eluting with (5-10%) hexane/EtOAc (5-10%) to obtain title compound as a yellow oil (8.7 g, 91%).

¹H NMR (300 MHz, CDC₃); 6 7.38 (d, 1H), 7.25 (d, 1H), 6.35 (d, 1H), 6.24 (m, 1H), 4.29 (d, 2H), 3.82 (s, 3H) ppm.

### Step 4: (E)-4-(3-Bromo-5-chloro-4-methoxyphenyl)-3-butenoic acid

Pd(dba)₃ (135 mg, 0.15 mmol), Xantphos (342 mg, 0.59 mmol), acetic anhydride (4.19 mL, 44.32 mmol) and formic acid (1.67 mL, 44.32 mmol) was added to a solution of (E)-3-(3-Bromo-5-chloro-4-methoxyphenyl)-2-propen-1-ol (4.10 g, 14.77 mmol) in toluene (20 mL) at ambient temperature. The mixture was flushed with nitrogen gas and heated at 80° C. for 16 h. Volatiles were removed under vacuum and 1N HCl (5 mL) was added, product was extracted with ethyl acetate (3×50 mL), dried (Na₂SO₄), concentrated, and purified by column chromatography on silica gel eluting with hexane/EtOAc (20-50%) to obtain the title compound as a white solid (3.2 g, 71%).

¹H NMR (300 MHz, CDC₃); 6 7.39 (d, 1H), 6 7.26 (d, 1H), 6 6.25 (d, 1H), 6 6.18 (m, 1H), 3.81 (s, 3H), 3.22 (d, 2H) ppm.

### Step 5: Methyl 4-(3-bromo-5-chloro-4-methoxyphenyl)butyrate

The catalyst PtO₂ (10% w/w), 370 mg) was added to a solution of (E)-4-(3-bromo-5-chloro-4-methoxyphenyl)-3-butenoic acid (3.37 g, 11.03 mmol) in CH₃OH (50 mL). The mixture was stirred for 2 h under a hydrogen atmosphere with balloon pressure, filtered through a pad of celite and concentrated to provide the title compound as a brown solid which was used in the next step without purification.

¹H NMR (300 MHz, CDC₃); 6 7.26 (d, 1H), 7.14 (d, 1H), 3.85 (s, 3H), 3.65 (s 3H), 2.55 (t, 2H), 2.31 (t, 2H), 1.90 (q, 2H) ppm.

### Step 6: 4-(3-Bromo-5-chloro-4-methoxyphenyl)butyric acid

NaOH (0.821 g, 20.52 mmol) was added to a solution of methyl 4-(3-bromo-5-chloro-4-methoxyphenyl)butyrate (3.30 g, 10.26 mmol) in CH₃OH/H₂O (10/5 mL). The reaction mixture was stirred at ambient temperature for 16 h, slowly quenched with 1 N HCl to pH ~2, extracted with ethyl acetate (3×50 mL). The combined extracts were dried (Na₂SO₄) and concentrated under reduced pressure to obtain the title compound as a white solid (3.1 g, 98% yield).

¹H NMR (300 MHz, CDC₃); 6 7.25 (d, 1H), 7.16 (d, 1H), 3.85 (s, 3H), 2.56 (t, 2H), 2.35 (t, 2H), 1.92 (q, 2H) ppm.

### Step 7: 8-Bromo-6-chloro-7-methoxy-3,4-dihydro-2H-naphthalen-1-one

A mixture of 4-(3-bromo-5-chloro-4-methoxyphenyl)butyric acid (3.30 g, 10.7 mmol) and CISO₃H (8 mL) was stirred at 0° C. for 2 h then at ambient temperature for 16 h. The mixture was carefully poured into ice cold water (50 mL) and treated with EtOAc (30 mL). The organic layer was separated and washed with 1 N HCl solution (2×50 mL), dried (Na₂SO₄), and concentrated under reduced pressure. The residue was purified by column chromatography on silica gel eluting with hexane/EtOAc (5-10%) to obtain the title compound as a brown solid (683 mg, 22%).

¹H NMR (300 MHz, CDC₃); 6 7.2 5(s, 1H), 3.88 (s, 3H), 2.90 (m, 2H), 2.66 (m, 2H), 2.08 (m, 2H) ppm.

### Step 8: 8′-Bromo-6′-chloro-7′-methoxy-3′,4′-dihydro-2′H-spiro[1,3-dithiolane-2,1′-naphthalene]

Boron trifluoride diethyl etherate (0.14 mL 1.14 mmol) and 1,2-ethanedithiol (0.39 mL, g, 4.58 mmol) was added slowly to a solution of 8-bromo-6-chloro-7-methoxy-3,4-dihydro-2H-naphthalen-1-one (636 mg, 2.29 mmol) in CH₂Cl₂ (10 mL) at 0° C. The mixture was stirred at ambient temperature for 16 h., quenched with saturated NaHCO₃ solution (15 mL) and stirred for 10 min before being extracted with CH₂Cl₂ (3×30 mL). The combined extracts were dried (Na₂SO₄), concentrated under reduced pressure and the residue purified by column chromatography on silica gel eluting with hexane/EtOAc (0-5%) to obtain the title compound as a white solid (420 mg, 50%).

¹H NMR (300 MHz, CDC₃) δ 7.25 (s, 1H), 3.90 (s, 3H), 3.69 (m, 2H), 3.50 (m, 2H), 2.64 (m, 2H), 2.45 (m, 2H), 1.95 (m, 2H) ppm.

### Step 9: 8-Bromo-6-chloro-1,1-difluoro-7-methoxy-1,2,3,4-tetrahydronaphthalene

N-lodiosuccinimide (583 mg, 2.59 mmol) in DCM (4 mL) and HF-pyridine solution (0.47 mL, 5.17 mmol) were added slowly to a solution of 8′-bromo-6′-chloro-7′-methoxy-3′,4′-dihydro-2′H-spiro[1,3-dithiolane-2,1′-naphthalene](172 mg, 0.65 mmol) in CH₂Cl₂ (5 mL) at −78° C. The mixture was stirred at −78° C. for 3 h., quenched with saturated NaHCO₃ solution (15 mL) and extracted with CH₂Cl₂ (3×20 mL). The combined extracts were dried (Na₂SO₄), concentrated under reduced pressure and purified by column chromatography on silica gel eluting with hexane/EtOAc 0-10% to afford the title compound as a light-yellow solid (132 mg, 65%).

¹H NMR (300 MHz, CDC₃); 6 7.24 (s, 1H), 3.83 (s, 3H), 2.77 (m, 2H), 2.24 (m, 2H), 1.85 (m, 2H) ppm. ¹⁹F NMR (300 MHz, CDC₃); δ −87.23 ppm.

### Step 10: 3-Chloro-8,8-difluoro-2-methoxy-5,6,7,8-tetrahydronaphthalene-1-carboxylic acid

n-BuLi (2.5 M in hexane) (0.197 mL, 0.49 mmol) was added to a solution of 8-bromo-6-chloro-1,1-difluoro-7-methoxy-1,2,3,4-tetrahydronaphthalene (128 mg, 0.41 mmol) in THF (6 mL) at −78° C. The reaction mixture was stirred at that temperature for 1 h and CO₂ (g) was bubbled through for 10 min and slowly brought to 0° C. Quenching with water (20 mL) and washing with CH₂Cl₂ (2×20 mL) provided an aqueous layer which was acidified with 1N HCl to pH 4-5. The mixture was extracted with CH₂Cl₂ (3×50 mL) then the combined extracts were dried (Na₂SO₄) and concentrated under reduced pressure. The residue product was purified by semi preparative HPLC using 10-100% CH₃CN/0.1% formic acid in water to obtain the title compound as a white solid (32 mg

28% yield).

¹H NMR (300 MHz, CD₃OD); δ 7.40 (s, 1H), 3.90 (s, 3H), 2.87 (m, 2H), 2.26 (m, 2H), 2.01 (m, 2H) ppm. ¹⁹F NMR (300 MHz, CD₃OD); δ −84.46 ppm.

ES-MS: 275.4 [M−1].

HPLC: Retention Time: 9.84 min., purity: >99% @280 nm.

### Example 32: 5-Methoxy-6-methyl-2,3-dihydro-1H-indene-4-carboxylic acid (A-47)

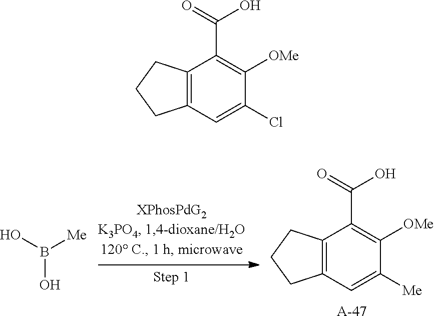

### Step 1: 5-Methoxy-6-methyl-2,3-dihydro-1H-indene-4-carboxylic acid

To a mixture of 6-chloro-5-methoxy-2,3-dihydro-1H-indene-4-carboxylic acid (60 mg, 0.265 mmol), methylboronic acid (31.7 mg, 0.53 mmol), XPhosPdG₂ (10.4 mg, 0.0133 mmol) and potassium phosphate (169.8 mg, 0.8 mmol), 1,4-dioxane/water (3/1 mL) was added. The reaction mixture was subjected to microwave conditions at 120° C. for 1 h. The reaction ixture was cooled and washed with EtOAc (2×5 mL). The aqueous layer was acidified to pH ~2.0 with 1.0 N HCl and extracted into EtOAc (2×15 mL).

The combined extracts were dried (Na₂SO₄) and evaporated to a residue was purified by semi-prep HPLC using 30-50% acetonitrile/0.1% formic acid in water to provide the title compound (32 mg, 59%) as a white solid.

¹H NMR (300 MHz, CD₃OD) δ 7.15 (s, 1H), 3.78 (s, 3H), 2.97 (t, 2H), 2.85 (t, 2H), 2.61 (s, 3H), 2.12-2.01 (m, 2H).

ES-MS: m/z 205.3 (M−H).

HPLC: Retention time 10.12 min., purity >99% at 280 nm.

### Example 33: 6-Chloro-7-fluoro-3,4-dihydro-2H-1-benzopyran-8-carboxylic acid (A-36)

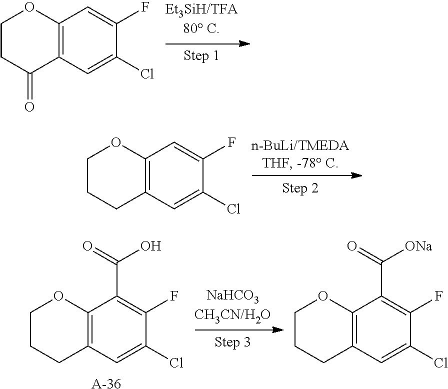

### Step 1: 6-Chloro-7-fluorochroman

A mixture of 6-chloro-7-fluorochroman-4-one (0.57 g, 2.84 mmol), triethylsilane (1.98 g, 17.05 mmol) and TFA (1.94 g, 17.05 mmol) was heated in a sealed tube at 80° C. for 16 h. The reaction mixture was cooled, evaporated and purified by column chromatography on silica gel eluting with EtOAc/hexane (0-10%) to obtain title compound (0.35 g, 66%) as an off-white solid.

¹H NMR (300 MHz, CDCl₃) δ 7.27 (d, 1H), 6.83 (d, 1H), 4.40 (t, 2H), 2.96 (t, 2H), 2.28-2.17 (m, 2H). ¹⁹F NMR (300 MHz, CDCl₃) δ− 117.56 ppm.

### Step 2: 6-Chloro-7-fluoro-3,4-dihydro-2H-1-benzopyran-8-carboxylic acid

To a solution of 6-chloro-7-fluorochroman (80 mg, 0.43 mmol) in THF (10 mL) at −78° C., n-BuLi (2.5 M in hexane) (0.2 mL, 0.47 mmol) was added. The reaction mixture was stirred at that temperature for 1.5 h. and CO₂ gas was bubbled for 10 min. The reaction mixture was stirred at −78° C. for 1 h., slowly brought to ambient temperature and quenched with 1 M NaOH (5 mL). The reaction mixture was washed with EtOAc (2×10 mL), the aqueous layer was acidified to pH ~2.0 using 1.0 N HCl and extracted with EtOAc (2×20 mL). The combined extracts were dried (Na₂SO₄) and concentrated to obtain the title compound (45 mg, 45%) as a white solid.

¹H NMR (300 MHz, CD₃OD) δ 7.25 (d, J_{H-F}=8.4 Hz, 1H), 4.25 (t, 2H), 2.80 (t, 2H), 2.08-1.96 (m, 2H). ¹⁹F NMR (300 MHz, CD₃OD) δ− 122.00 ppm.

ES-MS: m/z 229.5 (M−H).

HPLC Retention Time: 9.53 min.; Purity: >99% @280 nm Step 3: Sodium 6-chloro-7-fluoro-3,4-dihydro-2H-1-benzopyran-8-carboxylate

To a solution of 6-chloro-7-fluorochroman-8-carboxylic acid (44 mg, 0.191 mmol) in acetonitrile (5.0 mL), and water (2.0 mL), NaHCO₃ (15.2 mg, 0.181 mmol) was added and the reaction mixture was stirred at ambient temperature for 1 h. The solvent was removed under reduced pressure and triturated with CH₂Cl₂ (2×5 mL) to provide the title compound (37 mg, 77%).

¹H NMR (300 MHz, CD₃OD) δ 7.01 (d, J_{H-F}=8.2 Hz, 1H), 4.19 (t, 2H), 2.76 (t, 2H), 2.06-1.90 (m, 2H). ¹⁹F NMR (300 MHz, CDCl₃) δ− 124.14 ppm.

ES-MS: m/z 231.5 (M+H).

HPLC: Retention time 9.59 min., Purity: >99% @280 nm

### Example 34: 3-Chloro-2-ethenyl-5,6,7,8-tetrahydronaphthalene-1-carboxylic acid (A-35)

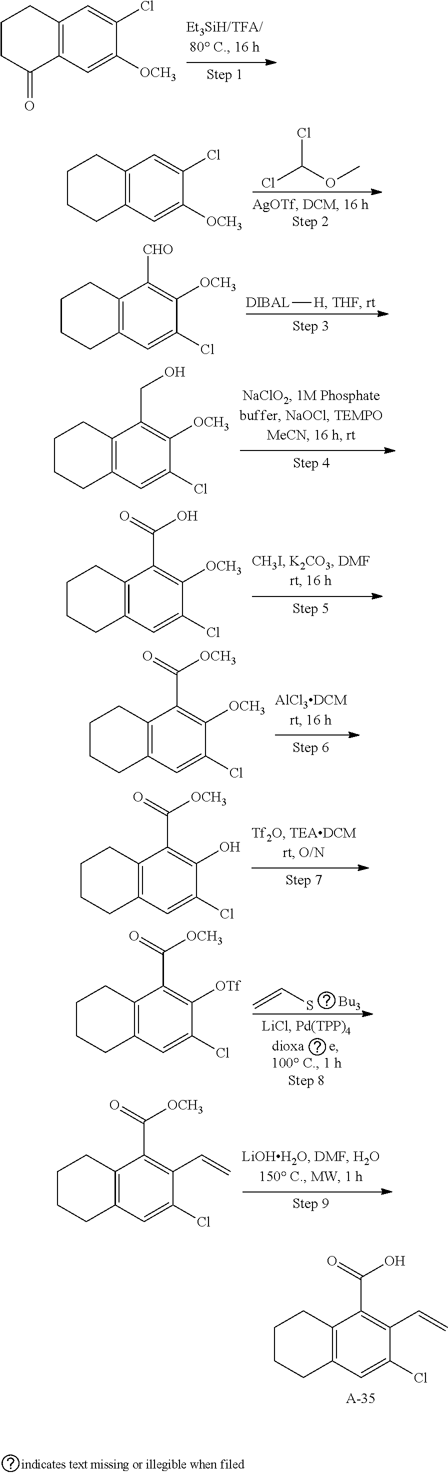

### Step 1: 6-Chloro-7-methoxy-1,2,3,4-tetrahydronaphthalene

A mixture of 6-chloro-7-methoxy-3,4-dihydronaphthalen-1 (2H)-one (4.04 g, 19.17 mmol), triethylsilane (18.37 ml, 115.06 mmol) and trifluoroacetic acid (8.08 ml, 115.06 mmol) was heated at 80° C. and stirred for 16 h. The reaction mixture was concentrated in vacuo and the crude product was purified by chromatography on silica gel (0-5% EtOAc/hexane) to afford the title compound (3.4 g, 90%) as a light yellow liquid.

¹H NMR (300 MHz, CDCl₃) δ 7.26 (s, 1H), 6.82 (s, 1H), 4.06 (s, 3H); 2.99-2.80 (m, 4H);

2.03-1.89 (m, 4H).

### Step 2: 3-Chloro-2-methoxy-5,6,7,8-tetrahydronaphthalene-1-carbaldehyde

AgOTf (2.0 g, 7.77 mmol) and dichloromethyl methyl ether (0.71 mL, 7.77 mmol) in CH₂Cl₂ (3.0 mL) was added at −78° C. to a solution of 6-chloro-7-methoxy-1,2,3,4-tetrahydronaphthalene (511 mg, 2.59 mmol) in CH₂Cl₂ (5 mL). The reaction mixture was stirred at ambient temperature for 16 h, cooled to 0° C. and quenched with water (15 mL). The mixture was filtered through a pad of Celite and the layers were separated. The organic layer was dried (Na₂SO₄) and concentrated in vacuo. The resultant residue was purified by column chromatography silica gel eluting with hexane/EtOAc (0-4%) to provide the title compound (377 mg, 64%) as a colourless liquid.

¹H NMR (300 MHz, CDCl₃) δ 10.44 (s, 1H), 7.27 (s, 1H), 3.86 (s, 3H), 3.03-2.92 (m, 2H), 2.71-2.60 (m, 2H); 1.76-1.60 (m, 4H).

### Step 3: 3-Chloro-2-methoxy-5,6,7,8-tetrahydronaphthalen-1-yl)methanol

DIBAL-H (2.49 mL, 2.49 mmol) was added at 0° C. to a solution of 3-chloro-2-methoxy-5,6,7,8-tetrahydronaphthalene-1-carbaldehyde (0.377 mg, 1.66 mmol) in THF (12.0 mL). The reaction mixture was stirred at ambient temperature for 2 h, cooled to 0° C., quenched with 1 N HCl (50 mL) and extracted with EtOAc (2×50 mL). The organic layer was dried (Na₂SO₄) and concentrated under reduced pressure. The crude product (450 mg, quantitative yield) was utilised in the next step without further purification.

¹H NMR (300 MHz, CDCl₃) δ 7.25 (s, 1H), 4.92 (s, 2H), 4.07 (s, 3H), 3.00 (t, 2H), 2.89 (t, 2H), 2.05-1.83 (m, 4H).

### Step 4: 3-Chloro-2-methoxy-5,6,7,8-tetrahydronaphthalene-1-carboxylic acid

NaClO2 (0.448 g, 4.96 mmol), NaOCl (0.2 mL, 4-4.99M solution), TEMPO (15.5 mg, 0.099 mmol) and 1.0 M sodium phosphate buffer (pH 6.0, 21.0 mL) were introduced to a solution of (3-chloro-2-methoxy-5,6,7,8-tetrahydronaphthalen-1-yl)methanol (0.45 g, 1.99 mmol) in acetonitrile (21 mL). The reaction mixture was stirred at ambient temperature for 16 h and additional NaOCl (0.2 mL, 4-4.99M solution) and TEMPO (15.5 mg, 0.099 mmol) were added. The reaction mixture was stirred for a further 2 h before being quenched with 1N NaOH (30 mL) and washed with EtOAc (2×30 mL).

The aqueous layer was acidified to pH ~2.0 using 1.0 N HCl and extracted with EtOAc (2×50 mL). The combined extracts were dried (Na₂SO₄) and concentrated under reduced pressure to afford the title compound (450 mg, 94%).

¹H NMR ¹H NMR (300 MHz, CDC₃) δ 11.39-10.94 (br, 1H), 7.25 (s, 1H), 4.02 (s, 3H), 2.95-2.73 (m, 4H), 1.94-1.77 (m, 4H).

ES-MS: 239 [M−1].

### Step 5: Methyl 3-chloro-2-methoxy-5,6,7,8-tetrahydronaphthalene-1-carboxylate

K₂CO₃ (517.9 mg, 3.74 mmol) and iodomethane (0.23 ml, 3.74 mmol) were added to a solution of 6-chloro-5-methoxy-2,3-dihydro-1H-indene-4-carboxylic acid (0.45 g, 1.87 mmol) in DMF (15 mL) at ambient temperature. The reaction mixture was stirred at the same temperature for 16 h. The reaction mixture was concentrated and extracted with EtOAc (2×30 mL) and the combined extracts washed with water (4×10 mL) and brine (2×10 mL). The organic layer was dried (Na₂SO₄) and concentrated in vacuo to afford the title compound (351 mg, 73%), which was used in the next step without purification.

¹H NMR ¹H NMR (300 MHz, CDC₃) δ 7.27 (s, 1H), 4.05 (s, 3H), 3.99 (s, 3H), 2.89-2.72 (m, 4H), 1.96-1.81 (m, 4H).

### Step 6: Methyl 3-chloro-2-hydroxy-5,6,7,8-tetrahydronaphthalene-1-carboxylate

AlCl₃ (734.9 mg, 5.52 mmol) was added to a solution of methyl 3-chloro-2-methoxy-5,6,7,8-tetrahydronaphthalene-1-carboxylate (351 mg, 1.38 mmol) in CH₂Cl₂ (6 mL) at 0° C. The reaction mixture was stirred at ambient temperature for 16h, quenched with 2N HCl, extracted with EtOAc (3×50 mL) then washed with water (2×20 mL) and brine (15 mL). The organic layer was dried over Na₂SO₄, concentrated and purified by column chromatography on silica gel eluting (0-30% EtOAc/hexane) to provide the title compound (297 mg, 90%).

¹H NMR ¹H NMR (300 MHz, CDC₃) δ 11.24 (s, 1H), 7.26 (s, 1H), 3.98 (s, 3H), 3.03-2.88 (m, 2H), 2.79-2.64 (m, 2H), 1.83-1.67 (m, 4H).

### Step 7: Methyl 3-chloro-2-(trifluoromethylsulfonyloxy)-5,6,7,8-tetrahydronaphthalene-1-carboxylate

Triethylamine (0.38 ml, 2.71 mmol) was added at 0° C. to a solution of methyl 3-chloro-2-hydroxy-5,6,7,8-tetrahydronaphthalene-1-carboxylate (297 mg, 1.23 mmol) in CH₂Cl₂ (4 mL), followed by dropwise addition of trifluoromethanesulphonic anhydride (0.41 ml, 2.46 mmol). The reaction mixture was stirred at ambient temperature for 16 h. The reaction mixture was quenched with water (20 mL), extracted with CH₂Cl₂ (2×25 mL) then washed with water (3×10 mL) and brine (15 mL). The organic layer was dried (Na₂SO₄) concentrated in vacuo and purified by column chromatography on silica gel using EtOAc/hexane (0-10%) to afford the title compound (340 mg, 76%).

¹H NMR ¹H NMR (300 MHz, CDC₃) δ 7.28 (s, 1H), 3.85 (s, 3H), 2.83-2.66 (m, 4H), 1.83-1.66 (m, 4H). ¹⁹F NMR (300 MHz, CDC₃) δ− 74.49 ppm.

### Step 8: Methyl 3-chloro-2-vinyl-5,6,7,8-tetrahydronaphthalene-1-carboxylate

A solution of methyl 3-chloro-2-(trifluoromethylsulfonyloxy)-5,6,7,8-tetrahydronaphthalene-1-carboxylate (161 mg, 0.43 mmol) in 1,4-dioxane (2 mL) was degassed with argon gas for 20 min. Vinyl tributyltin (0.19 ml, 0.65 mmol), Pd(PPh₃)₄ (49.9 mg, 0.043 mmol) and LiCl (54.9 mg, 1.29 mmol) were added. The reaction mixture was subjected to microwave conditions at 100° C. for 1 h and treated with EtOAc (30 mL). The mixture was washed with water (2×10 mL) and brine (2×15 mL) then the organic layer was dried (Na₂SO₄) concentrated in vacuo and the residue purified by column chromatography on silica gel eluting with EtOAc/hexane (0-5%) to afford the title compound (33 mg, 30%).

¹H NMR ¹H NMR (300 MHz, CDC₃) δ 7.27 (s, 1H), 6.92 (d, 1H), 5.57 (d, 1H), 5.52 (s, 1H), 3.95 (s, 3H), 2.93-2.81 (m, 2H), 2.81-2.72 (m, 2H), 1.97-1.81 (m, 4H).

### Step 9: 3-Chloro-2-ethenyl-5,6,7,8-tetrahydronaphthalene-1-carboxylic acid

LiOH·H₂O (55.2 mg, 1.3 mmol) was added to a solution of methyl 3-chloro-2-vinyl-5,6,7,8-tetrahydronaphthalene-1-carboxylate (33 mg, 0.13 mmol) in DMF (0.5 mL) and water (0.1 mL) and the reaction mixture was subjected to microwave conditions at 150° C. for 1 h. It was concentrated, dissolved in water (4 mL) and washed with CH₂Cl₂ (2×10 mL). The aqueous layer was acidified with 1N HCl to pH ~1 and extracted with EtOAc (20 mL), dried (Na₂SO₄) concentrated in vacuo and purified by semi-prep HPLC utilising 0-100% acetonitrile/0.1% formic acid in water to afford the title compound (22 mg, 71%) as an off-white solid.

¹H NMR ¹H NMR (300 MHz, CDCl₃) δ 7.26 (s, 1H), 6.94 (dd, 1H), 5.67 (d, 1H), 5.57 (d, 1H), 2.95-2.74 (m, 4H), 1.98-1.78 (m, 4H).

ES-MS: 235 [M−1].

HPLC Retention Time: 11.88 min, 96% purity @280 nm.

### Example 35: 2,3-Difluoro-5,6,7,8-tetrahydronaphthalene-1-carboxylic acid (A-44)

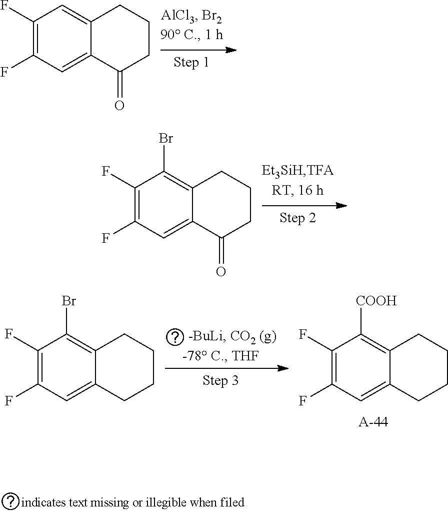

### Step 1: 5-Bromo-6,7-difluoro-3,4-dihydronaphthalen-1(2H)-one

A mixture of 6,7-difluoro-3,4-dihydronaphthalen-1(2H)-one (592 mg, 3.25 mmol) and AlCl₃ (866.6 mg, 6.5 mmol) was heated at 90° C. for 20 min and cooled to ambient temperature. Bromine (0.10 ml, 3.9 mmol) was added, and the resulting slurry was heated at 90° C. for 1 h, then cooled to ambient temperature. The reaction mixture was quenched with ice water (20 mL), extracted with EtOAc (2×30 mL), the combined extracts were washed with water (3×10 mL), and brine (15 mL). The organic layer was dried (Na₂SO₄), concentrated and the resultant residue purified by column chromatography on silica gel utilising EtOAc/hexane (0-5%) to provide the title compound (382 mg, 45%).

¹H NMR (300 MHz, CDCl₃) δ 7.99 (dd, 1H), 3.14 (t, 2H), 2.81 (t, 2H), 2.34 (q, 2H).

¹⁹F NMR (300 MHz, CDCl₃) δ− 127.34, 138.14 ppm.

### Step 2: 5-Bromo-6,7-difluoro-1,2,3,4-tetrahydronaphthalene

A mixture of 5-bromo-6,7-difluoro-3,4-dihydronaphthalen-1(2H)-one (382 mg, 1.46 mmol), triethylsilane (0.7 ml, 4.38 mmol) and trifluoroacetic acid (0.33 ml, 4.38 mmol) was stirred at ambient temperature for 16 h. The volatiles were removed under reduce pressure and the crude product was purified by chromatography on silica gel EtOAc/hexane (0-5%) to afford the title compound (150 mg, 64%) as a light-yellow oil.

¹H NMR (300 MHz, CDCl₃) δ 6.85 (dd, 1H), 2.79-2.61 (m, 4H), 1.88-1.66 (m, 4H).

¹⁹F NMR (300 MHz, CDCl₃) δ− 131.62, 139.45 ppm.

### Step 3: 2,3-Difluoro-5,6,7,8-tetrahydronaphthalene-1-carboxylic acid

n-BuLi (2.5 M in hexane) (0.267 mL, 0.671 mmol) was added to a solution of 5-bromo-6,7-difluoro-1,2,3,4-tetrahydronaphthalene (150 mg, 0.61 mmol) in THF (6 mL) at −78° C. The reaction mixture was stirred at same temperature for 1 h. After 1 h. CO₂ (g) was bubbled for 30 min and stirred for 1 h at −78° C. before slowly bringing the mixture to 0° C. followed by quenching with 1 M NaOH to pH ~11. Washing with EtOAc (10 mL) and separation of the aqueous layer and acidifying it with 1 N HCl to pH ~1 was followed by extraction with EtOAc (2×10 mL) and the combined extracts were washed with water (5 mL) and brine (5 mL). The organic layer was dried (Na₂SO₄), filtered and concentrated to provide the title compound (45 mg, 35%) as a white solid.

¹H NMR (300 MHz, CDCl₃) δ 11.33-9.98 (br, 1H), 6.99 (dd, J_{H-F}=10.5 Hz, J_{H-F}=10.5 Hz, 1H), 2.93-2.79 (m, 2H), 2.80-2.67 (m, 2H), 1.87-1.69 (m, 4H). ¹⁹F NMR (300 MHz, CDCl₃) δ− 141.32, 141.77 ppm.

ES-MS: 211 [M−1].

HPLC Retention Time: 10.63 min, 97.6% purity @280 nm.

### Example 36: 7-fluoro-6-methyl-3,4-dihydro-2H-1-benzopyran-8-carboxylic acid (A-50)

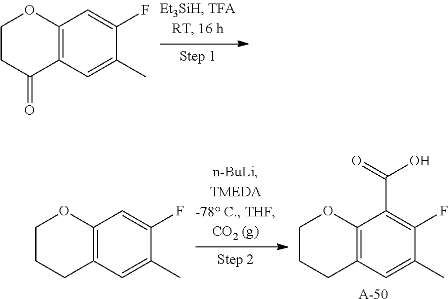

### Step 1: 7-Fluoro-6-methylchroman

A mixture of 7-fluoro-6-methylchroman-4-one (97 mg, 0.54 mmol), triethylsilane (0.52 ml, 3.24 mmol) and trifluoroacetic acid (0.25 ml, 3.24 mmol) was stirred at 80° C. for 16 h. The reaction mixture was evaporated under reduced pressure and the crude product was purified by chromatography on silica gel EtOAc/hexane (0-5%) to provide the title compound (51 mg, 64%) as a light brown solid.

¹H NMR (300 MHz, CDC₃) δ 6.81 (d, 1H), 6.47 (d, 1H), 4.14 (t, 2H); 2.71 (t, 2H); 2.17 (s, 3H), 2.04-1.92 (m, 2H). ¹⁹F NMR (300 MHz, CDC₃) δ− 119.29 ppm.

### Step 2: 7-Fluoro-6-methyl-3,4-dihydro-2H-1-benzopyran-8-carboxylic acid

n-BuLi (2.5 M in hexane) (0.13 mL, 0.36 mmol) and TMEDA (0.049 ml, 0.36 mmol) were added to a solution of 7-fluoro-6-methylchroman (51 mg, 0.30 mmol) in THF (4 mL) at −78° C. The reaction mixture was stirred at the same temperature for 1 h before CO₂ (g) was bubbled through it for 30 min. The mixture was stirred for 1 h at −78° C., slowly brought to 0° C., quenched with 1N NaOH to pH ~11 and washed with EtOAc (10 mL) to remove non-acid impurities. The aqueous layer was separated, acidified with 1 N HCl to pH ~1 and extracted with EtOAc (2×10 mL). The combined extracts were washed with water (5 mL), brine (5 mL), dried (Na₂SO₄) and concentrated in vacuo to provide the title acid (26 mg, 40%) as a white solid.

¹H NMR (300 MHz, CDC₃) δ 7.27 (d, J_{H-F}=8.1 Hz, 1H), 4.59 (t, 2H), 3.04 (t, 2H), 2.47-1.69 (s, 3H), 2.32 (q, 2H). ¹⁹F NMR (300 MHz, CDC₃) δ− 115.66 ppm.

ES-MS: 209 [M−1].

HPLC Retention Time: 9.09 min, 94.9% purity @280 nm.

### Example 37: 2-Benzyl-5,6-dichloro-2,3-dihydro-1H-isoindole-4-carboxylic acid (A-68)

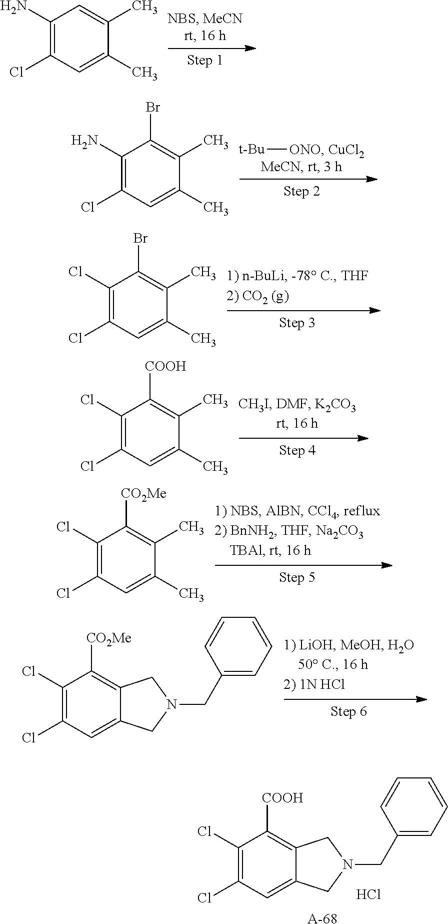

### Step 1: 2-Bromo-6-chloro-3,4-dimethylaniline

NBS (12.9 g, 72.86 mmol) was added to a solution of 2-chloro-4,5-dimethylaniline (10.8 g, 69.39 mmol) in acetonitrile (360 mL). The reaction mixture was stirred at ambient temperature for 16 h, quenched with water (300 mL) and extracted with EtOAc (2×150 mL). The combined extracts were dried (Na₂SO₄) and concentrated under reduced pressure. The resulting residue was purified by column chromatography on silica gel, eluting with EtOAc/hexane (0-10%) to obtain title compound (13.5 g, 83%).

¹H NMR (300 MHz, CDCl₃) δ 7.26 (s, 1H); 4.74-4.51 (br, 2H); 2.55 (s, 3H); 2.45 (s, 3H) ES-MS: 235 [M+1].

### Step 2: 3-Bromo-1,2-dichloro-4,5-dimethylbenzene

2-bromo-6-chloro-3,4-dimethylaniline (13.5 g, 57.6 mmol) in acetonitrile (140 mL) was added to a solution of copper chloride (15.5 g, 115.1 mmol) and tert-butyl nitrite (12.5 mL, 104.2 mmol) in acetonitrile (70 mL) at 0° C. The reaction mixture was stirred at ambient temperature for 3 h, quenched with water (200 mL) and extracted with diethyl ether (2×160 mL). The combined extracts were dried (Na₂SO₄) and concentrated under reduced pressure. The residue was purified by column chromatography on silica gel eluting with EtOAc/hexane (0-3%) to obtain the title compound (10.5 g, 72%).

¹H NMR (300 MHz, CDCl₃) δ 7.26 (s, 1H); 2.44 (s, 3H); 2.35 (s, 3H) ES-MS: 255 [M+1].

### Step 3: 2,3-Dichloro-5,6-dimethylbenzoic acid

n-BuLi (2.5 M in hexane) (10.7 mL, 26.85 mmol) was added to a solution of 3-bromo-1,2-dichloro-4,5-dimethylbenzene (6.2 g, 24.41 mmol) in THF (61 mL) at −78° C., and the reaction mixture was stirred at that temperature for 1 h before CO₂ gas was bubbled for 40 min. The reaction mixture was slowly brought to ambient temperature, quenched with water (15 mL) and washed with EtOAc (2×50 mL). The aqueous layer was acidified to pH ~2 using 1.0 N HCl and extracted with EtOAc (2×75 mL). The combined extracts were dried (Na₂SO₄) and concentrated under reduced pressure to afford the title compound (4.6 g, 84%).

¹H NMR (300 MHz, CDC₃) δ 7.26 (s, 1H); 2.22 (s, 3H); 2.21 (s, 3H)

ES-MS: 218 [M−1].

### Step 4: Methyl 2,3-dichloro-5,6-dimethylbenzoate

K₂CO₃ (5.80 g, 41.98 mmol) was added to a solution of 2,3-dichloro-5,6-dimethylbenzoic acid (4.6 g, 20.99 mmol) in DMF (12 mL) at ambient temperature and stirred for 30 min. CH₃I (1.96 mL, 31.49 mmol) was added and the reaction mixture was stirred at ambient temperature for a further 16 h. The reaction mixture was quenched with water (30 mL), extracted with EtOAc (2×100 mL) then the combined extracts were dried (Na₂SO₄) and concentrated under reduced pressure. The residue was purified by column chromatography on silica gel eluting with EtOAc/hexane (0-20%) to obtain title compound (3.11 g, 64%).

¹H NMR (300 MHz, CDC₃) δ 7.25 (s, 1H); 3.95 (s, 3H); 2.22 (s, 3H); 2.15 (s, 3H) ES-MS: 234 [M+1].

### Step 5: Methyl 2-benzyl-5,6-dichloroisoindoline-4-carboxylate

NBS (2.0 g, 72.86 mmol) and 2,2′-azobis(2-methylpropionitrile) (27.7 mg, 0.17 mmol) were added to a solution of methyl 2,3-dichloro-5,6-dimethylbenzoate (1.3 g, 5.62 mmol) in carbon tetrachloride (15 mL). The reaction mixture was heated to reflux for 16 h, cooled to ambient temperature, filtered and the filtrate was concentrated under reduced pressure to obtain the title compound (3.1 g, quantitative) as a yellow liquid which was used directly in the next step.

Benzylamine (0.87 mL, 7.96 mmol), Na₂CO₃ (1.68 g, 15.92 mmol) and tetrabutylammonium iodide (588 mg, 1.59 mmol) were added to a solution of methyl 2,3-bis(bromomethyl)-5,6-dichlorobenzoate (3.1 g, 7.96 mmol) in THF (26 mL) and the reaction mixture was stirred at ambient temperature for 16 h before quenching with water (300 mL) and extraction with EtOAc (2×150 mL). The combined extracts were dried (Na₂SO₄) and concentrated under reduced pressure. The resultant residue was purified by column chromatography on silica gel eluting with EtOAc/hexane (0-30%) to obtain the title compound (220 mg, 12%).

¹H NMR (300 MHz, CDCl₃) δ 7.33-7.11 (m, 6H); 3.92-3.81 (m, 6H); 3.80-3.73 (m, 3H) ES-MS: 337 [M+1].

### Step 6: 2-Benzyl-5,6-dichloro-2,3-dihydro-1H-isoindole-4-carboxylic acid hydrochloride

LiOH (126 mg, 2.94 mmol) was added to a solution of methyl 2-benzyl-5,6-dichloroisoindoline-4-carboxylate (72 mg, 0.21 mmol) in a mixture of CH₃OH (1 mL) and water (0.6 mL). The reaction mixture was stirred at 50° C. for 16 h and solvent was removed in vacuo before the water layer was washed with CH₂Cl₂ (10 mL). The aqueous layer was acidified with 1N HCl and concentrated under reduced pressure and the crude product was purified by semi-prep HPLC (0 to 100% MeOH/0.1% formic acid in water), then concentrated to give the pure acid. 1N HCl was added to 2-benzyl-5,6-dichloroisoindoline-4-carboxylic acid in dioxane and the mixture was stirred ambient temperature for 30 min. After 30 min, the solution was concentrated under reduced pressure and the solid product dried under vacuum to provide the title compound as an off white solid (2.2 mg, 3%).

¹H NMR (300 MHz, CDCl₃) δ 7.43-7.19 (m, 6H); 4.22-4.06 (m, 6H) ES-MS: m/z 323 [M+1].

HPLC: Retention time 9.77 min., Purity: 97.6% @280 nm.

### Example 38: 5-Chloro-6-fluoro-2,3-dihydro-1-benzofuran-7-carboxylic acid (A-43)

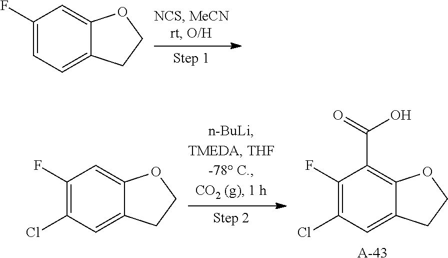

### Step 1: 5-Chloro-6-fluoro-2,3-dihydro-1-benzofuran

NCS (425 mg, 3.18 mmol) was added to a solution of 6-fluoro-2,3-dihydro-1-benzofuran (400 mg, 2.89 mmol) in acetonitrile (10 mL), and the reaction mixture was stirred at ambient temperature for 16 h. The solvent was removed under reduced pressure, the residue was dissolved in EtOAc (20 mL) and washed with water (2×10 mL). The organic layer was dried (Na₂SO₄) and concentrated under reduced pressure to yield a residue which was purified by column chromatography on silica gel. Elution with EtOAc/hexane (0-10%) provided the title compound (237 mg, 47%).

¹H NMR (300 MHz, CDCl₃) δ 7.27 (d, 1H), 6.71 (d, 1H), 4.75 (t, 2H), 3.30 (t, 2H). ¹⁹F NMR (300 MHz, CDCl₃) δ− 115.47 ppm.

### Step 2: 5-Chloro-6-fluoro-2,3-dihydro-1-benzofuran-7-carboxylic acid

n-BuLi (2.5 M in hexane) (0.59 mL, 1.47 mmol) was added to a solution of 5-chloro-6-fluoro-2,3-dihydro-1-benzofuran (230 mg, 1.33 mmol) and TMEDA (0.22 ml, 1.47 mmol) in THF (7 mL) at −78° C. The reaction mixture was stirred at the same temperature for 1 h and CO₂ (g) was bubbled through it for 10 min., stirring was continued for 1 h and it was slowly brought to ambient temperature before the pH was adjusted to ~11 with 1M NaOH. The mixture was washed with CH₂Cl₂ (2×10 mL), the aqueous layer was acidified to pH ~2.0 using 1.0 N HCl and extracted with EtOAc (2×20 mL). The combined organic extracts were dried (Na₂SO₄), concentrated in vacuo and the resutant residue was purified by semi-prep HPLC utilising 30 to 100% acetonitrile/0.1% formic acid in water to afford the title compound (80 mg, 28%).

¹H NMR (300 MHz, CD₃OD) δ 7.29 (d, J_{H-F}=7.03, 1H), 4.60 (t, 2H), 3.12 (t, 2H). ¹⁹F NMR (300 MHz, CD₃OD) δ− 117.65 ppm.

ES-MS: 215.06 [M−H]HPLC: Retention Time: 10.73 min., purity: >99% @280 nm.

### Example 39: 7-Chloro-6-fluoro-3,4-dihydro-2H-1-benzopyran-8-carboxylic acid (A-37)

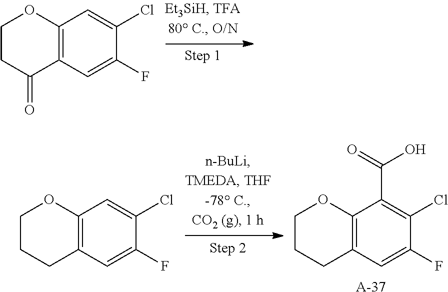

### Step 1: 7-Chloro-6-fluorochroman

A mixture of 7-chloro-6-fluoro-4-chromanone (425 mg, 2.12 mmol), triethylsilane (2.03 ml, 12.75 mmol) and TFA (0.98 ml, 12.75 mmol) was heated in a sealed tube at 80° C. for 16 h. The reaction mixture was cooled, evaporated and the resultant residue was purified by column chromatography on silica gel eluting with EtOAc/hexane (0-10%) to obtain the title compound (151 mg, 38%).

¹H NMR (300 MHz, CDCl₃) δ 6.81 (s, 1H), 6.78 (d, 1H), 4.13 (t, 2H), 2.73 (t, 2H), 1.97 (q, 2H). ¹⁹F NMR (300 MHz, CDCl₃) δ− 127.81 ppm.

### Step 2: 7-Chloro-6-fluoro-3,4-dihydro-2H-1-benzopyran-8-carboxylic acid

n-BuLi (2.5 M in hexane) (0.118 mL, 0.29 mmol) was added to a solution of 7-chloro-6-fluorochroman (50 mg, 0.27 mmol) and TMEDA (0.044 ml, 0.29 mmol) in THF (5 mL) at −78° C. The reaction mixture was stirred at the same temperature for 1 h and CO₂ (g) was bubbled for 10 min. and stirring was continued at −78° C. for 1 h., slowly brought to ambient temperature and the pH was adjusted to ~11 with 1M NaOH. The reaction mixture was washed with CH₂Cl₂ (2×10 mL), the aqueous layer was acidified to pH ~2.0 using 1.0 N HCl and extracted with EtOAc (2×20 mL). The combined extracts were dried (Na₂SO₄), concentrated and purified by semi-prep HPLC using 30 to 100% acetonitrile/0.1% formic acid in water to provide the title acid (43 mg, 69%).

¹H NMR (300 MHz, CD₃OD) δ 7.04 (d, J_{H-F}=9.6, 1H), 4.22 (t, 2H), 2.82 (t, 2H), 2.02 (q, 2H). ¹⁹F NMR (300 MHz, CD₃OD) δ− 128.81 ppm.

ES-MS: 229 [M−H]HPLC: Retention Time: 9.19 min., purity: >99% @280 nm.

### Example 40: 7-Chloro-6-fluoro-3,4-dihydro-2H-1-benzopyran-5-carboxylic acid (A-39)

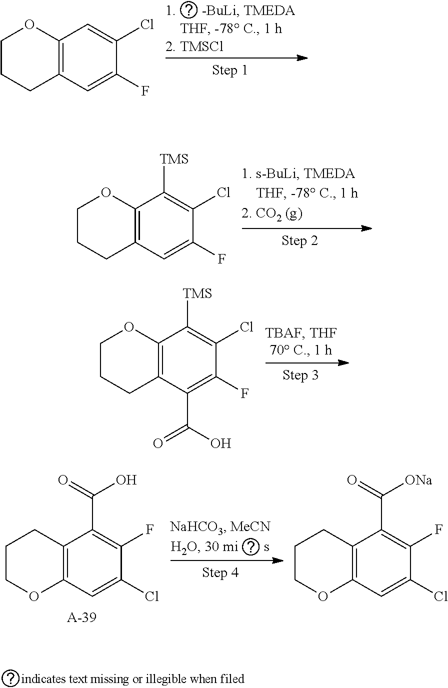

### Step 1: (3-Chloro-2-fluoro-5,6,7,8-tetrahydronaphthalen-1-yl)trimethylsilane

n-BuLi (2.5 M in hexane) (1.18 mL, 2.95 mmol) was added to a solution of 7-chloro-6-fluorochroman (500 mg, 2.68 mmol) and TMEDA (0.442 ml, 2.95 mmol) in THF (10 mL) at −78° C. The reaction mixture was stirred at the same temperature for 1 h and trimethylsilyl chloride (1.02 ml, 8.04 mmol) was added dropwise. The reaction mixture was stirred at −78° C. for 1 h, slowly brought to ambient temperature and stirred for 16 h.

The reaction mixture was quenched with saturated ammonium chloride solution (5 mL), extracted with EtOAc (2×15 mL) and the combined extracts were dried (Na₂SO₄), filtered and concentrated in vacuo. The crude product was purified by column chromatography on silica gel eluting with hexane/EtOAc (0-10%) to afford the title compound (441 mg, 71%).

¹H NMR (300 MHz, CDC₃) δ 6.79 (d, 1H), 4.11 (t, 2H), 2.74 (t, 2H), 1.96 (q, 2H), 0.40 (s, 9H). ¹⁹F NMR (300 MHz, CDC₃) δ− 124.31 ppm.

### Step 2: 2-Chloro-3-fluoro-4-(trimethylsilyl)-5,6,7,8-tetrahydronaphthalene-1-carboxylic acid

s-BuLi (1.4 M in hexane) (0.341 mL, 0.47 mmol) was added to a solution of (3-chloro-2-fluoro-5,6,7,8-tetrahydronaphthalen-1-yl)trimethylsilane (100 mg, 0.43 mmol) and TMEDA (0.072 ml, 0.47 mmol) in THF (5 mL) at −78° C. The reaction mixture was stirred at the same temperature for 1 h and CO₂ (g) was bubbled for 10 min, stirring was continued at −78° C. for 1 h and the mixture. was slowly brought to ambient temperature before the pH was adjusted to ~11 with 1N NaOH. The reaction mixture was washed with CH₂Cl₂ (2×10 mL), the aqueous layer was acidified to pH ~2.0 using 1.0 N HCl and extracted with EtOAc (2×20 mL). The combined extracts were dried (Na₂SO₄), concentrated, and crystallized with CH₂Cl₂/hexane to afford the title compound (47 mg, 39%).

¹H NMR (300 MHz, CD₃OD) δ 4.01 (t, 2H), 2.68 (t, 2H), 1.84 (q, 2H), 0.27 (s, 9H). ¹⁹F NMR (300 MHz, CD₃OD) δ− 127.34 ppm.

### Step 3: 7-chloro-6-fluoro-3,4-dihydro-2H-1-benzopyran-5-carboxylic acid

A mixture of 2-chloro-3-fluoro-4-(trimethylsilyl)-5,6,7,8-tetrahydronaphthalene-1-carboxylic acid (45 mg, 0.16 mmol) and TBAF (1.0 M in THF) (2.0 ml, 12.2 mmol) was heated at 70° C. for 1 h. The reaction mixture was cooled, evaporated and purified by semi-prep HPLC using 30 to 100% acetonitrile/0.1% formic acid in water to provide the title acid (18 mg, 47%).

¹H NMR (300 MHz, CD₃OD) δ 6.95 (d, J_{H-F}=6.5, 1H), 4.17 (t, 2H), 2.82 (t, 2H), 2.0 (q, 2H).

¹⁹F NMR (300 MHz, CD₃OD) δ− 130.78 ppm.

ES-MS: 229.16 [M−H]

HPLC: Retention Time: 9.88 min., purity: >99% @280 nm.

### Step 4: Sodium 7-chloro-6-fluoro-3,4-dihydro-2H-1-benzopyran-5-carboxylate

To a solution of 7-chloro-6-fluoro-3,4-dihydro-2H-1-benzopyran-5-carboxylic acid (125 mg, 0.54 mmol) in acetonitrile (4.0 mL) and water (3.0 mL), NaHCO₃ (43.2 mg, 0.52 mmol) was added and the reaction mixture was stirred at ambient temperature for 30 min. The solvent was removed under reduced pressure and triturated with diethyl ether (2×5.0 mL) to provide the title compound (150 mg, 71%).

¹H NMR (300 MHz, CD₃OD) δ 6.72 (d, J_{H-F}=6.4, 1H), 4.14 (t, 2H), 2.82 (t, 2H), 1.98 (q, 2H). ¹⁹F NMR (300 MHz, CD₃OD) δ− 133.68 ppm.

ES-MS: 229.25 [M−H]HPLC: Retention Time: 9.84 min., purity: 98.7% @280 nm.

### Example 41: 7-fluoro-6-methyl-3,4-dihydro-2H-1-benzothiopyran-8-carboxylic acid (A-48)

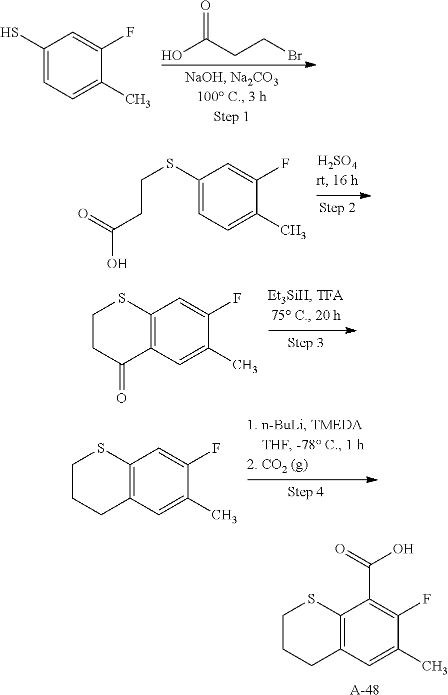

### Step 1: 3-(3-Fluorotolylthio)propionic acid

3-Bromopropionic acid (2.37 g, 15.1 mmol) was added to a solution of 3-fluoro-4-methylthiophenol (2.00 g, 14.1 mmol) in ethanol (25 mL) with 1.0 N NaOH (10 mL), 1.0 N Na₂CO₃ (10 mL) present. The reaction mixture was stirred at ambient temperature for 1 h and heated at 100° C. for 3 h. The solvent was removed under reduced pressure, the aqueous residue was washed with EtOAc (2×50 mL), acidified to pH ~2.0 using 1.0 N HCl and extracted with EtOAc (2×75 mL). The combined extracts were dried (Na₂SO₄), concentrated under reduced pressure and the residue triturated with hexanes (3×10 mL) to obtain the title compound (1.77 g, 59%).

¹H NMR (300 MHz, CDC₃) δ 7.14-7.03 (m, 3H), 3.13 (t, 2H), 2.67 (t, 2H), 2.24 (s, 3H).

### Step 2: 7-Fluoro-6-methyl-2,3-dihydro-1-benzothiin-4-one

3-(3-Fluorotolylthio)propionic acid (1.40 g, 6.53 mmol) in sulphuric acid (25 mL) was stirred at ambient temperature for 16 h. The reaction mixture was poured into ice cold water (50 mL) and extracted with EtOAc (2×50 mL). The combined extracts were dried (Na₂SO₄) and concentrated under reduced pressure to provide the title compound (1.03 g, 80%).

¹H NMR (300 MHz, CDC₃) δ 7.99 (d, 1H), 6.92 (d, 1H), 3.25-3.18 (m, 2H), 2.99-2.93 (m, 2H), 2.24 (s, 3H).

### Step 3: 7-Fluoro-6-methyl-3,4-dihydro-2H-1-benzothiin

A mixture of 7-fluoro-6-methyl-2,3-dihydro-1-benzothiin-4-one (0.93 g, 4.94 mmol), Et₃SiH (4.5 mL, 28.4 mmol) and TFA (2.2 mL, 28.4 mmol) was heated in a sealed tube at 75° C. for 20 h. The reaction mixture was evaporated under reduced pressure and the residue was purified by column chromatography on silica gel eluting with hexane to provide the desired compound (0.36 g, 42%).

¹H NMR (300 MHz, CDC₃) δ 6.82 (d, 1H), 6.75 (d, 1H), 3.01-2.97 (m, 2H), 2.73 (t, 2H), 2.17 (s, 3H), 2.13-2.04 (m, 2H).

### Step 4: 7-Fluoro-6-methyl-3,4-dihydro-2H-1-benzothiopyran-8-carboxylic acid

TMEDA (0.34 mL, 2.18 mmol), and n-BuLi (2.5 M in hexane) (0.88 mL, 2.18 mml) were added to a solution of 7-fluoro-6-methyl-3,4-dihydro-2H-1-benzothiin (0.36 g, 1.98 mmol) in THF (10 mL) at −78° C. The reaction mixture was stirred at that temperature for 1 h. and CO₂ was bubbled for 40 min. The reaction mixture was slowly brought to ambient temperature, quenched with water (15 mL) and washed with EtOAc (2×25 mL). The aqueous layer was acidified to pH ~2.0 using 1.0 N HCl and extracted with EtOAc (2×25 mL). The combined extracts were dried (Na₂SO₄) and concentrated under reduced pressure to afford the title acid (10 mg, 2%).

¹H NMR (300 MHz, CDCl₃) δ 6.97 (d, J_{H-F}=7.5 Hz, 1H), 2.98-2.94 (m, 2H), 2.77 (t, 2H), 2.20 (s, 3H), 2.09 (quint, 2H) ES-MS: m/z 225.2 (M-1) HPLC Retention time 10.0 min., purity: 98.2% at 254 nm.

### Example 42: 6,7-difluoro-3,4-dihydro-2H-1-benzothiopyran-8-carboxylic acid (A-42)

### Step 1: 3-(3,4-Difluorophenylthio)propionic acid

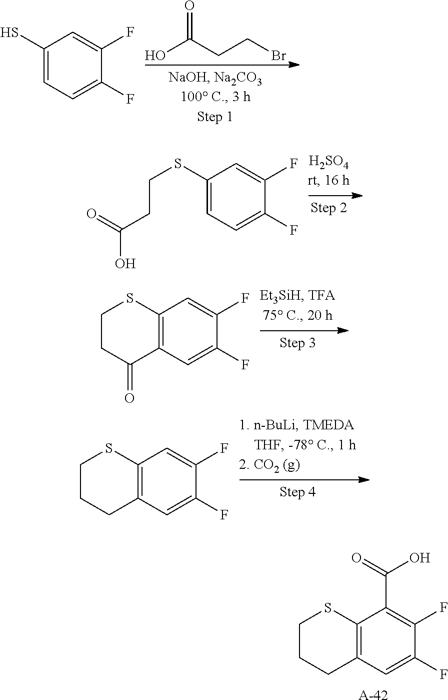

1.0 N NaOH (10 mL), 1.0 N Na₂CO₃ (10 mL) and 3-bromopropionic acid (2.30 g, 15.0 mmol) was added to a solution of 3,4-difluorothiophenol (2.00 g, 13.7 mmol) in ethanol (25 mL). The reaction mixture was stirred at ambient temperature for 1 h and then heated at 100° C. for 3 h. Volatile solvent was removed under reduced pressure and the aqueous residue washed with EtOAc (2×50 mL). The aqueous layer was acidified to pH ~2.0 using 1.0 N HCl and extracted with EtOAc (2×75 mL). The combined extracts were dried (Na₂SO₄), concentrated under reduced pressure and triturated with hexanes (3×10 mL) to obtain the title compound (1.38 g, 46%).

¹H NMR (300 MHz, CD₃OD) δ 7.39-7.22 (m, 3H), 3.19 (t, 2H), 2.61 (t, 2H).

### Step 2: 6,7-Difluoro-2,3-dihydro-1-benzothiin-4-one

3-(3,4-Difluorophenylthio)propionic acid (1.40 g, 6.32 mmol) in sulphuric acid (25 mL) was stirred at ambient temperature for 16 h. The reaction mixture was poured into ice cold water (50 mL) and extracted with EtOAc (2×50 mL). The combined extracts were dried (Na₂SO₄) and concentrated under reduced pressure to provide the title compound (1.18 g, 93%).

¹H NMR (300 MHz, CDC₃) δ 7.97-7.90 (m, 1H), 7.12-7.06 (m, 1H), 3.28-3.23 (m, 2H), 2.98-2.94 (m, 2H).

### Step 3: 6,7-Difluoro-3,4-dihydro-2H-1-benzothiin

A mixture of 6,7-difluoro-2,3-dihydro-1-benzothiin-4-one (1.00 g, 4.99 mmol), Et₃SiH (4.8 mL, 30.0 mmol) and TFA (2.3 mL, 30.0 mmol) in a sealed tube was heated at 75° C. for 20 h. The solvent was evaporated under reduced pressure and the residue was purified by column chromatography on silica gel eluting with hexane to obtain the title compound (0.88 g, 95%).

¹H NMR (300 MHz, CDC₃) δ 6.91-6.81 (m, 2H), 3.02-2.98 (m, 2H), 2.74 (t, 2H), 2.09 (quint, 2H).

### Step 4: 6,7-Difluoro-3,4-dihydro-2H-1-benzothiopyran-8-carboxylic acid

TMEDA (0.28 mL, 1.81 mmol), and n-BuLi (2.5 M in hexane) (0.72 mL, 1.81 mmol) were introduced to a solution of 6,7-difluoro-3,4-dihydro-2H-1-benzothiin (0.30 g, 1.61 mmol) in THF (10 mL) at −78° C. The reaction mixture was stirred at that temperature for 1 h and CO₂ was bubbled for 40 min. The reaction mixture was slowly brought to ambient temperature, quenched with water (15 mL) and washed with EtOAc (2×25 mL). The aqueous layer was acidified to pH ~2.0 using 1.0 N HCl and extracted with EtOAc (2×25 mL). The combined extracts were dried (Na₂SO₄) and concentrated under reduced pressure to afford the title compound (0.17 g, 46%).

¹H NMR (300 MHz, CDC₃) δ 7.04-6.98 (m, 1H), 2.96 (t, 2H), 2.80 (t, 2H), 2.11 (quint, 2H)

ES-MS: m/z 229.2 (M-1)

HPLC Retention time 9.43 min., purity: 96.3% at 280 nm.

### Example 43: 6-Chloro-7-fluoro-3,4-dihydro-2H-1-benzothiopyran-8-carboxylic acid (A-38)

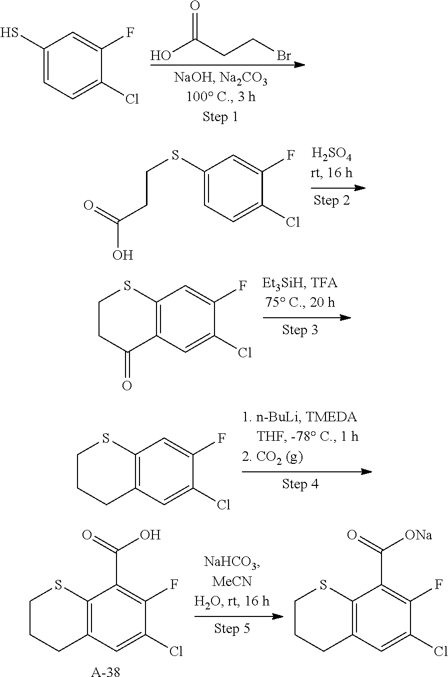

### Step 1: 3-(4-Chloro-3-fluorophenylthio)propionic acid

1.0 N NaOH (10 mL), 1.0 N Na₂CO₃ (10 mL), and 3-bromopropionic acid (2.10 g, 13.7 mmol) was added to a solution of 4-chloro-3-fluorobenzenethiol (2.00 g, 12.3 mmol) in ethanol (25 mL). The reaction mixture was stirred at ambient temperature for 1 h and then heated at 100° C. for 3 h. The solvent was removed under reduced pressure and the aqueous residue extracted with EtOAc (2×50 mL). The aqueous layer was acidified to pH ~2.0 using 1.0 N HCl and extracted with EtOAc (2×75 mL). The combined extracts were dried (Na₂SO₄), concentrated under reduced pressure and triturated with hexanes (3×10 mL) to obtain the title compound (1.82 g, 63%).

¹H NMR (300 MHz, CD₃OD) δ 7.42 (t, 1H), 7.28 (dd, 1H), 7.19-7.16 (m, 1H), 3.23 (t, 2H), 2.65 (t, 2H).

### Step 2: 6-Chloro-7-fluoro-2,3-dihydro-1-benzothiin-4-one

3-(4-Chloro-3-fluorophenylthio)propionic acid (1.82 g, 7.76 mmol) in sulphuric acid (25 mL) was stirred at ambient temperature for 16 h. The reaction mixture was poured into ice cold water (50 mL) and extracted with EtOAc (2×50 mL). The combined extracts were dried (Na₂SO₄) and concentrated under reduced pressure to provide the title compound (1.41 g, 83%).

¹H NMR (300 MHz, CDC₃) δ 8.17 (d, 1H), 7.07 (d, 1H), 3.28-3.24 (m, 2H), 2.99-2.95 (m, 2H).

### Step 3: 6-Chloro-7-fluoro-3,4-dihydro-2H-1-benzothiin

A mixture of 6-chloro-7-fluoro-2,3-dihydro-1-benzothiin-4-one (1.00 g, 4.62 mmol), Et₃SiH (4.5 mL, 27.6 mmol) and TFA (2.1 mL, 27.6 mmol) was heated at 75° C. in a sealed tube over a period of 20 h. The solvent was evaporated under reduced pressure and the residue was purified by column chromatography on silica gel eluting with hexane to obtain the desired compound (0.91 g, 97%).

¹H NMR (300 MHz, CDC₃) δ 7.03 (d, 1H), 6.88 (d, 1H) 3.00 (m, 2H), 2.74 (t, 2H), 2.09 (quint, 2H).

### Step 4: 6-Chloro-7-fluoro-3,4-dihydro-2H-1-benzothiopyran-8-carboxylic acid

TMEDA (0.20 mL, 1.36 mmol) and n-BuLi (2.5 M in hexane) (0.54 mL, 1.36 mmol) were added to a solution of 6-chloro-7-fluoro-3,4-dihydro-2H-1-benzothiin (0.25 g, 1.23 mmol) in THF (10 mL) at −78° C. The reaction mixture was stirred at the same temperature for 1 h and CO₂ was bubbled for 40 min. The reaction mixture was slowly brought to ambient temperature, quenched with water (15 mL) and washed with EtOAc (2×25 mL). The aqueous layer was acidified to pH ~2.0 using 1.0 N HCl and extracted with EtOAc (2×25 mL). The combined extracts were dried (Na₂SO₄) and concentrated under reduced pressure to afford the title compound (126 mg, 41%).

¹H NMR (300 MHz, CDC₃) δ 7.18 (d, J_{H-F}=7.2 Hz, 1H), 3.01-2.97 (m, 2H), 2.80 (t, 2H), 2.10 (quint, 2H) ES-MS: m/z 245.4 (M-1) HPLC Retention time 10.4 min., purity: 99.3% at 280 nm.

### Step 5: Sodium 6-chloro-7-fluoro-3,4-dihydro-2H-1-benzothiopyran-8-carboxylate

NaHCO₃ (34 mg, 0.40 mmol) was added to a solution of 6-chloro-7-fluoro-3,4-dihydro-2H-1-benzothiopyran-8-carboxylic acid (105 mg, 0.43 mmol) in acetonitrile (2.5 mL) and water (2.5 mL) and the reaction mixture was stirred at ambient temperature for 16 h. The solvent was removed under reduced pressure, the residue was dissolved in water (10 mL) and washed with EtOAc (3×25 mL). The aqueous layer was concentrated in vacuo to provide the title compound (91 mg, 80%).

¹H NMR (300 MHz, CD₃OD) δ 7.04 (d, J_{H-F}=7.5 Hz, 1H), 3.01-2.97 (m, 2H), 2.79 (t, 2H), 2.07 (quint, 2H) ES-MS: m/z 245.4 (M-1) HPLC Retention time 10.3 min., purity: 97.2% at 280 nm.

### Example 44: 3-Chloro-2-methyl-6,7,8,9-tetrahydro-5H-benzo[7]annulene-1-carboxylic acid (A-33) and 2-chloro-3-methyl-6,7,8,9-tetrahydro-5H-benzo[7]annulene-1-carboxylic acid (A-34)

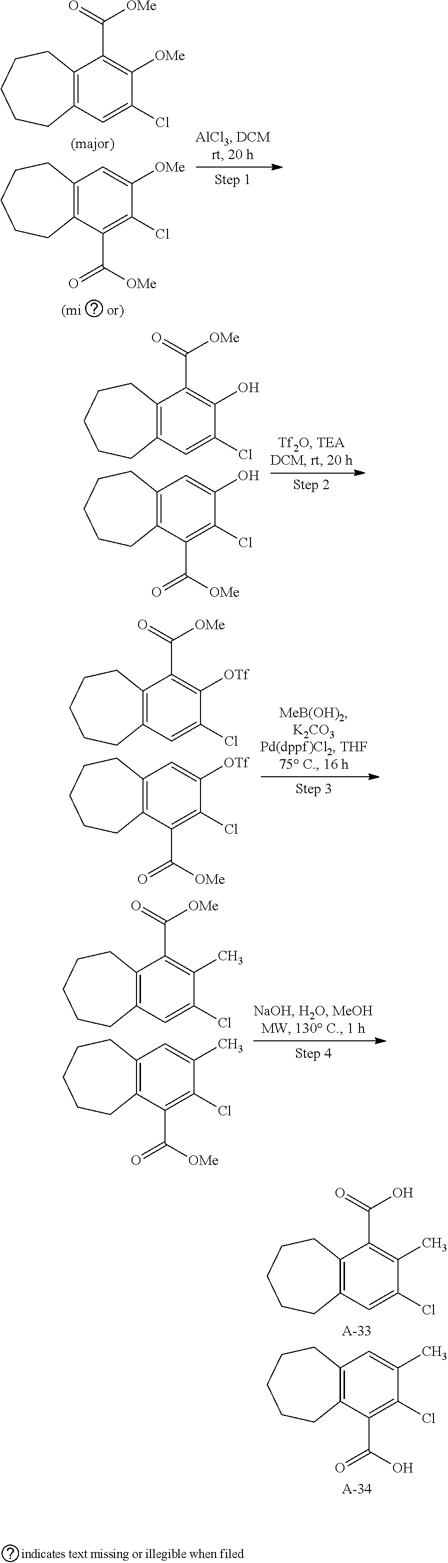

### Step 1: Methyl 2-chloro-3-hydroxy-6,7,8,9-tetrahydro-5H-benzo[7]annulene-4-carboxylate

The starting material methyl 2-chloro-3-methoxy-6,7,8,9-tetrahydro-5H-benzo[7]annulene-4-carboxylate was contaminated with a small amount of methyl 3-chloro-2-methoxy-6,7,8,9-tetrahydro-5H-benzo[7]annulene-4-carboxylate. The two compounds were carried through the synthesis and only separated at the final step. Due to the small amount of compound and overlapping signals, only intermediate data for the major compound are reported.

AlCl₃ (2.08 g, 15.6 mmol) was introduced to a solution of methyl 10-chloro-9-methoxybicyclo[5.4.0]undeca-1(7),8,10-triene-8-carboxylate (1.04 g, 3.87 mmol) in CH₂Cl₂ (20 mL) at 0° C. and the reaction mixture was stirred at ambient temperature for 20 h. The reaction mixture was cooled to 0° C. and quenched with 1.0 N HCl (50 mL). The layers were separated and the aqueous layer was extracted with CH₂Cl₂ (2×30 mL). The combined extracts were dried (Na₂SO₄) and concentrated under reduced pressure to provide the title compound (0.92 g, 93%) which was used in the next step without further purification.

¹H NMR (300 MHz, CDC₃) δ 9.50 (s, 1H), 7.29 (s, 1H), 3.99 (s, 3H), 2.94-2.91 (m, 2H), 2.79-2.76 (m, 2H), 1.87-1.79 (m, 2H), 1.73-1.60 (m, 4H).

### Step 2: Methyl 10-chloro-9-(trifluoromethylsulfonyloxy)bicyclo[5.4.0]undeca-1(7),8,10-triene-8-carboxylate

Triethylamine (1.26 mL, 9.02 mmol) was added to a solution of methyl 10-chloro-9-hydroxy-bicyclo[5.4.0]undeca-1(7),8,10-triene-8-carboxylate (0.92 g, 3.61 mmol) in CH₂Cl₂ (20 mL) at 0° C., followed by the slow addition of (CF₃SO₂)₂O (1.22 mL, 7.23 mmol). The reaction mixture was stirred at ambient temperature for 20 h, cooled to 0° C. and quenched with 1.0 N HCl (50 mL). The layers were separated, the aqueous layer was extracted with CH₂Cl₂ (2×30 mL) then the combined extracts were dried (Na₂SO₄) and concentrated under reduced pressure. The residue was purified by column chromatography on silica gel eluting with hexane/EtOAc (0-10%) to provide the title compound (1.27 g, 91%).

¹H NMR (300 MHz, CDC₃) δ 7.31 (s, 1H), 3.92 (s, 3H), 2.83-2.80 (m, 2H), 2.75-2.71 (m, 2H), 1.87-1.79 (m, 2H), 1.69-1.62 (m, 4H).

### Step 3: Methyl 10-chloro-9-methylbicyclo[5.4.0]undeca-1(7),8,10-triene-8-carboxylate

A mixture of methyl 10-chloro-9-(trifluoromethylsulfonyloxy)bicyclo[5.4.0]undeca-1(7),8,10-triene-8-carboxylate (300 mg, 0.78 mmol), methylboronic acid (185 mg, 3.09 mmol), Pd(dppf)Cl₂ (114 mg, 0.16 mmol) and K₃PO₄ (660 mg, 3.11 mmol) in THF (5.0 mL) was degassed for 30 min. The reaction mixture was heated in a sealed tube at 75° C. for 16 h. The solvent was removed under reduced pressure and the residue was purified by column chromatography on silica gel eluting with hexane/EtOAc (0-10%) to afford the title compound (110 mg, 56%).

¹H NMR (300 MHz, CDC₃) δ 7.14 (s, 1H), 3.89 (s, 3H), 2.76-2.72 (m, 2H), 2.63-2.59 (m, 2H), 2.25 (s, 3H), 1.84-1.77 (m, 2H), 1.65-1.58 (m, 4H).

### Step 4: 3-chloro-2-methyl-6,7,8,9-tetrahydro-5H-benzo[7]annulene-1-carboxylic acid (4) and 2-chloro-3-methyl-6,7,8,9-tetrahydro-5H-benzo[7]annulene-1-carboxylic acid (4a)

NaOH (52 mg, 1.30 mmol) was added to a solution of methyl 10-chloro-9-methylbicyclo[5.4.0]undeca-1 (7),8,10-triene-8-carboxylate (110 mg, 0.44 mmol) in methanol (3.0 mL) and water (1.0 mL) and the reaction mixture was heated at 130° C. under microwave conditions for 50 min. The solvent was removed under reduced pressure and the residue treated with water (15 mL) and EtOAc (15 mL). The layers were separated, the aqueous layer was acidified with 1.0 N HCl (10 mL) and extracted with EtOAc (2×25 mL). The combined extracts were dried (Na₂SO₄) and concentrated under reduced pressure. The mixture was purified by semi-prep HPLC using 25-100% acetonitrile/0.1% formic acid in water to obtain the title compounds 5A (16 mg, 15%) and 5B (5 mg, 5%). 3-Chloro-2-methyl-6,7,8,9-tetrahydro-5H-benzo[7]annulene-1-carboxylic acid (A-33) ¹H NMR (300 MHz, CDC₃) δ 7.18 (s, 1H), 2.78-2.74 (m, 4H), 2.36 (s, 3H), 1.86-1.78 (m, 2H), 1.68-1.59 (m, 4H).

ES-MS: m/z 237.5 (M-1)

HPLC Retention time 12.4 min., purity: >99% at 280 nm.

### 2-Chloro-3-methyl-6,7,8,9-tetrahydro-5H-benzo[7]annulene-1-carboxylic acid (A-34)

¹H NMR (300 MHz, CDC₃) δ 7.64 (s, 1H), 3.15-3.11 (m, 2H), 2.86-2.63 (m, 2H), 2.68 (s, 3H), 1.89-1.80 (m, 2H), 1.70-1.58 (m, 4H).

ES-MS: m/z 237.5 (M-1)

HPLC Retention time 13.4 min., purity: >99% at 280 nm.

### Example 45: 7-Chloro-6-fluoro-3,4-dihydro-2H-1-benzothiopyran-8-carboxylic acid (A-64) and 7-chloro-6-fluoro-3,4-dihydro-2H-1-benzothiopyran-5-carboxylic acid (A-65)

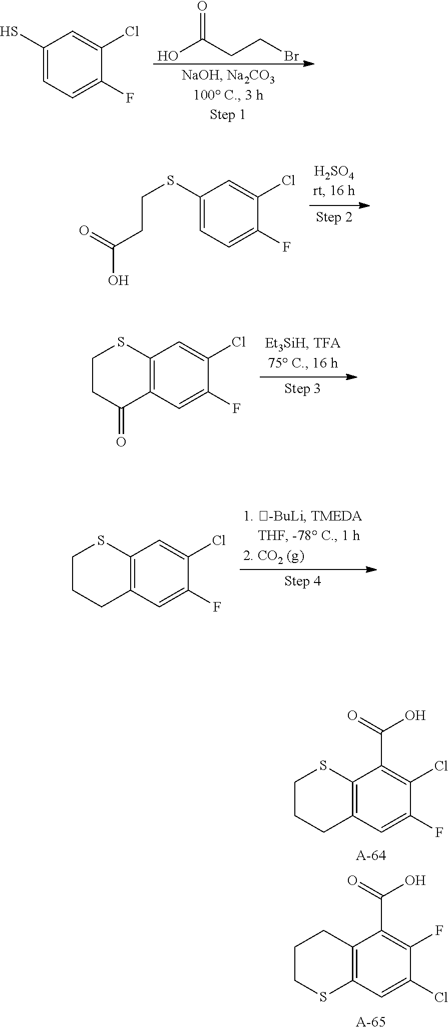

### Step 1: 3-(3-Chloro-4-fluorophenylthio)propionic acid

3-Bromopropionic acid (5.17 g, 33.8 mmol) in 1.0 N Na₂CO₃ (20 mL) was added to a solution of 3-chloro-4-fluorobenzenethiol (5.00 g, 30.7 mmol) in ethanol (50 mL) and 1.0 N NaOH (25 mL) was introduced. The reaction mixture was stirred at ambient temperature for 1 h, heated at 100° C. over a period of 3 h and the solvent was removed under reduced pressure. The resulted aqueous residue was washed with EtOAc (2×50 mL). The aqueous layer was acidified to pH ~2.0 using 1.0 N HCl and extracted with EtOAc (2×75 mL). The combined organic extracts were dried (Na₂SO₄), concentrated to a residue which was triturated with hexanes (3×20 mL) to obtain the title compound (5.70 g, 72%).

¹H NMR (300 MHz, DMSO-d6) δ 12.4 (br s, 1H) 7.61-7.58 (m, 1H), 7.42-7.37 (m, 2H), 3.16 (t, 2H), 2.55 (t, 2H).

### Step 2: 7-Chloro-6-fluoro-2,3-dihydro-1-benzothiin-4-one

3-(3-Chloro-4-fluorophenylthio)propionic acid (4.00 g, 17.0 mmol) was added to pre-cooled sulfuric acid (40 mL) at 0° C. The reaction mixture was stirred at ambient temperature for 16 h. The reaction mixture was poured into ice cold water (100 mL) and extracted with EtOAc (2×70 mL). The combined extracts were washed with 1.0 N NaOH (100 mL), dried (Na₂SO₄) and concentrated under reduced pressure to obtain the title compound (3.48 g, 94%).

¹H NMR (300 MHz, CDC₃) δ 7.93 (d, 1H), 7.41 (d, 1H), 3.33-3.29 (m, 2H), 3.05-3.01 (m, 2H).

### Step 3: 7-Chloro-6-fluoro-3,4-dihydro-2H-1-benzothiin

A mixture of 7-chloro-6-fluoro-2,3-dihydro-1-benzothiin-4-one (3.00 g, 13.8 mmol), Et₃SiH (11.1 mL, 69.2 mmol) and TFA (5.3 mL, 69.2 mmol) in a sealed tube was heated at 75° C. for 16 h. The solvent was evaporated under reduced pressure and the residue was purified by column chromatography on silica gel eluting with hexane to obtain the desired compound (2.80 g, 99%).

¹H NMR (300 MHz, CDC₃) δ 7.16 (d, 1H), 6.89 (d, 1H) 3.08-3.04 (m, 2H), 2.82 (t, 2H), 2.15 (quint, 2H).

### Step 4: 7-Chloro-6-fluoro-3,4-dihydro-2H-1-benzothiopyran-8-carboxylic acid (5A) and

7-chloro-6-fluoro-3,4-dihydro-2H-1-benzothiopyran-5-carboxylic acid (5B) TMEDA (0.44 mL, 2.92 mmol) and n-BuLi (2.5 M in hexane) (1.20 mL, 2.92 mmol) were added to a solution of 7-chloro-6-fluoro-3,4-dihydro-2H-1-benzothiin (0.54 g, 2.66 mmol) in THF (7.0 mL) at −78° C. The reaction mixture was stirred at that temperature for 1 h and CO₂ was bubbled over a period of 40 min. The reaction mixture was slowly brought to ambient temperature, quenched with water (25 mL) and washed with EtOAc (2×30 mL). The aqueous layer was acidified to pH ~2.0 using 1.0 N HCl and extracted into EtOAc (2×30 mL). The combined extracts were dried (Na₂SO₄) and concentrated under reduced pressure to afford the title compound as a mixture of regioisomers (0.49 g, 74%). 80 mg of the mixture of regioisomers was separated using semi-preparative HPLC (10 to 100% acetonitrile/0.1% formic acid in water) to obtain 15 mg of each isomer. 7-Chloro-6-fluoro-3,4-dihydro-2H-1-benzothiopyran-8-carboxylic acid (A-64): ¹H NMR (300 MHz, CD₃OD) δ 7.08 (d, J_{H-F}=9.6 Hz, 1H), 3.03 (t, 2H), 2.85 (t, 2H), 2.10 (quint, 2H) ES-MS: m/z 245.5 (M-1) HPLC retention time 9.57 min., purity: 98.3% at 280 nm.

### 7-Chloro-6-fluoro-3,4-dihydro-2H-1-benzothiopyran-5-carboxylic acid (A-65)

¹H NMR (300 MHz, CD₃OD) δ 7.26 (d, J_{H-F}=7.2 Hz, 1H), 3.06-3.03 (m, 2H), 2.83 (t, 2H), 2.12 (quint, 2H)

ES-MS: m/z 245.6 (M-1)

HPLC retention time 10.8 min., purity: 97.6% at 280 nm.

### Example 46: 6-Chloro-7-methoxy-3,4-dihydro-2H-1-benzopyran-8-carboxylic acid

(A-69)

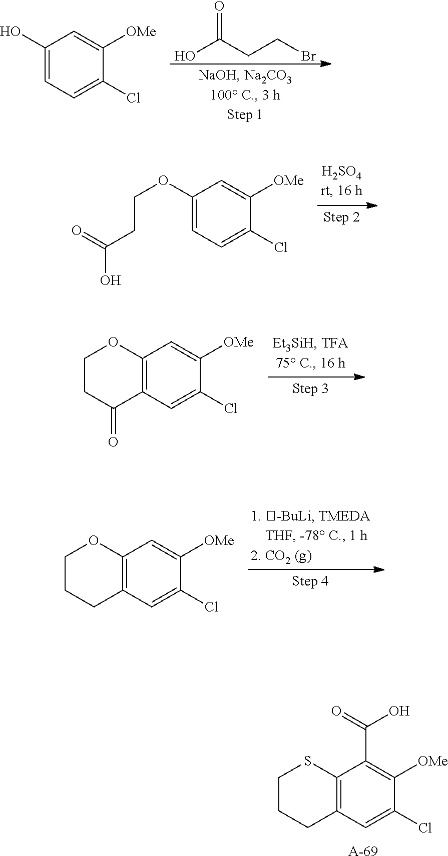

### Step 1: 3-(4-Chloro-3-methoxyphenoxy)propionic acid

4-chloro-3-methoxyphenol (3.00 g, 18.9 mmol) and 3-bromopropionic acid (2.89 g, 18.9 mmol) were added to a solution of NaOH (1.89 g, 47.2 mmol) in water (20 mL) and the reaction mixture heated at 95° C. for 16 h. The reaction mixture was cooled to 0° C., acidified to pH ~2.0 using 1.0 N HCl and extracted into EtOAc (2×50 mL). The combined extracts were dried (Na₂SO₄), concentrated to a residue which was triturated with hexanes (2×10 mL) to obtain the title compound (0.43 g, 10%).

¹H NMR (300 MHz, DMSO-d6) δ 12.4 (br s, 1H), 7.29 (d, 1H), 6.68 (d, 1H), 6.54 (dd, 1H), 4.17 (t, 2H), 3.83 (s, 3H), 2.69 (t, 2H).

### Step 2: 6-Chloro-7-methoxy-4-chromanone

Oxalyl chloride (0.32 mL, 3.70 mmol) and catalytic DMF (two drops) were added to a 0° C. solution of 3-(4-chloro-3-methoxyphenoxy)propionic acid (0.43 g, 1.86 mmol) in CH₂Cl₂ (8.0 mL) and the reaction mixture was stirred at ambient temperature for 2 h. The reaction mixture was again cooled to 0° C., AlCl₃ (0.37 g, 2.77 mmol) was added, and the mixture was stirred at ambient temperature over a period of 16 h. The reaction mixture was cooled to 0° C., quenched with water (20 mL) and diluted with CH₂Cl₂ (20 mL). The layers were separated, and the organic layer was washed with 1.0 N NaOH (20 mL). The organic layer was dried (Na₂SO₄), gravity filtered and concentrated under reduced pressure to obtain the title compound (0.21 g, 53%).

¹H NMR (300 MHz, CDC₃) δ 7.90 (s, 1H), 6.47 (s, 1H), 4.53 (t, 2H), 3.93 (s, 3H), 2.76 (t, 2H).

### Step 3: 6-Chloro-7-methoxychroman

A mixture of 6-chloro-7-methoxy-4-chromanone (0.21 g, 0.98 mmol), Et₃SiH (0.95 mL, 5.93 mmol) and TFA (0.45 mL, 5.93 mmol) were heated at 75° C. for 16 h. The solvent was evaporated under reduced pressure and the residue was purified by column chromatography on silica gel eluting with hexane to obtain the desired compound (150 mg, 77%).

¹H NMR (300 MHz, CDC₃) δ 7.00 (s, 1H), 6.39 (s, 1H), 4.16 (t, 2H), 3.83 (s, 3H), 2.69 (t, 2H), 2.01-1.93 (m, 2H).

### Step 4: 6-Chloro-7-methoxy-3,4-dihydro-2H-1-benzopyran-8-carboxylic acid

TMEDA (0.14 mL, 0.90 mmol) and n-BuLi (2.5 M in hexane) (0.36 mL, 0.90 mmol) were added to a solution of 6-chloro-7-methoxychroman (150 mg, 0.76 mmol) in THF (5.0 mL) at −78° C. The reaction mixture was stirred at that temperature for 1 h and CO₂ was bubbled over a period of 40 min. The reaction mixture was slowly brought to ambient temperature, quenched with water (20 mL) and washed with EtOAc (2×25 mL). The aqueous layer was acidified to pH ~2.0 using 1.0 N HCl and extracted into EtOAc (2×30 mL). The combined extracts were dried (Na₂SO₄) and concentrated under reduced pressure to afford the title compound (20 mg, 11%).

¹H NMR (300 MHz, CD₃OD) δ 7.17 (s, 1H), 4.22 (t, 2H), 3.87 (s, 3H), 2.79 (t, 2H), 2.05-2.97 (m, 2H)

ES-MS: m/z 243.7 (M-1)

HPLC retention time 9.20 min., purity: 98.6% at 280 nm.

### Example 47: 2-Fluoro-3-methyl-5,6,7,8-tetrahydronaphthalene-1-carboxylic acid (A-49)

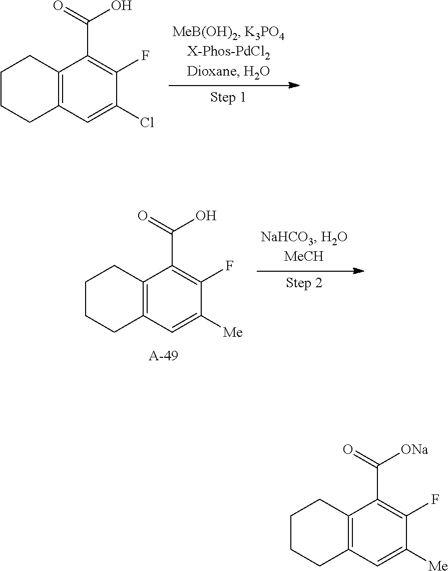

### Step 1: 2-Fluoro-3-methyl-5,6,7,8-tetrahydronaphthalene-1-carboxylic acid

Methyl boronic acid (53 mg, 0.87 mmol), K₃PO₄ (278 mg, 1.31 mmol), and X-phos-PdCl₂ (17 mg, 0.022 mmol) was added to a solution of 3-chloro-2-fluoro-5,6,7,8-tetrahydronaphthalene-1-carboxylic acid (100 mg, 0.24 mmol) in a mixture of dioxan (2 mL) and water (1 mL). The reaction mixture was subjected to microwave conditions at 125° C. for 1 h, quenched with 1N HCl (10 mL), extracted with EtOAc (2×10 mL) then the combined extracts were washed with water (2×5 mL), and brine (2×5 mL) before drying (Na₂SO₄). The filtrate was concentrated in vacuo and purified by semi-prep HPLC using 10-100% acetonitrile/0.1% formic acid in water to provide title product 23 mg (25%) as a white solid.

¹H NMR (300 MHz, CDCl₃); δ 7.02 (d, J_{H-F}=8.1 Hz, 1H), 2.75 (m, 4H), 2.22 (s, 3H), 1.79 (m, 4H) ppm. ¹⁹F NMR (300 MHz, CDCl₃); δ −126.49 ppm.

ES-MS: 207.21 [M−1].

HPLC: Retention Time: 13.72 min., purity: >99% @280 nm.

### Step 2: Sodium 2-fluoro-3-methyl-5,6,7,8-tetrahydronaphthalene-1-carboxylate

NaHCO₃ (5.6 mg, 0.067 mmol) was added to a solution of 2-fluoro-3-methyl-5,6,7,8-tetrahydronaphthalene-1-carboxylic acid (14 mg, 0.067 mmol) in acetonitrile (3.0 mL), and water (1.0 mL). The reaction mixture was stirred at ambient temperature for 15 min. The solvent was removed under reduced pressure and the residue was triturated with diethyl ether (3×10 mL) to afford the title compound (14.5 mg, 93%) as a white solid.

¹H NMR (300 MHz, CDCl₃); δ 6.81 (d, J_{H-F}=7.8 Hz, 1H), 2.80 (m, 4H), 2.19 (s, 3H), 1.79 (m, 4H) ppm.

¹⁹F NMR (300 MHz, CDCl₃); δ −128.94 ppm

ES-MS: 207.52 [M−1].

HPLC: Retention Time: 13.69 min., purity: >99% @280 nm.

### Example 48: 6-Chloro-7-fluoro-2,2-dimethyl-3,4-dihydro-2H-1-benzopyran-8-carboxylic acid (A-61)

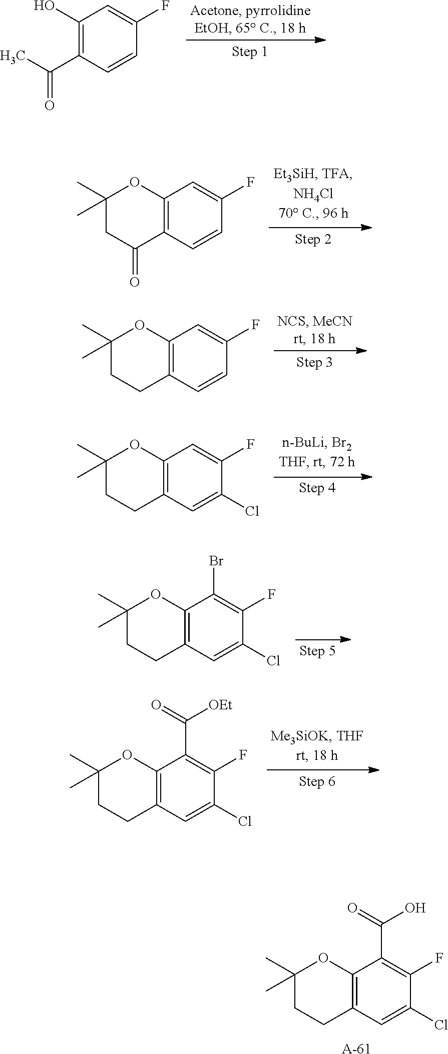

### Step 1: 7-fluoro-2,2-dimethyl-chroman-4-one

4′-Fluoro-2′-hydroxyacetophenone (2.20 g, 14.3 mmol) was dissolved in dry ethanol (20 mL) and the flask was flushed with nitrogen. Pyrrolidine (1.2 mL, 14.3 mmol) and acetone (11 mL) were added, and the reaction mixture was heated at reflux for 65° C. for 18 h and the reaction mixture concentrated in vacuo. The residue was dissolved in CH₂Cl₂ (50 mL), washed with 1 N HCl (3×50 mL) and the organic later was washed with brine (50 mL), dried (Na₂SO₄), then concentrated in vacuo. The crude material was purified by column chromatography on silica gel eluting with a EtOAc/iso-hexane (0%-30%) to provide the title compound (1.73 g, 57%) as an orange solid.

¹H NMR (400 MHz, CDCl₃) δ 7.90-7.83 (m, 1H), 6.71-6.65 (m, 1H), 6.65-6.57 (m, 1H), 2.71 (s, 2H), 1.46 (s, 6H).

### Step 2: 7-fluoro-2,2-dimethyl-chromane

7-fluoro-2,2-dimethyl-chroman-4-one (1.4 g, 7.21 mmol), triethylsilane (4.6 mL, 28.8 mmol) and NH₄Cl (1.54 g, 28.8 mmol) were added to a solution of TFA (10 mL, 132 mmol) and the reaction mixture was heated to 70° C. for 24 h. The reaction mixture was cooled to ambient temperature then TFA (5.0 mL, 7.21 mmol), triethylsilane (2.3 mL, 14.4 mmol) and NH₄Cl (770 mg, 14.4 mmol) were added. The reaction mixture was heated at 70° C. for 96 h. The reaction mixture was cooled to ambient temperature and TFA (5.0 mL, 7.21 mmol) triethylsilane (2.3 mL, 14.4 mmol) and NH₄Cl (770 mg, 14.4 mmol) were added. The reaction mixture was stirred at 70° C. for 72 h. The reaction mixture was concentrated in vacuo and dissolved in EtOAc (50 mL). Aqueous NaHCO₃ and solid Na₂CO₃ were added to pH 7 and aqueous layer was extracted with EtOAc (200 mL). The organic layer was washed with brine (200 mL), dried (Na₂SO₄) and concentrated in vacuo. The residue was purified by column chromatography over silica eluting with EtOAc/iso-hexane (1% to 30%) to afford the title compound (787 mg, 53%) as a green oil.

¹H NMR (400 MHz, CDCl₃) δ 7.03-6.95 (m, 1H), 6.57-6.45 (m, 2H), 2.80-2.68 (m, 2H), 1.79 (t, J=6.8 Hz, 2H), 1.33 (s, 6H).

### Step 3: 6-chloro-7-fluoro-2,2-dimethyl-chromane

NCS (596 mg, 4.46 mmol) was added to a solution of 7-fluoro-2,2-dimethyl-chromane (670 mg, 3.72 mmol) in CH₃CN (40 mL) and the reaction mixture was stirred at ambient temperature for 18 h. The reaction mixture was concentrated in vacuo. The residue was dissolved in water (20 mL) and extracted with EtOAc (30 mL). The organic layer was dried (Na₂SO₄) and concentrated in vacuo. The residue was purified by column chromatography over silica gel, eluting with a gradient of EtOAc/isohexane (1% to 10%) to provide the title compound (350 mg, 38%) as a colourless oil.

¹H NMR (400 MHz, CDCl₃) δ 7.05 (dt, J=8.4, 1.1 Hz, 1H), 6.57 (d, J=10.5 Hz, 1H), 2.71 (tt, J=6.8, 1.4 Hz, 2H), 1.78 (t, J=6.8 Hz, 2H), 1.32 (s, 6H).

### Step 4: 8-bromo-6-chloro-7-fluoro-2,2-dimethyl-chromane

6-chloro-7-fluoro-2,2-dimethyl-chromane (236 mg, 1.10 mmol) was dissolved in dry THF (10 mLand stirred at −78° C. under nitrogen. n-BuLi (2.5 M in hexanes) (0.66 mL, 1.65 mmol) was added dropwise and the reaction mixture was stirred at −78° C. for 1.5 h. Bromine (0.084 mL, 1.65 mmol) was added dropwise, the mixture was warmed to ambient temperature and stirred at ambient temperature for 72 h. The reaction mixture was quenched with saturated Na₂S₂O₃ solution (10 mL) and water (10 mL) and extracted with EtOAc (20 mL). The organic layer was washed with brine (20 mL), dried (Na₂SO₄) filtered and concentrated in vacuo. The residue was purified by column chromatography over silica gel eluting with EtOAc/iso-hexane (1% to 5%) to give the title compound (182 mg, 52%) as a colourless oil.

¹H NMR (400 MHz, CDCl₃) δ 7.05 (dt, J=8.0, 1.0 Hz, 1H), 2.75 (m, 2H), 1.81 (t, J=6.8 Hz, 2H), 1.37 (s, 6H).

### Step 5: Ethyl 6-chloro-7-fluoro-2,2-dimethyl-chromane-8-carboxylate

The reaction was carried out in a COWare reactor consisting of 2 discrete chambers, linked to allow gas transfer. Chamber A was charged with 9-methyl-9H-fluorene-9-carbonyl chloride (281 mg, 1.16 mmol), Pd(dba)₂ (25 mg, 0.043 mmol) and tri-tert-butylphosphine tetrafluoroborate (13 mg, 0.043 mmol). Chamber B was charged with Pd(dppf)Cl₂ (32 mg, 0.043 mmol) and 8-bromo-6-chloro-7-fluoro-2,2-dimethyl-chromane (85 mg, 0.290 mmol). The dry reagents were suspended in dioxan (1.92 mL) for chamber A; in dioxan (0.96 mL) and ethanol (0.96 mL) in chamber B. Both chambers were sparged with nitrogen then N,N-diisopropylethylamine (0.16 mL, 0.92 mmol) was added to chamber A and triethylamine (0.12 mL, 0.84 mmol) was added to chamber B. The vessel was sealed and was stirred at 80° C. for 48 h. The vessel was cooled to ambient temperature and both chambers sparged with nitrogen. The reaction mixture from chamber B was concentrated in vacuo and the residue purified by column chromatography over silica eluting with a gradient of EtOAc/iso-hexane (1% to 10%) to afford the title compound (18 mg, 22%) as a green oil.

¹H NMR (400 MHz, CDCl₃) δ 7.11 (ddd, J=8.2, 1.3, 0.7 Hz, 1H), 4.39 (q, J=7.2 Hz, 2H), 2.72 (tt, J=6.8, 1.4 Hz, 2H), 1.80 (t, J=6.8 Hz, 2H), 1.37 (t, J=7.1 Hz, 4H), 1.32 (s, 6H).

### Step 6: 6-chloro-7-fluoro-2,2-dimethyl-3,4-dihydro-2H-1-benzopyran-8-carboxylic acid

Potassium trimethylsilanolate (0.078 mL, 0.157 mmol, 2M in THF) was added to ethyl 6-chloro-7-fluoro-2,2-dimethyl-chromane-8-carboxylate (18 mg, 0.063 mmol) in THF (1 mL), and the reaction mixture was stirred at ambient temperature for 1.5 h. Further potassium trimethylsilanolate (0.035 mL, 0.070 mmol, 2M in THF) was added and the reaction mixture was stirred at ambient temperature for a further 18 h. The reaction mixture was heated to 45° C. for 1 h. at 50° C. for 1 h, then at 40° C. for 4 h. The reaction mixture was concentrated in vacuo, dissolved in water (10 mL) and washed with EtOAc (2×10 mL). The aqueous layer was acidified with 1 N HCl to pH ~1 and extracted with EtOAc (2×10 mL). The combined extracts were then washed with brine (15 mL), dried (Na₂SO₄), filtered then concentrated in vacuo to yield the title compound (7.2 mg, 44%) as a white solid.

¹H NMR (400 MHz, CDCl₃) δ 7.29-7.26 (m, 1H), 2.80 (tt, J=6.8, 1.3 Hz, 2H), 1.90 (t, J=6.8 Hz, 2H), 1.44 (s, 6H).

UPLC-MS analysis (4 min, Acidic) rt=1.79, m/z=259.1 [M+H]+, >99% purity.

### Example 49: 3-Chloro-4-fluoro-5,6,7,8-tetrahydronaphthalene-1-carboxylic acid (A-17)

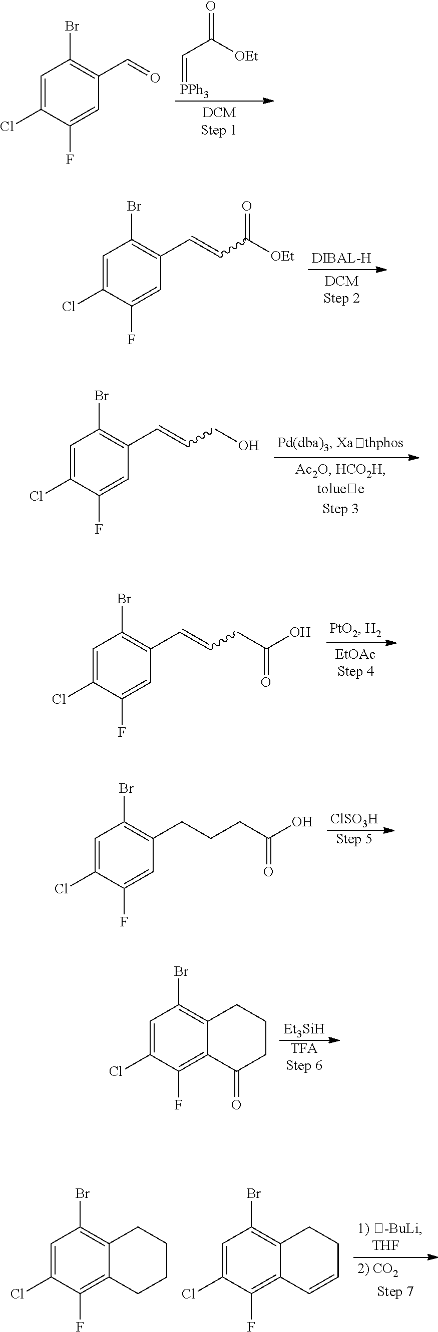

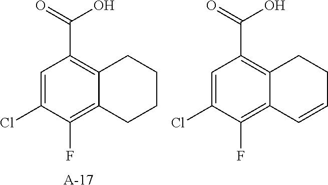

### Step 1: Ethyl 3-(2-bromo-4-chloro-5-fluorophenyl)acrylate

To a solution of 2-bromo-4-chloro-5-fluorobenzaldehyde (2.50 g, 10.5 mmol) in CH₂Cl₂ (50 mL), ethyl(triphenylphosphoranylidene)acetate (3.85 g, 11.1 mmol) was introduced in several portions and the reaction mixture was stirred at ambient temperature for 2 h.

The solvent was removed under reduced pressure and the residue was purified by column chromatography on silica gel eluting with hexane/EtOAc (0-10%) to provide the title compound (2.98 g, 92%).

¹H NMR data is reported for the major trans isomer: (300 MHz, CDC₃) δ 7.90 (dd, 1H), 7.67 (d, 1H), 7.38 (d, 1H), 6.35 (d, 1H), 4.29 (q, 2H), 1.35 (t, 3H).

### Step 2: 3-(2-Bromo-4-chloro-5-fluorophenyl)prop-2-en-1-ol

To a solution of ethyl 3-(2-bromo-4-chloro-5-fluorophenyl)acrylate (1.00 g, 3.25 mmol) in CH₂Cl₂ (10.0 mL) at −78° C., DIBAL-H (1N in toluene, 8.20 mL, 8.16 mmol) was added and the reaction mixture stirred at ambient temperature for 1 h. The mixture was cooled to 0° C., quenched with saturated sodium potassium tartrate (20 mL) and the layers were separated. The organic layer was dried (Na₂SO₄) and concentrated under reduced pressure. The residue was purified by column chromatography on silica gel eluting with hexane/EtOAc (20-30%) to provide the title compound (0.77 g, 90%).

¹H NMR (300 MHz, CDC₃) δ 7.60 (d, 1H), 7.30 (d, 1H), 6.90-6.84 (m, 1H), 6.35-6.26 (m, 1H), 4.40-4.36 (m, 2H).

### Step 3: 4-(2-bromo-4-chloro-5-fluorophenyl)but-3-enoic acid

A mixture of 3-(2-bromo-4-chloro-5-fluorophenyl)prop-2-en-1-ol (1.00 g, 3.77 mmol), Pd₂(dba)₃ (35 mg, 0.04 mmol), Xantphos (87 mg, 0.15 mmol), Ac₂O (1.07 mL, 11.3 mmol) and formic acid (0.43 mL, 11.3 mmol) in toluene (12 mL) was degassed for 30 min. The reaction mixture was heated at 80° C. for 20 h. The solvent was removed under reduced pressure and the resultant residue purified by column chromatography on silica gel eluting with hexane/EtOAc (30-40%) to afford the title compound (0.87 g, 79%).

¹H NMR (300 MHz, CDC₃) δ 7.59 (d, 1H), 7.32 (d, 1H), 6.78-6.73 (m, 1H), 6.30-6.20 (m, 1H), 3.37 (dd, 2H).

### Step 4: 4-(2-Bromo-4-chloro-5-fluorophenyl)butanoic acid

To a solution of 4-(2-bromo-4-chloro-5-fluorophenyl)but-3-enoic acid (0.87 g, 2.96 mmol) in EtOAc (12.0 mL) was added PtO₂ (0.60 g, 2.64 mmol) and the reaction mixture was stirred at ambient temperature for 16 h under an atmosphere of H₂. The mixture was filtered through a pad of Celite®, washed with EtOAc and the filtrate was concentrated under reduced pressure. The residue was triturated with hexanes (2×4.0 mL) to provide the title compound (0.50 g, 57%).

¹H NMR (300 MHz, CDC₃) δ 7.58 (d, 1H), 7.04 (d, 1H), 2.78-2.73 (m, 2H), 2.43 (t, 2H), 1.95 (quint, 2H).

### Step 5: 5-Bromo-7-chloro-8-fluoro-3,4-dihydronaphthalen-1(2H)-one

A mixture of 4-(2-bromo-4-chloro-5-fluorophenyl)butanoic acid (0.50 g, 1.70 mmol) and chlorosulfonic acid (5.0 mL, precooled at 0° C.) was stirred at ambient temperature for 36 h. The reaction mixture was poured carefully dropwise into a mixture of crushed ice and water. After cooling it was extracted EtOAc (2×50 mL) and the combined extracts washed with water (3×30 mL). The combined extracts were dried (Na₂SO₄), filtered and concentrated under reduced pressure to obtain the title compound (0.41 g, 87%).

¹H NMR (300 MHz, CDC₃) δ 7.80 (d, 1H), 2.97 (t, 2H), 2.66 (t, 2H), 2.15 (quint, 2H).

### Step 6: 8-Bromo-6-chloro-5-fluoro-1,2,3,4-tetrahydronaphthalene and 8-Bromo-6-chloro-5-fluoro-1,2-dihydronaphthalene

A mixture of 5-bromo-7-chloro-8-fluoro-3,4-dihydronaphthalen-1(2H)-one (0.21 g, 0.76 mmol), triethylsilane (0.73 mL, 4.54 mmol) and TFA (0.35 mL, 4.54 mmol) was heated in a sealed tube at 75° C. for 16 h. The reaction mixture was cooled, evaporated and purified by column chromatography on silica gel eluting with hexane/EtOAc (0-10%) to obtain a 2:1 mixture of the title compounds 8-bromo-6-chloro-5-fluoro-1,2,3,4-tetrahydronaphthalene and 8-bromo-6-chloro-5-fluoro-1,2-dihydronaphthalene (180 mg, 90%). The mixture was carried forward to the next step without further purification.

### Step 7: 3-Chloro-4-fluoro-5,6,7,8-tetrahydronaphthalene-1-carboxylic acid and 3-Chloro-4-fluoro-7,8-dihydronaphthalene-1-carboxylic acid

To a solution of 8-bromo-6-chloro-5-fluoro-1,2,3,4-tetrahydronaphthalene (7) and 8-bromo-6-chloro-5-fluoro-1,2-dihydronaphthalene (8) (0.18 g, 0.68 mmol) in THF (3.0 mL) at −78° C., n-BuLi (2.5 M in hexane) (0.41 mL, 1.02 mmol) was added. The reaction mixture was stirred at the same temperature for 1 h, CO₂ (g) was bubbled through it for 30 min. and the mixture was slowly brought to 0° C. The reaction mixture was quenched with water (50 mL) and washed with EtOAc (2×15 mL) to remove impurities. The aqueous layer was acidified to pH ~2.0 using 1.0 N HCl and extracted with EtOAc (3×15 mL). The combined organic layers were dried (Na₂SO₄) and concentrated under reduced pressure to afford the title compounds 3-chloro-4-fluoro-5,6,7,8-tetrahydronaphthalene-1-carboxylic acid (9) (7.5 mg, 5%) and 3-chloro-4-fluoro-7,8-dihydronaphthalene-1-carboxylic acid (10) (4.3 mg, 2%), which were separated utilising semi-preparative HPLC using 10-100% acetonitrile/0.1% formic acid in water. 3-Chloro-4-fluoro-5,6,7,8-tetrahydronaphthalene-1-carboxylic acid (A-17): ¹H NMR (300 MHz, CDCl₃) δ 7.96 (d, J_{H-F}=7.5 Hz, 1H), 3.15-3.07 (m, 2H), 3.82-3.12 (m, 2H), 1.84-1.73 (m, 4H).

¹⁹F NMR (300 MHz, CDCl₃); δ −110.64 ppm.

ES-MS: 227.6 [M−1].

HPLC: Retention Time: 12.2 min., purity: >99% at 280 nm.

3-Chloro-4-fluoro-7,8-dihydronaphthalene-1-carboxylic acid: ¹H NMR (300 MHz, CDCl₃) δ 7.93 (d, J_{H-F}=7.5 Hz, 1H), 6.74-6.71 (m, 1H), 6.30-6.25 (m, 1H), 3.26 (t, 2H), 3.37-2.30 (m, 2H).

¹⁹F NMR (300 MHz, CDCl₃); δ −117.02 ppm.

ES-MS: 225.4 [M−1].

HPLC: Retention Time: 11.8 min., purity: >99% at 280 nm.

### Example 50: 6-Chloro-7-fluoro-1-methyl-1,2,3,4-tetrahydroquinoline-8-carboxylic acid (A-40)

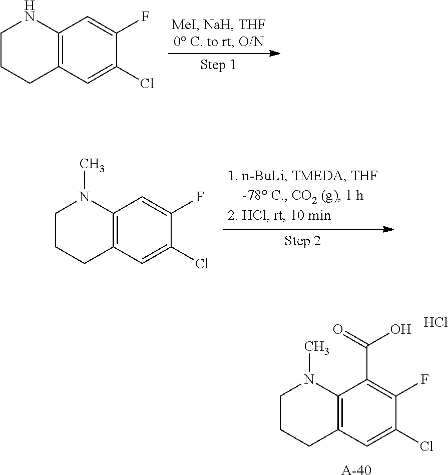

### Step 1: 6-Chloro-7-fluoro-1-methyl-1,2,3,4-tetrahydroquinoline

To a solution of 6-chloro-7-fluoro-1,2,3,4-tetrahydroquinoline (85 mg, 0.46 mmol) in THF (5 mL), NaH (60% in mineral oil) (28 mg, 0.69 mmol) at 0° C. was added and stirred for 30 minutes. Methyl iodide (0.05 ml, 0.81 mmol) was added at 0° C. and the reaction mixture stirred at ambient temperature for 16 h. Extracted with EtOAc (2×20 mL), evaporated and purified by column chromatography on silica gel eluting with EtOAc/hexane (0-20%) to provide 6-chloro-7-fluoro-1-methyl- 1,2,3,4-tetrahydroquinoline (80 mg, 87%).

¹H NMR (300 MHz, CDCl₃) δ 6.88 (d, 1H), 6.30 (d, 1H), 3.22 (t, 2H), 2.85 (s, 3H), 2.68 (t, 2H), 1.94 (q, 2H). ¹⁹F NMR (300 MHz, CDCl₃) δ− 123.47 ppm.

### Step 2: 6-Chloro-7-fluoro-1-methyl-1,2,3,4-tetrahydroquinoline-8-carboxylic acid hydrochloride

To a solution of 6-chloro-7-fluoro-1-methyl-1,2,3,4-tetrahydroquinoline (80 mg, 0.40 mmol) and TMEDA (0.07 ml, 0.44 mmol) in THF (5 mL) at −78° C., n-BuLi (2.5 M in hexane) (0.176 mL, 0.44 mmol) was added. The reaction mixture was stirred at that temperature for 1 h and CO₂ (g) was bubbled for 10 min. The reaction mixture was stirred at −78° C. temperature for 1 h., slowly brought to ambient temperature and quenched with 1M NaOH to pH ~11. The reaction mixture was extracted with CH₂Cl₂ (2×10 mL), the aqueous layer was acidified to pH ~2.0 using 1.0 N HCl, concentrated and purified by semi-prep HPLC using 30 to 100% methanol/0.1% formic acid in water.

The product obtained was treated 1N HCl (2 ml), stirred for 10 min, and concentrated in vacuo to provide the title acid (8 mg, 7%).

¹H NMR (300 MHz, CD₃OD) δ 7.58 (d, J_{H-F}=7.53, 1H), 3.52 (t, 2H), 3.17 (s, 3H), 2.92 (t, 2H), 2.17 (q, 2H). ¹⁹F NMR (300 MHz, CD₃OD) δ− 111.36 ppm.

ES-MS: 244.04 [M+H]HPLC: Retention Time: 10.59 min., purity: >99% @280 nm.

### Example 51: 6-Chloro-7-fluoro-1,2,3,4-tetrahydroquinoline-8-carboxylic acid (A-41)

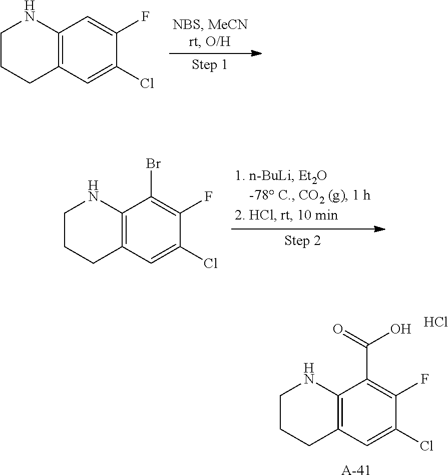

### Step 1: 8-Bromo-6-chloro-7-fluoro-1,2,3,4-tetrahydroquinoline

NBS (527 mg, 2.96 mmol) was added to a solution of 6-chloro-7-fluoro-1,2,3,4-tetrahydroquinoline (500 mg, 2.69 mmol) in acetonitrile (10 mL) and the reaction mixture was stirred at ambient temperature for 16 h. The solvent was removed under reduced pressure, then the residue was dissolved in EtOAc (30 mL) and washed with water (2×10 mL). The organic layer was dried (Na₂SO₄) and concentrated under reduced pressure. The resultant residue was purified by column chromatography on silica gel eluting with hexane/EtOAc (0-10%) to provide the title compound (471 mg, 66%).

¹H NMR (300 MHz, CDCl₃) δ 6.88 (d, 1H), 4.53 (s, 1H), 3.37 (t, 2H), 2.70 (t, 2H), 1.89 (q, 2H). ¹⁹F NMR (300 MHz, CDCl₃) δ− 110.28 ppm.

### Step 2: 6-Chloro-7-fluoro-1,2,3,4-tetrahydroquinoline-8-carboxylic acid hydrochloride

n-BuLi (2.5 M in hexane) (0.17 mL, 0.42 mmol) was added to a solution of 8-bromo-6-chloro-7-fluoro-1,2,3,4-tetrahydroquinoline (100 mg, 0.38 mmol) in diethyl ether (5 mL) at −78° C. The reaction mixture was stirred at that temperature for 1 h and CO₂ (g) was bubbled through it for 10 min. and stirring was continued at −78° C. for 1 h. The mixture was slowly brought to ambient temperature, the pH was adjusted to ~11 with 1M NaOH and the mixture washed with CH₂Cl₂ (2×10 mL). The aqueous layer was separated and acidified to pH ~2.0 using 1.0 N HCl, concentrated then purified by semi-prep HPLC using 30 to 100% methanol/0.1% formic acid in water. The product obtained was treated with 1N HCl (2 ml), stirred for 10 min, and concentrated in vacuo to get 6-chloro-7-fluoro-1,2,3,4-tetrahydroquinoline-8-carboxylic acid hydrochloride (8 mg, 8%).

¹H NMR (300 MHz, CD₃OD) δ 7.09 (d, J_{H-F}=7.75, 1H), 3.40 (t, 2H), 2.76 (t, 2H), 1.89 (q, 2H). ¹⁹F NMR (300 MHz, CD₃OD) δ− 112.66 ppm.

ES-MS: 230.16 [M+H]HPLC: Retention Time: 14.01 min., purity: >99% @254 nm.

### Example 52: 7-Chloro-6-fluoro-1-methyl-1,2,3,4-tetrahydroquinoline-5-carboxylic acid (A-45)

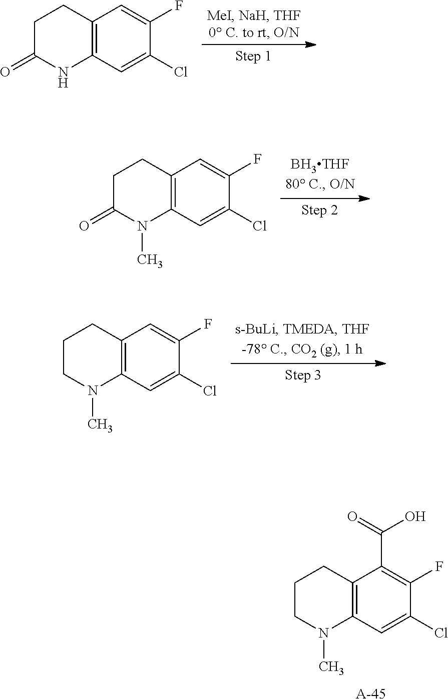 [SMILES: O=C1CCc2cc(F)c(Cl)cc2N1 ; CN1C(=O)CCc2cc(F)c(Cl)cc21 ; CN1CCCc2cc(F)c(Cl)cc21 ; CN1CCCc2c1cc(Cl)c(F)c2C(=O)O ; "A-45"]

### Step 1: 7-Chloro-6-fluoro-1-methyl-3,4-dihydro-1H-quinolin-2-one

NaH (60% in mineral oil) (204.4 mg, 5.11 mmol) was added to a solution of 7-chloro-6-fluoro-3,4-dihydro-1H-quinolin-2-one (680 mg, 3.41 mmol) in DMF (10 mL) at 0° C. and the reaction mixture was stirred for 30 min. Methyl iodide (0.32 ml, 5.11 mmol) was introduced at 0° C. and the reaction mixture stirred at ambient temperature for 16 h before extraction with EtOAc (2×20 mL). The combined extracts were evaporated in vacuo and purified by column chromatography on silica gel eluting with EtOAc/hexane (5-50%) to provide the title compound (275 mg, 37%).

¹H NMR (300 MHz, CDC₃) δ 7.25 (d, 1H), 7.23 (d, 1H), 3.59 (s, 3H), 3.15 (t, 2H), 2.91 (dd, 2H).

¹⁹F NMR (300 MHz, CDC₃) δ− 123.47 ppm.

### Step 2: 7-Chloro-6-fluoro-1-methyl-1,2,3,4-tetrahydroquinoline

7-Chloro-6-fluoro-1-methyl-3,4-dihydro-1H-quinolin-2-one (275 mg, 1.28 mmol), and 1M BH₃·THF (5.15 mL, 5.15 mmol) were heated at reflux for 16 h. The reaction mixture was cooled, carefully quenched with methanol (10 mL), evaporated in vacuo and the resultant residue was purified by column chromatography on silica gel eluting with EtOAc/hexane (0-20%) to obtain 7-chloro-6-fluoro-1-methyl-1,2,3,4-tetrahydroquinoline (210 mg, 81%).

¹H NMR (300 MHz, CDC₃) δ 6.73 (d, 1H), 6.49 (d, 1H), 3.17 (t, 2H), 2.83 (s, 3H), 2.69 (t, 2H), 1.99-1.89 (m, 2H). ¹⁹F NMR (300 MHz, CDC₃) δ− 133.38 ppm.

ES-MS: 200.16 [M+H]

### Step 3: 7-Chloro-6-fluoro-1-methyl-1,2,3,4-tetrahydroquinoline-5-carboxylic acid (4A) and 7-chloro-6-fluoro-1-methyl-1,2,3,4-tetrahydroquinoline-8-carboxylic acid (4B)

s-BuLi (1.4 M in hexane) (0.83 mL, 1.15 mmol) was added to a solution of 7-chloro-6-fluoro-1-methyl-1,2,3,4-tetrahydroquinoline (210 mg, 1.05 mmol) and TMEDA (0.174 ml, 1.15 mmol) in THF (5 mL) at −78° C. The reaction mixture was stirred at the same temperature for 1 h and CO₂ (g) was bubbled through it for 10 min. Stirring was continued at −78° C. for 1 h and the mixture was slowly brought to ambient temperature and the pH was adjusted with 1 M NaOH to ~11. The reaction mixture was washed with CH₂Cl₂ (2×10 mL), the aqueous layer was acidified to pH ~2 with 1.0 N HCl and extracted with EtOAc (2×20 mL). The combined extracts were dried (Na₂SO₄), concentrated, and purified by semi-prep HPLC using 30 to 100% methanol/0.1% formic acid in water to provide 7-chloro-6-fluoro-1-methyl-1,2,3,4-tetrahydroquinoline-5-carboxylic acid (4A) (19 mg, 7%) and 7-chloro-6-fluoro-1-methyl-1,2,3,4-tetrahydroquinoline-8-carboxylic acid (4B) (15 mg, 6%). 7-Chloro-6-fluoro-1-methyl-1,2,3,4-tetrahydroquinoline-5-carboxylic acid (A-45) ¹H NMR (300 MHz, CD₃OD) δ 6.90 (d, J_{H-F}=5.84, 1H), 3.09 (t, 2H), 2.79 (s, 3H), 2.72 (t, 2H), 1.90 (q, 2H). ¹⁹F NMR (300 MHz, CD₃OD) δ− 128.42 ppm.

ES-MS: 242.20 [M−H]HPLC: Retention Time: 14.04 min., purity: 97.9% @254 nm.

### Example 53: 3-Ethyl-2-fluoro-5,6,7,8-tetrahydronaphthalene-1-carboxylic acid (A-51)

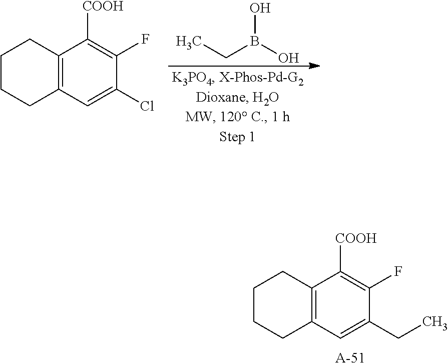

### Step 1: 3-Ethyl-2-fluoro-5,6,7,8-tetrahydronaphthalene-1-carboxylic acid

3-Chloro-2-fluoro-5,6,7,8-tetrahydronaphthalene-1-carboxylic acid (140 mg, 0.61 mmol), ethylboronic acid (90.5 mg, 1.22 mmol), X-Phos-Pd-G2 (24.1 mg, 0.031 mmol), K₃PO₄ (389.8 mg, 1.83 mmol) were suspended in a mixture of dioxan (3.8 mL) and water (1.27 ml) before being degassed for 30 min. The reaction mixture was heated in a sealed tube at 120° C. for 1 h under microwave conditions. The solvent was removed under reduced pressure and the residue was purified by semi-prep HPLC using 10-100% CH₃CN/0.1% formic acid in water to provide the title compound (11.3 mg, 8.3%).

¹H NMR (300 MHz, CDCl₃) δ 7.00 (d, 1H); 2.91-2.78 (br, 2H); 2.77-2.68 (br, 2H); 2.63 (q, 2H); 1.85-1.69 (m, 4H); 1.22 (t, 3H); ES-MS: 221.23 [M−1].

HPLC Retention Time: 11.80 min, 96.3% purity @280 nm.

### Example 54: 3-Fluoro-2-methyl-5,6,7,8-tetrahydronaphthalene-1-carboxylic acid (A-52)

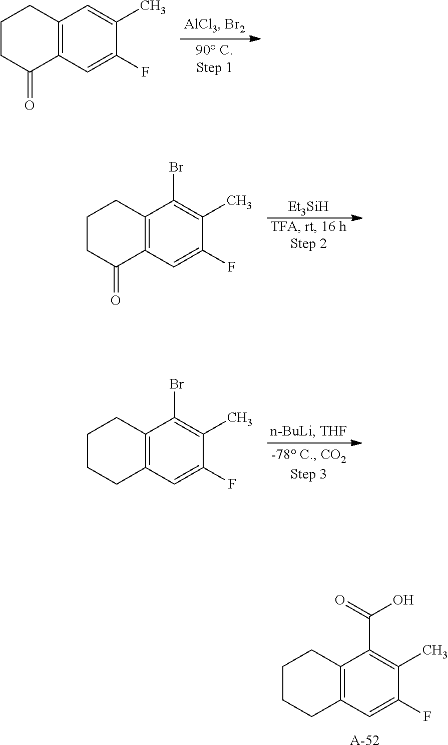

### Step 1: 5-Bromo-7-fluoro-6-methyl-3,4-dihydronaphthalen-1(2H)-one

A mixture of 7-fluoro-6-methyl-3,4-dihydronaphthalen-1(2H)-one (0.4 g, 2.25 mmol) and AlCl₃ (0.6 g, 4.49 mmol) was heated at 90° C. for 20 min., then cooled to 0° C. Bromine (0.138 mL, 2.69 mmol) was added dropwise and the reaction mixture stirred for 10 min. at ambient temperature. The reaction mixture was heated at 90° C. for 16 h, evaporated, and the solid residue was purified by column chromatography on silica gel, eluting with hexane/EtOAc (0-15%) to provide the title compound as a white solid (0.52 g, 90%).

¹H NMR (300 MHz, CDCl₃) δ 7.66 (d, 1H), 3.02 (m, 2H), 2.69 (m, 2H), 2.64 (d, 3H).2.14 (m, 2H) ppm. ¹⁹F NMR (300 MHz, CDCl₃) δ− 113.0 ppm.

### Step 2: 5-Bromo-7-fluoro-6-methyl-1,2,3,4-tetrahydronaphthalene

A mixture of 5-bromo-7-fluoro-6-methyl-3,4-dihydronaphthalen-1(2H)-one (0.6 g, 2.33 mmol), Et₃SiH (2.23 mL, 14.0 mmol) and TFA (1.07 mL, 14.0 mmol) in a sealed tube was stirred at ambient temperature for 24 h. The solvent was removed in vacuo and the residue was purified by column chromatography on silica gel, eluting with hexane/EtOAc (0-5%) to provide the title compound as a white solid (0.47 g, 83%) ¹H NMR (300 MHz, CDCl₃) δ 7.07 (d, 1H), 3.87 (d, 3H), 2.78 (t, 2H), 2.71 (t, 2H). 1.77 (m, 4H) ppm. ¹⁹F NMR (300 MHz, CDCl₃) δ− 116.6 ppm.

### Step 3: 3-Fluoro-2-methyl-5,6,7,8-tetrahydronaphthalene-1-carboxylic acid

To a solution of 5-bromo-7-fluoro-6-methyl-1,2,3,4-tetrahydronaphthalene (0.470 g, 1.93 mmol) in THF (10 mL) at −78° C., n-BuLi (2.5 M in hexane) (1.55 mL, 3.87 mmol) was added and the reaction mixture was stirred at that temperature for 1 h. After 1 h., CO₂ (g) was bubbled for 10 min., the mixture was slowly brought to 0° C., quenched with water and washed with CH₂Cl₂ (2×20 mL) to remove impurities. The aqueous layer was acidified to pH ~2.0 using 1.0 N HCl and extracted with CH₂Cl₂ (3×50 mL).

The combined organic layers were dried (Na₂SO₄) and concentrated under reduced pressure to afford the title compound (0.32 g, 80%).

¹H NMR (300 MHz, CDCl₃) δ 6.83 (d, J_{H-F}=10.50 Hz, 1H), 2.78 (m, 4H), 2.20 (d, 3H), 1.79 (m, 4H) ppm. ¹⁹F NMR (300 MHz, CDCl₃) δ− 123.32 ES-MS: 207.4 [M−1]HPLC: Retention Time: 10.93 min., purity: >99% @280 nm

### Example 55: 6-Chloro-2,2-dimethyl-3,4-dihydro-2H-1-benzopyran-8-carboxylic acid (A-53)

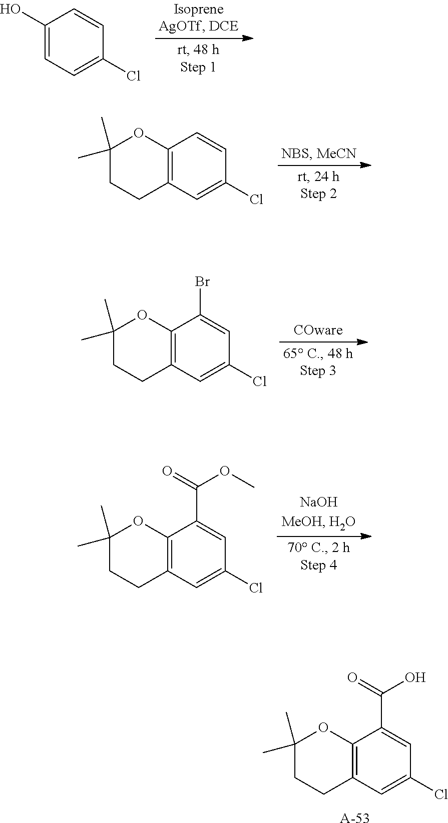

### Step 1: 6-chloro-2,2-dimethyl-chromane

4-chlorophenol (2.45 g, 19.1 mmol) was dissolved in DCE (191 mL) and stirred under nitrogen. isoprene (2.90 mL, 28.6 mmol) was added dropwise. Silver trifluoromethanesulfonate (245 mg, 0.953 mmol) was added and the reaction mixture was stirred for 24 hours. Silver trifluoromethanesulfonate (245 mg, 0.953 mmol) was added and the reaction mixture was stirred at room temperature for 24 more hours.

The reaction was then concentrated in vacuo and the crude material was purified by column chromatography over silica (80 g cartridge) eluting with a gradient of DCM (1% to 7%; v/v) in iso-hexane to afford the title compound (2.58 g, 54%) as a green oil.

¹H NMR (400 MHz, CDCl₃) δ 7.07-6.99 (m, 2H), 6.73-6.65 (m, 1H), 2.74 (tt, J=6.8, 1.1 Hz, 2H), 1.78 (t, J=6.8 Hz, 2H), 1.32 (s, 6H).

### Step 2: 8-bromo-6-chloro-2,2-dimethyl-chromane

To a solution of 6-chloro-2,2-dimethyl-chromane (1.25 g, 6.36 mmol) in dry MeCN (130 mL) was added N-bromosuccinimide (1.35 g, 7.59 mmol) and the reaction mixture was stirred at room temperature for 6 hours. N-bromosuccinimide (1.35 g, 7.59 mmol) was added and the reaction mixture was stirred at room temperature for 24 hours.

The reaction mixture was concentrated in vacuo and the crude material was purified by column chromatography over silica (220 g cartridge) eluting with a gradient of DCM (1% to 5%; v/v) in cyclohexane to afford the title compound (1.76 g, 90%) as a colourless oil.

¹H NMR (400 MHz, CDCl₃) δ 7.34 (dt, J=2.6, 0.8 Hz, 1H), 7.00 (dt, J=2.6, 1.0 Hz, 1H), 2.77 (tt, J=6.8, 0.9 Hz, 2H), 1.80 (t, J=6.8 Hz, 2H), 1.36 (s, 6H).

### Step 3: methyl 6-chloro-2,2-dimethyl-chromane-8-carboxylate

A two chambered CO-Ware tube was used. To chamber A was added 9-methyl-9H-fluorene-9-carbonyl chloride (1.0 g, 4.12 mmol), Pd(dba)₂ (125 mg, 0.218 mmol) and tri-tert-butylphosphine tetrafluoroborate (63 mg, 0.218 mmol). To chamber B was added Pd(dppf)Cl₂ (159 mg, 0.218 mmol) and 8-bromo-6-chloro-2,2-dimethyl-chromane (400 mg, 1.45 mmol) in dry 1,4-dioxane (1.5 mL) was added. 1,4-Dioxane (3 mL) was added to chamber A and MeOH (1.5 mL) was added to chamber B before flushing the rection vessel with nitrogen. N,N-diisopropylethylamine (0.80 mL, 4.59 mmol) was added to chamber A and triethylamine (0.61 mL, 4.35 mmol) was added to chamber B. The reaction mixture was stirred at 65° C. for 48 hours.

The reaction mixture was cooled to room temperature and the reaction mixture was concentrated in vacuo. The crude material was purified by column chromatography over silica (12 g cartridge) eluting with a gradient of DCM (1% to 100%; v/v) in iso-hexane to afford the title compound (100 mg, 26%) as a colourless oil.

¹H NMR (400 MHz, CDCl₃) δ 7.57 (dt, J=2.7, 0.7 Hz, 1H), 7.17 (dt, J=2.7, 1.0 Hz, 1H), 3.86 (s, 3H), 2.79 (tt, J=6.8, 0.9 Hz, 2H), 1.82 (t, J=6.8 Hz, 2H), 1.36 (s, 6H).

### Step 4: 6-chloro-2,2-dimethyl-3,4-dihydro-2H-1-benzopyran-8-carboxylic acid

Methyl 6-chloro-2,2-dimethyl-chromane-8-carboxylate (20 mg, 0.0785 mmol) was dissolved in MeOH (2 mL) and a solution of NaOH (3.5 mg, 0.0864 mmol) in water (1 mL) was added. The reaction mixture was stirred at room temperature for 2 hours followed by a further 2 hours at 70° C. The reaction mixture was then concentrated in vacuo. The residue was dissolved in water (5 mL) and extracted with DCM (5 mL). The aqueous layer was acidified with 1 N HCl until pH 2. The aqueous layer was extracted with DCM (10 mL) and EtOAc (10 mL). The organic layers were combined, dried over Na₂SO₄, filtered and concentrated in vacuo. The residue was dissolved in acetonitrile (1 mL), extracted with isohexane (1 mL), concentrated in vacuo.

The residue was transferred to a vial and dissolved in water (1 mL) and extracted with EtOAc (1 mL). The aqueous layer was acidified with conc. HCl until pH 2. The mixture was then extracted with EtOAc (2×1 mL). Both EtOAc extracts were concentrated in vacuo. The residue was purified by prep-HPLC to afford the title compound (6.5 mg, 34%) as a pink solid.

UPLC-LCMS analysis (6 min, Basic) rt=1.37, m/z=239.1 [M−H]-, 100% purity.

¹H NMR (400 MHz, CDCl₃) δ 7.99 (dt, J=2.8, 0.9 Hz, 1H), 7.29 (dt, J=2.8, 1.1 Hz, 1H), 2.86 (tt, J=6.8, 0.9 Hz, 2H), 1.95 (t, J=6.8 Hz, 2H), 1.49 (s, 6H).

### Example 56: 3-Cyclopropyl-2-fluoro-5,6,7,8-tetrahydronaphthalene-1-carboxylic acid (A-54)

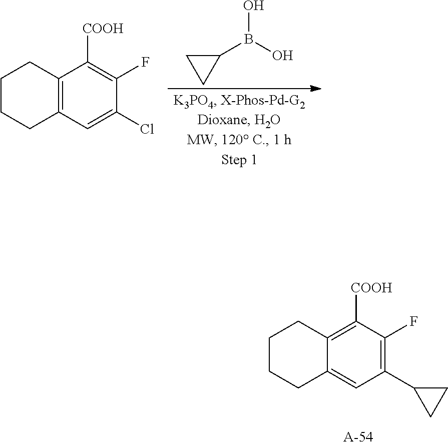

### Step 1: 3-Cyclopropyl-2-fluoro-5,6,7,8-tetrahydronaphthalene-1-carboxylic acid

3-Fluoro-2-chloro-5,6,7,8-tetrahydro-1-naphthoic acid (94 mg, 0.41 mmol), cyclopropylboronic acid (70.6 mg, 0.82 mmol), X-Phos-Pd-G2 (16.2 mg, 0.02 mmol) and K₃PO₄ (261.7 mg, 1.23 mmol) in dioxan (2.6 mL) and water (0.85 ml) was degassed for 30 min. The reaction mixture was heated in a sealed tube at 120° C. for 1 h under microwave conditions. The solvent was removed under reduced pressure and the residue was purified by semi-prep HPLC with 10-100% CH₃CN/0.1% formic acid in water to provide the title compound (15.3 mg, 10.6%).

¹H NMR (300 MHz, CDCl₃) δ 6.69 (d, 1H); 2.92-2.77 (br, 2H); 2.74-2.60 (br, 2H); 2.10-1.95 (m, 1H); 1.84-1.67 (br, 4H); 1.02-0.88 (m, 2H); 0.74-0.62 (m, 2H); ES-MS: 233.71 [M−1].

HPLC Retention Time: 11.92 min, 97.7% purity @280 nm.

### Example 57: 2-Fluoro-3-iodo-5,6,7,8-tetrahydronaphthalene-1-carboxylic acid (A-55)

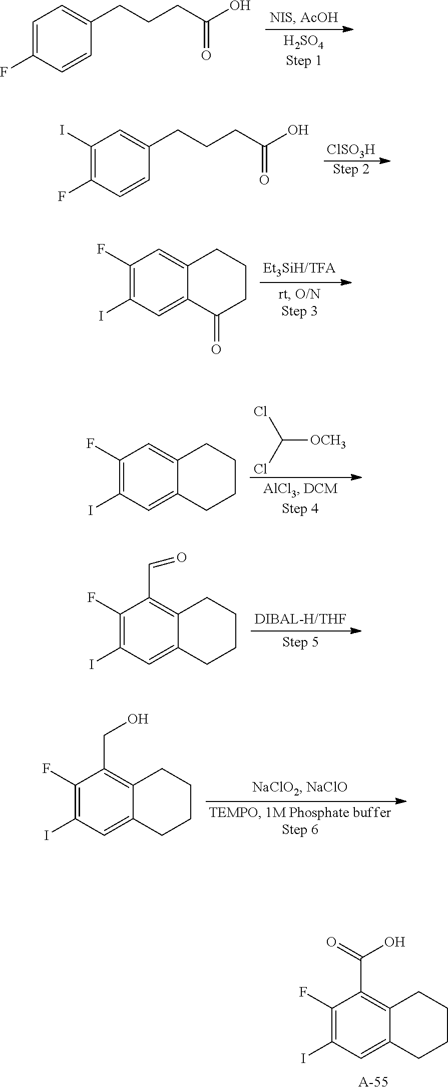

### Step 1: 4-(4-Fluoro-3-iodophenyl)butanoic acid

N-iodosuccinimide (2.22 g, 9.87 mmol) was added to a solution of 4-(4-fluorophenyl)butanoic acid (2.0 g, 10.98 mmol) in a mixture of acetic acid (15 mL) and sulphuric acid (6.25 mL) at 0° C. The reaction mixture was stirred at that temperature for 4 h. and the orange solution was poured into a mixture of crushed ice and water; a solid formed, which was collected by filtration after the ice had melted. The white solid was dried to provide the title compound (2.90 g, 86%).

¹H NMR (300 MHz, CDCl₃) δ 8.71 (Br s, 1H), 7.85 (dd, 1H), 7.44-7.36 (m, 1H), 7.25 (t, 1H), 2.90 (t, 2H), 2.66 (t, 2H), 2.30-2.12 (m, 2H). ¹⁹F NMR (300 MHz, CDCl₃); δ −98.15 ppm.

ES-MS: 307.24 [M−1].

### Step 2: 6-Fluoro-7-iodo-3,4-dihydronaphthalen-1(2H)-one

A mixture of 4-(4-fluoro-3-iodophenyl)butanoic acid (2.90 g, 9.4 mmol), and chlorosulphonic acid (15 mL, precooled to 0° C.) was stirred at ambient temperature for 4 h. The dark brown solution was poured carefully dropwise into a mixture of crushed ice and water. The reaction mixture was extracted into EtOAc (2×50 mL), the combined extracts washed with water (3×10 mL), brine (20 mL), the solution was dried (Na₂SO₄) and concentrated in vacuo to obtain the title compound (2.20 g, 80%) as a brown solid.

¹H NMR (300 MHz, CDCl₃) δ 7.71 (d, 1H), 7.63 (d, 1H), 2.89 (t, 2H), 2.62 (t, 2H), 2.12-2.05 (m, 2H). ¹⁹F NMR (300 MHz, CDCl₃); δ −96.12 ppm.

### Step 3: 6-Fluoro-7-iodo-1,2,3,4-tetrahydronaphthalene

A mixture of 6-fluoro-7-iodo-3,4-dihydronaphthalen-1(2H)-one (2.20 g, 7.58 mmol), triethyl-silane (5.29 g, 45.5 mmol) and TFA (5.20 g, 45.5 mmol) was heated in a sealed tube at 80° C. for 16 h. The reaction mixture was cooled, evaporated and purified by column chromatography on silica gel eluting with hexane/EtOAc (0-10%) to obtain the title compound (1.08 g, 51%) as a colourless oil.

¹H NMR (300 MHz, CDCl₃) δ 7.42 (d, 1H), 6.76 (d, 1H), 2.80-2.60 (m, 4H), 1.82-1.72 (m, 4H). ¹⁹F NMR (300 MHz, CDCl₃); δ −99.98 ppm.

### Step 4: 2-Fluoro-3-iodo-5,6,7,8-tetrahydronaphthalene-1-carbaldehyde

Aluminium chloride (0.78 g, 5.87 mmol) was introduced to a solution of 6-fluoro-7-iodo-1,2,3,4-tetrahydronaphthalene (1.08 g, 3.90 mmol) in dry CH₂Cl₂ (10 mL) at −78° C. under an argon atmosphere. A solution of C₁₂CHOMe (0.52 mL, 5.87 mmol) in dry CH₂Cl₂ (10 mL) was slowly added to the reaction mixture at −78° C. The reaction mixture was stirred at that temperature for 1 h and at 0° C. for 1 h., quenched with saturated aqueous NaHCO₃ (10 mL) and stirred at ambient temperature for 30 min.

The organic layer was separated and the aqueous layer extracted with CH₂Cl₂ (2×30 mL). The combined organic extracts were washed with brine (15 mL), dried (Na₂SO₄) and concentrated to obtain (0.55 g, 46%) to provide a yellow oil. The crude mixture was used for the next step without purification.

¹H NMR (300 MHz, CDCl₃) δ 10.46 (s, 1H), 7.67 (d, 1H), 3.15-2.98 (m, 2H), 2.80-2.65 (m, 2H), 1.83-1.66 (m, 4H). ¹⁹F NMR (300 MHz, CDC₃); δ −105.43 ppm.

### Step 5: (2-Fluoro-3-iodo-5,6,7,8-tetrahydronaphthalen-1-yl)methanol

DIBAL-H (1N in toluene, 9.04 mL, 9.04 mmol) was added to a solution of 2-fluoro-3-iodo-5,6,7,8-tetrahydronaphthalene-1-carbaldehyde (0.55 g, 1.81 mmol) in THF (3.0 mL) at 0° C., and stirred at ambient temperature for 16 h. The reaction mixture was cooled to 0° C., quenched with saturated sodium potassium tartrate (5 mL), stirred for 30 min., and extracted into EtOAc (2×25 mL). The combined extracts were dried (Na₂SO₄), evaporated and the residue purified by column chromatography on silica gel eluting with hexane/EtOAc (0-100%) to provide (2-fluoro-3-iodo-5,6,7,8-tetrahydronaphthalen-1-yl)methanol (0.46 g, 83%) as a white solid.

¹H NMR (300 MHz, CDC₃) δ 7.42 (d, 1H), 4.74 (d, 2H), 2.84 (t, 2H), 2.70 (t, 2H), 1.85-1.68 (m, 4H). ¹⁹F NMR (300 MHz, CDC₃); δ −103.76 ppm.

### Step 6: 2-Fluoro-3-iodo-5,6,7,8-tetrahydronaphthalene-1-carboxylic acid

1.0 M Sodium phosphate buffer (pH 6.0, 12.0 mL) was added to a mixture of (2-fluoro-3-iodo-5,6,7,8-tetrahydronaphthalen-1-yl)methanol (0.46 g, 1.50 mmol) in CH₃CN (12.0 mL), followed by addition of NaClO₂ (0.34 g, 3.75 mmol), NaOCl (0.2 mL, 4-4.99 M solution) and TEMPO (0.012 g, 0.075 mmol) at ambient temperature. The mixture was stirred for 16 h. and additional NaOCl (0.2 mL, 4-4.99 M solution) and TEMPO (0.012 g, 0.075 mmol) were introduced then stirring was continued at ambient temperature for

2 h. The reaction mixture was quenched with 1.0 N NaOH (15 mL) and washed with EtOAc (2×25 mL). The aqueous layer was acidified to pH ~2.0 using 1.0 N HCl and extracted into EtOAc (2×25 mL). The combined extracts were dried (Na₂SO₄) and evaporated to obtain crude title compound (0.20 g). Of this sample, 0.08 g was purified by semi-prep HPLC, eluting with 10-100% CH₃CN/0.1% formic acid in water.

¹H NMR (300 MHz, CDC₃) δ 8.53-7.64 (br, 1H); 7.55 (d, J_{H-F}=6.03 Hz, 1H); 2.89-2.67 (m, 4H); 1.84-1.68 (m, 4H). ¹⁹F NMR (300 MHz, CDC₃); δ −98.116 ppm.

ES-MS: 319.6 [M−1].

HPLC: Retention Time: 11.89 min., purity: 99.2% @280 nm.

### Example 58: 6-Fluoro-7-methyl-3,4-dihydro-2H-1-benzopyran-5-carboxylic acid (A-58)

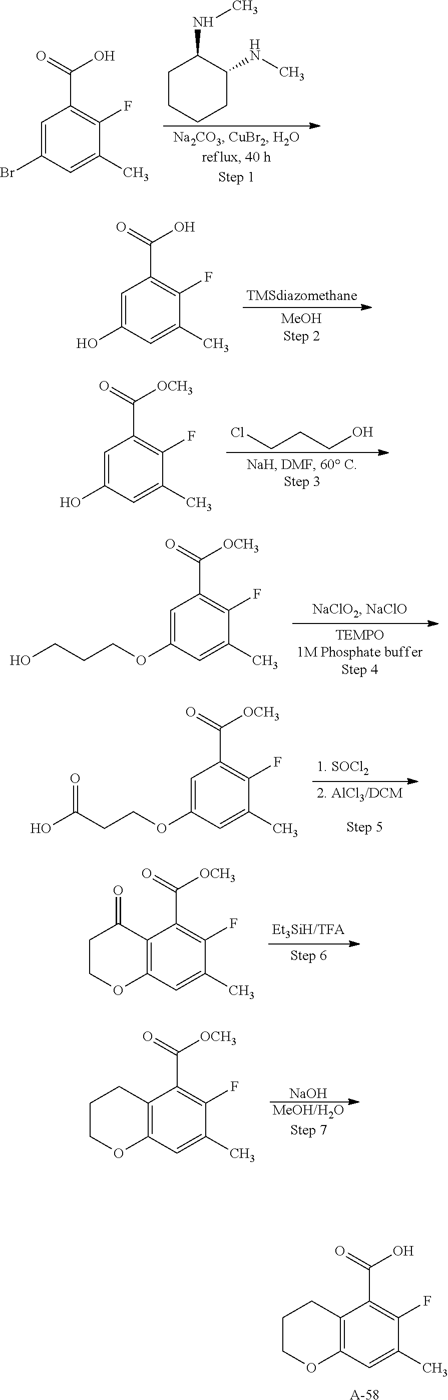

Step 1: 2-Fluoro-5-hydroxy-3-methylbenzoic acid

Na₂CO₃ (2.27 g, 21.45 mmol) was added to a solution of 5-bromo-2-fluoro-3-methylbenzoic acid (2.0 g, 8.58 mmol) in degassed H₂O (25.0 mL) and heated at 100° C. for 30 min. After 30 min., CuBr₂ (0.2 g, 0.858 mmol) and trans-N,N″-dimethylcyclohexane-1,2-diamine (0.244 g, 1.72 mmol) in degassed H₂O (10.0 mL) were introduced into the reaction mixture at 100° C., and heated at same temperature for 40 h. The reaction mixture was cooled and acidified to pH ~2.0 with concentrated HCl, extracted with EtOAc (2×40 mL) and the combined extracts were washed with H₂O (2×25 mL). The organic layer was dried (Na₂SO₄) and concentrated under reduced pressure to obtain the crude product (1.30 g, 89%) as a white solid, which was utilised directly in the next step.

¹H NMR (300 MHz, DMSO-d₆) δ 10.48-12.62 (br s, 1H), 9.54 (s, 1H), 7.00-6.95 (m, 1H), 6.88-6.82 (m, 1H), 2.2 (s, 3H). ¹⁹F NMR (300 MHz, CDCl₃); δ −127.82 ppm.

ES-MS: 169.1 [M−1].

### Step 2: Methyl 2-fluoro-5-hydroxy-3-methylbenzoate

TMS-diazomethane (7.64 mL, 15.28 mmol) was added to a solution of 2-fluoro-5-hydroxy-3-methylbenzoic acid (1.30 g, 7.64 mmol) in dry CH₃OH (15 mL) at 0° C. under an argon atmosphere. The reaction mixture was stirred at ambient temperature for 16 h, concentrated, and the residue purified by column chromatography on silica gel eluting with hexane/EtOAc (0-50%) to obtain title compound as a colourless oil (1.1 g, 78%).

¹H NMR (300 MHz, CDCl₃) δ 7.29-7.22 (m, 1H); 6.94-6.88 (m, 1H); 3.94 (s, 3H);

2.23 (s, 3H). ¹⁹F NMR (300 MHz, CDCl₃); δ −124.47 ppm.

### Step 3: Methyl 2-fluoro-5-(3-hydroxypropoxy)-3-methylbenzoate

NaH (0.311 g, 7.80 mmol) was added to a solution of methyl 2-fluoro-5-hydroxy-3-methylbenzoate (1.1 g, 5.97 mmol) in DMF (10.0 mL) at ambient temperature and the reaction mixture stirred at ambient temperature for 30 min. 3-Chloro-1-propanol (0.713 g, 7.16 mmol) was added dropwise and the mixture heated at 60° C. for 16 h., cooled, quenched with H₂O (5 mL) and extracted into EtOAc (2×30 mL). The combined extracts were dried (Na₂SO₄), concentrated and the resultant residue purified by column chromatography on silica gel eluting with hexane/EtOAc (0-100%) to provide the title compound as a yellow oil (0.5 g, 34%).

¹H NMR (300 MHz, CDCl₃) δ 7.25-7.18 (m, 1H); 6.95-6.89 (m, 1H); 4.10 (t, 2H); 3.91 (s, 3H), 3.85 (t, 2H); 2.27 (s, 3H); 2.09-1.96 (m, 2H). ¹⁹F NMR (300 MHz, CDCl₃); δ −123.96 ppm.

### Step 4: 3-(4-Fluoro-3-(methoxycarbonyl)-5-methylphenoxy)propanoic acid

To a solution of methyl 2-fluoro-5-(3-hydroxypropoxy)-3-methylbenzoate (0.5 g, 2.06 mmol) in CH₃CN (10.0 mL), 1.0 M sodium phosphate buffer (pH 6.0, 10.0 mL) was added followed by NaClO₂ (0.466 g, 5.15 mmol), NaOCl (0.2 mL, 4-4.99M solution) and TEMPO (16 mg, 0.103 mmol). The reaction mixture was stirred at ambient temperature for 16 h. To this mixture was added an additional NaOCl (0.2 mL, 4-4.99M solution) and TEMPO (16 mg, 0.103 mmol) and the reaction mixture was stirred for a further 2 h. The reaction mixture was quenched with 1.0 N NaOH (20 mL) and washed with EtOAc (2×30 mL). The aqueous layer was acidified to pH ~2.0 using 1.0 N HCl and extracted with EtOAc (2×50 mL). The combined organic layers were dried (Na₂SO₄) and concentrated under reduced pressure to afford the title compound (0.4 g, 76%).

¹H NMR (300 MHz, CDCl₃) δ 7.25-7.16 (m, 1H); 6.96-6.88 (m, 1H); 4.23 (t, 2H); 3.92 (s, 3H), 2.72 (t, 2H); 2.28 (s, 3H). ¹⁹F NMR (300 MHz, CDCl₃); δ −124.12 ppm.

ES-MS: 255.6 [M−1].

### Step 5: Methyl 6-fluoro-7-methyl-4-oxochroman-5-carboxylate

A mixture of 3-(4-fluoro-3-(methoxycarbonyl)-5-methylphenoxy)propanoic acid (0.40 g, 1.56 mmol), and thionyl chloride (3 mL) was heated at reflux for 1.5 h., cooled and evaporated. AlCl₃ (0.27, 2.03 mmol) was introduced at 0° C. to the intermediate acid chloride in 1,2-dichloroethane (10 mL) and the mixture stirred at ambient temperature for 16 h before being poured carefully dropwise onto a mixture of crushed ice and a 1 N HCl solution (10 mL). After cooling, the reaction mixture was extracted with CH₂Cl₂ (2×30 mL) and the combined extracts washed with H₂O (3×10 mL) and brine (20 mL).

The combined extracts were dried (Na₂SO₄) and concentrated in vacuo to obtain the solid title compound (0.35 g, 94%).

¹H NMR (300 MHz, CDCl₃) δ 6.87 (d, 1H); 4.49 (t, 2H); 3.98 (s, 3H); 2.77 (t, 2H), 2.29 (s, 3H). ¹⁹F NMR (300 MHz, CDCl₃); δ −129.70 ppm.

### Step 6: Methyl 6-fluoro-7-methylchroman-5-carboxylate

A mixture of methyl 6-fluoro-7-methyl-4-oxochroman-5-carboxylate (0.25 g, 1.05 mmol), triethylsilane (0.73 g, 6.30 mmol) and TFA (0.72 g, 6.30 mmol) was heated in a sealed tube at 80° C. for 16 h. The reaction mixture was cooled, evaporated and purified by column chromatography on silica gel eluting with hexane/EtOAc (0-10%) to give the title compound (0.2 g, 85%) as a colourless oil.

¹H NMR (300 MHz, CDCl₃) δ 6.69 (d, 1H); 4.11 (t, 2H); 3.91 (s, 3H); 2.76 (t, 2H), 2.19 (s, 3H); 2.00-1.90 (m, 2H). ¹⁹F NMR (300 MHz, CDCl₃); δ −128.44 ppm.

### Step 7: 6-Fluoro-7-methyl-3,4-dihydro-2H-1-benzopyran-5-carboxylic acid

NaOH (0.027 g, 0.67 mmol) was added to a solution of methyl 6-fluoro-7-methylchroman-5-carboxylate (0.075 g, 0.334 mmol) in CH₃OH (3 mL) and H₂O (1 mL).

The reaction mixture was heated in microwave at 150° C. for 50 min and concentrated. The residue was dissolved in H₂O (5 mL), washed with CH₂Cl₂ (10 mL). The aqueous layer was acidified with 1 N HCl to pH ~2, extracted with EtOAc (2×20 mL) and the combined extracts washed with H₂O (10 mL) and brine (10 mL). The organic layer was dried (Na₂SO₄) and concentrated to obtain title compound (66 mg, 94% yield) as a white solid.

¹H NMR (300 MHz, CD₃OD) δ 6.72 (d, J_{H-F}=6.92 Hz, 1H); 4.13 (t, 2H); 2.79 (t, 2H), 2.21 (s, 3H); 2.02-1.92 (m, 2H). ¹⁹F NMR (300 MHz, CDCl₃); δ −132.00 ppm.

ES-MS: 209.6[M−1].

HPLC: Retention Time: 9.07 min., purity: >99% @280 nm.

### Example 59: 2-Fluoro-3-(trifluoromethyl)-5,6,7,8-tetrahydronaphthalene-1-carboxylic acid (A-60)

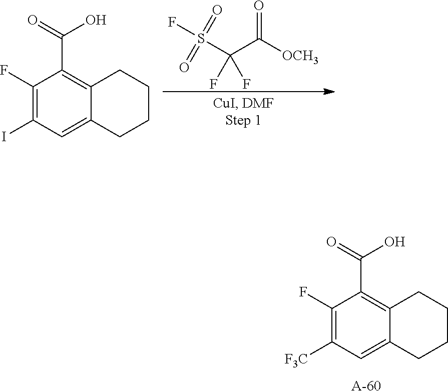

### Step 1: 2-Fluoro-3-(trifluoromethyl)-5,6,7,8-tetrahydronaphthalene-1-carboxylic acid

Methyl 2,2-difluoro-2-(fluorosulfonyl)acetate (0.16 mL, 1.25 mmol) and Cul (0.086 g, 0.45 mmol) were added to a solution of 2-fluoro-3-iodo-5,6,7,8-tetrahydronaphthalene-1-carboxylic acid (0.08 g, 0.25 mmol) in DMF (1.0 mL) and HMPA (0.22 mL, 1.25 mmol). The reaction mixture was heated at 80° C. for 16 h, cooled and EtOAc (40 mL) was added while stirring and the aqueous layer was acidified to pH ~2.0 using 1.0 N HCl. The mixture was extracted with EtOAc (2×20 mL) and the combined extracts washed with H₂O (2×15 mL). The organic layer was dried (Na₂SO₄) and concentrated.

The residue was subjected to semi-preparative HPLC using 10-100% CH₃CN/0.1% formic acid in H₂O to afford and again purified by column chromatography on silica gel eluting with hexane/EtOAc (10-100%) to provide the title compound (2.3 mg, 3.5%).

¹H NMR (300 MHz, CD₃OD) δ 7.31 (d, J_{H-F}=7.2 Hz, 1H); 2.92-2.77 (m, 4H); 1.87-1.78 (m, 4H). ¹⁹F NMR (300 MHz, CDCl₃); δ −62.34 and -126.35 ppm.

ES-MS: 261.7 [M−1].

HPLC: Retention Time: 11.70 min., purity: 94.9% @280 nm.

### Example 60: 5-Chloro-6-fluoro-2,3-dihydro-1H-indene-4-carboxylic acid (A-62) and 6-Chloro-5-fluoro-2,3-dihydro-1H-indene-4-carboxylic acid (A-63)

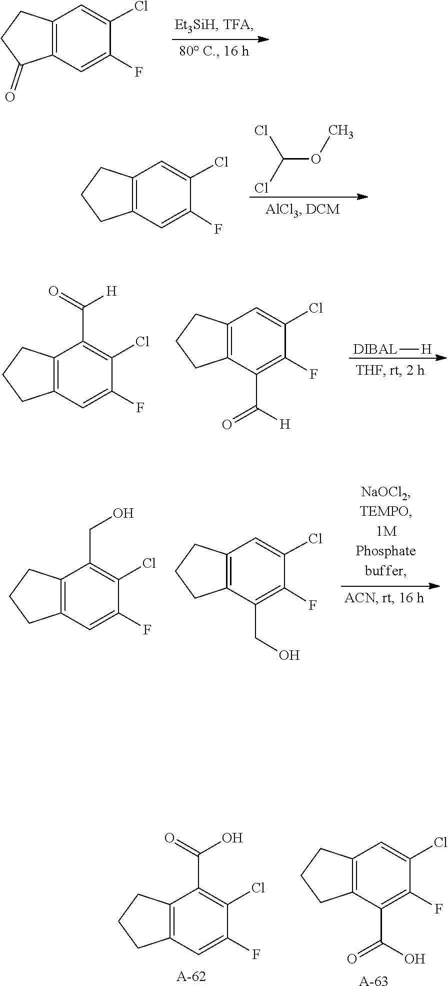

### Step 1: 5-chloro-6-fluoro-2,3-dihydro-1H-inden-1-one

A mixture of 5-chloro-6-fluoro-2,3-dihydro-1H-inden-1-one (1.0 g, 5.42 mmol) in Et₃SiH (5.19 mL, 32.52 mmol) and TFA (2.49 mL, 32.52 mmol) in a sealed tube was heated at 80° C. over a period of 16 h. After completion, the solvent was removed under reduced pressure and the residue subjected to column chromatography on silica gel (0-5% hexanes/EtOAc) to obtain 5-chloro-6-fluoro-2,3-dihydro-1H-indene (900 mg, 97% yield) as a white solid.

¹H NMR (300 MHz, CDCl₃) δ 7.47 (d, 1H); 7.24 (d, 1H); 3.19-3.12 (m, 4H); 2.43-2.35 (m, 2H).

### Step 2: 5-Chloro-6-fluoro-2,3-dihydro-1H-inden-4-carbaldehyde and 6-Chloro-5-fluoro-2,3-dihydro-1H-inden-4-carbaldehyde

To a solution of 5-chloro-6-fluoro-2,3-dihydro-1H-indene (900 mg, 5.28 mmol) in dry CH₂Cl₂ (10 ml) was added aluminium chloride (985 mg, 7.39 mmol) at −78° C. under an argon atmosphere. A solution of Cl₂CHOMe (0.7 ml, 7.39 mmol) in dry CH₂Cl₂ (5 mL) was slowly added to the reaction mixture at −78° C. The reaction mixture was stirred at −78° C. for 1 hr and at 0° C. for 2 hr. The reaction mixture was quenched with saturated aqueous NaHCO₃ (10 mL) and stirred at ambient temperature for 30 min. The organic layer was separated, and the aqueous layer extracted with CH₂Cl₂ (2×30 mL). The combined organic extracts were washed with saturated brine (15 mL), dried (Na₂SO₄) and filtered. The filtrate was concentrated, and a mixture of 5-chloro-6-fluoro-2,3; dihydro-1H-inden-4-carbaldehyde (major isomer) and 6-chloro-5-fluoro-2,3-dihydro-1H-inden-4-carbaldehyde (minor isomer) (720 mg, 69% yield) was obtained as a yellow oil. The crude mixture was used for the next step without purification.

Major isomer: ¹H NMR (300 MHz, CDC₃) δ 10.52 (s, 1H); 7.21 (d, 1H); 3.20-3.18 (m, 2H); 2.90-2.81 (m, 2H); 2.18-2.08 (m, 2H).

Minor isomer: ¹H NMR (300 MHz, CDC₃) δ 10.40 (s, 1H); 7.42 (d, 1H); 3.20-3.18 (m, 2H); 2.9-2.81 (m, 2H); 2.18-2.08 (m, 2H).

### Step 3: (5-Chloro-6-fluoro-2,3-dihydro-1H-inden-4-yl) methanol and 6-Chloro-5-fluoro-2,3-dihydro-1H-inden-4-yl) methanol

To a solution of 3-chloro-2-fluoro-5,6,7,8-tetrahydronapthalene-1-carbaldehyde and 2-chloro-3-fluoro-5,6,7,8-tetrahydronapthalene-1-carbaldehyde (720 mg, 3.63 mmol) in THF (10.0 mL) at 0° C., DIBAL-H (1M in toluene, 5.44 mL, 5.44 mmol) was added and the reaction mixture was stirred at ambient temperature for 2 h. The reaction mixture was cooled to 0° C., quenched with saturated sodium potassium tartrate (5 mL) and stirred for 30 min. The reaction mixture was extracted with EtOAc (2×50 mL). The organic layer was dried (Na₂SO₄), concentrated under reduced pressure to a residue. The mixture of title compounds (640 mg, 88%) was reacted directly in the next step.

Major isomer: ¹H NMR (300 MHz, CDC₃) δ 7.00 (d, 1H); 4.78 (d, 2H); 3.03-2.89 (m, 4H); 2.20-2.08 (m, 2H).

Minor isomer: ¹H NMR (300 MHz, CDC₃) δ 7.20 (d, 1H); 4.72 (s, 2H); 3.03-2.89 (m, 4H); 2.20-2.08 (m, 2H).

### Step 4: 5-Chloro-6-fluoro-2,3-dihydro-1H-indene-4-carboxylic acid and 6-chloro-5-fluoro-2,3-dihydro-1H-indene-4-carboxylic acid

To a mixture of (5-chloro-6-fluoro-2,3-dihydro-1H-inden-4-yl) methanol and 6-chloro-5-fluoro-2,3-dihydro-1H-inden-4-yl) methanol (640 mg, 3.19 mmol) in acetonitrile (30.0 mL), 1.0 M sodium phosphate buffer (pH 6.0, 30.0 mL) was introduced. This was followed by addition of NaClO₂ (721.04 mg, 7.97 mmol), NaOCl (0.2 mL, 4-4.99 M solution) and TEMPO (25 mg, 0.16 mmol) at ambient temperature and the reaction mixture was stirred for 16 h. The reaction mixture was quenched with 1.0 N NaOH (30 mL), extracted with EtOAc (2×30 mL) and the aqueous layer was acidified to pH ~2.0 with 1 N HCl before being extracted into EtOAc (2×50 mL). The combined organic layers were dried (Na₂SO₄) and concentrated under reduced pressure to obtain a mixture of 5-chloro-6-fluoro-2,3-dihydro-1H-inden-4-carboxylic acid (major isomer) and 6-chloro-5-fluoro-2,3-dihydro-1H-inden-4-carboxylic acid (minor isomer) (205 mg, 34%). The mixture was separated by semi-prep HPLC eluting with 25-100% acetonitrile/0.1% formic acid in water.

5-Chloro-6-fluoro-2,3-dihydro-1H-indene-4-carboxylic acid (A-62) - ¹H NMR (300 MHz, CDC₃) δ 7.16 (d, J_{H-F}=9.0 Hz, 1H); 3.00-2.92 (m, 1H); 3.00-2.92 (m, 4H); 2.19-2.09 (m, 2H).

ES-MS: 213.7 [M−1].

HPLC: Retention Time: 10.07 min., Purity: 97.3% @254 nm.

6-chloro-5-fluoro-2,3-dihydro-1H-indene-4-carboxylic acid (A-63) - H NMR (300 MHz, CDC₃) δ 7.44 (d, J_{H-F}=6.7 Hz, 1H); 3.13-3.08 (m, 2H); 2.95-2.90 (m, 2H); 2.18-2.08 (m, 2H).

ES-MS: 213.6 [M−1].

HPLC: Retention Time: 10.80 min., Purity: 98.8% @254 nm.

### Example 61: 2-Bromo-3-chloro-5,6,7,8-tetrahydronaphthalene-1-carboxylic acid (A-66) and 3-bromo-2-chloro-5,6,7,8-tetrahydronaphthalene-1-carboxylic acid (A-67)

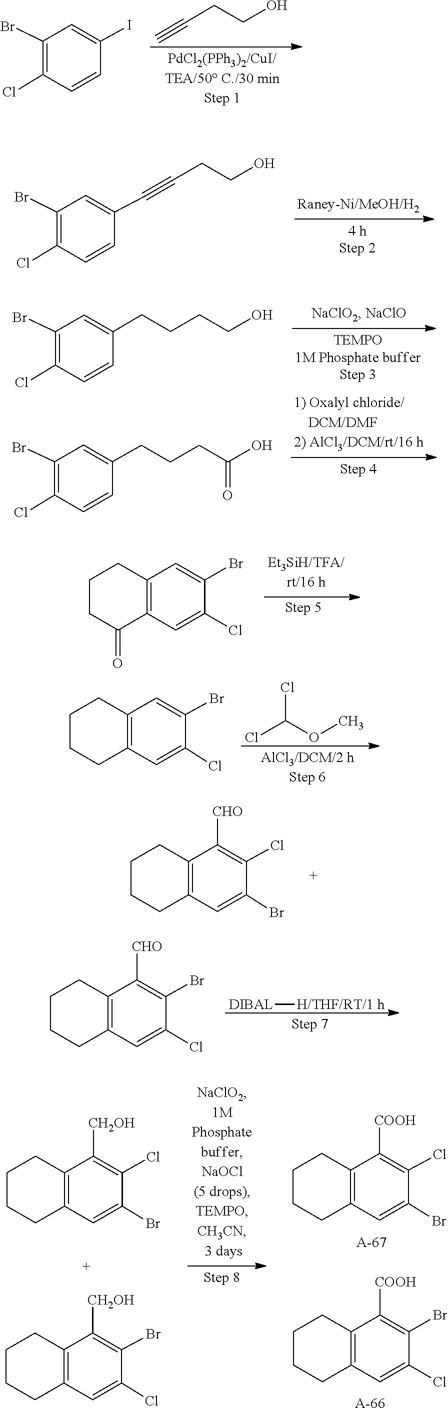

### Step 1: -(3-Bromo-4-chlorophenyl)-3-butyn-1-ol

A mixture of 2-bromo-1-chloro-4-iodobenzene (3.98 g, 12.54 mmol), PdCl₂(PPh₃)₂ (352.2 mg, 0.50 mmol), copper iodide (167.2 mg, 0.88 mmol) and but-3-yn-1-ol (1.14 ml, 15.0 mmol) in triethylamine (101 ml) was purged 3x with argon and heated at 50° C. for 30 min. The reaction mixture was cooled to ambient temperature and evaporated in vacuo and the residue purified by chromatography on silica gel eluting with hexane/EtOAc (0-10%) to provide 4-(3-bromo-4-chlorophenyl)but-3-yn-1-ol (2.9 g, 89%) as an oil.

¹H NMR (300 MHz, CDCl₃) δ 7.58 (d, 1H); 7.27 (d, 1H); 7.18 (dd, 1H); 3.73 (t, 2H); 2.57 (t, 2H); 1.85-1.63 (br, 1H); ES-MS: 258 [M+1].

### Step 2: 4-(3-Bromo-4-chlorophenyl)-1-butanol

To a solution of 4-(3-bromo-4-chlorophenyl)-3-butyn-1-ol (2.9 g, 11.24 mmol) in methanol (60 mL), Raney-Ni (50% slurry in water, 1.3 g) was added, and the mixture stirred under hydrogen for 4 h. The catalyst was removed by filtration and the filtrate evaporated under reduced pressure to provide 4-(3-bromo-4-chlorophenyl)-1-butanol (quantitative yield) as colourless oil.

¹H NMR (300 MHz, CDCl₃) δ 7.52 (s, 1H); 7.39 (d, 1H); 7.19 (dd, 1H); 3.56 (t, 2H); 2.61 (t, 2H); 1.75-1.45 (m, 4H); ES-MS: 264 [M+1].

### Step 3: 4-(3-Bromo-4-chlorophenyl)butanoic acid

To a stirred solution of 4-(3-bromo-4-chlorophenyl)-1-butanol (2.8 g, 10.62 mmol) in CH₃CN (89 mL), 1M sodium phosphate buffer (pH-6, 89 mL) was introduced, followed by NaClO2 (2.4 g, 26.55 mmol), NaOCl (5 drops, 4-4.99M solution) and TEMPO (82.9 mg, 0.53 mmol) at ambient temperature. The reaction mixture was stirred for 2 h and further NaOCl (4 drops, 4-4.99M solution) and TEMPO (82.9 mg, 0.53 mmol) were introduced; the addition was then repeated three more times in 8 h intervals. The reaction mixture was quenched with 1.0 N NaOH (30 mL) and extracted into EtOAc (2×30 mL). The aqueous layer was acidified to pH ~2.0 with 1.0 N HCl and extracted into EtOAc (2×50 mL). The combined organic layers were dried (Na₂SO₄) and evaporated under reduced pressure to give 4-(3-bromo-4-chlorophenyl) butanoic acid as a brown solid (2.9 g, 98%).

¹H NMR (300 MHz, CDCl₃) δ 7.37 (s, 1H); 7.17 (d, 1H); 7.01 (dd, 1H); 2.55 (t, 2H); 2.30 (t, 2H); 1.87 (q, 2H); ES-MS: 276 [M−1].

### Step 4: 6-Bromo-7-chloro-3,4-dihydronaphthalen-1(2H)-one

To a solution of 4-(3-bromo-4-chlorophenyl)butanoic acid (2.9 g, 10.45 mmol) in CH₂Cl₂ (50 mL) at 0° C., cat. DMF (two drops) was added followed by oxalyl chloride (0.98 mL, 11.39 mmol) and the reaction mixture was stirred at ambient temperature for 2 h. The reaction mixture was cooled to 0° C., AlCl₃ (4.17 g, 31.35 mmol) was added, then stirred at ambient temperature over a period of 16 h. The reaction mixture was cooled to 0° C., quenched with water (20 mL), extracted with CH₂Cl₂ (20 mL), washed with 1.0 N NaOH (20 mL), dried (Na₂SO₄) and evaporated under reduced pressure to provide 6-bromo-7-chloro-3,4-dihydronaphthalen-1(2H)-one (2.62 g, 95%).

¹H NMR (300 MHz, CDCl₃) δ 8.13 (s, 1H); 7.62 (s, 1H); 2.97 (t, 2H); 2.70 (t, 2H); 2.19 (q, 2H); ES-MS: 260 [M+1].

### Step 5: 6-Bromo-7-chloro-1,2,3,4-tetrahydronaphthalene

A mixture of 6-bromo-7-chloro-3,4-dihydronaphthalen-1(2H)-one (3.41 g, 13.15 mmol) in Et₃SiH (6.29 ml, 39.45 mmol) and TFA (11 mL) in a sealed tube was stirred at ambient temperature for 16 h. The solvent was removed under reduced pressure and the residue was purified by column chromatography on silica gel eluting with hexane/EtOAc (0-5%) to provide 6,7-dichloro-1,2,3,4-tetrahydronaphthalene (3.2 g, 99% yield) as a yellow solid.

¹H NMR (300 MHz, CDC₃) δ 7.37 (s, 1H); 7.20 (s, 1H); 2.87-2.67 (m, 4H); 1.91-1.74 (m, 4H); ES-MS: 246 [M+1].

### Step 6: 3-Bromo-2-chloro-5,6,7,8-tetrahydronaphthalene-1-carbaldehyde and 2-bromo-3-chloro-5,6,7,8-tetrahydronaphthalene-1-carbaldehyde

To a solution of 6-bromo-7-chloro-1,2,3,4-tetrahydronaphthalene (595 mg, 2.42 mmol) in dry CH₂Cl₂ (11 mL), AlCl₃ (485 mg, 3.63 mmol) was introduced at −78° C. under an argon atmosphere. A solution of Cl₂CHOMe (0.33 ml, 3.63 mmol) in dry CH₂Cl₂ (5 mL) was slowly added to the reaction mixture at −78° C., which was stirred at that temperature for 1 h and at 0° C. for 2 h, quenched with saturated aqueous NaHCO₃ (10 mL) and stirred at ambient temperature for 30 min. The organic layer was separated and the aqueous layer extracted with CH₂Cl₂ (2×30 mL). The combined organic extracts were washed with brine (15 mL) and dried (Na₂SO₄,). The filtrate was concentrated and the residue was subjected to column chromatography on silica gel eluting with hexane/EtOAc (0-5%) to provide a mixture of 3-bromo-2-chloro-5,6,7,8-tetrahydronaphthalene-1-carbaldehyde (major) and 2-bromo-3-chloro-5,6,7,8-tetrahydro naphthalene-1-carbaldehyde (minor) (288 mg, 43% yield) as a light brown solid.

Major isomer: ¹H NMR (300 MHz, CDC₃) δ 10.57 (s, 1H); 7.55 (s, 1H); 3.06-2.94 (m, 2H); 2.85-2.71 (m, 2H); 1.87-1.69 (m, 4H); ES-MS: 274 [M+1].

Minor isomer: ¹H NMR (300 MHz, CDC₃) δ 10.46 (s, 1H); 7.39 (s, 1H); 3.06-2.94 (m, 2H); 2.85-2.71 (m, 2H); 1.87-1.69 (m, 4H); ES-MS: 274 [M+1].

### Step 7: 3-Bromo-2-chloro-5,6,7,8-tetrahydronaphthalen-1-yl)methanol and (2-bromo-3-chloro-5,6,7,8-tetrahydronaphthalen-1-yl)methanol

To a solution of 3-bromo-2-chloro-5,6,7,8-tetrahydronaphthalene-1-carbaldehyde and 2-bromo-3-chloro-5,6,7,8-tetrahydro naphthalene-1-carbaldehyde (288 mg, 1.05 mmol) in THF (6.0 mL) at 0° C., DIBAL-H (1M in toluene, 1.58 ml, 1.58 mmol) was added and the reaction mixture stirred at ambient temperature for 2 h. The mixture was cooled to 0° C., quenched with saturated sodium potassium tartrate (5 mL), stirred for 30 min. and extracted into EtOAc (2×50 mL). The combined extracts were dried (Na₂SO₄) and concentrated under reduced pressure. The resultant mixture of (3-bromo-2-chloro-5,6,7,8-tetrahydronaphthalen-1-yl)methanol (major) and (2-bromo-3-chloro-5,6,7,8-tetrahydronaphthalen-1-yl)methanol (minor) (310 mg, quantitative) was utilised in the next step without further purification.

Major isomer: ¹H NMR (300 MHz, CDC₃) δ 7.41 (s, 1H); 4.92 (s, 2H); 3.01-2.87 (m, 2H); 2.84-2.73 (m, 2H); 1.99-1.73 (m, 4H); ES-MS: 276 [M+1].

Minor isomer: ¹H NMR (300 MHz, CDC₃) δ 7.33 (s, 1H); 4.93 (s, 2H); 3.01-2.87 (m, 2H); 2.84-2.73 (m, 2H); 1.99-1.73 (m, 4H); ES-MS: 276 [M+1].

### Step 8: 3-Bromo-2-chloro-5,6,7,8-tetrahydronaphthalene-1-carboxylic acid and 2-bromo-3-chloro-5,6,7,8-tetrahydronaphthalene-1-carboxylic acid

To a mixture of (3-bromo-2-chloro-5,6,7,8-tetrahydronaphthalen-1-yl)methanol and (2-bromo-3-chloro-5,6,7,8-tetrahydronaphthalen-1-yl)methanol (310 mg, 1.12 mmol) in CH₃CN (10.0 mL), 1.0 M sodium phosphate buffer (pH 6.0, 10.0 mL) was added. Addition of NaClO2 (254 mg, 2.8 mmol), NaOCl (5 drops, 4-4.99M solution) and TEMPO (8.8 mg, 0.056 mmol) took place at ambient temperature and the reaction mixture stirred for 16 h. Additional NaOCl (4 drops, 4-4.99M solution) and TEMPO (7.7 mg, 0.049 mmol) were introduced and the addition repeated three more times in 8 h intervals. The reaction mixture was quenched with 1.0 N NaOH (30 mL), extracted into EtOAc (2×30 mL) and the aqueous layer was acidified to pH ~2.0 with 1.0 N HCl before being extracted into EtOAc (2×50 mL). The combined organic layers were dried (Na₂SO₄) and evaporated under reduced pressure. The resultant residue was purified by semi-prep HPLC with 10-100% CH₃CN/0.1% formic acid in water to provide desired acids (9A: 9.7 mg, 3% & 9B: 5.4 mg, 1.6%).

3-Bromo-2-chloro-5,6,7,8-tetrahydronaphthalene-1-carboxylic acid (A-67): ¹H NMR (300 MHz, CDC₃) δ 7.32 (s, 1H); 2.90-2.69 (m, 4H); 1.93-1.76 (m, 4H); ES-MS: 287 [M−1].

HPLC Retention Time: 11.69 min, >99% purity @280 nm.

2-Bromo-3-chloro-5,6,7,8-tetrahydronaphthalene-1-carboxylic acid (A-66): ¹H NMR (300 MHz, CDCl₃) δ 7.41 (s, 1H); 2.84-2.64 (m, 4H); 1.88-1.69 (m, 4H); ES-MS: 287 [M−1].

HPLC Retention Time: 11.81 min, >99% purity @280 nm.

### Example 62: 7-Chloro-6-fluoro-1-methyl-1,2,3,4-tetrahydro-8-quinolinecarboxylic acid (A-77)

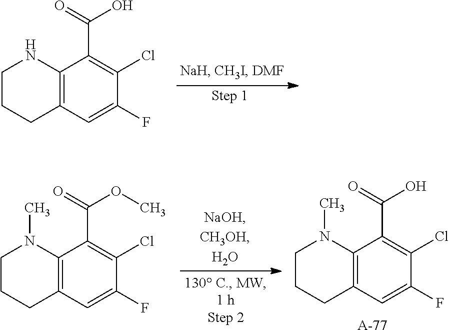

### Step 1: Methyl 7-chloro-6-fluoro-1-methyl-1,2,3,4-tetrahydroquinoline-8-carboxylate

NaH (60% dispersion in mineral oil) (0.35 g, 8.71 mmol) and CH₃I (1.24 g, 8.71 mmol) were added to a solution of 7-chloro-6-fluoro-1,2,3,4-tetrahydroquinoline-8-carboxylic acid (0.2 g, 0.87 mmol) in DMF (10 mL). The reaction mixture was stirred at ambient temperature for 16 h, quenched with water (10 mL), extracted with EtOAc (2×20 mL), dried (Na₂SO₄) and evaporated under reduced pressure. The residue was purified by column chromatography on silica gel eluting with hexane/EtOAc (0-50%) to give the title compound (90.0 mg, 40%).

¹H NMR (300 MHz, CDCl₃) δ 6.80 (d, 1H), 3.93 (s, 3H), 3.12 (t, 2H), 2.82 (s, 3H), 2.68 (t, 2H), 1.91-1.80 (m, 2H).

¹⁹F NMR (300 MHz, CDCl₃); δ −127.58 ppm.

ES-MS: m/z 258.6 (M+1).

### Step 2: 7-Chloro-6-fluoro-1-methyl-1,2,3,4-tetrahydro-8-quinolinecarboxylic acid

To a solution of methyl 7-chloro-6-fluoro-1-methyl-1,2,3,4-tetrahydroquinoline-8-carboxylate (90 mg, 0.35 mmol) in CH₃OH (2 mL) and water (2 mL), NaOH (69.8 mg, 1.75 mmol) was added. The reaction mixture was subjected to microwave conditions at 130° C. for 50 min. Solvent was evaporated and the crude product was dissolved in water (5 mL), washed with CH₂Cl₂ (10 mL), neutralized with 1N HCl to pH ~2, extracted with EtOAc (2×20 mL), dried (Na₂SO₄) and concentrated in vacuo. The residue was purified by semi-prep HPLC eluting with CH₃CN/0.1% formic acid (30-100% in water) to provide the title compound (41 mg, 49%).

¹H NMR (300 MHz, CD₃OD) δ 6.96 (d, J_{H-F}=9.33 Hz, 1H), 3.18 (t, 2H), 2.94 (s, 3H), 2.76 (t, 2H); 1.97-1.85 (m, 2H).

¹⁹F NMR (300 MHz, CD₃OD); δ −128.16 ppm.

ES-MS: 242.6 [M−1].

HPLC: Retention Time: 6.36 min., purity: 99% @254 nm.

### Example 63: 3-Chloro-5,5-difluoro-2-methoxy-5,6,7,8-tetrahydro-1-naphthoic acid (A-73)

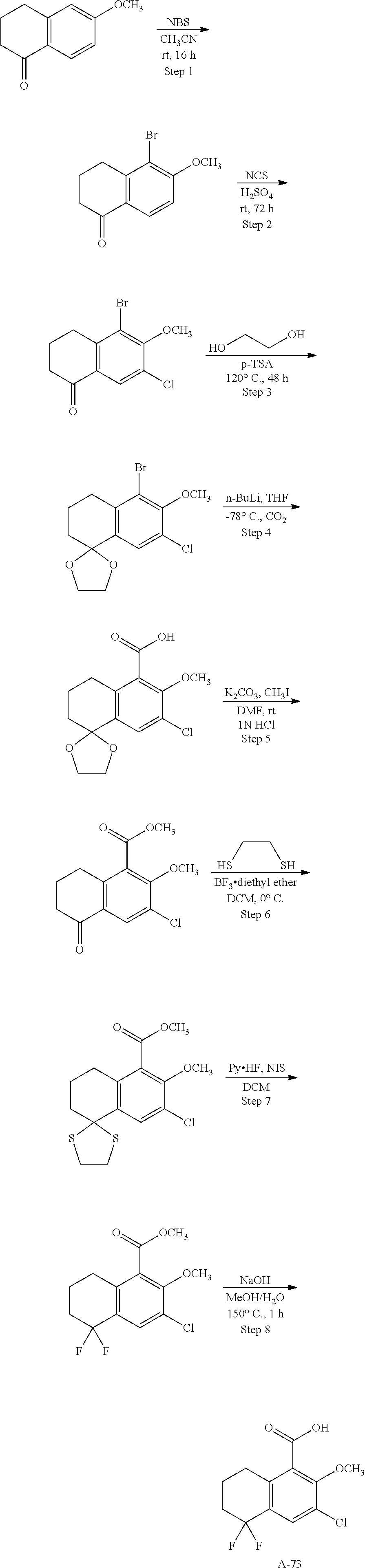

### Step 1: 5-Bromo-6-methoxy-3,4-dihydronaphthalen-1(2H)-one

To a solution of 6-methoxy-3,4-dihydro-2H-naphthalen-1-one (10.0 g, 56.75 mmol) in CH₃CN (40 mL) at ambient temperature, NBS (11.1 g, 62.4 mmol) was added. The reaction mixture was stirred at ambient temperature for 16 h. The solvent was evaporated to a residue under reduced pressure which was treated with a saturated solution of NaHCO₃ (200 mL) followed by CH₂Cl₂ (200 mL). The mixture was stirred briefly, the organic layer was separated and the aqueous layer extracted with CH₂Cl₂ (3×100 mL). The combined organic extracts were dried (Na₂SO₄), evaporated and the residue was purified by column chromatography on silica gel, eluting with hexane/EtOAc (0-20%) to provide the title compound (8.40 g, 58%) as a white solid.

¹H NMR (300 MHz, CDC₃) δ 8.06 (d, 1H), 6.89 (d, 1H), 3.96 (s, 3H), 3.03 (t, 2H), 2.60 (t, 2H). 2.14 (t, 2H) ppm.

### Step 2: 5-Bromo-7-chloro-6-methoxy-3,4-dihydronaphthalen-1(2H)-one

To a solution of 5-bromo-6-methoxy-3,4-dihydronaphthalen-1(2H)-one (3.20 g, 12.54 mmol) in H₂SO₄ (20 mL), NCS (1.84 g, 13.70 mmol) was introduced at 0° C. and the reaction mixture stirred at ambient temperature for 72 h. before being was poured onto a mixture of crushed ice and water. A solid formed which was collected by filtration after the ice melted. The solid was washed with water (2×25 mL), dried, evaporated and purified by column chromatography on silica gel eluting with hexane/EtOAc (0-20%) to provide the title compound (3.30 g, 91%) as a yellow solid.

¹H NMR (300 MHz, CDC₃) δ 8.07 (s, 1H), 3.94 (s, 3H), 2.98 (m, 2H), 2.62 (m, 2H).

2.15 (m, 2H) ppm.

### Step 3: 5′-Bromo-7′-chloro-6′-methoxy-3′,4′-dihydro-2′H-spiro[[1,3]dioxolane-2,1′-naphthalene

A mixture of 5-bromo-7-chloro-6-methoxy-3,4-dihydronaphthalen-1(2H)-one (2.05 g, 7.08 mmol), ethane-1,2-diol (1.6 mL, 28.32 mmol) and PTSA (0.122 g, 0.71 mmol) was heated at 140° C. for 16 h. The reaction mixture was cooled and quenched with a saturated aqueous solution of NaHCO₃ (15 mL). The product was extracted with EtOAc (3×50 mL), dried over Na₂SO₄, and concentrated to provide the title as a yellow solid (1.16, 49% crude yield). The crude product was used for the next step without purification.

¹H NMR (300 MHz, CDC₃) δ 8.07 (s, 1H), 3.94 (s, 3H), 3.76 (s, 4H). 2.99 (t, 2H). 2.69 (t, 2H). 2.15 (t, 2H) ppm.

### Step 4: 7′-Chloro-6′-methoxy-3′,4′-dihydro-2′H-spiro[[1,3]dioxolane-2,1′-naphthalene]-5′-carboxylic acid

n-BuLi (2.5 M in hexane) (2.1 mL, 5.27 mmol) was added to a solution of 5′-bromo-7′-chloro-6′-methoxy-3′,4′-dihydro-2′H-spiro[[1,3]dioxolane-2,1′-naphthalene](1.16 g, 3.48 mmol) in THF (5 mL) at −78° C. The reaction mixture was stirred at that temperature for 1 h. After a further 1 h., CO₂ (g) was bubbled for 15 min., the mixture was slowly brought to 0° C. and quenched with water (15 mL). The reaction mixture was washed with CH₂Cl₂ (2×20 mL), the water layer was separated and acidified with 1 N HCl to pH 4-5 before being extracted with CH₂Cl₂ (3×50 mL). The combined extracts were dried (Na₂SO₄), filtered, and concentrated in vacuo to provide the title compound (0.80 g, 77% crude yield), used in the next step without purification.

¹H NMR (300 MHz, CDC₃) δ 7.39 (s, 1H), 4.12 (m, 4H), 3.92 (s, 3H), 2.12 (m, 2H), 1.64 (m, 2H). 0.98 (m, 2H) ppm.

ES-MS: 297.6 [M−1].

### Step 5: Methyl 3-chloro-2-methoxy-5-oxo-5,6,7,8-tetrahydronaphthalene-1-carboxylate

A mixture of 7′-chloro-6′-methoxy-3′,4′-dihydro-2′H-spiro[1,3-dioxolane-2,1′-naphthalene]-5′-carboxylic acid (0.80 g, 2.68 mmol), K₂CO₃ (0.74 g, 5.36 mmol), and CH₃I (1.14 mL, 8.03 mmol) in DMF (10 mL) was stirred at ambient temperature for 16 h. The reaction mixture was extracted with EtOAc (3×50 mL), the combined extracts washed with H₂O (3×20 mL) and brine (25 mL). 1 N HCl (20 mL) was introduced the mixture stirred for 5 min. The organic layer was separated, dried (Na₂SO₄) and concentrated in vacuo to give the title compound as yellow oil (0.706 g, 98%).

¹H NMR (300 MHz, CDC₃) δ 8.02 (s, 1H), 3.96 (s, 3H), 3.69 (s, 3H), 2.93 (m, 2H), 2.64 (m, 2H). 2.13 (m, 2H) ppm.

### Step 6: Methyl 7′-chloro-6′-methoxy-3′,4′-dihydro-2′H-spiro[1,3-dithiolane-2,1′-naphthalene]-5′-carboxylate

A solution of methyl 3-chloro-2-methoxy-5-oxo-5,6,7,8-tetrahydronaphthalene-1-carboxylate (0.317 g, 1.20 mmol) in CH₂Cl₂ (5 mL) was cooled down to 0° C., boron trifluoride diethyl etherate (0.078 mL, 0.62 mmol) was added slowly, followed by 1,2-ethanedithiol (0.090 mL, 2.49 mmol). The mixture was stirred at ambient temperature for 16 h. The reaction was quenched with a saturated NaHCO₃ solution and stirred for 10 min. The product was extracted with CH₂Cl₂ (3×30 mL), dried (Na₂SO₄) and purified by column chromatography on silica gel, eluting with hexane/EtOAc (0-15%) to obtain the title compound as a white solid (320 mg, 77%).

¹H NMR (300 MHz, CDC₃) δ 8.01 (s, 1H), 3.91 (s, 3H), 3.86 (s, 3H), 3.59 (m, 2H), 3.45 (m, 2H). 2.64 (m, 2H). 2.33 (m, 2H), 1.99 (m, 2H) ppm.

### Step 7: Methyl 3-chloro-5,5-difluoro-2-methoxy-5,6,7,8-tetrahydronaphthalene-1-carboxylate

To a solution of methyl 7′-chloro-6′-methoxy-3′,4′-dihydro-2′H-spiro[1,3-dithiolane-2,1′-naphthalene]-5′-carboxylate (0.1 g, 0.29 mmol) in CH₂Cl₂ (5 mL), N-iodosuccinimide (0.256 g, 1.14 mmol), and HF-Pyridine solution (0.21 mL, 2.32 mmol) were added slowly in CH₂Cl₂ (5 mL) at −78° C. The reaction mixture was stirred at −78° C. for 3 h., quenched with saturated NaHCO₃ solution, extracted with CH₂Cl₂ (3×20 mL), dried (Na₂SO₄) and purified by column chromatography on silica gel eluting with (0-15%) hexane/EtOAc to afford methyl 3-chloro-5,5-difluoro-2-methoxy-5,6,7,8-tetrahydronaphthalene-1-carboxylate as a light-yellow solid (56 mg, 66%).

¹H NMR (300 MHz, CDCl₃) δ 7.75 (s, 1H), 3.93 (s, 3H), 3.90 (s, 3H), 2.70 (m, 2H).

2.21 (m, 2H). 1.98 (m, 2H) ppm. ¹⁹F NMR (300 MHz, CDCl₃) δ −84.05 ppm.

### Step 8: 3-chloro-5,5-difluoro-2-methoxy-5,6,7,8-tetrahydro-1-naphthoic acid

A 1 N solution of NaOH (0.21 mL, 0.21 mmol) was added to a solution of methyl 3-chloro-5,5-difluoro-2-methoxy-5,6,7,8-tetrahydronaphthalene-1-carboxylate (0.055 g, 0.19 mmol) in CH₃OH (1.5 mL) and water (0.5 mL). The reaction mixture was heated at 100° C. under microwave conditions for 1 h., cooled, and evaporated. The resultant residue was separated by semi-prep HPLC using 10-100% acetonitrile/0.1% formic acid in water to provide the title compound (7 mg, 13%) as a colourless oil.

¹H NMR (300 MHz, CD₃OD) δ 7.70 (s, 1H), 3.94 (s, 3H), 2.84 (m, 2H), 2.25 (m, 2H).

2.01 (m, 2H). ppm.

¹⁹F NMR (300 MHz, CD₃OD) δ− 85.3 ppm.

ES-MS: 275.5 [M−1].

HPLC: Retention Time: 10.64 min., purity: 96.5% @280 nm

### Example 64: 6-Chloro-7-fluoro-1-[(p-fluorophenyl)methyl]-1,2,3,4-tetrahydro-8-quinolinecarboxylic acid (A-72)

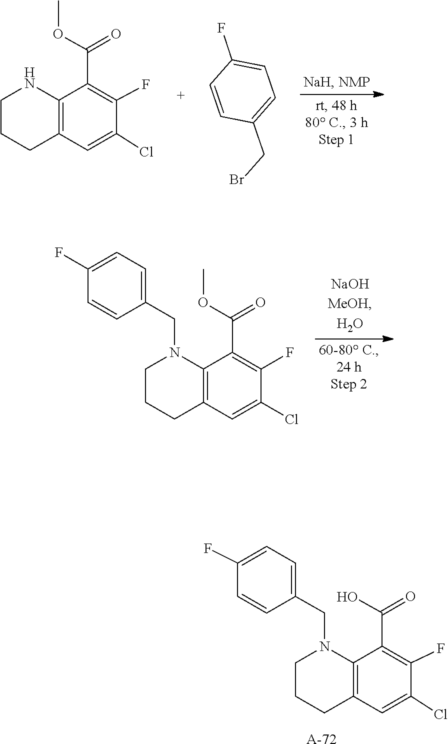

### Step 1: Methyl 6-chloro-7-fluoro-1-[(4-fluorophenyl)methyl]-3,4-dihydro-2H-quinoline-8-carboxylate

Methyl 6-chloro-7-fluoro-1,2,3,4-tetrahydroquinoline-8-carboxylate (48 mg, 0.197 mmol) was dissolved in dry NMP (1.5 mL). Sodium hydride, 60% in mineral oil (16 mg, 0.394 mmol) was added and the reaction mixture was stirred at room temperature for 10 minutes. p-fluorobenzyl bromide (0.037 mL, 0.295 mmol) was added, and the reaction mixture was stirred at 80° C. for 18 hours.

The reaction mixture was quenched with NH₄Cl (5 mL) and extracted with DCM (2×5 mL). The organic layers were combined, washed with brine (10 mL), dried over Na₂SO₄, filtered then concentrated in vacuo.

The crude material was purified by column chromatography over silica (12 g cartridge) eluting with a gradient of DCM (0% to 20%; v/v) in iso-hexane to afford the title compound (46 mg, 44%) as a green oil.

¹H NMR (400 MHz, CDC₃) δ 7.31-7.22 (m, 2H), 7.12-7.00 (m, 2H), 4.24 (s, 2H), 3.62 (s, 3H), 3.21-3.00 (m, 2H), 2.75-2.67 (m, 2H), 1.86-1.73 (m, 2H).

### Step 2: 6-Chloro-7-fluoro-1-[(P-fluorophenyl)methyl]-1,2,3,4-tetrahydro-8-quinolinecarboxylic acid

Methyl 6-chloro-7-fluoro-1-[(4-fluorophenyl)methyl]-3,4-dihydro-2H-quinoline-8-carboxylate (24 mg, 0.0682 mmol) was dissolved in MeOH (1 mL) and water (1 mL).

NaOH (5.5 mg, 0.136 mmol) was added and the reaction mixture was stirred at 70° C. for 72 hours.

The reaction mixture was concentrated in vacuo and taken up in water (10 mL) and EtOAc (10 mL). The organic layer was extracted and the aqueous layer was acidified to pH 1 using 1 N HCl. The aqueous layer was then extracted with EtOAc (2×10 mL), dried over Na₂SO₄, filtered then concentrated in vacuo to afford the title compound (8.2 mg, 33%) as a white solid.

UPLC-LCMS analysis (4 min, Basic): rt=1.26, m/z=338.0 [M+H]⁺, 95% purity.

¹H NMR (400 MHz, CDC₃) δ 7.41-7.36 (m, 2H), 7.32 (dt, J=7.7, 1.0 Hz, 1H), 7.12-7.04 (m, 2H), 4.04 (s, 2H), 3.17-2.97 (m, 2H), 2.89 (tdd, J=7.1, 1.7, 1.2 Hz, 2H), 2.01-1.89 (m, 2H).

### Example 65: 1-Benzyl-6-chloro-7-fluoro-1,2,3,4-tetrahydro-8-quinolinecarboxylic acid (A-71)

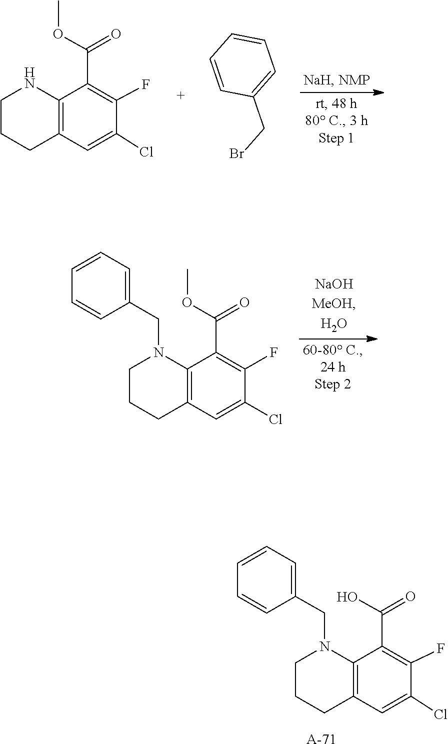

### Step 1: Methyl 1-benzyl-6-chloro-7-fluoro-3,4-dihydro-2H-quinoline-8-carboxylate

Methyl 6-chloro-7-fluoro-1,2,3,4-tetrahydroquinoline-8-carboxylate (50 mg, 0.205 mmol) was dissolved in NMP (1.5 mL). Sodium hydride, 60% in mineral oil (16 mg, 0.410 mmol) was added and the reaction mixture was stirred at room temperature for 10 minutes. Benzyl bromide (0.040 mL, 0.337 mmol) was added and the reaction mixture was stirred at room temperature for 48 hours.

Sodium hydride, 60% in mineral oil (16 mg, 0.410 mmol) and benzyl bromide (0.040 mL, 0.337 mmol) were added and the reaction mixture was stirred at 80° C. for 3 hours. The reaction mixture was diluted with DCM (5 mL) and quenched with NH₄Cl (10 mL). The mixture was extracted with DCM (2×10 mL). The organic layers were combined and washed with brine (10 mL), dried over Na₂SO₄, filtered then concentrated in vacuo. The crude material was purified by column chromatography over silica (12 g cartridge) eluting with a gradient of EtOAc (5% to 10%; v/v) in iso-hexane. The material was re-purified by column chromatography over silica (4 g cartridge) eluting with a gradient of DCM (5% to 45%; v/v) in iso-hexane to afford the title product (14 mg, 17%) as a colourless oil.

¹H NMR (400 MHz, CDC₃) δ 7.40-7.31 (m, 5H), 7.12-7.01 (m, 1H), 4.30 (s, 2H), 3.59 (s, 3H), 3.39-3.02 (m, 2H), 2.75-2.64 (m, 2H), 2.13-1.73 (m, 2H).

### Step 2: 1-benzyl-6-chloro-7-fluoro-1,2,3,4-tetrahydro-8-quinolinecarboxylic acid

Methyl 1-benzyl-6-chloro-7-fluoro-3,4-dihydro-2H-quinoline-8-carboxylate (14 mg, 0.0419 mmol) was dissolved in MeOH (1 mL). A solution of NaOH (3.4 mg, 0.0839 mmol) in water (1 mL) was added and the reaction mixture was stirred at 70° C. for 5 hours. A solution of NaOH (3.4 mg, 0.0839 mmol) in water (1 mL) and MeOH (1 mL) was added and the reaction mixture was stirred at 70° C. for 24 hours. A solution of NaOH (3.4 mg, 0.0839 mmol) in water (1 mL) and MeOH (1 mL) was added and the reaction mixture was stirred at 80° C. for 18 hours.

The reaction mixture was concentrated in vacuo and taken up in water (5 mL) and EtOAc (5 mL). The aqueous layer was acidified to pH 1 using 1 N HCl and extracted with EtOAc (2×10 mL) and DCM (10 mL). The organic layers were dried using Na₂SO₄, filtered then concentrated in vacuo. The crude product was purified by prep-HPLC to afford the title compound (3.3 mg, 24%) as a brown oil.

UPLC-LCMS analysis (4 min, Acidic): rt=1.97, m/z=320.0 [M+H]*, 97% purity.

¹H NMR (400 MHz, DMSO-D₆) δ 7.48-7.28 (m, 4H), 7.28-7.22 (m, 1H), 7.16 (d, J=8.2 Hz, 1H), 4.35 (s, 2H), 2.91-2.84 (m, 2H), 2.66 (t, J=6.3 Hz, 2H), 1.98-1.58 (m, 2H).

### Example 66: 6-Chloro-7-fluoro-1-[(3-thienyl)methyl]-1,2,3,4-tetrahydro-8-quinolinecarboxylic acid (A-70)

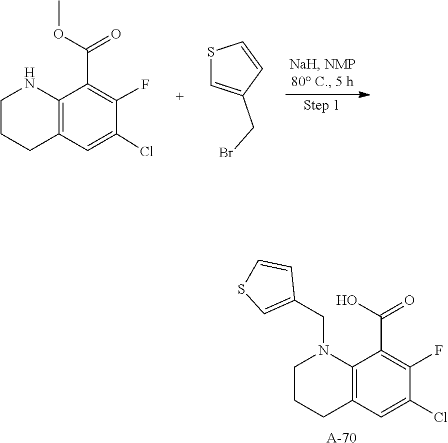

### Step 1: 6-Chloro-7-fluoro-1-[(3-thienyl)methyl]-1,2,3,4-tetrahydro-8-quinolinecarboxylic acid

Methyl 6-chloro-7-fluoro-1,2,3,4-tetrahydroquinoline-8-carboxylate (50 mg, 0.205 mmol) was dissolved in NMP (1.5 mL). 3-(bromomethyl)thiophene (55 mg, 0.308 mmol) and sodium hydride, 60% in mineral oil (16 mg, 0.410 mmol) were added at room temperature. The reaction mixture was stirred at 80° C. for 5 hours then was quenched with NH₄Cl (10 mL) and extracted with DCM (2×10 mL). The organic layers were combined, dried over Na₂SO₄, filtered then concentrated in vacuo. The crude material was purified by column chromatography over silica (12 g cartridge) eluting with a gradient of DCM (5% to 100%; v/v) in iso-hexane, followed by DCM:MeOH (20%; v/v). The residue was purified by prep-HPLC to afford the title product (1.5 mg, 2%) as a white solid.

UPLC-LCMS analysis (4 min, Acidic): rt=1.88, m/z=326.0 [M+H]*, 96% purity.

¹H NMR (400 MHz, DMSO-D₆) δ 8.40 (s, 1H), 7.39-7.34 (m, 2H), 7.26 (dd, J=4.5, 1.8 Hz, 1H), 6.82 (d, J=8.4 Hz, 1H), 4.46 (s, 2H), 3.04-2.78 (m, 2H), 2.57 (t, J=6.5 Hz, 2H), 1.63 (p, J=6.3 Hz, 2H).

### Example 67: 1-Benzyl-6-chloro-5-fluoro-4-indolinecarboxylic acid (A-76)

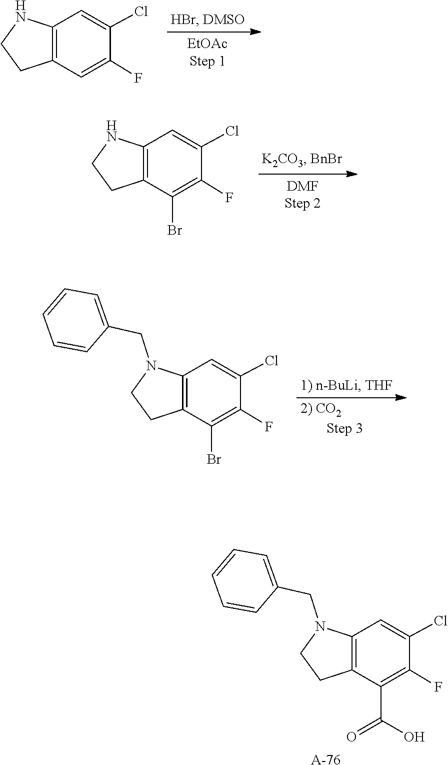

### Step 1: 4-Bromo-6-chloro-5-fluoroindoline

To a mixture of 6-chloro-5-fluoroindoline (0.30 g, 1.75 mmol) and DMSO (0.14 mL, 1.92 mmol) in EtOAc (50 mL), 62% HBr (0.47 mL, 3.50 mmol) was added and the reaction mixture was stirred at 50° C. for 2 h. The reaction mixture was cooled to room temperature, then diluted with water (10 mL) and EtOAc (15 mL). The layers were separated and the aqueous layer was extracted with EtOAc (2×15 mL). The combined organic layers were dried (Na₂SO₄) and concentrated under reduced pressure. The crude product was used in the next step without further purification.

¹H NMR (300 MHz, CDCl₃) δ 6.88 (d, J_{H-F}=7.8 Hz, 1H), 3.65 (t, 2H), 3.14 (t, 2H).

### Step 2: 1-Benzyl-4-bromo-6-chloro-5-fluoroindoline

To a solution of 4-bromo-6-chloro-5-fluoroindoline (0.30 g, 1.20 mmol) in DMF (5.0 mL), K₂CO₃ (0.33 g, 2.39 mmol) was introduced at room temperature followed by the addition of benzyl bromide (0.28 mL, 2.39 mmol). The reaction mixture was stirred at room temperature for 16 h, quenched with water (20 mL) and extracted into EtOAc (2×20 mL). The combined organic layers were dried (Na₂SO₄) and concentrated under reduced pressure. The residue was purified by column chromatography on silica gel, eluting with hexane/EtOAc (20-30%) to provide the title compound (0.30 g, 73%).

¹H NMR (300 MHz, CDCl₃) δ 7.38-7.29 (m, 5H), 6.88 (d, J_{H-F}=7.7 Hz, 1H), 4.67 (s, 2H), 3.46 (t, 2H), 2.95 (t, 2H).

### Step 3: 1-Benzyl-6-chloro-5-fluoro-4-indolinecarboxylic acid

To a solution of 1-benzyl-4-bromo-6-chloro-5-fluoroindoline (0.25 g, 0.73 mmol) in THF (5.0 mL) at −78° C., n-BuLi (2.5 M in hexane) (0.59 mL, 1.47 mmol) was added. The reaction mixture was stirred at the same temperature for 1 hour. CO₂ (g) was bubbled for 30 minutes and the mixture was slowly brought to 0° C. The reaction mixture was quenched with water and washed with EtOAc (2×15 mL) to remove impurities. The aqueous layer was acidified to pH ~2.0 using 1.0 N HCl and extracted with EtOAc (3×15 mL). The combined organic layers were dried (Na₂SO₄) and concentrated under reduced pressure. The residue was subjected to semi-preparative HPLC using 10-100% acetonitrile/0.1% formic acid in water to afford the title compound (9.0 mg, 4%).

¹H NMR (300 MHz, CDCl₃) δ 7.40-7.26 (m, 5H), 6.95 (d, J_{H-F}=8.0 Hz, 1H), 4.27 (s, 2H), 3.41 (t, 2H), 2.91 (t, 2H). ¹⁹F NMR (300 MHz, CDCl₃); δ −125.03 ppm.

ES-MS: 306.7 [M+1].

HPLC: Retention Time: 11.4 min., purity: 87.9% at 280 nm.

### Example 68: 7-Chloro-6-fluoro-2,2-dimethyl-8-chromancarboxylic acid (A-75)

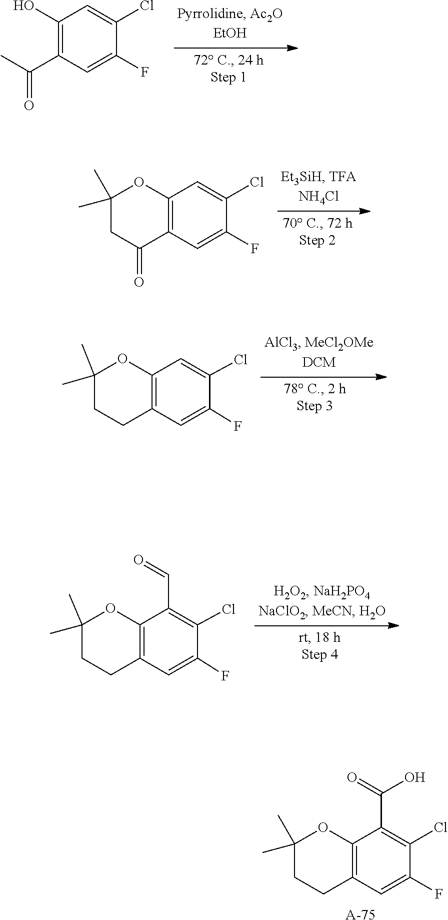

### Step 1: 7-chloro-6-fluoro-2,2-dimethyl-chroman-4-one

1-(4-chloro-5-fluoro-2-hydroxyphenyl)ethan-1-one (4.0 g, 21.2 mmol) was added to a dry flask and flushed with nitrogen. Ethanol (40 mL) was added, and the reaction mixture was stirred at room temperature. Acetone (16 mL, 212 mmol) and pyrrolidine (3.5 mL, 42.4 mmol) were added and the reaction mixture was stirred at 72° C. for 24 hours.

The reaction mixture was concentrated in vacuo. The residue was dissolved in 1 N HCl (30 mL) and EtOAc (30 mL). The mixture was then diluted with brine (100 mL) and extracted with EtOAc (2×100 mL) and DCM (100 mL). The organic layers were combined, dried over Na₂SO₄, filtered then concentrated in vacuo. The crude material was purified by column chromatography over silica (220 g cartridge) eluting with a gradient of EtOAc (0% to 10%; v/v) in iso-hexane to afford the title compound (1.46 g, 29%) as an orange oil.

¹H NMR (400 MHz, CDC₃) δ 7.59 (dd, J=8.5, 0.4 Hz, 1H), 7.03-7.01 (m, 1H), 2.71 (s, 2H), 1.45 (s, 6H).

### Step 2: 7-chloro-6-fluoro-2,2-dimethyl-chromane

7-chloro-6-fluoro-2,2-dimethyl-chroman-4-one (1.1 g, 4.81 mmol) was dissolved in trifluoroacetic acid (22 mL, 290 mmol) and triethylsilane (11 mL, 69.4 mmol). NH₄Cl (1.33 g, 24.9 mmol) was added, and the reaction mixture was stirred at 70° C. for 24 hours. Trifluoroacetic acid (10 mL, 131 mmol), triethylsilane (5.0 mL, 31.3 mmol) and NH₄Cl (1.33 g, 24.9 mmol) were added, and the reaction mixture was stirred at 70° C. for 24 hours. Trifluoroacetic acid (10 mL, 131 mmol), triethylsilane (5.0 mL, 31.3 mmol) and NH₄Cl (1.33 g, 24.9 mmol) were added, and the reaction mixture was stirred at 70° C. for 24 hours.

The reaction mixture was concentrated in vacuo. The reaction mixture was diluted in

NaHCO₃ saturated solution until the pH reached 7, then extracted with EtOAc (2×100 mL). The organic layers were washed with brine (50 mL), dried over Na₂SO₄, filtered then concentrated in vacuo. The crude material was purified by column chromatography over silica (80 g cartridge) eluting with a gradient of EtOAc (0% to 10%; v/v) in iso-hexane. The residue was re-purified by column chromatography over silica (40 g cartridge) eluting with a gradient of iso-hexane as eluent to afford 7-chloro-6-fluoro-2,2-dimethyl-chromane (890 mg, 86%) as a colourless oil.

¹H NMR (400 MHz, CDC₃) δ 6.83 (dt, J=9.4, 1.0 Hz, 1H), 6.80 (d, J=6.5 Hz, 1H), 2.72 (tt, J=6.8, 1.1 Hz, 2H), 1.77 (t, J=6.8 Hz, 2H), 1.31 (s, 6H).

### Step 3: 7-chloro-6-fluoro-2,2-dimethyl-chromane-8-carbaldehyde

7-chloro-6-fluoro-2,2-dimethyl-chromane (50 mg, 0.233 mmol) was dissolved in dry DCM (1 mL) under nitrogen and the reaction mixture was cooled to −78° C. AlCl₃ (47 mg, 0.349 mmol) was added at that temperature, then α,α-dichloromethyl methyl ether (0.032 mL, 0.349 mmol) in dry DCM (0.5 mL) was added dropwise and the reaction mixture was stirred at −78° C. for 1 hour, then 0° C. for 1 hour. The mixture was then quenched with NaHCO₃ saturated solution (2 mL) and stirred at room temperature for 30 minutes. The reaction mixture was extracted with DCM (2×20 mL). The organic layers were combined and washed with brine (30 mL), dried over Na₂SO₄, filtered then concentrated in vacuo to afford the title compound (49 mg, 40%) as a yellow oil.

¹H NMR (400 MHz, CDCl₃) δ 10.45 (d, J=1.6 Hz, 1H), 7.06 (dt, J=9.0, 1.0 Hz, 1H), 2.89-2.75 (m, 2H), 1.85 (t, J=6.8 Hz, 2H), 1.38 (s, 6H).

### Step 4: 7-Chloro-6-fluoro-2,2-dimethyl-8-chromancarboxylic acid

7-chloro-6-fluoro-2,2-dimethyl-chromane-8-carbaldehyde (48 mg, 0.198 mmol) was dissolved in MeCN (1.8 mL) and Water (1.2 mL). Hydrogen peroxide, 35 wt. % in water (0.030 mL, 0.297 mmol) and sodium phosphate monobasic (47 mg, 0.396 mmol) were added at 0° C. Sodium chlorite (89 mg, 0.989 mmol) in water (0.5 mL) was added dropwise and the reaction mixture was stirred at room temperature for 18 hours. The reaction mixture was quenched with sodium bisulfite solution (20 mL, sat. aq.) and extracted with diethyl ether (2×20 mL). The organic layers were combined, washed with brine (20 mL), dried over Na₂SO₄, filtered then concentrated in vacuo. The residue was purified by prep-HPLC to afford the title compound (5.0 mg, 10%) as a white solid. UPLC-LCMS analysis (4 min, Acidic): rt=1.48, m/z=257.1 [M−H]⁻, 100% purity.

¹H NMR (400 MHz, DMSO-D₆) δ 7.20 (d, J=9.8 Hz, 1H), 2.74 (t, J=6.8 Hz, 2H), 1.76 (t, J=6.8 Hz, 2H), 1.24 (s, 6H).

### Example 69: 7-Chloro-6-methoxy-5-chromancarboxylic acid (A-79)

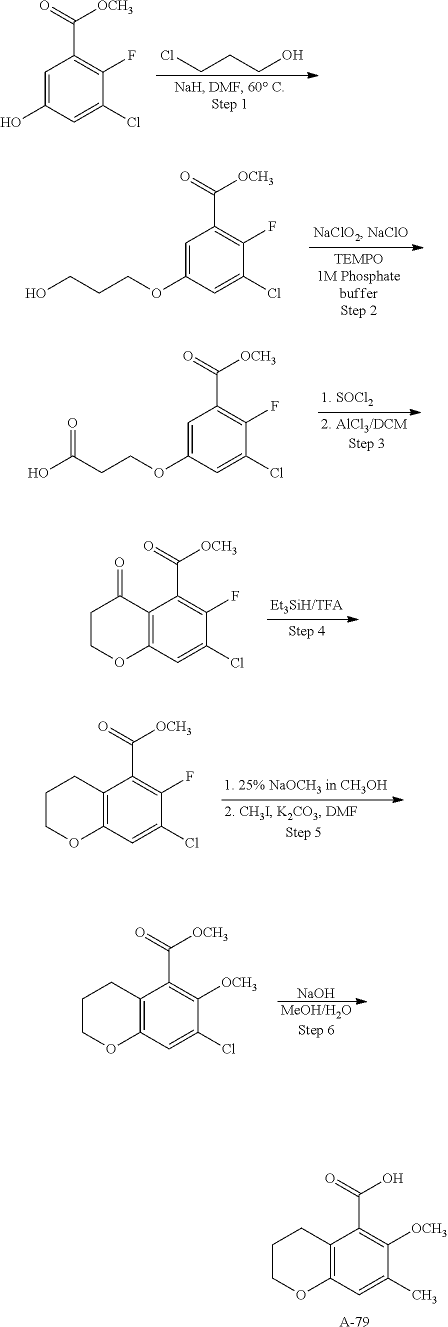

### Step 1: Methyl 3-chloro-2-fluoro-5-(3-hydroxypropoxy)benzoate

NaH (0.352 g, 8.79 mmol) was added to a solution of the methyl 3-chloro-2-fluoro-5-hydroxybenzoate (1.2 g, 5.86 mmol) in DMF (10.0 mL) and the reaction mixture stirred at ambient temperature for 30 min. 3-Chloro-1-propanol (0.67 g, 7.04 mmol) was added dropwise and heated at 60° C. for 16 h., quenched with water (5 mL) and extracted into EtOAc (2×30 mL). The combined extracts were dried (Na₂SO₄), concentrated and the residue purified by column chromatography on silica gel eluting with hexane/EtOAc (0-100%) to provide the title compound as an oil (0.5 g, 32%).

¹H NMR (300 MHz, CDCl₃) δ 7.32 (t, 1H); 7.13 (t, 1H); 4.10 (t, 2H); 3.93 (s, 3H), 3.85 (t, 2H); 2.10-1.99 (m, 2H). ¹⁹F NMR (300 MHz, CDCl₃); δ −122.17 ppm.

### Step 2: 3-(3-Chloro-4-fluoro-5-(methoxycarbonyl)phenoxy)propanoic acid

1.0 M Sodium phosphate buffer (pH 6.0, 10.0 mL) was added to a solution of methyl 3-chloro-2-fluoro-5-(3-hydroxypropoxy)benzoate (0.5 g, 1.90 mmol) in CH₃CN (10.0 mL), followed by NaClO₂ (0.430 g, 4.76 mmol), NaOCl (0.2 mL, 4-4.99 M solution) and TEMPO (15 mg, 0.095 mmol). The reaction mixture was stirred at ambient temperature for 16 h., additional NaOCl (0.2 mL, 4-4.99 M solution) and TEMPO (15 mg, 0.095 mmol) were introduced and stirring continued for a further 2 h. The reaction mixture was quenched with 1.0 N NaOH (20 mL) and washed with EtOAc (2×30 mL). The aqueous layer was acidified to pH ~2.0 using 1.0 N HCl and extracted into EtOAc (2×40 mL). The combined extracts were dried (Na₂SO₄) and concentrated under reduced pressure to give the title compound (0.45 g, 85%).

¹H NMR (300 MHz, CD₃OD) δ 7.40-7.36 (m, 1H); 7.35-7.30 (m, 1H); 4.27 (t, 2H); 3.94 (s, 3H), 2.79 (t, 2H). ¹⁹F NMR (300 MHz, CDCl₃); δ −125.10 ppm.

ES-MS: 275.6[M−1].

### Step 3: Methyl 7-chloro-6-fluoro-4-oxochroman-5-carboxylate

A mixture of 3-(3-chloro-4-fluoro-5-(methoxycarbonyl)phenoxy)propanoic acid (0.25 g, 0.90 mmol), and thionyl chloride (3 mL) was refluxed for 1.5 h., cooled and concentrated.) AlCl₃ (0.16, 1.17 mmol) was added at 0° C. to the acid chloride in 1,2-DCE (10 mL) and the mixture stirred at ambient temperature for 16 h. The resultant solution was poured into a mixture of crushed ice and 1M HCl (10 mL). The reaction mixture was extracted with CH₂Cl₂ (2×30 mL), washed with H₂O (3×10 mL), brine (20 mL) then the combined extracts were dried (Na₂SO₄), filtered and concentrated in vacuo to give the title compound (0.17 g, 73%) as a solid.

¹H NMR (300 MHz, CDC₃) δ 7.13 (d, 1H); 4.54 (t, 2H); 3.99 (s, 3H); 2.81 (t, 2H). ¹⁹F NMR (300 MHz, CDC₃); δ −127.61 ppm.

### Step 4: Methyl 7-chloro-6-fluorochroman-5-carboxylate

A mixture of methyl 7-chloro-6-fluoro-4-oxochroman-5-carboxylate (0.170 g, 0.70 mmol), triethylsilane (0.488 g, 4.20 mmol) and TFA (0.480 g, 4.20 mmol) was heated in a sealed tube at 80° C. for 16 h. The reaction mixture was cooled, evaporated and purified by column chromatography on silica gel eluting with hexane/EtOAc (0-10%) to obtain the title compound (0.095 g, 55%) as a colourless oil.

¹H NMR (300 MHz, CDC₃) δ 6.62 (d, 1H); 4.14 (t, 2H); 3.93 (s, 3H); 2.76 (t, 2H); 2.03-1.91 (m, 2H). ¹⁹F NMR (300 MHz, CDC₃); δ −126.98 ppm.

### Step 5: Methyl 7-chloro-6-methoxychroman-5-carboxylate

A mixture of methyl 7-chloro-6-fluorochroman-5-carboxylate (0.065 g, 0.266 mmol), and 25% NaOCH₃ in CH₃OH (1 mL) was heated at 120° C. for 36 h. in a sealed tube.

The reaction mixture was cooled, evaporated and acidified to pH ~2 with 1 N HCl and extracted into EtOAc (2×15 mL). The combined organic layers were dried (Na₂SO₄) and concentrated to provide 7-chloro-6-hydroxychroman-5-carboxylic acid. The crude material in DMF (2 mL) was treated with K₂CO₃ (0.92 g, 0.665 mmol) and CH₃I (0.189 g, 1.33 mmol) at 0° C. and stirred at ambient temperature for 16 h. The reaction mixture was extracted with EtOAc (2×20 mL) and the extracts washed with H₂O (3×10 mL), brine (20 mL) then the combined extracts were dried (Na₂SO₄), filtered and concentrated in vacuo to obtain the title compound (0.05 g, 73%) as a colourless oil.

¹H NMR (300 MHz, CDC₃) δ 6.89 (s, 1H); 4.12 (t, 2H); 3.91 (s, 3H); 3.82 (s, 3H); 2.66 (t, 2H); 2.00-1.91 (m, 2H).

### Step 6: 7-Chloro-6-methoxy-5-chromancarboxylic acid

NaOH (0.016 g, 0.39 mmol) was added to a solution of methyl 7-chloro-6-methoxychroman-5-carboxylate (0.005 g, 0.195 mmol) in CH₃OH (3 mL) and H₂O (1 mL). The reaction mixture was heated under microwave conditions at 130° C. for 50 min. The solvent was removed and the residue dissolved in H₂O (5 mL) before being washed with CH₂Cl₂ (10 mL). The aqueous layer was acidified with 1N HCl to pH ~2, extracted with EtOAc (2×20 mL) then washed with H₂O (10 mL) and brine (10 mL).

The combined extracts were dried (Na₂SO₄) filtered and concentrated to obtain title compound (33 mg, 70%) as a solid.

¹H NMR (300 MHz, CDCl₃) δ 10.78-9.78 (Br s, 1H); 6.92 (s, 1H); 4.13 (t, 2H); 3.87 (s, 3H); 2.83 (t, 2H); 2.04-1.90 (m, 2H).

ES-MS: 241.6[M−1].

HPLC: Retention Time: 9.36 min., purity: 98.7% @280 nm.

### Example 70: 4-Benzyl-7-fluoro-6-methoxy-5-chromancarboxylic acid (A-81)

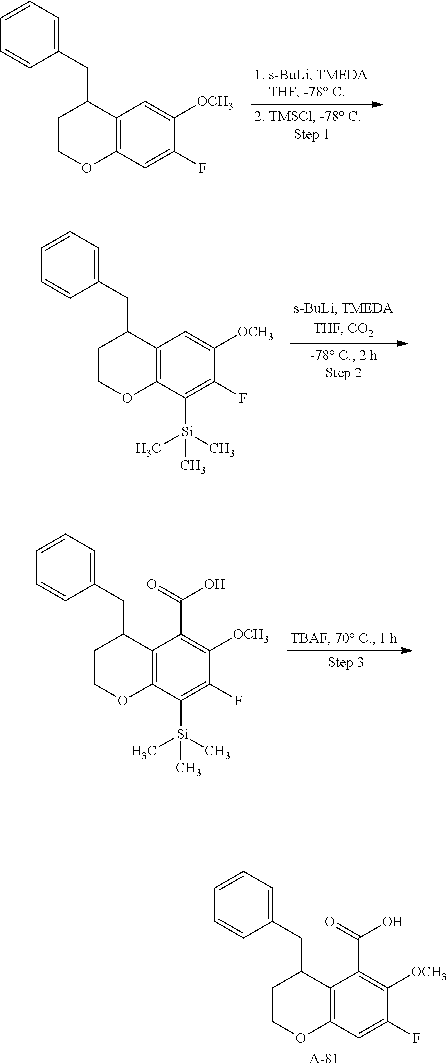

### Step 1: (4-Benzyl-7-fluoro-6-methoxychroman-8-yl)trimethylsilane

s-BuLi (1.4 M in cyclohexane) (0.38 mL, 0.53 mmol) was added to a solution of 4-benzyl-7-fluoro-6-methoxychroman (0.13 g, 0.48 mmol) and TMEDA (0.08 mL, 0.53 mmol) in THF (10 mL) at −78° C. The reaction mixture was stirred at that temperature for 2 h and TMSCl (0.182 mL, 1.43 mmol) was added. The reaction mixture was stirred at −78° C. for 1 h., slowly brought to ambient temperature, stirred for 16 h and the reaction mixture was quenched with saturated ammonium chloride solution (5 mL). It was extracted with EtOAc (2×20 mL), the combined organic layers were dried (Na₂SO₄) and concentrated in vacuo. The residue was purified by preparative TLC eluting with hexane/10% EtOAc to afford the title compound (112.0 mg, 68%).

¹H NMR (300 MHz, CD₃OD) δ 7.24-6.99 (m, 5H), 6.52 (d, 1H), 4.06-3.91 (m, 2H), 3.53 (s, 3H), 3.03-2.83 (m, 2H), 2.71-2.55 (m, 1H), 1.83-1.66 (m, 1H), 1.65-1.54 (m, 1H), 0.17 (s, 9H). ¹⁹F NMR (300 MHz, CD₃OD); δ −127.72 ppm.

### Step 2: 4-Benzyl-7-fluoro-6-methoxy-8-(trimethylsilyl)chroman-5-carboxylic acid

s-BuLi (1.4 M in cyclohexane) (0.342 mL, 0.48 mmol) was added to a solution of (4-benzyl-7-fluoro-6-methoxychroman-8-yl)trimethylsilane (0.11 g, 0.32 mmol) and TMEDA (0.072 ml, 0.47 mmol) in THF (5 mL) at −78° C. The reaction mixture was stirred at that temperature for 3 h and CO₂ gas was bubbled for 20 min. The reaction mixture was stirred at −78° C. temperature for 2 h, slowly brought to ambient temperature, quenched with water (5 mL) and extracted with EtOAc (2×20 mL). The combined extracts were dried (Na₂SO₄) and evaporated to provide the title compound (80 mg, 64%), utilised directly in the next step.

¹H NMR (300 MHz, CDCl₃) δ 7.33-7.15 (m, 5H), 4.29-4.18 (m, 2H), 3.91 (s, 3H), 3.61-3.51 (m, 1H), 3.18 (d, 1H), 2.69-2.55 (m, 1H), 1.85-1.55 (m, 2H), 0.33 (s, 9H). ¹⁹F NMR (300 MHz, CDCl₃); δ −117.48 ppm.

ES-MS: m/z 387.9 (M-1).

### Step 3: 4-Benzyl-7-fluoro-6-methoxy-5-chromancarboxylic acid

A mixture of 4-benzyl-7-fluoro-6-methoxy-8-(trimethylsilyl)chroman-5-carboxylic acid (80 mg, 0.21 mmol) and TBAF (1.0 M in THF) (3.0 ml, 3 mmol) was heated at 70° C. for 1 h. The reaction mixture was cooled, evaporated and purified by semi-prep HPLC eluting with CH₃CN/0.1% formic acid (30-100% in water) to provide the title compound compound (40 mg, 60%).

¹H NMR (300 MHz, CDCl₃) δ 11.60-10.42 (br s, 1H), 7.39-7.19 (m, 5H), 6.81 (d, J_{H-F}=12.2 Hz, 1H), 4.42-4.23 (m, 2H), 4.02 (s, 3H), 3.62-3.50 (m, 1H), 3.29 (dd, 1H), 2.73 (t, 1H), 1.98-1.73 (m, 2H).

¹⁹F NMR (300 MHz, CDCl₃); δ −129.65 ppm.

ES-MS: m/z 315.8 (M-1).

HPLC: Retention time 11.81 min., purity 98.4% at 280 nm.

### Example 71: 4-Benzyl-7-fluoro-6-methoxy-8-chromancarboxylic acid (A-80)

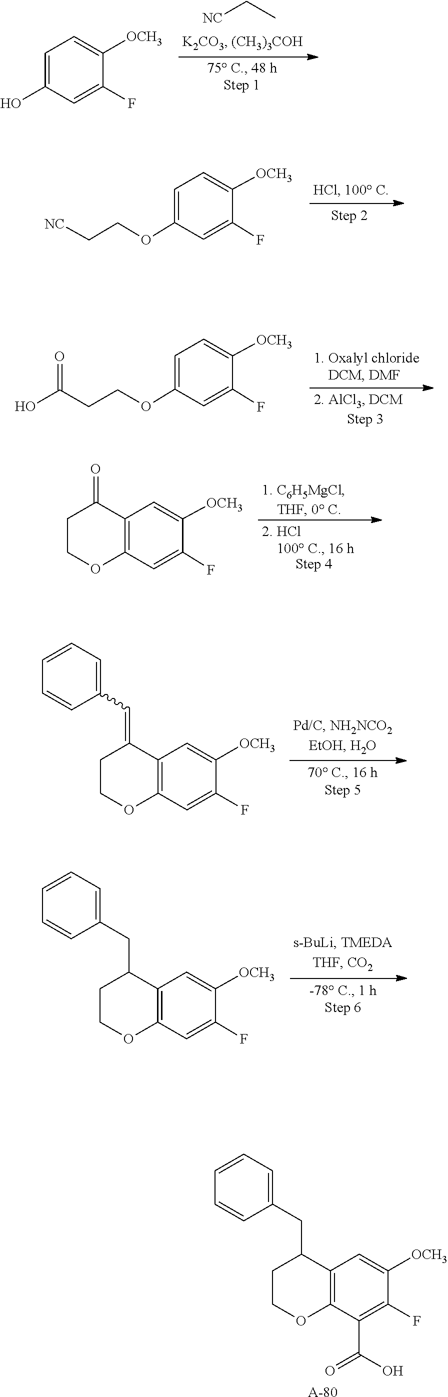

### Step 1: 3-(3-Fluoro-4-methoxyphenoxy)propanenitrile

A mixture of 3-fluoro-4-methoxyphenol (2.0 g, 14.07 mmol), tert-butanol (0.136 mL, 1.43 mmol), K₂CO₃ (99.1 mg, 0.72 mmol) and acrylonitrile (9.22 mL, 140.75 mmol) was heated at 75° C. for 48 h. The reaction mixture was cooled and extracted with EtOAc (2×50 mL), evaporated and purified by column chromatography on silica gel eluting with hexane/EtOAc (0-50%) to obtain the title compound (1.7 g, 62%).

¹H NMR (300 MHz, CDCl₃) δ 6.85 (t, 1H), 6.67 (dd, 1H), 6.58 (d, 1H), 4.07 (t, 2H), 3.79 (s, 3H), 2.76 (t, 2H). ¹⁹F NMR (300 MHz, CDCl₃); δ −131.98 ppm.

### Step 2: 3-(3-Fluoro-4-methoxyphenoxy)propanoic acid

3-(3-Fluoro-4-methoxyphenoxy)propanenitrile (1.2 g, 6.15 mmol) in 5 N HCl (30 mL) was heated at 100° C. for 16 h. The reaction mixture was cooled, quenched with 5.0 N NaOH (50 mL) and washed with EtOAc (2×30 mL). The aqueous layer was acidified to pH ~2.0 with 1.0 N HCl and extracted with EtOAc (2×50 mL). The combined organic extracts were dried (Na₂SO₄) and evaporated under reduced pressure to obtain the title compound (1.0 g, 76%), reacted in the next step without purification.

¹H NMR (300 MHz, CDCl₃) δ 6.87 (t, 1H), 6.70 (dd, 1H), 6.60 (d, 1H), 4.17 (t, 2H), 3.82 (s, 3H), 2.81 (t, 2H).

¹⁹F NMR (300 MHz, CDCl₃); δ −132.15 ppm.

ES-MS: m/z 213.19 (M-1).

### Step 3: 7-Fluoro-6-methoxychroman-4-one

To a solution of 3-(3-fluoro-4-methoxyphenoxy)propanoic acid (0.93 g, 4.34 mmol) in CH₂Cl₂ (12 mL) at 0° C. DMF (6 drops) was introduced, followed by oxalyl chloride (0.74 mL, 8.68 mmol) and the reaction mixture was stirred at ambient temperature for 1 h.

The reaction mixture was cooled again at 0° C., AlCl₃ (1.16 g, 8.68 mmol) was added and stirred at ambient temperature for 48 h. It was cooled to 0° C., quenched with water (20 mL), extracted with CH₂Cl₂ (2×20 mL), washed with 1.0 N NaOH (20 mL), dried (Na₂SO₄) and evaporated under reduced pressure. The crude product was purified by column chromatography on silica gel eluting with hexane/EtOAc (0-30%) to afford the title compound (0.35 g, 41%).

¹H NMR (300 MHz, CDCl₃) δ 7.27 (d, 1H), 6.57 (d, 1H), 4.37 (t, 2H), 3.73 (s, 3H), 2.64 (t, 2H). ¹⁹F NMR (300 MHz, CDCl₃); δ −120.55 ppm.

### Step 4: 4-Benzylidene-7-fluoro-6-methoxychroman

7-Fluoro-6-methoxychroman-4-one (0.345 g, 1.76 mmol) in THF (12 mL) at 0° C. was added to a solution of benzylmagnesium chloride (2.OM in THF, 3.52 mL, 7.03 mmol) and the reaction mixture was stirred at the same temperature for 1 h. The reaction was quenched with saturated ammonium chloride solution (5 mL), extracted with EtOAc (2×20 mL), dried (Na₂SO₄) and evaporated under reduced pressure. The resultant residue was treated with HCl (6N, 10 mL) was heated at 100° C. for 16 h, and the reaction mixture was cooled to ambient temperature, neutralized with 5.0 N NaOH (15 mL), extracted with EtOAc (2×20 mL) then dried (Na₂SO₄). The filtrate was evaporated under reduced pressure to provide the title compound (0.35 g, 74%), reacted directly in the next step without purification.

¹H NMR (300 MHz, CDC₃) δ 7.48-7.18 (m, 6H), 7.03 (s, 1H), 6.70 (d, 1H), 4.17 (t, 2H), 3.94 (s, 3H), 2.94 (t, 2H).

¹⁹F NMR (300 MHz, CDC₃); δ −132.10 ppm.

### Step 5: 4-Benzyl-7-fluoro-6-methoxychroman

A solution of ammonium formate (0.54 g, 8.51 mmol) in water (2 mL) was added to a mixture of 4-benzylidene-7-fluoro-6-methoxychroman (0.23 g, 0.85 mmol) and Pd/C catalyst (23.0 mg, 10%, w/w) in ethanol (10 mL) at 70° C., and the reaction mixture was stirred at the same temperature for 16 h. The mixture was filtered through a pad of Celite® and washed with CH₃OH/EtOAc (20:80, v/v), dried (Na₂SO₄) and concentrated under reduced pressure. The residue was purified by column chromatography silica gel eluting with hexane/EtOAc (0-20%) to provide the title compounds (0.22 g, 95%).

¹H NMR (300 MHz, CDC₃) δ 7.44-7.19 (m, 5H), 6.65 (d, 1H), 6.56 (d, 1H), 4.24-4.15 (m, 2H), 3.77 (s, 3H), 3.22-3.02 (m, 2H), 2.88-2.77 (m, 1H), 2.07-1.92 (m, 1H), 1.88-1.75 (m, 1H). ¹⁹F NMR (300 MHz, CDC₃); δ −135.55 ppm.

### Step 6: 4-Benzyl-7-fluoro-6-methoxy-8-chromancarboxylic acid

s-BuLi (1.4 M in cyclohexane) (0.462 mL, 0.65 mmol) was introduced to a solution of 4-benzyl-7-fluoro-6-methoxychroman (80.0 mg, 0.29 mmol) and TMEDA (0.097 mL, 0.65 mmol) in THF (8 mL) at −78° C. The reaction mixture was stirred at that temperature for

2 h, CO₂ gas was bubbled through it for 20 min., stirred at −78° C. for 1 h. then slowly brought to ambient temperature and quenched with 1M NaOH (5 mL). The mixture was washed with EtOAc (2×10 mL), the aqueous layer was acidified to pH ~2.0 with 1.0 N HCl and extracted with EtOAc (2×20 mL). The combined organic layers were dried (Na₂SO₄), concentrated and the residue was separated by semi-prep HPLC eluting with CH₃CN/0.1% formic acid (30-100% in water) to provide the title compound (8.5 mg, 9.0%).

¹H NMR (300 MHz, CDC₃) δ 7.38-7.09 (m, 5H), 6.58 (d, J_{H-F}=8.98 Hz, 1H), 4.41-4.21 (m, 2H), 3.67 (s, 3H), 3.14-2.97 (m, 2H), 2.88-2.71 (m, 1H), 2.13-1.95 (m, 1H), 1.92-1.76 (m, 1H).

¹⁹F NMR (300 MHz, CDC₃); δ −132.0 ppm.

ES-MS: m/z 317.7 (M+1).

HPLC: Retention time 11.04 min., purity 96.1% at 280 nm.

### Example 72: 1-Benzyl-7-chloro-6-fluoro-1,2,3,4-tetrahydro-8-quinolinecarboxylic acid (A-74)

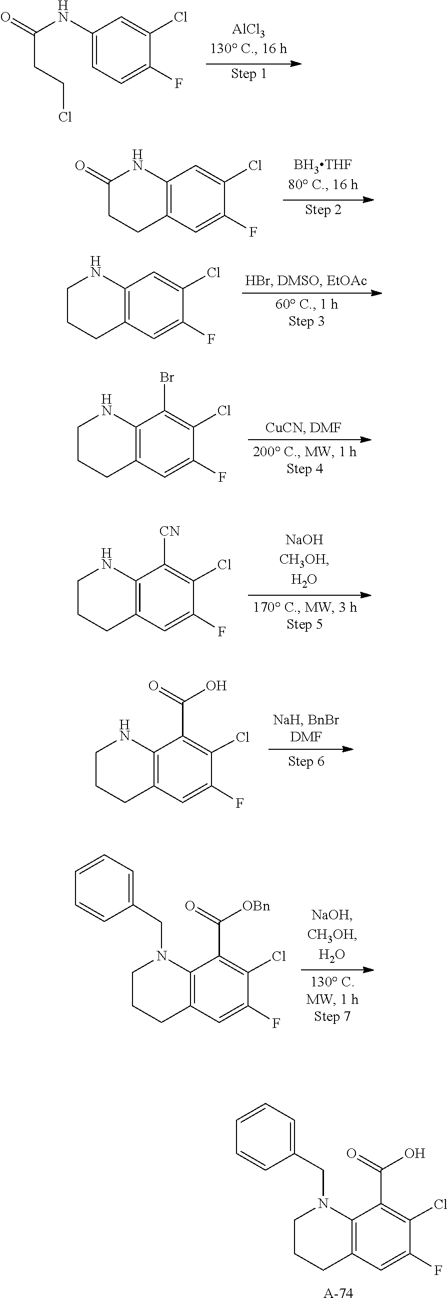

### Step 1: 7-Chloro-6-fluoro-3,4-dihydroquinolin-2(1H)-one

A mixture of 3-chloro-N-(3-chloro-4-fluorophenyl)propanamide (22.0 g, 93.19 mmol) and AlCl₃ (43.49 g, 326.17 mmol) was heated at 130° C. for 16 h. The reaction mixture was cooled, quenched with water (200 mL), extracted with EtOAc (2×200 mL), dried (Na₂SO₄) and evaporated under reduced pressure to provide the title compound (18 g, 97%).

¹H NMR (300 MHz, CDCl₃) δ 9.79-9.38 (br s, 1H), 7.27 (d, 1H), 7.19 (d, 1H), 3.25 (t, 2H), 2.93 (t, 2H). ¹⁹F NMR (300 MHz, CDCl₃); δ −122.78 ppm.

### Step 2: 7-Chloro-6-fluoro-1,2,3,4-tetrahydroquinoline

A mixture of 7-chloro-6-fluoro-3,4-dihydroquinolin-2(1H)-one (18.0 g, 90.17 mmol), and 1M BH₃.THF (360.7 mL, 360.7 mmol) was refluxed for 16 h. The reaction mixture was cooled, and quenched with methanol (10 mL), evaporated and purified by column chromatography on silica gel eluting with methanol/dichloromethane (0-5%) to give the title compound (8.6 g, 51%).

¹H NMR (300 MHz, CDCl₃) δ 6.70 (d, 1H), 6.39 (d, 1H), 3.85-3.70 (br s, 1H), 3.22 (t, 2H), 2.67 (t, 2H), 1.93-1.82 (m, 2H).

¹⁹F NMR (300 MHz, CDCl₃); δ −132.55 ppm.

### Step 3: 8-Bromo-7-chloro-6-fluoro-1,2,3,4-tetrahydroquinoline

HBr (62% in water) (1.29 mL, 23.70 mmol) was added to a solution of 7-chloro-6-fluoro-1,2,3,4-tetrahydroquinoline (2.0 g, 10.77 mmol) and DMSO (0.842 mL, 11.85 mmol) in EtOAc (45 mL) and the reaction mixture was heated at 60° C. for 2 h. The reaction mixture was cooled, quenched with water (20 mL), extracted with EtOAc (2×20 mL), dried (Na₂SO₄) and evaporated under reduced pressure. The resultant residue was purified by column chromatography on silica gel eluting with hexane/EtOAc (0-50%) to afford the title compound (1.74 g, 61%).

¹H NMR (300 MHz, CDC₃) δ 6.75 (d, 1H), 4.47-4.29 (br s, 1H), 3.40-3.32 (m, 2H), 2.73 (t, 2H), 1.98-1.82 (m, 2H). ¹⁹F NMR (300 MHz, CDC₃); δ −126.27 ppm.

### Step 4: 7-Chloro-6-fluoro-1,2,3,4-tetrahydroquinoline-8-carbonitrile

A mixture of 8-bromo-7-chloro-6-fluoro-1,2,3,4-tetrahydroquinoline (1.7 g, 6.43 mmol) and CuCN (0.58 g, 6.43 mmol) in DMF (9 mL) was subjected to microwave conditions at 200° C. for 1 h. The reaction mixture was cooled, quenched with water (20 mL), extracted with EtOAc (2×20 mL), dried (Na₂SO₄) and evaporated under reduced pressure. The crude product was purified by column chromatography on silica gel eluting with hexane/EtOAc (0-50%) to give the title compound (1.1 g, 81%).

¹H NMR (300 MHz, CDC₃) δ 6.91 (d, 1H), 4.85-4.71 (br s, 1H), 3.44-3.33 (m, 2H), 2.72 (t, 2H), 1.97-1.86 (m, 2H). ¹⁹F NMR (300 MHz, CDC₃); δ −130.59 ppm.

### Step 5: 7-Chloro-6-fluoro-1,2,3,4-tetrahydroquinoline-8-carboxylic acid

A solution of NaOH (0.95 g, 23.74 mmol) in water (5 mL) was added to 7-chloro-6-fluoro-1,2,3,4-tetrahydroquinoline-8-carbonitrile (1.0 g, 4.75 mmol) in CH₃OH (5 mL) and the reaction mixture was subjected to microwave conditions at 200° C. for 1 h. The CH₃OH was removed in vacuo, the water layer was washed with CH₂Cl₂ (2×10 mL), acidified to pH ~2.0 with 1.0 N HCl and extracted into EtOAc (2×50 mL). The combined organic extracts were dried (Na₂SO₄) and evaporated under reduced pressure to obtain the title compound (0.44 g, 40%).

¹H NMR (300 MHz, DMSO-d₆) δ 10.21-9.18 (br s, 1H), 7.04 (d, 1H), 3.22 (t, 2H), 2.68 (t, 2H), 1.81-1.68 (m, 2H).

¹⁹F NMR (300 MHz, DMSO-d₆); δ −132.48 ppm.

ES-MS: m/z 228.6 (M-1).

### Step 6: Benzyl 1-benzyl-7-chloro-6-fluoro-1,2,3,4-tetrahydroquinoline-8-carboxylate

Benzyl bromide (0.37 g, 2.18 mmol) was added to a suspension of 7-chloro-6-fluoro-1,2,3,4-tetrahydroquinoline-8-carboxylic acid (0.1 g, 0.43 mmol) and NaH (60% dispersion in mineral oil) (0.17 g, 4.35 mmol) in DMF (10 mL). The reaction mixture was stirred at ambient temperature for 16 h, quenched with water (10 mL). Extraction into EtOAc (2×20 mL), drying (Na₂SO₄) and evaporation provide a residue, which was purified by column chromatography on silica gel eluting with hexane/EtOAc (0-50%) to give the title compound (54 mg, 31%).

¹H NMR (300 MHz, CDC₃) δ 7.33-7.12 (m, 10H), 6.80 (d, 1H), 4.97 (s, 2H), 4.17 (s, 2H), 2.89 (t, 2H), 2.69 (t, 2H), 1.78-1.66 (m, 2H). ¹⁹F NMR (300 MHz, CDC₃); δ −124.60 ppm.

ES-MS: m/z 410.7 (M+1).

### Step 7: 1-Benzyl-7-chloro-6-fluoro-1,2,3,4-tetrahydro-8-quinolinecarboxylic acid

NaOH (24.4 mg, 0.61 mmol) was added to a solution of benzyl 1-benzyl-7-chloro-6-fluoro-1,2,3,4-tetrahydroquinoline-8-carboxylate (50.0 mg, 0.12 mmol) in MeOH (2 mL) and water (2 mL). The reaction mixture was subjected to microwave conditions at 130° C. for 50 min. and evaporated. The residue was dissolved in water (5 mL), washed with CH₂Cl₂ (10 mL), acidified with 1N HCl to pH ~2, extracted into EtOAc (2×20 mL), dried (Na₂SO₄) and evaporated in vacuo to provide the title compound (30 mg, 78%).

¹H NMR (300 MHz, CD₃OD) δ 7.38-7.07 (m, 5H), 6.91 (d, J_{H-F}=9.3 Hz, 1H), 4.16 (s, 2H), 2.86-2.75 (m, 2H), 2.71 (t, 2H), 1.79-1.65 (m, 2H). ¹⁹F NMR (300 MHz, CD₃OD); δ −126.0 ppm.

ES-MS: 318.7 [M−1].

HPLC: Retention Time: 11.84 min., purity: 98.8% @254 nm.

### Example 73: 4-Benzyl-6-chloro-7-fluoro-8-chromancarboxylic acid (A-78)

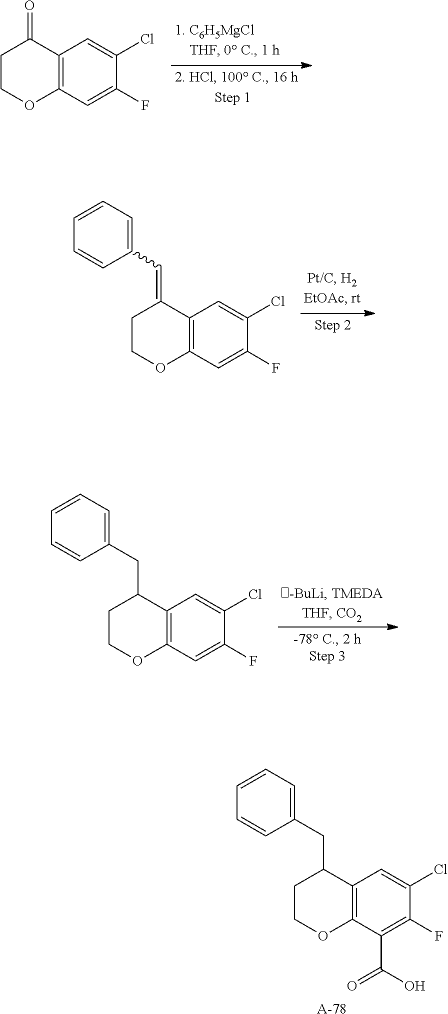

### Step 1: 4-Benzylidene-6-chloro-7-fluorochroman

6-chloro-7-fluorochroman-4-one (0.1 g, 0.49 mmol) in THF (5 mL) was added To a solution of benzylmagnesium chloride (2.OM in THF, 1.0 mL, 1.99 mmol) at 0° C., and the reaction mixture was stirred at that temperature for 1 h. The reaction was quenched with sat. ammonium chloride solution (5 mL), extracted with EtOAc (2×20 mL), dried (Na₂SO₄) and evaporated under reduced pressure. The resultant residue and HCl (6N, 10 ml) were heated at 100° C. for 16 h, then the reaction mixture cooled to ambient temperature, neutralized with NaOH (50% in water, 5 mL), extracted with EtOAc (2×20 mL), dried (Na₂SO₄) and evaporated under reduced pressure to provide the title compound (93.0 mg, 69%), used for the next step without purification.

¹H NMR (300 MHz, CDCl₃) δ 7.61 (d, 1H), 7.43-7.01 (m, 5H), 6.95 (s, 1H), 6.64 (d, 1H), 4.11 (t, 2H), 3.00-2.71 (m, 2H). ¹⁹F NMR (300 MHz, CDCl₃); 5-114.38 ppm.

### Step 2: 4-Benzyl-6-chloro-7-fluorochroman

A mixture of 4-benzylidene-6-chloro-7-fluorochroman (90.0 mg, 0.33 mmol) and Pd/C (20.0 mg, 22%: w/w) in EtOAc (5 mL) was stirred under a hydrogen atmosphere at ambient temperature for 16 h. The mixture was filtered through a pad of Celite®, washed with EtOAc, concentrated under reduced pressure and the residue was purified by column chromatography silica gel eluting with hexane/EtOAc (0-20%) to provide the title compound (56.0 mg, 61%).

¹H NMR (300 MHz, CDC₃) δ 7.42-7.12 (m, 5H), 7.07 (d, 1H), 6.62 (d, 1H), 4.25-4.07 (m, 2H), 3.14 (dd, 1H), 3.07-2.96 (m, 1H), 2.75-2.62 (m, 1H), 1.95-1.81 (m, 1H), 1.79-1.66 (m, 1H). ¹⁹F NMR (300 MHz, CDC₃); δ −116.97 ppm.

### Step 3: 4-Benzyl-6-chloro-7-fluoro-8-chromancarboxylic acid

n-BuLi (2.5 M in hexane) (0.08 mL, 0.19 mmol) was added to a solution of 4-benzyl-6-chloro-7-fluorochroman (50.0 mg, 0.18 mmol) and TMEDA (0.03 mL, 0.19 mmol) in THF (5 mL) at −78° C. The reaction mixture was stirred at that temperature for 2 h and CO₂ gas was bubbled for 10 min. The reaction mixture was stirred at −78° C. for 1 h., slowly brought to ambient temperature and quenched with 1M NaOH (5 mL). The mixture was washed with EtOAc (2×10 mL), the aqueous layer was acidified to pH ~2.0 with 1.0 N HCl. Extraction with EtOAc (2×20 mL), drying of the combined extracts (Na₂SO₄) and evaporation provided a residue which was purified by semi-prep HPLC, eluting with CH₃CN/0.1% formic acid (30-100% in water) to provide the title compound (18 mg, 31%).

¹H NMR (300 MHz, CD₃OD) δ 7.38-7.20 (m, 5H), 7.17 (d, J_{H-F}=8.3 Hz, 1H), 4.36-4.18 (m, 2H), 3.19-3.04 (m, 2H), 2.85-2.70 (m, 1H), 2.00-1.72 (m, 2H). ¹⁹F NMR (300 MHz, CD₃OD); δ −121.6 ppm.

ES-MS: m/z 321.7 (M+1).

HPLC: Retention time 14.34 min., purity 97.4% at 280 nm.

### Example 74: 3-Chloro-2-ethoxy-5,6,7,8-tetrahydro-1-naphthoic acid (A-82)

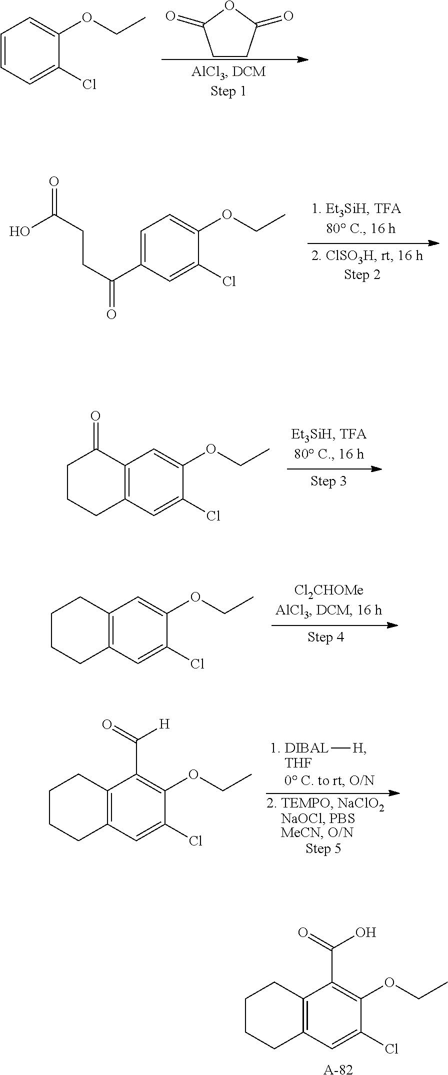

3-Chloro-2-ethoxy-5,6,7,8-tetrahydro-1-naphthoic acid was prepared following the route given and using the techniques described herein.

¹H NMR (300 MHz, CDCl₃) δ 11.00-8.27 (Br s, 1H); 7.25 (s, 1H); 4.22 (q, 2H); 2.93-2.85 (m, 2H); 2.84-2.76 (m, 2H); 1.94-1.80 (m, 4H); 1.49 (t, 3H).

ES-MS: 253.7 [M−1].

HPLC Retention Time: 12.86 min, purity 98.3% @280 nm.

### Example 75: Electrophysiological Measurement of Compound Inhibition of CIC-1 in Rat Muscle

The investigatory goal of these experiments was to evaluate whether compounds inhibit CIC-1 channels in native tissue of rat skeletal muscle fibres. Apparent CIC-1 affinity was reported by the concentration of compound at which 50% of the compound's full inhibition of CIC-1 was observed (EC₅₀) (see also WO2019/115777).

Experimentally, Gₘ was measured in individual fibres of whole rat soleus muscles using a three micro-electrodes technique described in this example and in full detail elsewhere (Riisager et al., Determination of cable parameters in skeletal muscle fibres during repetitive firing of action potentials. Journal of Physiology, 2014, 592, 4417-4429). Briefly, intact rat soleus muscles were dissected out from 12-14 week old Wistar rats and placed in an experimental chamber that was perfused with a standard Krebs Ringer solution containing 122 mM NaCl, 25 mM NaHCO₃, 2.8 mM KCl, 1.2 mM KH₂PO₄, 1.2 mM MgSO₄, 1.3 mM CaCl₂, 5.0 mM D-glucose. During experiments, the solution was kept at approx. 30° C. and continuously equilibrated with a mixture of 95% O₂ and 5% CO₂, pH ~7.4. The experimental chamber was placed in Nikon upright microscope that was used to visualize individual muscle fibres and the three electrodes (glass pipettes filled with 2 M potassium citrate). For Gₘ measurements, the electrodes were inserted into the same fibre with known inter-electrode distances of 0.35-0.5 mm (V1-V2, X1) and 1.1-1.5 mm (V1-V3, X3). The membrane potential of the impaled muscle fibre was recorded by all electrodes. Two of the electrodes were furthermore used to inject 50 ms current pulses of −30 nA. Given the positions of the electrodes, three different inter-electrode distances could be identified (X1-X2, X1-X3, X2-X3) and hence the membrane potential responses to the current injections could be obtained at three distances from the point of current injection. The steady state voltage deflection at each distance was divided by the magnitude of current injected (−30 nA) and the resulting transfer resistances were plotted against inter-electrode distance and the data was fitted to a mono-exponential function from which Gₘ could be calculated using linear cable theory.

To establish a dose response relationship, Gₘ was first determined in 10 muscle fibres in the absence of compound and then at four increasing compound concentrations with Gₘ determinations in 5-10 fibres at each concentration. The average Gₘ values at each concentration were plotted against compound concentration and the data was fitted to sigmoidal function to obtain an EC₅o value. Table 1 shows the EC₅o values for a range of compounds with n values referring to number of experiments that each reflect recordings from around 50 fibres.

TABLE 2
Inhibition of CIC-1 ion channel using compounds of the disclosure

| Compound investigated | Potency Range |
|---|---|
| A-1 | A |
| A-3 | B |
| A-4 | A |
| A-5 | A |
| A-6 | C |
| A-8 | C |
| A-9 | B |
| A-10 | B |
| A-13 | A |
| A-14 | A |
| A-15 | B |
| A-16 | C |
| A-17 | A |
| A-18 | C |
| A-19 | C |
| A-20 | B |
| A-21 | A |
| A-22 | C |
| A-23 | B |
| A-24 | B |
| A-25 | C |
| A-26 | C |
| A-27 | B |
| A-28 | B |
| A-29 | B |
| A-30 | C |
| A-31 | C |
| A-32 | C |
| A-33 | B |
| A-35 | C |
| A-36 | B |
| A-37 | B |
| A-38 | C |
| A-39 | C |
| A-40 | C |
| A-41 | B |
| A-42 | A |
| A-44 | B |
| A-47 | B |
| A-48 | B |
| A-49 | C |
| A-50 | B |
| A-51 | B |
| A-52 | B |
| A-53 | C |
| A-54 | A |
| A-56 | B |
| A-59 | B |
| A-61 | C |
| A-63 | B |
| A-65 | C |
| A-69 | C |
| A-70 | C |
| A-71 | C |
| A-72 | C |
| A-73 | C |
| A-74 | C |

Potency range: A means an EC₅₀ between 10 and 20 μM; B means an EC₅₀ between 2 and 10 μM; C means an EC₅₀ between 0.2 and 2 μM.

In conclusion, this example demonstrates that the compounds of the present disclosure inhibit the CIC-1 channel.

### Example 76: Measurement of Force in an In Vitro Model

The current disclosure relates to compounds that inhibit CIC-1 ion channels and increase muscle excitability and thereby improve muscle function in clinical conditions where muscle activation is failing. Such conditions result in loss of contractile function of skeletal muscle, weakness and excessive fatigue. In this series of experiments the compounds were tested for their ability to restore contractile function of isolated rat muscle when the neuromuscular transmission had been compromised akin to neuromuscular disorders.

Experimentally, soleus muscles from 4-5 week old rats were isolated with the motor nerve remaining attached. The nerve-muscle preparations were mounted in experimental setups that enabled electrical stimulation of the motor nerve. Stimulation of the motor nerve led to activation of the muscle fibres and ensuing force production that was recorded. The nerve-muscle preparations were also in these experiments incubated in the standard Krebs Ringer (see example 15) and the solution was heated to 30° C. and continuously equilibrated with a mixture of 95% O₂ and 5% CO₂, pH ~7.4.

After mounting the nerve-muscle preparation in the experimental setup, the contractile function of the muscle was initially assessed under the control conditions. Sub-maximal concentration of tubocurarine (115 nM), an acetylcholine receptor antagonist, was then added to the experimental bath to impose partial inhibition of the ability of the motor nerve to activate the muscle fibres. The experimental condition mimics the failing neuromuscular transmission in a range of neuromuscular disorders. After addition of tubocurarine the contractile force declined over the next 90 minutes to 10-50% of the control force. The test compound was then added to obtain the required compound concentration in the bath and the contractile force recovered was measured. To quantify the ability of the compound to restore force the percentage of the initial force that was restored was determined after 40 mins of compound exposure and the point increase is reported in Tables 3 to 7.

TABLE 3
Percentage increase of initial force that was
restored when using 100 μM of test compound

| Compound investigated | Point increase (%) |
|---|---|
| A-45 | 38 |
| A-68 | 40 |

TABLE 4
Percentage increase of initial force that was
restored when using 50 μM of test compound

| Compound investigated | Point increase (%) |
|---|---|
| A-1 | 23 |
| A-2 | 19 |
| A-4 | 30 |
| A-5 | 43 |
| A-7 | 36 |
| A-11 | 31 |
| A-12 | 19 |
| A-13 | 26 |
| A-14 | 39 |
| A-15 | 40 |
| A-17 | 41 |
| A-19 | 41 |
| A-20 | 38 |
| A-21 | 42 |
| A-23 | 44 |
| A-24 | 43 |
| A-28 | 39 |
| A-29 | 42 |
| A-34 | 20 |
| A-37 | 48 |
| A-42 | 47 |
| A-43 | 25 |
| A-44 | 59 |
| A-47 | 37 |
| A-50 | 51 |
| A-54 | 47 |
| A-55 | 42 |
| A-58 | 48 |
| A-60 | 21 |
| A-80 | 22 |
| A-81 | 19 |

TABLE 5
Percentage increase of initial force that was
restored when using 25 μM of test compound

| Compound investigated | Point increase (%) |
|---|---|
| A-62 | 14 |
| A-64 | 34 |
| A-76 | 45 |
| A-78 | 35 |

TABLE 6
Percentage increase of initial force that was
restored when using 10 μM of test compound

| Compound investigated | Point increase (%) |
|---|---|
| A-3 | 38 |
| A-6 | 42 |
| A-8 | 50 |
| A-9 | 14 |
| A-10 | 36 |
| A-16 | 43 |
| A-18 | 46 |
| A-22 | 33 |
| A-25 | 43 |
| A-26 | 52 |
| A-27 | 38 |
| A-30 | 45 |
| A-31 | 48 |
| A-32 | 24 |
| A-33 | 17 |
| A-35 | 16 |
| A-36 | 43 |
| A-38 | 48 |
| A-39 | 59 |
| A-40 | 39 |
| A-41 | 49 |
| A-48 | 25 |
| A-49 | 40 |
| A-51 | 29 |
| A-52 | 18 |
| A-53 | 43 |
| A-56 | 31 |
| A-59 | 53 |
| A-61 | 63 |
| A-63 | 28 |
| A-65 | 49 |
| A-66 | 25 |
| A-67 | 25 |
| A-69 | 55 |
| A-70 | 25 |
| A-71 | 23 |
| A-72 | 20 |
| A-73 | 23 |
| A-74 | 11 |
| A-75 | 10 |
| A-77 | 18 |
| A-79 | 34 |
| A-82 | 61 |

In conclusion, this example demonstrates that the compounds of the present disclosure are able to increase muscle excitability and thereby improve muscle function in clinical conditions.

1. A compound of Formula (1): 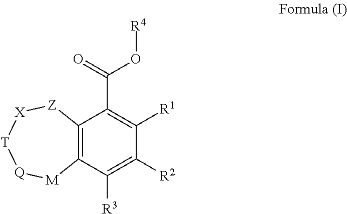
wherein:
M is CR⁵R⁶, NR⁹, O or S;
Q is CR⁷R⁶, NR¹⁰, O or S;
T is absent, CR⁷R⁶, NR¹⁰, O or S;
X is absent, CR⁷R⁶, NR¹⁰, O or S;
Z is CR⁸R⁶, NR¹¹, O or S;
wherein no more than one of M, Q, T, X or Z is selected from the group consisting of NR⁹, NR¹⁰, NR¹¹, O or S;
wherein R⁵ and R⁸, or R⁵ and R¹¹, or R⁹ and R³ are optionally joined together to form a ring by —CH₂—, —CH₂CH₂- or —CH₂CH₂CH₂—;
R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
R³ is selected from the group consisting of H; F and Cl;
R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
R⁵ is selected from the group consisting of H, deuterium, F, and C₁₋₃ alkyl optionally substituted with one or more, identical or different, substituents R¹², or when R⁵ is linked to R³ or R¹¹, then R⁵ is a bond or C₁₋₃ alkanediyl;
R⁶ is independently selected from the group consisting of H, deuterium, F, C₁₋₃ alkyl substituted with R¹⁴ and C₁₋₃ alkyl optionally substituted with one or more, identical or different, substituents R¹²;
R⁷ is independently selected from the group consisting of H, deuterium, F and C₁₋₃ alkyl optionally substituted with one or more, identical or different, substituents R¹²;
R³ is selected from the group consisting of H, deuterium, F and C₁₋₃ alkyl optionally substituted with one or more, identical or different, substituents R¹², or when R³ is linked to R⁵ or R⁹, then R³ is a bond or C₁₋₃ alkanediyl;
R⁹ is selected from the group consisting of H, C₁₋₃ alkyl optionally substituted with one or more, identical or different, substituents R¹², and C₁₋₃ alkyl substituted with R¹⁴ or when R⁹ is linked to R⁸, then R⁹ is a bond or C₁₋₃ alkanediyl;
R¹⁰ is independently selected from benzyl optionally substituted with one or more, identical or different, substituents R¹³;
R¹¹ is selected from the group consisting of H, C₁₋₅ alkyl optionally substituted with one or more, identical or different, substituents R¹², and C₁₋₃ alkyl substituted with R¹⁵, or when R¹¹ is linked to R⁵, then R¹¹ is a bond or C₁₋₃ alkanediyl;
R¹² is independently selected from the group consisting of deuterium, F and OMe;
R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F;
R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; and
R¹⁵ is independently selected from the group consisting of C₃₋₆ cycloalkyl optionally substituted with one or more, identical or different, substituents R¹², C₆10 aryl optionally substituted with one or more, identical or different, substituents R¹³ and 5- to 10-membered heteroaryl optionally substituted with one or more, identical or different, substituents R¹³;
or a pharmaceutically acceptable salt, hydrate, polymorph, tautomer, or solvate thereof provided that the compound is not a compound selected from the group consisting of:
10-chloro-9-methoxybicyclo[5.4.0]undeca-1(7),8,10-triene-8-carboxylic acid;
methyl 10-chloro-9-methoxybicyclo[5.4.0]undeca-1(7),8,10-triene-8-carboxylate;
ethyl 10-chloro-9-methoxybicyclo[5.4.0]undeca-1(7),8,10-triene-8-carboxylate;
3-chloro-2-methoxy-5,6,7,8-tetrahydro-1-naphthoic acid;
methyl 3-chloro-2-methoxy-5,6,7,8-tetrahydro-1-naphthoate;
ethyl 3-chloro-2-methoxy-5,6,7,8-tetrahydro-1-naphthoate;
methyl 2-methoxy-3-(trifluoromethyl)-5,6,7,8-tetrahydro-1-naphthoate;
2,3-dimethyl-5,6,7,8-tetrahydro-1-naphthoic acid;
2,3,4-trichloro-5,6,7,8-tetrahydro-1-naphthoic acid;
5,6-dimethyl-4-indancarboxylic acid;
methyl 5,6-dimethyl-4-indancarboxylate;
6-chloro-5-methoxy-1,3-dihydro-4-isobenzofuroic acid;
methyl 6-chloro-5-methoxy-1,3-dihydro-4-isobenzofuroate;
ethyl 6-chloro-5-methoxy-1,3-dihydro-4-isobenzofuroate;
5,6-dichloro-1,3-dihydro-4-isobenzofuroic acid;
5-methoxy-6-methyl-1,3-dihydro-4-isobenzofuroic acid;
5-methoxy-6-methyl-2,3-dihydro-1-benzofuran-4-carboxylic acid;
methyl 5-methoxy-6-methyl-2, 3-dihydro-1-benzofuran-4-carboxylate;
ethyl 5-methoxy-6-methyl-2,3-dihydro-1-benzofuran-4-carboxylate;
5-ethyl-6-methoxy-2,3-dihydro-1-benzofuran-7-carboxylic acid;
5,6-difluoro-2,2-dimethyl-2,3-dihydro-1-benzofuran-7-carboxylic acid;
5-fluoro-6-methyl-2,3-dihydro-1-benzothiophene-4-carboxylic acid;
5-chloro-6-methyl-2,3-dihydro-1-benzothiophene-4-carboxylic acid;
5,6-dichloro-2,3-dihydro-1-benzothiophene-4-carboxylic acid;
6-chloro-5-methyl-2,3-dihydro-1-benzothiophene-7-carboxylic acid; and
5,6-dimethyl-2,3-dihydro-1-benzothiophene-7-carboxylic acid.

2. The compound according to claim 1, wherein compound is of formula (III): 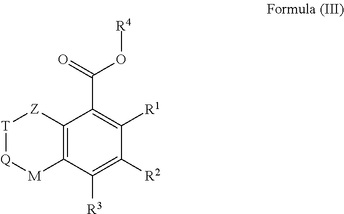
wherein:
M is CR^{5M}R^{6M}, NR⁹, O or S;
Q is CR^{7Q}R^{6Q}, NR¹⁰, O or S;
T is CR^{7T}R^{6T}, NR^{10T}, O or S;
Z is CR^{8Z}R^{6Z}, NR¹¹, O or S;
wherein no more than one of M, Q, T or Z is selected from the group consisting of NR^{9M}, NR^{10Q}, NR^{10T}, NR¹¹, O or S;
R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
R³ is selected from the group consisting of H; F and Cl;
R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
R^{5M} is selected from the group consisting of H, deuterium, F and C₁₋₃ alkyl optionally substituted with one or more, identical or different, substituents R¹²;
R^{6M} R^{6Q} R^{6T} and R^{6Z} are independently selected from the group consisting of H, deuterium, F, C₁₋₃ alkyl substituted with R¹⁴, and C₁₋₃ alkyl optionally substituted with one or more, identical or different, substituents R¹²;
R^{7Q} and R^{7T} are independently selected from the group consisting of H, deuterium, F and C₁₋₃ alkyl optionally substituted with one or more, identical or different, substituents R¹²;
R^{8Z} is selected from the group consisting of H, deuterium, F and C₁₋₃ alkyl optionally substituted with one or more, identical or different, substituents R¹²;
R⁹ is selected from the group consisting of H, C₁₋₃ alkyl optionally substituted with one or more, identical or different, substituents R¹² and C₁₋₃ alkyl substituted with R¹⁴;
R^{10Q} and R^{10T} are independently selected from benzyl optionally substituted with one or more, identical or different, substituents R¹³;
R¹¹ is selected from the group consisting of H, C₁₋₅ alkyl optionally substituted with one or more, identical or different, substituents R¹² and C₁₋₃ alkyl substituted with R¹⁵;
R¹² is independently selected from the group consisting of deuterium, F and OMe;
R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F;
R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; and
R¹⁵ is independently selected from the group consisting of C₃₋₆ cycloalkyl optionally substituted with one or more, identical or different, substituents R¹², C₆10 aryl optionally substituted with one or more, identical or different, substituents R¹³ and 5- to 10-membered heteroaryl optionally substituted with one or more, identical or different, substituents R¹³;
or a pharmaceutically acceptable salt, hydrate, polymorph, tautomer, or solvate thereof provided that the compound is not a compound selected from the group consisting of:
3-chloro-2-methoxy-5,6,7,8-tetrahydro-1-naphthoic acid;
methyl 3-chloro-2-methoxy-5,6,7,8-tetrahydro-1-naphthoate;
ethyl 3-chloro-2-methoxy-5,6,7,8-tetrahydro-1-naphthoate;
2,3,4-trichloro-5,6,7,8-tetrahydro-1-naphthoic acid;
methyl 2-methoxy-3-(trifluoromethyl)-5,6,7,8-tetrahydro-1-naphthoate; and
methyl 2-methoxy-3-(trifluoromethyl)-5,6,7,8-tetrahydro-1-naphthoate.

3. The compound according to claim 1, wherein the compound is selected from the group consisting of Formula (IV), Formula (V), Formula (VI), Formula (VII), Formula (VIII), Formula (IX), Formula (X), Formula (XI), Formula (XII), Formula (XIII), Formula (XIV), Formula (XV), Formula (XVI), Formula (XVII), Formula (XVIII), Formula (XIX), Formula (XX), Formula (XXI), Formula (XXII), Formula (XXIII), Formula (XXIV), Formula (XXV), Formula (XXVI) and Formula (XXVII): 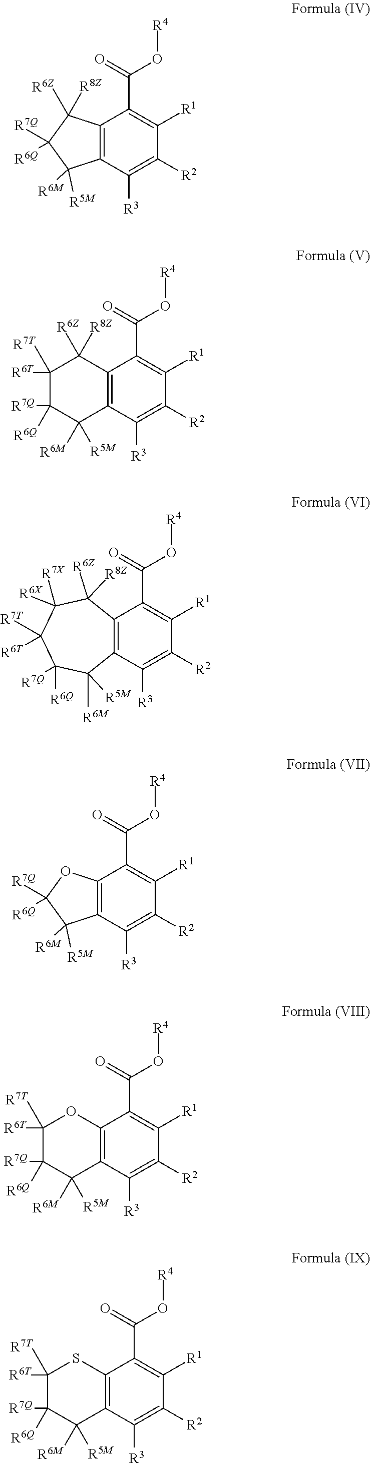 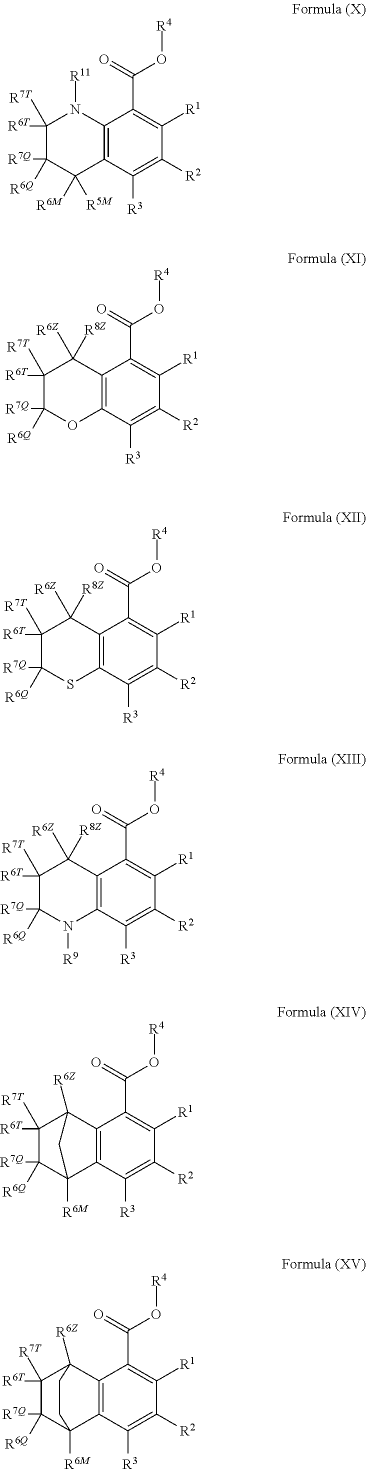 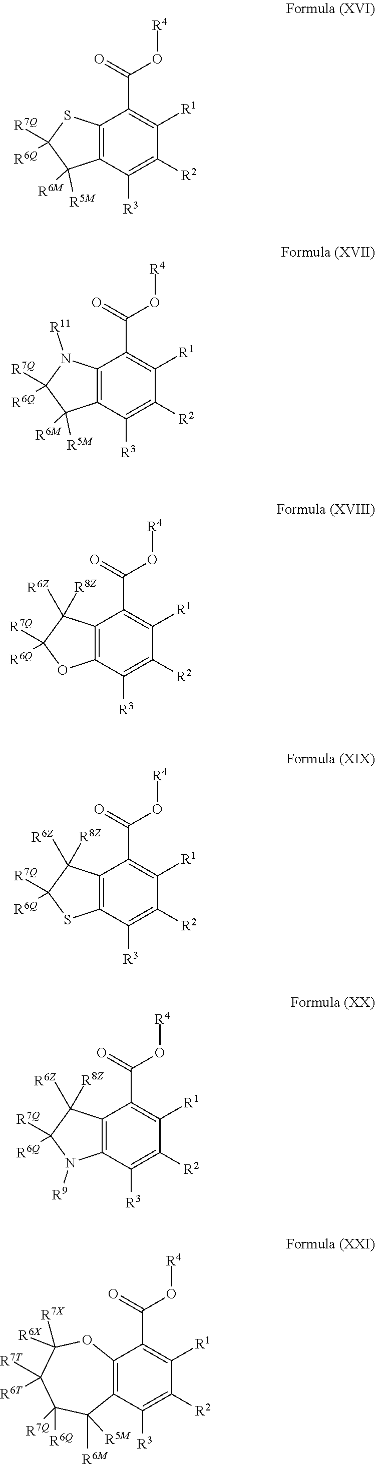 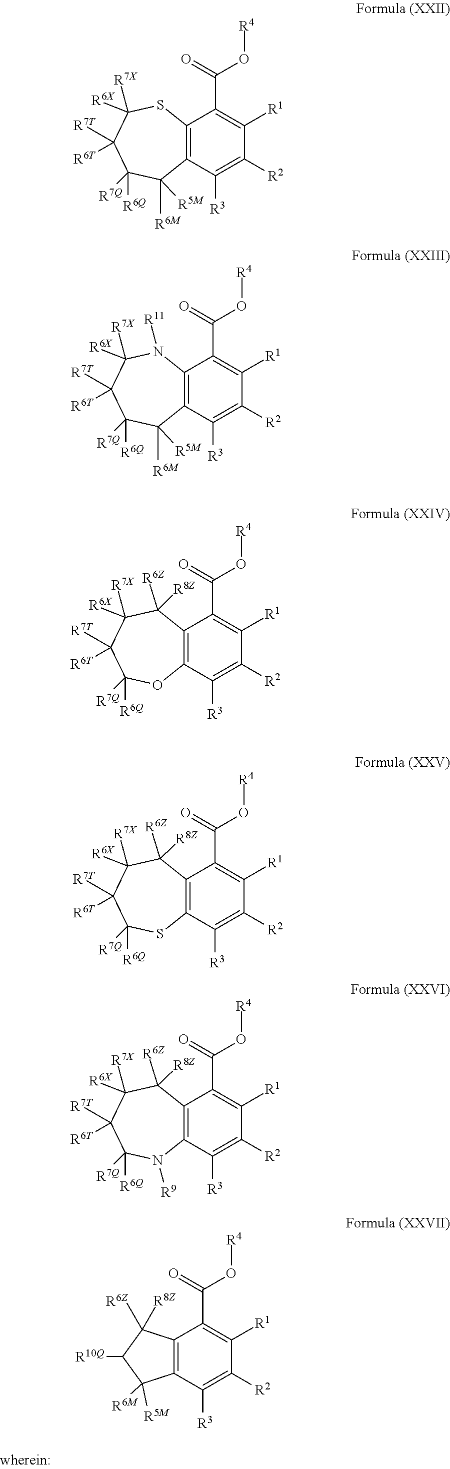
R¹ is selected from the group consisting of F; Cl; C₁₋₃ alkyl optionally substituted with one or more deuterium; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
R² is selected from the group consisting of F; Cl; C₁₋₃ alkyl optionally substituted with one or more, identical or different, substituents R¹² and C₃ cycloalkyl optionally substituted with one or more, identical or different, substituents R¹²;
R³ is selected from the group consisting of H; F and Cl;
R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
R^{5M} is selected from the group consisting of H, deuterium, F and C₁₋₃ alkyl optionally substituted with one or more, identical or different, substituents R¹²;
R^{6M} R^{6Q} R^{6T} R^{6X} and R^{6Z} are independently selected from the group consisting of H, deuterium, F, C₁₋₃ alkyl substituted with R¹⁴ and C₁₋₃ alkyl optionally substituted with one or more, identical or different, substituents R¹²;
R^{7Q}, R^{7T} and R^{7X} are independently selected from the group consisting of H, deuterium, F and C₁₋₃ alkyl optionally substituted with one or more, identical or different, substituents R¹²;
R^{8Z} is selected from the group consisting of H, deuterium, F and C₁₋₃ alkyl optionally substituted with one or more, identical or different, substituents R¹²;
R⁹ is selected from the group consisting of H, C₁₋₃ alkyl optionally substituted with one or more, identical or different, substituents R¹² and C₁₋₃ alkyl substituted with R¹⁴;
R¹⁰ is selected from benzyl optionally substituted with one or more, identical or different, substituents R¹³;
R¹¹ is selected from the group consisting of H, C₁₋₃ alkyl optionally substituted with one or more, identical or different, substituents R¹² and C₁₋₃ alkyl substituted with R¹⁵;
R¹² is independently selected from the group consisting of deuterium, F and OMe;
R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F;
R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; and
R¹⁵ is independently selected from the group consisting of C₃₋₆ cycloalkyl optionally substituted with one or more, identical or different, substituents R¹², C₆10 aryl optionally substituted with one or more, identical or different, substituents R¹³ and 5- to 10-membered heteroaryl optionally substituted with one or more, identical or different, substituents R¹³;
or a pharmaceutically acceptable salt, hydrate, polymorph, tautomer, or solvate thereof.

4. The compound according to claim 1, wherein R¹ is selected from the group consisting of F, Cl, Me, OMe, OCH₂F, OCHF₂, OCF₃ and SMe.

5. The compound according to claim 1, wherein R² is selected from the group consisting of F, Cl and Me.

6. The compound according to claim 1, wherein R³ is H.

7. The compound according to claim 1, wherein R⁴ is H.

8. The compound according to claim 1, wherein R⁵, R⁶, R⁷ and R³ are H.

9. The compound according to claim 1, wherein the compound is selected from the group consisting of:
6-chloro-5-methyl-2,3-dihydro-1H-indene-4-carboxylic acid;
6-chloro-5-methoxy-2,3-dihydro-1H-indene-4-carboxylic acid;
6-chloro-5-(fluoromethoxy)-2,3-dihydro-1H-indene-4-carboxylic acid;
3-chloro-2-methyl-5,6,7,8-tetrahydronaphthalene-1-carboxylic acid;
6-chloro-5-(difluoromethoxy)-2,3-dihydro-1H-indene-4-carboxylic acid;
6-chloro-5-(trifluoromethoxy)-2,3-dihydro-1H-indene-4-carboxylic acid;
5-chloro-6-methyl-2,3-dihydro-1-benzofuran-7-carboxylic acid;
6-chloro-5-ethyl-2,3-dihydro-1H-indene-4-carboxylic acid;
6-chloro-7-methyl-3,4-dihydro-2H-1-benzopyran-8-carboxylic acid;
5-chloro-6-methoxy-2,3-dihydro-1-benzofuran-7-carboxylic acid;
5-chloro-4-methoxytricyclo[6.2.1.0^{2,7}]undeca-2(7),3,5-triene-3-carboxylic acid;
3-fluoro-2-methoxy-5,6,7,8-tetrahydronaphthalene-1-carboxylic acid;
3-chloro-4-fluoro-2-methoxy-5,6,7,8-tetrahydronaphthalene-1-carboxylic acid;
4-chloro-5-methyltricyclo[6.2.1.0^{2,7}]undeca-2,4,6-triene-3-carboxylic acid;
5-chloro-4-methyltricyclo[6.2.1.0^{2,7}]undeca-2(7),3,5-triene-3-carboxylic acid;
6-chloro-5-(methylsulfanyl)-2,3-dihydro-1H-indene-4-carboxylic acid;
3-chloro-2-(difluoromethoxy)-5,6,7,8-tetrahydronaphthalene-1-carboxylic acid;
3-chloro-2-fluoro-5,6,7,8-tetrahydronaphthalene-1-carboxylic acid;
2-chloro-3-fluoro-5,6,7,8-tetrahydronaphthalene-1-carboxylic acid;
2-chloro-3-methyl-5,6,7,8-tetrahydronaphthalene-1-carboxylic acid;
5-chloro-4-methoxytricyclo[6.2.2.0^{2,7}]dodeca-2,4,6-triene-3-carboxylic acid;
2,3-dichloro-5,6,7,8-tetrahydronaphthalene-1-carboxylic acid;
6-chloro-7-methoxy-3,4-dihydro-2H-1-benzothiopyran-8-carboxylic acid;
3-chloro-2-(methylsulfanyl)-5,6,7,8-tetrahydronaphthalene-1-carboxylic acid;
3-chloro-2-methyl-6,7,8,9-tetrahydro-5H-benzo[7]annulene-1-carboxylic acid;
2-chloro-3-methyl-6,7,8,9-tetrahydro-5H-benzo[7]annulene-1-carboxylic acid;
3-chloro-2-ethenyl-5,6,7,8-tetrahydronaphthalene-1-carboxylic acid;
6-chloro-7-fluoro-3,4-dihydro-2H-1-benzopyran-8-carboxylic acid;
7-chloro-6-fluoro-3,4-dihydro-2H-1-benzopyran-8-carboxylic acid;
6-chloro-7-fluoro-3,4-dihydro-2H-1-benzothiopyran-8-carboxylic acid;
7-chloro-6-fluoro-3,4-dihydro-2H-1-benzopyran-5-carboxylic acid;
6-chloro-7-fluoro-1-methyl-1,2,3,4-tetrahydroquinoline-8-carboxylic acid;
6-chloro-7-fluoro-1,2,3,4-tetrahydroquinoline-8-carboxylic acid;
6,7-difluoro-3,4-dihydro-2H-1-benzothiopyran-8-carboxylic acid;
5-chloro-6-fluoro-2,3-dihydro-1-benzofuran-7-carboxylic acid;
2,3-difluoro-5,6,7,8-tetrahydronaphthalene-1-carboxylic acid;
7-chloro-6-fluoro-1-methyl-1,2,3,4-tetrahydroquinoline-5-carboxylic acid;
7-chloro-6-fluoro-1-methyl-1,2,3,4-tetrahydroquinoline-8-carboxylic acid;
5-methoxy-6-methyl-2,3-dihydro-1H-indene-4-carboxylic acid;
7-fluoro-6-methyl-3,4-dihydro-2H-1-benzothiopyran-8-carboxylic acid;
2-fluoro-3-methyl-5,6,7,8-tetrahydronaphthalene-1-carboxylic acid;
7-fluoro-6-methyl-3,4-dihydro-2H-1-benzopyran-8-carboxylic acid;
3-ethyl-2-fluoro-5,6,7,8-tetrahydronaphthalene-1-carboxylic acid;
3-fluoro-2-methyl-5,6,7,8-tetrahydronaphthalene-1-carboxylic acid;
3-cyclopropyl-2-fluoro-5,6,7,8-tetrahydronaphthalene-1-carboxylic acid;
8-chloro-7-fluoro-2,3,4,5-tetrahydro-1-benzoxepine-9-carboxylic acid;
6-fluoro-7-methyl-3,4-dihydro-2H-1-benzopyran-5-carboxylic acid;
8-chloro-7-fluoro-2,3,4,5-tetrahydro-1-benzoxepine-6-carboxylic acid;
3-chloro-8,8-difluoro-2-methoxy-5,6,7,8-tetrahydronaphthalene-1-carboxylic acid;
2-fluoro-3-(trifluoromethyl)-5,6,7,8-tetrahydronaphthalene-1-carboxylic acid;
6-chloro-7-fluoro-2,2-dimethyl-3,4-dihydro-2H-1-benzopyran-8-carboxylic acid;
5-chloro-6-fluoro-2,3-dihydro-1H-indene-4-carboxylic acid;
6-chloro-5-fluoro-2,3-dihydro-1H-indene-4-carboxylic acid;
7-chloro-6-fluoro-3,4-dihydro-2H-1-benzothiopyran-8-carboxylic acid;
7-chloro-6-fluoro-3,4-dihydro-2H-1-benzothiopyran-5-carboxylic acid;
2-benzyl-5,6-dichloro-2,3-dihydro-1H-isoindole-4-carboxylic acid;
6-chloro-7-methoxy-3,4-dihydro-2H-1-benzopyran-8-carboxylic acid;
6-chloro-7-fluoro-1-[(3-thienyl)methyl]-1,2,3,4-tetrahydro-8-quinolinecarboxylic acid;
1-benzyl-6-chloro-7-fluoro-1,2,3,4-tetrahydro-8-quinolinecarboxylic acid;
6-chloro-7-fluoro-1-[(p-fluorophenyl)methyl]-1,2,3,4-tetrahydro-8-quinolinecarboxylic acid;
3-chloro-5,5-difluoro-2-methoxy-5,6,7,8-tetrahydro-1-naphthoic acid;
1-benzyl-7-chloro-6-fluoro-1,2,3,4-tetrahydro-8-quinolinecarboxylic acid;
7-chloro-6-fluoro-2,2-dimethyl-8-chromancarboxylic acid;
1-benzyl-6-chloro-5-fluoro-4-indolinecarboxylic acid;
7-chloro-6-fluoro-1-methyl-1,2,3,4-tetrahydro-8-quinolinecarboxylic acid;
4-benzyl-6-chloro-7-fluoro-8-chromancarboxylic acid;
7-chloro-6-methoxy-5-chromancarboxylic acid;
4-benzyl-7-fluoro-6-methoxy-8-chromancarboxylic acid;
4-benzyl-7-fluoro-6-methoxy-5-chromancarboxylic acid;
6-chloro-7-fluoro-5-chromancarboxylic acid; and
3-chloro-2-ethoxy-5,6,7,8-tetrahydro-1-naphthoic acid.

10. A composition comprising the compound according to claim 1 and a pharmaceutically acceptable carrier.

11. A method for treating a neuromuscular disorder comprising administering the composition according to claim 10 to a subject in need thereof.

12. The method according to claim 11, wherein the neuromuscular disorder is selected from the group consisting of myasthenia gravis, autoimmune myasthenia gravis, congenital myasthenic syndrome, seronegative myasthenia gravis, muscle specific kinase myasthenia gravis (MuSK-MG), Lambert-Eaton Syndrome, critical illness myopathy, amyotrophic lateral sclerosis (ALS), spinal muscular atrophy (SMA), critical illness myopathy (CIM), Charcot-Marie Tooth disease, diabetic polyneuropathy, periodic paralysis, hypokalemic periodic paralysis, hyperkalemic periodic paralysis, myotubular myopathy, Duchenne muscular dystrophy, Guillain-Barre syndrome, poliomyelitis, post-polio syndrome, chronic fatigue syndrome, critical illness polyneuropathy, metabolic myopathy, Kennedy's disorder, multiple sclerosis and multifocal motor neuropathy.

13. The method according to claim 11, wherein the neuromuscular disorder is sarcopenia.

14. A method for reversing and/or ameliorating a neuromuscular blockade comprising administering the composition according to claim 10.

15. A compound of Formula (1): 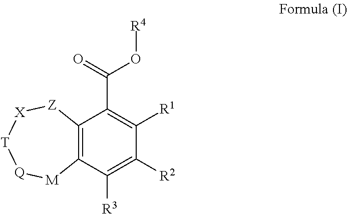
wherein:
M is CR⁵R⁶, NR⁹, O or S;
Q is CR⁷R⁶, NR¹⁰, O or S;
T is absent, CR⁷R⁶, NR¹⁰, O or S;
X is absent, CR⁷R⁶, NR¹⁰, O or S;
Z is CR⁸R⁶, NR¹¹, O or S;
wherein no more than one of M, Q, T, X or Z is selected from the group consisting of NR⁹, NR¹⁰, NR¹¹, 0 or S;
wherein R⁵ and R⁸, or R⁵ and R¹¹, or R⁹ and R³ are optionally joined together to form a ring by —CH₂—, —CH₂CH₂- or —CH₂CH₂CH₂—;
R¹ is selected from the group consisting of H; F; Cl; Br; I; C₁₋₃ alkyl optionally substituted with one or more, identical or different, substituents R¹²; C₂₋₃ alkenyl optionally substituted with one or more, identical or different, substituents R¹²; —OC₁₋₃ alkyl optionally substituted with one or more, identical or different, substituents R¹²; —OC₃₋₅ cycloalkyl optionally substituted with one or more, identical or different, substituents R¹²; —SC₁₋₃ alkyl optionally substituted with one or more, identical or different, substituents R¹² and —SC₃₋₅ cycloalkyl optionally substituted with one or more, identical or different, substituents R¹²;
R² is selected from the group consisting of H; F; Cl; Br; I; C₁₋₃ alkyl optionally substituted with one or more, identical or different, substituents R¹²; —OC₁₋₃ alkyl optionally substituted with one or more, identical or different, substituents R¹² and C₃ cycloalkyl optionally substituted with one or more, identical or different, substituents R¹²;
R³ is selected from the group consisting of H; F and Cl;
R⁴ is selected from the group consisting of H; C₁₋₅ alkyl optionally substituted with one or more, identical or different, substituents R¹²; C₂₋₅ alkenyl optionally substituted with one or more, identical or different, substituents R¹²; C₂₋₅ alkynyl optionally substituted with one or more, identical or different, substituents R¹²; C₃₋₆ cycloalkyl optionally substituted with one or more, identical or different, substituents R¹²; phenyl optionally substituted with one or more, identical or different, substituents R¹³; and benzyl optionally substituted with one or more, identical or different, substituents R¹³;
R⁵ is selected from the group consisting of H, deuterium, F, and C₁₋₃ alkyl optionally substituted with one or more, identical or different, substituents R¹², or when R⁵ is linked to R³ or R¹¹, then R⁵ is a bond or C₁₋₃ alkanediyl;
R⁶ is independently selected from the group consisting of H, deuterium, F, C₁₋₃ alkyl substituted with R¹⁴ and C₁₋₃ alkyl optionally substituted with one or more, identical or different, substituents R¹²;
-R⁷ is independently selected from the group consisting of H, deuterium, F and C₁₋₃ alkyl optionally substituted with one or more, identical or different, substituents R¹²;
R³ is selected from the group consisting of H, deuterium, F and C₁₋₃ alkyl optionally substituted with one or more, identical or different, substituents R¹², or when R³ is linked to R⁵ or R⁹, then R³ is a bond or C₁₋₃ alkanediyl;
R⁹ is selected from the group consisting of H, C₁₋₅ alkyl optionally substituted with one or more, identical or different, substituents R¹², and C₁₋₃ alkyl substituted with R¹⁴ or when R⁹ is linked to R⁸, then R⁹ is a bond or C₁₋₃ alkanediyl;
R¹⁰ is independently selected from benzyl optionally substituted with one or more, identical or different, substituents R¹³;
R¹¹ is selected from the group consisting of H, C₁₋₅ alkyl optionally substituted with one or more, identical or different, substituents R¹², and C₁₋₃ alkyl substituted with R¹⁴, or when R¹¹ is linked to R⁵, then R¹¹ is a bond or C₁₋₃ alkanediyl;
R¹² is independently selected from the group consisting of deuterium, F and OMe;
R¹³ is independently selected from the group consisting of deuterium, methoxy, —OCF₃, Me, CF₃, CF₂Cl, CF₂H, CFH₂, CD₃, cyclopropyl, NH₂, —NHAc, —C(═O)—NH₂, nitro, cyano, Cl, Br, I, and F;
R¹⁴ is independently selected from the group consisting of C₃₋₅ cycloalkyl optionally substituted with one or more, identical or different, substituents R¹², phenyl optionally substituted with one or more, identical or different, substituents R¹³ and 5-membered heteroaryl optionally substituted with one or more, identical or different, substituents R¹³; and
-R¹⁵ is independently selected from the group consisting of C₃₋₆ cycloalkyl optionally substituted with one or more, identical or different, substituents R¹², C₆10 aryl optionally substituted with one or more, identical or different, substituents R¹³ and 5- to 10-membered heteroaryl optionally substituted with one or more, identical or different, substituents R¹³;
or a pharmaceutically acceptable salt, hydrate, polymorph, tautomer, or solvate thereof for use in treating, ameliorating and/or preventing a neuromuscular disorder, and/or for use in reversing and/or ameliorating a neuromuscular blockade.
